(19) United States
(12) Patent Application Publication
(10) Pub. No.: US 2025/0087340 A1
(43) Pub. Date: Mar. 13, 2025

(54) System and Method for Calculating a Profit/Loss or a Surgery Center

(71) Applicant: ZYSC, llc, Tampa, FL (US)

(72) Inventor: Robert C. Nucci, Tampa, FL (US)

(73) Assignee: ZYSC, llc, Tampa, FL (US)

(21) Appl. No.: 18/463,043

(22) Filed: Sep. 7, 2023

(51) Int. Cl. G16H 40/20

(52) U.S. Cl. CPC G16H 40/20

(57) ABSTRACT

A system and method of calculating profit/loss of a surgery center includes accepting inputs regarding costs and revenue for items regarding each aspect of the surgery center. For each client experience, algorithms are run to calculate a professional component, surgery center component (e.g., implants and high-cost disposables), an anesthesia component, other ancillary revenue, and direct costs (e.g., labor costs, supply costs and administrative claim costs) associated with a single client experience. The profit/loss for the single client experience is then calculated based upon a sum of the professional component, the surgery center component, the anesthesia component, the other ancillary revenue, and the direct costs and the profit/loss for the single client experience is outputted (e.g., saved to a file, printed, displayed).

### BACKGROUND OF THE INVENTION

Profit is the key factor for the success of any business including an ambulatory surgery center. The surgical cases performed at the ambulatory surgery center are the individual profit centers that together determine the profitability of the center as a whole.

Recently, pricing transparency in health care has become more important. In certain areas, ambulatory surgery centers (ASC) have been mandated to post the prices of the surgical cases done at their facilities on their website. For an ambulatory surgery center to be profitable as a business as well as to accurately and fairly set prices for their surgical cases, the revenue, cost, and profit associated with those surgical cases must be clearly understood. On the cost side, the direct costs to perform a surgical case include direct labor and supply costs. To date, the multiple variables involved in these direct costs from the time a patient enters the pre-operative area through the surgery and to final discharge have not been clearly outlined.

Because of the complexity of allocating direct costs to surgical cases, the allocation has historically been done broadly and retrospectively. Costs for similar surgical cases done at different times and under different circumstances have been averaged together to estimate the average surgical case cost. Pricing for a specific surgical case was therefore based on this broad estimate. Furthermore, the retrospective nature of the analysis does not afford a clear understanding of the true cost variables involved or how changes in these variables affected cost in any given surgical case. The retrospective analysis is limited to what was done in the past and whatever values the variables took on in these circumstances. In practice, these variables are often poorly understood, unavailable, or both.

Given these limitations, the cost of any surgical case or the likely range of costs given the variables involved is not clear. As a result, the pricing of any surgical case is likely to be set higher to cover these uncertainties.

No algorithm currently exists to prospectively define and monitor the multiple direct cost variables involved in a surgical case performed in an ASC. There is a need for a system that will allow for those variables to be identified, classified, and dynamically changed to reflect the complex nature of a surgical case being considered and the true range of likely costs.

On the revenue side, potential ambulatory surgery center revenue sources for surgical cases are several. They include facility reimbursements, anesthesia professional reimbursement, surgeon professional reimbursement, and reimbursements for some supplies, particularly higher-priced implants. Which reimbursements an ambulatory surgery center is actually entitled to recover in any particular surgical case depends on several factors such as 3^{rd} party contracts, prevailing billing rules, as well as ambulatory surgery center business policy for cash patients. Other internal surgical case variables such as actual operative time may also affect anesthesia reimbursement. Given these potentially very different circumstances, the ultimate reimbursement even for similar surgical cases can vary considerably.

Typically, when surgical case revenue is analyzed, it is done retrospectively once reimbursement has been received. The analysis is done in terms of whether a surgical case was reimbursed fully and correctly given what procedures were done, the way it was billed, and what reimbursements the ambulatory surgery center was entitled to receive. Although this method of analysis is important from a collection standpoint, there is a need to look at the profitability of a surgical case prospectively controlling for the multiple variables involved in reimbursement. If the variables for reimbursement are prospectively defined and manipulated in a controlled manner, then the range of revenue possibilities becomes clear for any given surgical case.

A retrospective analysis looking at these variables is not sufficient as it is limited to what was done in the past and cannot easily assess the opportunities and pitfalls of what can be done or will be done in the future using a range of variables not yet actualized. Knowing with specificity the cost drivers and the revenue potential of any surgical case given the many reimbursement and cost variables is essential to determine surgical case profitability and by extension profitability of the ambulatory surgery center as whole.

In some embodiments, overhead needs to be considered in the profitability of an ambulatory surgical center as in addition to being an individual profit center, the surgical cases performed at an ambulatory surgery center must also cover the overhead expenses and other business costs of the ambulatory surgery center for the business to be Financially sound. Allocation of overhead to any given surgical case is important to see both that case's net profitability when overhead is considered as well as that case's contribution to ambulatory surgery center costs in general. Unfortunately, the term “Overhead” when used in the ambulatory surgery center setting is often used informally to mean some combination of indirect business expenses. Indirect Business Expenses can include operating expenses, general and administrative expenses, research and development expenses, amortization, and depreciation. Any or all of these are considered overhead.

Which indirect business expenses are considered overhead in any particular ambulatory surgery center setting and thereby can be allocated to individual surgical cases in that ambulatory surgery center setting is poorly defined. There is a need for a method to define and clarify what is meant by overhead in any particular ambulatory surgery center setting and to allocate those expenses to individual surgical cases. This formal allocation of overhead to surgical cases will allow for the determination of net profit for any particular surgical case. Once calculated, any particular surgical case or aggregate of surgical cases can be considered in terms of their contribution to other ambulatory surgery center costs.

Overhead, however defined, was typically calculated retrospectively on expenses already incurred. Although it is important to know what business expenses were in the past and the resulting effect they had on net profit for past surgical cases, it is as important to be able to predict net profit for future surgical cases. To accomplish this there is a need for a method to calculate overhead prospectively and allocate that prospective overhead to potential future surgical cases taking into account the multiple revenue and cost variables for those potential surgical cases. In this way, the range of potential net profit for any given future surgical case or cases can be calculated.

This type of predictive analysis for a range of net profits for different surgical cases has important business implications for an ambulatory surgery center. It also has implications for ambulatory surgery center contract negotiations with payors and broader policy implications for reimbursements for surgical cases in the ambulatory surgery center setting in general.

In some embodiments, renting time to a 3^{rd} party in an ambulatory surgery center is becoming more common. Determining a fair rental price that is also profitable for the ambulatory surgery center is not well defined. The common conception is that the ambulatory surgery center is renting physical space and attention is therefore focused on the size of the physical space to be rented. Usually, the size of the operating room is considered as well as a portion of the space in the pre-operative area and the waiting room. Previously, the price per square foot was decided either ad hoc or by using some ill-defined market parameters and the space is valued in that way. Often, the premise is that the space is not being used currently and therefore there are no additional associated costs for the rental time. Any revenue received for renting time in the ambulatory surgery center is simply more revenue than the ambulatory surgery center had to begin with.

Staffing costs were often not considered. Again, there is the conception that the staff is there anyway and that it is difficult to divide out their contribution to the surgical cases occurring during the rental period as they are already being compensated for the regular surgical cases that are usually being performed in the ambulatory surgery center at the same time.

These past conceptions were misconceptions. The ambulatory surgery center is not just renting dormant space. What is actually being rented is the ability to perform certain types of surgical cases in a setting that is legally allowed to do so along with the appropriate staffing and liability coverage to make that possible. What should also be considered is that the ambulatory surgery center has made and will continue to make the appropriate business decisions with their associated costs so that surgical cases, including surgical cases that will occur during the rental period, can continue to be performed there.

Once these factors are considered, it becomes clear that there is more than physical space being rented for a time period. A method to formally value the rental period taking these factors into consideration has not been done in the past. From a cost standpoint, when valuing rental time and space, the main cost factor to consider is overhead. How to allocate overhead to the rental has not been previously described and there is a need for a method to formally accomplish this.

The other main cost factors to consider when an ambulatory surgery center is renting time and space are direct labor and supply costs. How to calculate direct labor and supply costs in the rental setting has not been previously described and there is need for a method to accomplish this.

From a revenue standpoint, the price for the rental time and space is the main revenue source. However, supplies and labor, which are predominately cost elements, are also potential additional revenue sources.

There is a need to formally determine the costs for renting time and space in an ambulatory surgery center considering the overhead and direct costs to the ambulatory surgery center, as well as the potential revenue sources available to the ambulatory surgery center including revenue for the rental time and space itself as well as potential additional revenue for direct labor and supplies. In this way, the potential net profit of renting time and space by the ambulatory surgery center to a third party can be formally determined and subsequent pricing for that time and space can be fair, transparent, and profitable for all parties.

As will be shown, there are a huge number of variables that feed into the profitability of each client experience at a surgery center and, therefore, into the overall profitability of the surgery center. The industry lacks a tool (system and method) that has tables of expenses and revenues associated with an ambulatory surgery center, generating profit or loss predictions for various surgical cases and providing the ability to continuously adjust the associated expenses and revenues to fully understand the profit or loss of each surgical cases.

What is needed is a system and method for predicting costs, and therefore, profitability of the surgical cases in an ambulatory surgery center that is continuously adjustable.

### SUMMARY OF THE INVENTION

The present invention includes a method of performing predictive, quantitative analytics on the cost, revenue and gross profit of any surgical case performed in an ambulatory surgery center. The method overcomes the previous challenges of equivocal terminology in medical practice by clearly defining all terms. The method also overcomes the challenge of allocating cost by clearly defining the events that surround and make up a surgical case and allocating costs to the entire series of events. In some embodiments, the system and method has 4 modules. The first module defines the formal parameters of a surgical case and associated cost and revenue drivers. The second module calculates the expected revenue including any discounts that will be generated by a surgical case from its revenue components. These revenue components are the professional component, the surgery center component including implants and high-cost disposables, the anesthesia component and other ancillary revenue. The third module defines and calculates the direct costs associated with the surgical case. These direct costs include labor costs, supply costs and administrative claim costs. The fourth module calculates the gross profit for a surgical case based upon the expected revenue and its defined costs. The fourth module allows for relevant variables to be changed using a graphic toggle function with the effect of these changes dynamically displayed on revenue, cost, and gross profit. These variables include the time for the events surrounding the surgical case as well as surgical case itself. Other variables include direct supply costs, direct labor costs and the time value of money on the reimbursement for high-cost supplies including implants. In some embodiments, the method includes creating a supply table that can be used in the predictive quantitative analytics for other surgical cases. Additionally, once the predictive quantitative analytics are calculated for one surgical case, these analytics are saved and later edited to create a related surgical case with different variables and different resulting analytics.

Once sufficient data is inputted into the disclosed system, a user has the tools to determine which surgical cases are profitable and which are not. Further, the user has the tools to make changes to the data to see how the changes affect the profits/losses. For example, if a Medicare reimbursement changes or the federal government changes the FICA or Medicare costs for employees, the user is able to see how such changes affect the overall profit/loss of the surgical cases and, therefore, the user is able to react by proposing changes that will effect the revenues or costs associated with certain surgical cases to keep those surgical cases profitable (or to stop performing surgical cases that are not profitable).

in some embodiments, the system has a single database made up of numerus tables that organize data into specific records. Each record encompasses a set of fields, which coordinate with the input fields throughout this document. In one embodiment, a system for calculating a profit/loss of a surgery center is disclosed including a computer that has software stored in a non-transient memory and has storage. During an input stage, the software causes the computer to accept inputs regarding costs and revenue for items and storing the inputs as records in the storage of the computer. Each of the items regard an aspect of the surgery center. After the input stage, the software causes the computer to execute a plurality of algorithms. Each algorithm calculates a subset of the profit/loss associated with a single client experience as determined from at least one record. The software combines each of the subset of the profit/loss into a profit/loss for a client experience and causes the computer to output the profit/loss.

In another embodiment, a method of calculating a profit/loss of a surgery center is disclosed including accepting inputs regarding costs and revenue for a set of items. Each of the items regards an aspect of the surgery center. Each of the inputs are stored as records in a storage of a computer. After accepting all inputs, one or more algorithms are executed. Each algorithm calculates a subset of the profit/loss associated with a single client experience determined from at least one record. Each of the subset of the profit/loss are combined into a profit/loss for a client experience and the profit/loss is outputted (e.g., to a file or display).

In another embodiment, a method of calculating a profit/loss of a surgery center is disclosed including accepting inputs regarding costs and revenue for one or more items, each of the items regarding an aspect of the surgery center and storing the inputs as records in a storage of a computer. After accepting all of the inputs, a first algorithm is executed to calculate a professional component associated with a single client experience, a second algorithm is executed to calculate a surgery center component that includes implants and high cost disposables associated with the single client experience, a third algorithm is executed to calculate an anesthesia component associated with the single client experience,, a fourth algorithm is executed to calculate other ancillary revenue associated with the single client experience, and a fifth algorithm is executed to calculate direct costs associated with the single client experience, the direct costs include labor costs, supply costs and administrative claim costs. The profit/loss for the single client experience is then calculated based upon a sum of the professional component, the surgery center component, the anesthesia component, the other ancillary revenue, and the direct costs and the profit/loss for the single client experience is outputted (e.g., saved to a file, printed, displayed).

### BRIEF DESCRIPTION OF DRAWINGS

The Invention can be Best Understood by Those Having Ordinary Skill in the Art by Reference to the Following Detailed Description when Considered in Conjunction with the Accompanying Drawings in which:

FIG. 1 illustrates a schematic view of profit/loss generation tool.

FIG. 2 illustrates a flow chart of an iterative profit/loss generation system.

FIG. 3 illustrates a schematic view of a typical computer system.

### DETAILED DESCRIPTION OF THE INVENTION

Reference will now be made in detail to the presently preferred embodiments of the invention, examples of which are illustrated in the accompanying drawings. Throughout the following detailed description, the same reference numerals refer to the same elements in all figures.

Throughout this description, the following definitions are used. ASC refers to an Ambulatory surgery center. The patient is referred to as the “client.” The term “user” refers to a person or entity that uses the disclosed system and method to predict the costs of surgeries performed at the ASC, and hence, profits or losses.

Player is an individual (Labor) that is engaged (actively or passively) with the CLIENT for some length of time during the “Client Experience” (CE) or directly engaged with an event that occurs during a CE. This is part of Direct Labor Cost.

Professional Provider is a player who generates Professional Component revenue (Their revenue component would be included as part of Professional Component on the HCFA/UB form). In the same CE, a Professional Provider can generate anesthesia component revenue but must generate professional component revenue.

In some embodiments, there is a non-player actor. A non-player Actor (NPA) is labor that is not involved in the direct Client Experience at any time. They do not engage with clients during the Client Experience. The NPAs will be part of the “Indirect Labor Overhead” outlined in the later “Overhead Module”.

Team member is a type of Player that has only a Cost component and not a revenue component in the Surgery center Cost Module. An example of a Team Member would be Nurse in Pre-op and a Circulating nurse in O.R.

Bot, when present, is a Player (or Team member which is Player subtype) that for a period of time is not involved in any Client experience and is engaged in General Admin work for Surgery Center as a whole. A Bot is similar to an NPA as their labor cost will be part of “Indirect labor overhead” but they are different than NPA because they engage with the client at some point during the Client Experience.

Active (active involvement in Client Experience) is when a player is engaged and focused on one or more Clients. Note: Players who are Active are either “Active Locked Engaged” (ALE) or “Active Combine Time” (ACT).

Active Locked Engaged Time (ALE) is the time a Player is engaged with a client one on one and cannot engage with any other client.

Active Combined time (ACT) is the time a Player is not in “Active Locked Engaged Time” and can engage with more than one client at the same time.

AO Time is the time the Player that was active during the CE spends doing the pure administrative work outside one of 4 stages of the CE. The time does not need to be consecutive.

Administration work done Outside Client Experience (AO) is Pure Administrative work such as charting and dictation done outside Pre-op, O.R., Post-op #1, Post-op #2. This administrative work can only be done by a Player that was active during the Client Experience.

Passive-Locked (PLE) is a Player who is passive and not actively involved with a Client but is locked to that client experience. The Player is not doing administrative work for that client (not doing AO). Additionally, the Player is not doing administrative work for the center and is not actively engaged with another Client. In other words, they are inactive but are tied to the Client.

Temporal Period (TP) is a segment of time during which the Client Experience(s) occurs.

Professional Time (PT) is the time in hours the professional player who is part of the professional component of the revenue module spends with the client in ALE or PLE, in the client experience and any additional time spent in AO outside of client experience.

Therefore: (PT) (professional player hourly rate)=cost of a professional player

Claim Cost is the cost to submit a claim or claims for client experience to a payor. Each module will have its own claim cost.

Initial module claim cost is the cost to submit a claim or claims for a client experience to a payor for the first time. Each module has its own claim cost.

Total module Claim Cost is the initial claim cost+resubmission claim cost+other costs. Each module will have its own total claim cost.

Total Client Experience claim cost is the total claim cost for all modules combined for a client experience.

An anesthesia Provider is the person who is qualified to give anesthesia to a client. An Anesthesia Provider can be MDA, CRNA, or AA. An Anesthesia Provider is a Player that generates anesthesia revenue and only anesthesia revenue.
MDA is the anesthesia provider who is an Anesthesiologist
CRNA is the anesthesia provider who is a certified registered nurse anesthetist
AA is the anesthesiologist assistant, an anesthesia provider who is a non-physician and practices under MDA
Intern is the Medical school graduate in 1st post-graduate year
Resident is the physician licensed to practice medicine
Nurse in training is a registered nurse training to be nurse anesthetist

Ancillary Services are added services that generate revenue but the revenue is not included in the Professional Revenue Submodule, Surgery Center Revenue Submodule, Anesthesia Revenue Submodule.

Ancillary Provider is a Player who generates Ancillary Revenue. The Ancillary Provider is either directly engaged with Client during the CE or directly engaged with an event that occurs during the CE.

Collection Discount is a percentage discount applied to expected revenue in any client experience due to historical inability to collect expected revenue 100% of the time in aggregate across multiple client experiences.

Total Corrected Revenue (TCR) is the revenue that is collected after collection discount. If the user chooses not to use the collection discount, then the total corrected revenue will be equal to the total revenue. Each module has a TCR.

Sum Total Corrected Revenue (STCR) is the sum of all TCR for all the Modules. This sum is used in the profit Module.

Staff is the group of people who work for the business.

Staff Member is the single person who works for the business. Staff members can be Players, Non-player actors or other workers that are part of Indirect Labor Expenses.

A worker is a person who does particular job to earn money.

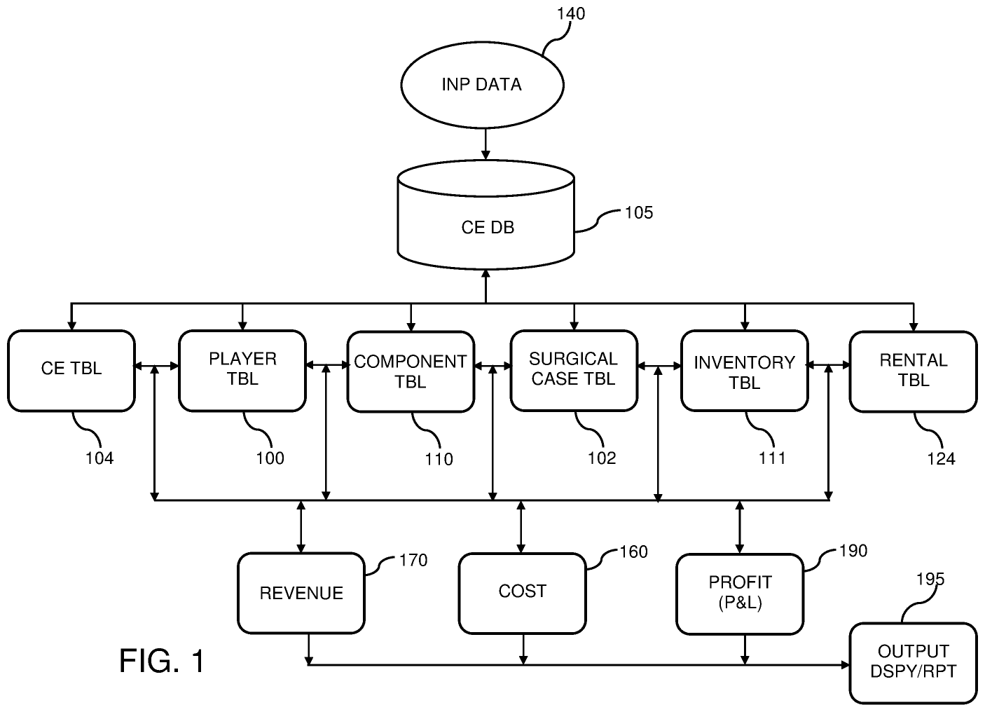

Referring to FIG. 1, a schematic view of a data input process 140 is shown. Throughout this description, many different data items are described in peculiar detail showing a complete system with thought given to almost every possible cost, expense, and revenue associated with a surgery center of any type. It should be noted that this detail is shown to provide insight into the complexity of estimating or predicting profit and lost for individual procedures and/or the surgery center on a whole, while in some embodiments, other additional costs, expenses, and revenues are anticipated. Further, in some embodiments, some of the disclosed costs, expenses, and revenues are excluded as certain surgery centers often do not perform all possible types of surgery (e.g., some surgery centers don't perform implants).

The data input process 140 accepts many data items in any known input method including, but not limited to, traditional computer input user interfaces, voice input, scanning inputs, bulk inputs, downloaded inputs, etc. The data input process 140 uses these inputs populate the CE database 105 that includes a revenue table 170 and the cost table 160 as well as a series of tables that define details that feed into the profit/loss calculation. These details are captured and stored as records or lines in the players table 100, procedures table 102, client experiences table 104, components table 110, inventory table 111, payors table, professional revenues table, collection discounts table, payment rates table, taxes table, customer experience mix table and rental costs table 124. Once all (or most) of these databases and tables are populated, the profit/loss generation tool predicts profits/losses for each client experience and for the overall surgery center.

The players table 100 is populated with details regarding each player (e.g., surgeon, registered nurse) available in the surgery center. Such details include the name/type of player, job description, payment type (e.g., hourly, salary), payment rate, etc.

The procedures table 102 is populated with the types of surgical cases that are possible at the surgery center (e.g., knee replacements, hip replacements, colonoscopies, breast augmentation).

The client experiences table 104 is populated with the name and details of all anticipated client experiences. A client experience includes a name of the client experience (which can be the same name or a different name as the surgical case contained within it e.g., cervical fusion, ACDF), the time for each stage of the client experience (e.g., pre-op, operation, post-op) the players required for the client experience in each stage, the components required (e.g., implants, medications), and all associated revenue and cost variables and calculated profit.

The components table 110 is populated with parameters associated with components that generate revenue such as orthotics.

The payors table (included in the CE table 104 for brevity) is populated with names and details of the possible payors for each client experience.

The professional revenues table (included in the revenue table 170 for brevity) is populated with revenues that are experienced from professional providers.

The collection discounts table (included in the cost table 160 for brevity) is populated with anticipated collection discounts. A collection discount is an expected percentage of billed amounts that will be realized based upon the payor type (e.g., private insurance, Medicare) and historical norms.

The payment rates table (included in the players table 100 for brevity) is populated with payment rates of professional providers in view of taxes (e.g., FICA).

The taxes table is populated with details of overheads on salaries due to certain city, state, or federal taxes such as FICA and Medicare which employers are required to pay as part of payroll.

The customer experience mix table (included in the CE table 104 for brevity) is populated with a surgery center's expected mix of customer experiences. For example, one surgery center might perform only orthotic customer experiences while another might perform 30% general surgeries and 70% orthotic surgeries, etc.

The rental costs table 124 is populated with costs of rentals such as special equipment, real estate, etc.

It is anticipated that in some embodiments, the profit/loss generation tool generates an output 195 such as a graphical user interface showing the profit/loss for the surgery center and/or individual client experiences and, in some embodiments, this generation is performed in a loop, constantly updating the profit/loss based upon changes to any of the parameters as inputted.

The profit/loss generation tool receives inputs from the players table 100, procedures table 102, client experiences table 104, components table 110, payors table, professional revenues table, collection discounts table, payment rates table, taxes table, customer experience mix table and rental costs table 124 as well as the cost table 160 and the revenue table 170 and uses algorithms as will be describe to generate profits and losses 190 of the surgery center and/or individual client experiences.

The profit/loss generation tool generate a graphical user interface showing the profits and losses 190 for the surgery center and/or individual client experiences in an iterative fashion such that, the user of the profit/loss generation tool has the ability to make one or more changes to the inputs (and hence the players table 100, procedures table 102, client experiences table 104, components table 110, payors table, professional revenues table, collection discounts table, payment rates table, taxes table, customer experience mix table and rental costs table 124). Once the base set of data is entered by way of the data input process 140, a loop begins.

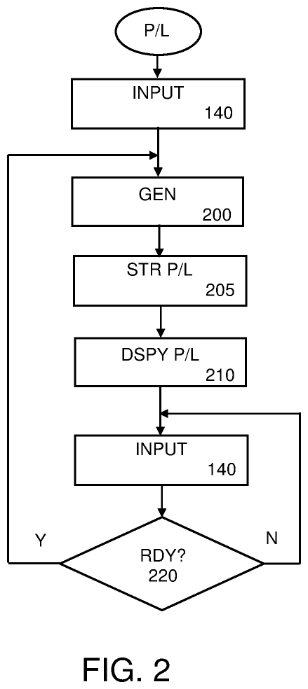

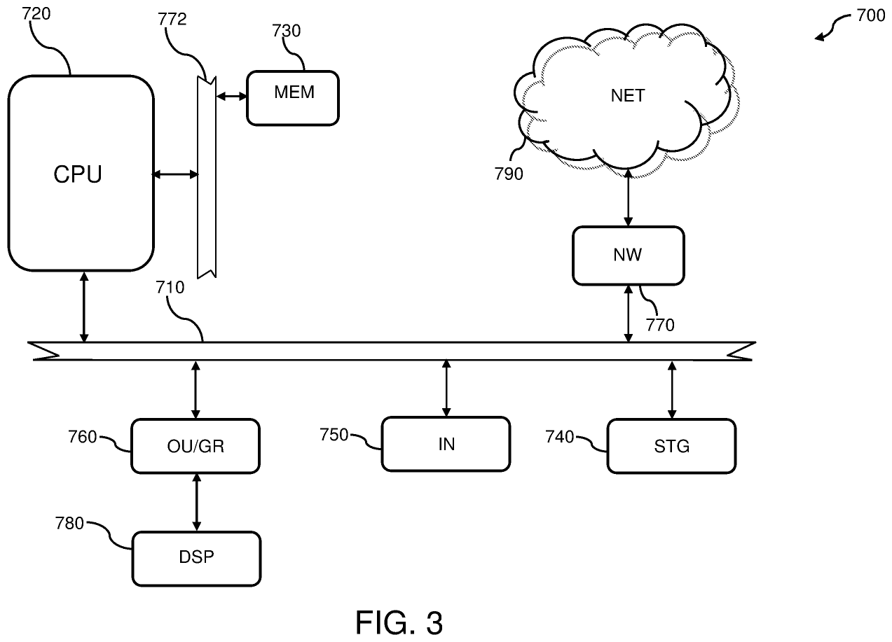

In FIG. 2, the loop starts by using the profit/loss generation tool to generate 200 profits and losses 190, store the profits and losses in the profits and losses table 190 and then displays 210 the profits and losses190, for example on the display 780 (see FIG. 3), either as text or graphics. In some embodiments, the profits and losses 190 that are generated by the profit/loss generation tool are stored 205 for comparison to prior generated profits and losses. Now the profits and losses 190 are displayed 210. In some embodiments, prior profits and losses are also displayed 210 to show changes based upon the current data/inputs.

Now, still in the loop, the data input process 140 is again used to accept changes to the data that feeds the profit/loss generation tool. As multiple changes are envisioned, a test 220 is made to determine if the user has completed their inputs. If the user is not ready 220, the data input process 140 is repeated to accept additional inputs. If the user is ready 220, the loop iterates to generate and display updated profits and losses 190.

Referring to FIG. 3, a schematic view of a typical computer system 700 is shown. The computer system 700 is anticipated to correspond to any system used to perform the system and methods disclosed. As shown in FIG. 3, the computer system 700 includes a bus 710, a processor 720, a memory 730 interfaced to the processor by a memory bus 772, a storage component 740, an input component 750, an output component 760, and a communication interface 770.

Bus 710 includes components that permit communication between the processor 720 and other components of computer system 700. The memory 730 may is any type and quantity of a random-access memory (RAM), a read only memory (ROM), and/or another type of dynamic or static storage (e.g., flash memory, optical memory) that stores information and/or instructions for use by the processor 720.

Storage component 740 stores information and/or software related to the operation and use of computer system 700 in a non-transitory way. For example, the storage component 740 includes a hard disk (flash memory, a magnetic memory, an optical memory, etc.), a compact disc (CD), a digital versatile disc (DVD), a cartridge, a magnetic tape, and/or another type of computer-readable medium, along with a corresponding drive.

Input component 750 includes any component that permits the computer system 700 to receive information, such as user input (e.g., a touch screen display, a keyboard, a keypad, a mouse, a button, a switch, a microphone, etc.). The output component 760 includes, for example, a component that provides output information from the computer system 700 (e.g., a display 780, a speaker).

The communication interface 770 includes, for example, a transceiver (e.g., a transceiver, a separate receiver and transmitter, etc.) that provides communications services for the computer system 700 to communicate with other systems through a network 790, such as via a wired connection, a wireless connection, or a combination of wired and wireless connections. The communication interface 770 allows the computer system 700 to receive information from other devices and/or provide information to other devices. For example, in some embodiments, the communication interface 770 includes an Ethernet interface, a coaxial interface, an infrared interface, a radio frequency (RF) interface, a universal serial bus (USB) interface, a Wi-Fi interface, a cellular network interface, or the like.

The computer system 700 executes processes of the present invention in response to the processor 720 executing software instructions stored by a computer-readable medium, such as the memory 730 and/or the storage component 740. Such computer-readable medium is a non-transitory memory device. Such memory devices include memory space within a single physical storage device or memory space spread across multiple physical storage devices.

Software instructions are typically read into the memory 730 from the storage component 740 or from another computer-readable medium or from another device via the communication interface 770. When executed, software instructions stored in the memory 730 cause the processor 720 to perform one or more processes described herein. Additionally, or alternatively, hardwire circuitry may be used in place of or in combination with software instructions to perform one or more processes described herein. Thus, implementations described herein are not limited to any specific combination of hardware circuitry and software.

The sub-components of the computer system 700 shown in FIG. 4 are provided as an example. In practice, it is anticipated that the computer system 700 includes additional components, fewer components, different components, or differently arranged components than those shown in FIG. 3. Additionally, or alternatively, in some embodiments, a set of components of the computer system 700 performs one or more function described as being performed by another set of components of the computer system 700.

The remainder of this document describes one embodiment of the disclosed system and method that takes into account many of the details often needed to correctly predict the profit/loss of a surgical case.

The Client Experience (CE) is defined as the series of events involving the client that occur in the Pre-op, operating room (O.R.), Post-op #1, Post-op #2 stages. Although technically different, the CE is usually named using the same name as it is used for the surgical case as that is the main event occurring during CE. For example, “Two Level ACDF” can be used to describe both a Surgical Case and Client Experience as the surgical event of a “Two Level ACDF” is the main event that affects all other events in CE. However, if the CE involves two separate two separate surgical cases as would be the situation of two separate trips to OR during the same CE, the CE can have a different name than the two separate surgical cases. The Pre-op stage usually occurs in the pre-op area. The O.R. stage usually occurs in the operating room The Post-op #1, Post-op #2 usually occurs in the recovery area. A surgical case, however, can occur in any area. For purposes of this system, a surgical case (surgical event) occurs in the O.R. stage and all other stages follow from the O.R. stage by their respective definitions.

A surgical case is defined as a surgical procedure or procedures (surgical events) grouped together and broadly categorized from the surgical standpoint under one common and inclusive heading. The same surgical case can be described using different headings dependent upon the user. And the same surgical case can also be made up of different surgical procedures as the surgical procedures are based on billing and billing may vary depending on the payor.

The client experience set up consists of 5 parts. Those parts are the client experience (CE) name, the payor, the 4 time periods of the stage time and the total time for all the time periods together, the players and the notes. The CE set up algorithm allows the entry of data into each of these 5 parts. Data from the CE set up algorithm is used in all the modules and, the CE set up algorithm links all the modules together. The CE set up algorithm also creates the header that is present across the top of all the modules. The CE header consists of the CE name, the payor and the notes.

The first part of CE Set up is the CE name. The CE name is a free text field that is entered by the user. When the CE is saved to the CE table 104, the CE is saved using the CE name. The second part of CE set up is the payor. The payor is a free text field that is entered by the user or pulled from the payor table. The payor table is described below. The third part of CE set up is the 4 time periods of the stage time. The 4 time periods of the stage time are pre-op, O.R., post op #1 and post op #2. The CE set up algorithm will allow for time to be inputted into each of these 4 time periods of the stage time as free text fields in minutes. The time period inputted for Post-op #1 using the CE set up algorithm must be equal to the time period inputted using the direct labor algorithm for the anesthesia provider in ALE in Post-op #1. The direct labor algorithm for the anesthesia provider in ALE in Post-op #1 is described in the cost module [Part 1] direct labor cost, anesthesia provider section C.3.

In some embodiments, time in minutes is entered as whole numbers.

The CE set up algorithm will calculate the total time for all 4 time periods added together. Total time is defined as the Temporal Period (TP).

The fourth part of CE set up is the players. A sub algorithm of the CE set up algorithm is the player entry sub algorithm which allows for the addition of player type, payment type and name/description. The player entry sub algorithm saves the player type, payment type and name/description in the players table 100.

Players are one of 4 types: professional providers, team members, anesthesia providers and ancillary providers. The player entry sub algorithm adds players to the CE table 104 and, in some embodiments, adds additional players of the same type to the CE table 104.

The user is not required to pick any particular player type or combination of player types for a CE table 104. However, once a player type is inputted using the player entry sub algorithm, the sub algorithm requires that a job description be inputted for that player. Players of each type have different job descriptions.

Professional provider and team members' job descriptions are a single-select list box option for each type of player with an “other” option. The “other” option, when chosen, is a “free form text field”.

Ancillary providers' job descriptions are a single-select list box option with an “other” option. The “other” option, when chosen, is a “free form text field.” For ancillary provider job descriptions including the “other” option, the player entry sub algorithm requires that once the job description is inputted, it also must be allocated to one of two mutually exclusive options of “inside the CE” or “outside the CE”.

The anesthesia providers' job descriptions are single-select list box option for MDA acting alone, CRNA acting alone, MDA-Supervising, MDA-Directing, CRNA-Supervised, CRNA-Directed, AA-Supervised, AA-Directed. There is no “other” option for anesthesia provider job descriptions.

The payment type for each player is entered using player entry sub algorithm and pushed to a separate algorithm called the payment rate algorithm, which considers the multiple ways the 4 types of players can be paid.

The name/description is a free form text field where the user names the player or further describes the player.

The fifth part of CE Set up is the notes. The notes is a free form text field where the user adds any additional helpful information about the CE.

The CE name, the payor and the notes are editable in any module and the changes are carried across all the modules including the CE set up.

payment type can be edited only in the CE set up. When saved, changes will carry across to all the modules.

Stage time can be edited in CE set up and dynamically changed in the profit module using the graphic toggle function described below. These functions are different. Editing stage time in the CE set up will require reallocation of all players' times in the cost module. Changing stage time using the graphic toggle function in the profit module will not require reallocation of players times to the different stage times. Stage time in CE set up is the ideal time for the CE to occur and stage time in the profit module is time that is relative to changes that are internal to the CE.

### Payment Rate Algorithm:

For all players, the payment rate algorithm has a FICA option when the player is paid on an hourly or salaried basis. When the FICA option is chosen for a player, additional direct FICA costs are added to the calculated direct player costs in a CE.

The FICA algorithm option considers the FICA tax of 7.65% on top of a player's hourly rate for which the employer is responsible. The algorithm will convert salaried employees to an hourly rate for purposes of calculating FICA. For most employees, FICA is composed of two things: Social Security tax of 6.2% and Medicare tax of 1.45%. These rates are adjustable should the Internal revenue Service change the rates.

The FICA algorithm also considers high-cost players that are earning over two distinct dollar amounts per year as the FICA tax will be different for these employees. In 2022, these two distinct dollar amounts are 146k and 200k per year. Although FICA is calculated on hourly rate, for the purpose of determining those employees for which the different FICA rates apply, the hourly rates for employees will be converted to a yearly salary if needed. For all players that make up to 146k per year, a FICA tax of 7.65% is applied to each dollar earned. For players that make greater than 146k per year up to 200k per year, a FICA tax of 7.65% applies to each dollar earned up to 146k. Between 146k to 200k, only a Medicare tax of 1.65% applies to each dollar earned. These thresholds are adjustable should the Internal revenue Service change the amounts.

For players that make greater than 200k per year, a FICA tax of 7.65% applies to each dollar earned up to 146k. Between 146k to 200k only a Medicare tax of 1.65% applies to each dollar earned. Above 200k an additional 0.9% Medicare tax applies to each dollar earned, again settable.

### Professional Provider:

There is a formal definition of a “professional provider.” The algorithm considers 5 different professional provider methods of payment. As there can be more than one professional provider in a surgical case with each having separate job descriptions, there can be more than one method of payment for each of the different professional providers.

The first method the payment rate algorithm considers is when the professional provider is paid on an hourly basis. The hourly rate is entered. The FICA algorithm option can be chosen, and the hourly rate will be adjusted. The hourly rate is pushed to the cost module for that player to be allocated by the direct labor algorithm in the cost module. The second method the payment rate algorithm considers is when the professional provider is paid by salary. The algorithm will convert the salary to an hourly rate with input of the average hours the professional provider works per week and the number of weeks the professional provider works per year. The data being entered to calculate the hourly rate is prospective but can be based on retrospective data. Once the hourly rate is determined, the FICA algorithm option is chosen, and hourly rate will be adjusted. The hourly rate is pushed to the cost module for that player to be allocated by the direct labor algorithm in the cost module. The third method the payment rate algorithm considers is when the professional provider is paid per day. The algorithm will convert the payment per day to an hourly rate with input of the number of hours the professional provider works in a day and the payment per day. The FICA algorithm is not an option as this possibility precludes employees. The hourly rate is pushed to the cost module for that player to be allocated by the direct labor algorithm in the cost module. The fourth method the payment rate algorithm considers is when the professional provider is paid per case. The payment per case is entered. The FICA algorithm is not an option as this possibility precludes employees. The payment per case is pulled to cost table in the cost module for that player by the cost table algorithm. For payment per case, the direct cost for the player in the CE table 104 is determined in CE set up by payment per case input.

The fifth method the payment rate algorithm considers is when the professional provider is paid a percentage of the professional revenue collected. The percentage is entered. The algorithm will have an open link to the professional revenue submodule. The user will identify in the professional revenue submodule the professional revenue lines of code to which the percentage applies. If the collection discount professional sub algorithm option is used to calculate the “corrected line revenue” for any line of code of the professional revenue, the percentage will apply to this corrected revenue. The collection discount algorithms are discussed in the revenue module. The cost module and the profit module will be flagged as INCOMPLETE until the professional revenue submodule is completed with the user identified professional revenue lines of codes linked to the professional provider that is paid as a percentage of professional revenue collected. The FICA algorithm is not an option for payment of a percentage of the professional revenue collected as this possibility precludes employees. The percentage of the professional revenue collected is pulled to cost table 160 in cost module for that payer by cost table algorithm. For percentage of the professional revenue collected, the direct cost for the player in the CE is determined in CE set up by percentage of the professional revenue collected input.

The payment rate algorithm for payment as a percentage of professional revenue collected also allows for one percentage to be paid on one line of code or codes and a different percentage to be paid on a different line of code or codes for the same professional provider. This allows the professional provider, with one job description such as Surgeon, to be paid a percentage of their own professional revenue or “corrected line revenue” as well as the professional revenue or “corrected line revenue” for a different professional provider with the different job description such as First Assistant for the same surgical case. The other professional providers will have their own payment type.

### Team Member:

The team member algorithm considers 3 different team members' methods of payment. As there is more than one team member in a surgical case with each having separate job descriptions, there is more than one method of payment for each of the different team members. In the first method of payment, the payment rate algorithm considers that the Team Member is paid on an hourly basis. The hourly rate is entered. The FICA algorithm option can be chosen, and hourly rates are adjusted. The hourly rate is pushed to the cost module for that team member to be allocated by the direct labor algorithm in the cost module. In the second method of payment, the payment rate algorithm considers that the team member is paid by salary. The algorithm will convert the salary to an hourly rate with input of the average hours the team member works per week and the number of weeks the team member works per year. The data being entered to calculate the hourly rate is prospective but can be based on retrospective data. Once the hourly rate is determined, the FICA algorithm is chosen, and the hourly rate is adjusted. The hourly rate is pushed to the cost module for that Team Member to be allocated by the Direct Labor algorithm in the cost module. In the third method of payment, the payment rate algorithm considers that the team member is paid per day. The algorithm will convert the payment per day to an hourly rate with input of the number of hours the team member works in a day and the payment per day. The FICA algorithm is not an option as this possibility precludes employees. The hourly rate is pushed to the cost module for that team member to be allocated by the direct labor algorithm in the cost module.

### Anesthesia Provider:

An anesthesia provider is an MDA, CRNA or AA that function in the CE with various job descriptions. For all job descriptions, the anesthesia algorithm considers 5 different anesthesia provider methods of payment. As there can be more than one anesthesia provider in a surgical case, there can be more than one method of payment for each of the different anesthesia providers with their various job descriptions. The job descriptions for the anesthesia providers are MDA acting alone, CRNA acting alone, MDA-Supervising, MDA-Directing, CRNA-Supervised, CRNA-Directed, AA-Supervised, AA-Directed.

When inputting anesthesia provider(s) in CE set up, certain combinations of job descriptions are allowed, and certain combinations are prohibited. Certain job descriptions can be inputted by themselves, and other job descriptions must be inputted in combination. There will be an ERROR displayed if the user tries to input a prohibited combination. There will be an ERROR if the user tries to input a combination for a job description which functions by itself. If the job descriptions require a combination, the user will be required to enter an allowed combination for that job description.

The following job descriptions function alone, and the player entry sub algorithm will allow only one anesthesia provider job description of this type to be inputted:
MDA acting alone job descriptions.
CRNA acting alone job descriptions.

The following anesthesia provider job descriptions must be used in combination with a second anesthesia provider job description. The player entry sub algorithm will require the user to input an allowed combination. The anesthesia provider job descriptions and the allowed combinations are as follows:
MDA directing a CRNA with MDA directing 1 case.
CRNA directed by MDA, with MDA directing 1 case.
MDA directing a CRNA with MDA directing 2-4 cases.
CRNA directed by MDA, with MDA directing 2-4 cases.
MDA directing an AA with MDA directing 2-4 cases.
AA directed by MDA, with MDA directing 2-4 cases.
MDA supervising CRNA with MDA present at induction.
CRNA supervised by MDA with MDA present at induction.
MDA supervising CRNA with MDA NOT present at induction.
CRNA supervised by MDA with MDA NOT present at induction.
MDA supervising AA with MDA present at induction.
AA supervised by MDA with MDA present at induction.
MDA supervising AA with MDA NOT present at induction.
AA supervised by MDA with MDA NOT present at induction.

The first method the payment rate algorithm considers is when the anesthesia provider (with any job description) is paid on an hourly basis. The hourly rate is entered. The FICA algorithm option can be chosen, and the hourly rate will be adjusted. The hourly rate is pushed to the cost module for that player to be allocated by the Direct Labor algorithm in the cost module. The second method the payment rate algorithm considers is when the anesthesia provider is paid by salary. The algorithm will convert the salary to an hourly rate with input of the average hours the anesthesia provider works per week and number of weeks the anesthesia provider works per year. The data being entered to calculate the hourly rate is prospective but can be based on retrospective data. Once the hourly rate is determined, the FICA algorithm option can be chosen, and hourly rate will be adjusted. The hourly rate is pushed to the cost module for that player to be allocated by the direct labor algorithm in the cost module.

The third method the payment rate algorithm considers is when the anesthesia provider is paid per day. The algorithm will convert the payment per day to an hourly rate with input of the number of hours the anesthesia provider works in a day and the payment per day. The FICA algorithm is not an option as this possibility precludes employees. The hourly rate is pushed to the cost module for that player to be allocated by the direct labor algorithm in the cost module. The fourth method the payment rate algorithm considers is when the anesthesia provider is paid per case. The payment per case is entered. The FICA algorithm is not an option as this possibility precludes employees. Payment per case for that Player is pulled to the cost table in the cost module by cost table algorithm. For payment per case, the direct cost for the player in the CE is determined in CE set up by payment per case input. The fifth method the payment rate algorithm considers is when the anesthesia provider is paid a percentage of the anesthesia revenue collected. The percentage is entered. The algorithm will have an open link to the anesthesia revenue submodule. The user will identify in the anesthesia revenue submodule the anesthesia revenue line or lines of code to which the percentage applies. If the collection discount anesthesia sub algorithm option is used to calculate the “corrected line revenue” for any line of code of the anesthesia revenue, the percentage will apply to this corrected revenue. The cost module and the profit module will be flagged as INCOMPLETE until the anesthesia revenue submodule is completed with the user identified anesthesia revenue line or lines of codes linked to the anesthesia provider that is paid as a percentage of anesthesia revenue collected. The FICA algorithm is not an option for payment of a percentage of the anesthesia revenue collected as this possibility precludes employees. The percentage of the anesthesia revenue collected for that player is pulled to cost table 160 in the cost module by cost table algorithm. For percentage of the anesthesia revenue collected, the direct cost for the player in the CE is determined in CE set up by percentage of the anesthesia revenue collected input.

The payment rate algorithm for payment as a percentage of anesthesia revenue collected also allows for one percentage to be paid on one line of code or codes and a different percentage to be paid on a different line of code or codes for the same anesthesia provider. This will allow the anesthesia provider, with one job description such as MDA, to be paid a percentage of their own anesthesia revenue or “corrected line revenue” as well as the anesthesia revenue or “corrected line revenue” for a different anesthesia provider with the different job description such as anesthesia assistant for the same surgical case. The other anesthesia provider will have their own payment type.

### Ancillary Provider:

Ancillary providers are either present during one or more stages of the CE or directly engaged with the event that occurs in one or more stages of the CE. This is discussed in detail below in the cost module. The algorithm considers 5 different ancillary provider methods of payment. As there can be more than one ancillary provider in a surgical case with each having separate job descriptions, there can be more than one method of payment for each of the different ancillary providers.

The first method the payment rate algorithm considers is when the ancillary provider is paid on an hourly basis. The hourly rate is entered. The FICA algorithm option can be chosen, and the hourly rate will be adjusted. The hourly rate is pushed to the cost module for that player to be allocated by the direct labor algorithm in the cost module. The second method the payment rate algorithm considers when the ancillary provider is paid by salary. The algorithm will convert the salary to an hourly rate with input of the average hours the ancillary provider works per week and the number of weeks the ancillary provider works per year. The data being entered to calculate the hourly rate is prospective but can be based on retrospective data. Once the hourly rate is determined, the FICA algorithm option can be chosen, and hourly rate will be adjusted. The hourly rate is pushed to the cost module for that player to be allocated by the direct labor algorithm in the cost module. The third method the payment rate algorithm considers when the ancillary provider is paid per day. The algorithm will convert the payment per day to an hourly rate with input of the number of hours the ancillary provider works in a day and the payment per day. The FICA algorithm is not an option as this possibility precludes employees. The hourly rate is pushed to the cost module for that player to be allocated by the direct labor algorithm in the cost module. The fourth method the payment rate algorithm considers when the ancillary provider is paid per case. Ancillary providers are sometimes present during the CE or not present during the CE. If the ancillary provider is not present during the CE and paid for an event that occurs during one of the stages of the CE, payment for the event is considered payment per case. For both instances, the payment per case is entered. The FICA algorithm is not an option as this possibility precludes employees. Payment per case for that player is pulled to the cost table in the cost module by cost table algorithm. For payment per case, the direct cost for the player in the CE is determined in CE set up by payment per case input. The fifth method the payment rate algorithm considers when the ancillary provider is paid a percentage of the ancillary revenue collected. The percentage is entered. The algorithm will have an open link to the ancillary revenue submodule. The user will identify in the ancillary revenue submodule the ancillary revenue line or lines of code to which the percentage applies. If the collection discount ancillary sub algorithm option is used to calculate the “corrected line revenue” for any line of code of the ancillary revenue, the percentage will apply to this corrected revenue. The cost module and the profit module will be flagged as INCOMPLETE until the ancillary revenue submodule is completed with the user identified ancillary revenue line or lines of codes linked to the ancillary provider that is paid as a percentage of ancillary revenue collected. The FICA algorithm is not an option for payment of a percentage of the ancillary revenue collected as this possibility precludes employees. The percentage of the ancillary revenue collected for that player is pulled to the cost table in the cost module by cost table algorithm. For percentage of the ancillary revenue collected, the direct cost for the player in the CE is determined in CE set up by percentage of the ancillary revenue collected input.

### There

In the revenue model, the professional revenue submodule has two components that potentially generate professional revenue. These are the professional provider and DME/Orthotics. Both revenue sources have potential collection discounts discussed below.

The user will select from the CE Set up algorithm the Professional Provider or Providers that generate revenue within the professional component of the surgical case. Only professional providers are selectable. All other players are prohibited. For each professional provider, the professional revenue submodule algorithm accepts user input of the procedure code or codes (CPT/HCPSC codes) for the professional revenue component of the surgical case for that provider. This algorithm accepts the CPT/HCPCS codes as user input directly or pulls from the provider's fee schedule, associated modifiers, procedure code descriptions as well as the billed amount and the allowable amount by payor for each line of code. The user will input all lines of code for the surgical case, the description for each line of code as well as the associated billed and allowable amounts for each line of code sequentially as they would be formally billed and submitted to a payor on a HCFA form.

From the revenue standpoint, each line of code generates revenue in one of two mutually exclusive ways. The first way is by the “billed amount” for that line of code or a percentage of the billed amount. The second way is by the “allowable amount” for that line of code or a percentage of the allowable amount for that line of code. Using the professional revenue submodule algorithm, the user selects one of the two mutually exclusive options of the “billed amount” or the “allowable amount”. The first selection will apply to all lines of codes for the surgical case for that Professional Provider. Once the first selection is made the algorithm will require a second selection to be made. This second selection is made separately for each line of code and will apply only to that line of code. The percentage choice is from 0-100%. The user will select a percentage for each line of code that will apply to the billed amount or the allowable amount for that line of code depending on the first selection that was made. The application of the percentage to each line of code will be the “base revenue” for that line of code.

After the base revenue is calculated, the professional revenue submodule algorithm accepts user inputs of units to the base revenue with the default unit being 1. With the choice of units, the algorithm will multiply the base revenue for that line of code by the number of units chosen up to 10. The algorithm will then calculate the “line revenue” for that line of code by multiplying the number of units by the base revenue. The revenue table algorithm pulls the “line revenue” or “line revenues” for the professional provider to the revenue table in the revenue module.

After user selects the base revenue and units for each of the lines of codes, the professional revenue submodule algorithm calculates the “component revenue” for all lines of code for that professional provider. The revenue table algorithm pulls the “component revenue” for the professional provider to the revenue table in the revenue module. The dynamic revenue table algorithm pulls the “component revenue” for the professional provider to the dynamic revenue table in the profit module.

The user has the option to add additional professional providers that generate revenue using CE set up algorithm within the professional component of the surgical case. Each professional provider added will have its own professional provider “component revenue” calculated by the professional revenue submodule algorithm in the same manner as above. The revenue table algorithm pulls the “component revenue” for each additional professional provider to the revenue table in the revenue module. The dynamic revenue table algorithm pulls the “component revenue” for each additional professional provider to the dynamic revenue table in the profit module.

The professional revenue submodule DME/Orthotic sub algorithm also has a DME/Orthotics component. DME/Orthotics lines of code can, on occasion, be billed and generate revenue along with the professional procedure lines of code for a surgical case. The professional revenue submodule DME/Orthotic sub algorithm accepts user input of DME/Orthotic codes (HCPCS codes) for the DME/Orthotic component of the surgical case. The DME/Orthotic must be linked to a professional provider that generates the professional revenue. As with revenue generated by the lines of procedure codes for a professional provider, when DME/Orthotics lines of codes generate revenue, they do so in one of two mutually exclusive ways. The first way is by the “billed amount” or a percentage of the billed amount for that line of code. The second way is by the “allowable amount” or a percentage of the allowable amount for that line of code. Using the Professional Revenue Submodule DME/Orthotic sub algorithm, the user will select one of the two mutually exclusive options. Once the first selection is made the algorithm will require a second selection to be made. This second selection is made separately for each line of DME/Orthotic code and will apply only to that line of code. The percentage is from 0-100%. The user will select a percentage for each line of code that will apply to the billed amount or the allowable amount for that line of code depending on the first selection that was made. The application of the percentage to each line of code will be the “base revenue” for that line of code.

After the base revenue is calculated, the professional revenue submodule DME/Orthotic sub algorithm accepts user input of units for each code, the default unit being 1. The algorithm multiplies the base revenue for that line of code by the number of units chosen up to 10. The algorithm then calculates the revenue for that line of code by multiplying the number of units by the base revenue. Once the user chooses the base revenue and units for each of the lines of codes for the DME/Orthotic, the algorithm will calculate the “line revenue” for each line of code. The revenue table algorithm pulls the “line revenue” or “line revenues” for the DME/Orthotics to the revenue table in the revenue module.

The system considers all DME/Orthotics as one component. The sum of the “line revenues” for all lines of code is the “component revenue” for the DME/Orthotics. The revenue table algorithm pulls the “component revenue” for the DME/Orthotics to the revenue table in the revenue module. The dynamic revenue table algorithm pulls the “component revenue” for the DME/Orthotics to the dynamic revenue table in the profit module.

The DME/Orthotic lines of code and their component revenue are placed below the professional provider lines of codes and their component revenue.

The cost component of the DME/Orthotics will be linked to the revenue table in the professional revenue submodule. The creation of this link is discussed in the Inventory section of the surgery center cost submodule (VI-5).

The professional revenue submodule also has a collection discount professional sub algorithm that will calculate a collection discount on the professional revenue. The collection discount reflects the expected revenue the ASC is entitled to collect for the professional component of a surgical case to the actual revenue collected. This difference can be for a variety of reasons and the algorithm accepts user inputs of revenue discount percentages from 0-100%.

When the collection discount professional sub algorithm is applied, the percentage discount is applied equally to all lines of codes for the professional component and for the DME/Orthotic component of the professional revenue submodule. The “line revenue” generated by each line of code that was originally calculated using the professional revenue submodule algorithm is displayed alongside the newly calculated discounted revenue for that line of code. The discounted revenue for each line of code is defined as the “corrected line revenue” for that line of code.

As discussed in CE set up, the fifth method the Payment Rate algorithm considers is for a professional provider paid as a percentage of professional revenue collected. The user selects the lines of code for which the percentage applies. The revenue for the lines of code selected are calculated to be the corrected revenue if the collection discount professional sub algorithm has been applied.

For the DME/Orthotic(s) and the professional provider(s), the component revenue for all lines of corrected revenue is defined as the “corrected component revenue”. The “component revenue” is displayed alongside the “corrected component revenue” for the DME/Orthotic(s) and professional provider(s).

The sum revenue for all of the “component revenue” in the professional submodule is called the “total professional submodule revenue”.

The sum of the revenue for all “corrected component revenue” in the professional submodule is called “corrected total professional submodule revenue”.

The “total professional submodule revenue” and “corrected total professional submodule revenue” are calculated by the revenue table algorithm from data pulled from the professional revenue submodule and inputted into the revenue table. This is discussed below in the revenue table submodule. The dynamic revenue table algorithm pulls the “total professional submodule revenue” and if present “corrected total professional submodule revenue” to the dynamic revenue table.

The surgery center revenue submodule has 4 components that relate to generated surgery center revenue. The surgery center revenue source components are the surgery/procedure component, the DME/Orthotic component, the HCD component and the Implant component. All 4 components potentially have a collection discount.

The surgery center revenue submodule algorithm accepts user inputs of the procedure code(s) (CPT/HCPSC codes) for the surgery center revenue component of the surgical case. The user enters either directly or from a fee schedule the CPT/HCPCS codes and associated modifiers, procedure code descriptions as well as the billed amount and the allowable amount by payor for each line of code. The user will input all lines of code for the surgical case as well as the associated billed and allowable amounts for each line of code sequentially as they would be formally billed and submitted to a payor on a HCFA/UB4 form.

From the revenue standpoint, each line of code generates revenue in one of two mutually exclusive ways. The first way is by the “billed amount” or a percentage of the billed amount. The second way is by the “allowable amount” or a percentage of the allowable amount. The user selects one of the two mutually exclusive options which will apply to all lines of code for the surgical case. Next, for each line of code, the user enters a percentage choice from 0-100%. The percentage for each line of code applies to the billed amount or the allowable amount for that line of code. The application of the percentage to each line of code will be the “base revenue” for that line of code.

After the base revenue is calculated, the surgery center revenue submodule algorithm accepts user inputs of units for each line of code, the default unit being 1. The algorithm multiplies the base revenue for that line of code by the number of units chosen (up to 10). The revenue table algorithm pulls the “line revenue” or “line revenues” for the surgical case to the revenue table in the revenue module.

The algorithm calculates the total revenue for all lines of code. The total revenue for all lines of code is the “component revenue” for that surgical case and by definition, the “component revenue” for all the procedure code or codes that make up that surgical case. If there is no revenue for DME/Orthotics, Implants or HCDs, the “component revenue” for the surgical case would be the same as “total surgery center submodule revenue”. The revenue table algorithm pulls the “component revenue” for the surgical case to the revenue table in the revenue module. The dynamic revenue table algorithm pulls the “component revenue” to dynamic revenue table in the profit module.

The surgery center revenue submodule algorithm also has a user section for DME/Orthotics. DME/Orthotics lines of code are optionally billed and generate revenue along with the surgery center procedure lines of code.

As with revenue generated by the lines of procedure codes, when DME/Orthotics lines of codes generate revenue, they do so in one of two mutually exclusive ways. The first way is by the “billed amount” or a percentage of the billed amount. The second way is by the “allowable amount” or the percentage of the allowable amount. Using the surgery center revenue submodule algorithm, the user selects one of the two mutually exclusive options. Next, for each line of DME/Orthotic code(s), a percentage choice is entered from 0-100%, which will apply only to that line of code. The percentage is applied to the billed amount or the allowable amount for that line of code. Applying the percentage to each line of code generates the “base revenue” for that line of code. After the base revenue is calculated, the surgery center revenue submodule algorithm accepts user input of units (default unit being 1). The algorithm multiplies the base revenue for that line of code by the number of units (e.g., up to 10). The algorithm then calculates the “line revenue” for that line of code by multiplying the number of units by the base revenue. The revenue table algorithm pulls the “line revenue” or “line revenues” for the DME/Orthotics to the revenue table in the revenue module.

The algorithms calculate the total revenue for all lines of code. The total revenue for all lines of code is the “component revenue” for the DME/Orthotics. The revenue table algorithm pulls the “component revenue” for the DME/Orthotics to the revenue table in the revenue module. The dynamic revenue table algorithm pulls the “component revenue” to the dynamic revenue table in the profit module.

The cost component of the DME/Orthotics will be linked to revenue table in the surgery center revenue submodule. The creation of this link is discussed in the Inventory Section of the surgery center cost submodule (VI-5).

The surgery center revenue submodule algorithm also has a third section for implants. Implant lines of code are billed and generate revenue along with the surgery center procedure lines of code.

As with revenue generated by the lines of procedure codes, when implant lines of codes generate revenue, they do so in one of two mutually exclusive ways. The first way is by the “billed amount”, or a percentage of the billed amount. The second way is by the “allowable amount” or a percentage of the allowable amount. Using the surgery center revenue submodule algorithm, the user selects one of the two mutually exclusive options. Next, for each line of implant code, the user enters a percentage (0-100%) that applies only to that line of code. The percentage is applied to the billed amount or the allowable amount for that line of code. The application of the percentage to each line of code generates the “base revenue” for that line of code. After the base revenue is calculated, the surgery center revenue submodule algorithm accepts user input of units (default unit being 1). The algorithm multiplies the base revenue for that line of code by the number of units entered (e.g., up to 20). The algorithm then calculates the “line revenue” for that line of code by multiplying the number of units by the base revenue. The revenue table algorithm pulls the “line revenue” or “line revenues” for the implant to the revenue table in the revenue module. The algorithm then calculates the total revenue for all lines of code, which is the “component revenue” for the implants. The revenue table algorithm pulls the “component revenue” for the implants to the revenue table in the revenue module. The dynamic revenue table algorithm pulls the “component revenue” to the dynamic revenue table in the profit module.

The cost component of the implants is then linked to revenue table in the surgery center revenue submodule. The creation of this link is discussed in the Inventory Section of the surgery center cost submodule:
(VA-5)
(VB-s′-5)
(VB-s″-5).

The surgery center revenue submodule algorithm also has a fourth section for HCDs. HCD lines of code, by definition, generate revenue. As with revenue generated by the lines of procedure codes, when HCD lines of codes generate revenue, they do so in one of two mutually exclusive ways. The first way is by the “billed amount”, or a percentage of the billed amount. The second way is by the “allowable amount” or the percentage of the allowable amount. Using the surgery center revenue submodule algorithm, the user selects one of the two mutually exclusive options. Next, for each line of HCD code and applying only to that line of code, a percentage from 0-100% is entered. The application of the percentage to each line of code generates the “base revenue” for that line of code. After the base revenue is calculated, the user enters units into the surgery center revenue submodule algorithm (default unit being 1). The algorithm multiplies the base revenue for that line of code by the number of units chosen (e.g., up to 10) to calculate the “line revenue” for that line of code. The revenue table algorithm pulls the “line revenue” or “line revenues” for the HCD to the revenue table in the revenue module. The algorithm then calculates the total revenue for all lines of code, or the “component revenue” for the HCD. The revenue table algorithm pulls the “component revenue” for the HCD to the revenue table in the revenue module. The dynamic revenue table algorithm pulls the “component revenue” to the dynamic revenue table in the profit module.

The cost component of the HCD is linked to the revenue table in the surgery center revenue submodule. The creation of this link is discussed in the Inventory Section of the surgery center cost submodule:
(IVA-5)
(IVB-5)
(IVB-d′-5)
(IVB-d″-5)
(IVC-5).

The surgery center revenue submodule has a collection discount surgery center sub algorithm that calculates a collection discount on the surgery center revenue. The collection discount reflects the expected revenue the ASC is entitled to collect for the surgery center component of a surgical case to the actual revenue collected. This difference can be for a variety of reasons and the algorithm allows the user to input a range of revenue discount percentages from 0-100%. When the collection discount surgery center sub algorithm is applied, the discount is applied to each revenue component of the surgery center separately. The 4 revenue components are the surgery/procedure component, DME/Orthotic component, HCD component and the implant component. For each component the percentage discount is applied equally to all lines of codes for that component. The revenue generated by each line of code that was originally calculated using surgery center submodule algorithm is displayed alongside the newly calculated discounted revenue for that line of code. The discounted revenue for that line of code is defined as the “corrected line revenue”. Corrected line revenue applies to individual lines of code.

In the surgery center revenue submodule, all components have a “component revenue” for all their respective lines of code. If collection discount surgery center sub algorithm is applied to a component, the component revenue for that component is called “corrected component revenue”. The “component revenue” is displayed alongside the “corrected component revenue”. The total revenue of all components in the surgery center revenue submodule is called the “total surgery center submodule revenue”. If the collection discount surgery center sub algorithm is applied to one or more of the revenue components, the total of all the components is called the “corrected total surgery center submodule revenue”. The “total surgery center submodule revenue” is then calculated for all lines of codes for the surgery center revenue submodule using the surgery center revenue submodule algorithm and is displayed alongside the “corrected total surgery center submodule revenue” calculated using collection discount surgery center sub algorithm.

The anesthesia revenue submodule has only one component that optionally generates anesthesia revenue. This revenue component is the anesthesia provider with associated job descriptions. This anesthesia revenue component has two mutually exclusive options for line or lines of revenue code that reflect the two types of anesthesia that the anesthesia provider or providers can provide to the client. These two types of anesthesia are general anesthesia/MAC (Monitored anesthesia Care) and conscious sedation. The anesthesia revenue lines of code for general anesthesia/MAC and conscious sedation are different. In any one CE, the revenue lines of code can be for general anesthesia/MAC or conscious sedation but not both.

The anesthesia revenue submodule algorithm functions on the anesthesia revenue lines of code in the same manner as the professional revenue submodule algorithm functions on the professional revenue lines of code and the Surgery center revenue submodule algorithm functions on the surgery center revenue lines of code. All 3 algorithms process the billed and allowable amounts for each line of revenue code in their respective submodules.

In the professional revenue submodule, the surgery center revenue submodule and the anesthesia revenue submodule for both general anesthesia/MAC and conscious sedation, the billed amounts are inputted by the user or pulled from the fee schedule.

In the professional revenue submodule, the surgery center revenue submodule and the anesthesia revenue submodule for conscious sedation the allowable amounts are inputted by the user or pulled from the fee schedule.

In the anesthesia revenue submodule for general anesthesia/MAC the allowable amount is calculated using the anesthesia allowable algorithm.

For general anesthesia/MAC revenue, the user selects/enters the anesthesia provider or providers and their job descriptions that generate general anesthesia/MAC revenue within the anesthesia component of the surgical case from the CE set up algorithm. Only anesthesia providers are selectable.

For each anesthesia provider, the anesthesia revenue submodule algorithm the user inputs the procedure code (CPT/HCPSC code) along with any modifiers and the associated billed amount for that code directly or pulled from a fee schedule. The user inputs a percentage of the billed amount for that code (0-100%). The algorithm then calculates the “line revenue” for that code by multiplying the percentage chosen by the billed amount and the revenue table algorithm pulls the “line revenue” or “line revenues” for each anesthesia provider to the revenue table in the revenue module. The total revenue of the “line revenue” or “line revenues” for each anesthesia provider is the “component revenue” for that anesthesia provider.

The revenue table algorithm pulls the “component revenue” for each anesthesia provider to the revenue table in the revenue module. The dynamic revenue table algorithm pulls the “component revenue” for each anesthesia provider to the dynamic revenue table in the profit module.

The anesthesia allowable is calculated using the anesthesia allowable algorithm. The user inputs the procedure code (CPT/HCPSC code) for the anesthesia provider or providers, either directly or pulled from their fee schedule. The anesthesia allowable algorithm described below calculates the anesthesia allowable and enter the appropriate associated modifiers

Once the anesthesia allowable is calculated, the user selects a percentage of the allowable amount for that code (0-100%). The algorithm calculates the “line revenue” for that code by multiplying the percentage chosen by the Allowable Amount. The revenue table algorithm pulls the “line revenue” or “line revenues” for each anesthesia provider to the revenue table in the revenue module. The “line revenue” for each anesthesia provider is the “component revenue” for that anesthesia provider.

The revenue table algorithm pulls the “component revenue” for each anesthesia provider to the revenue table in the revenue module. The dynamic revenue table algorithm pulls the “component revenue” for each anesthesia provider to the dynamic revenue table in the profit module.

For general anesthesia/MAC, the anesthesia allowable algorithm calculates the anesthesia Allowable for the range of possible anesthesia provider or providers. There is a systematic approach of entering data into the algorithms for each possible combination of anesthesia provider or providers that allows for the anesthesia allowable to be calculated and the appropriate anesthesia modifiers to be linked to those allowable. The anesthesia provider job description is selected from: MDA acting alone (anesthesiologist), CRNA acting alone (Certified Nurse Anesthetist), MDA supervising a CRNA with MDA present/not present at induction, MDA supervising an AA (Anesthesia Assistant) with MDA present/not present at induction, MDA directing a CRNA, MDA directing an AA or MDA directing an RN (Register Nurse), Resident, Intern. The possibilities are numbered below. A link to the criteria for determining whether an MDA is supervising or directing is provided.

The anesthesia allowable algorithm for the MDA acting alone (anesthesiologist) calculates the anesthesia allowable by sequentially filling out the anesthesia allowable formula with user input. First, the user selects/enters an anesthesia CPT code for the surgical case or pulls it from the fee schedule. Second, the anesthesia time in minutes for the surgical case is pulled from the third part of CE set up. When the anesthesia allowable algorithm is used, the anesthesia time entered in minutes must be equal to the sum of O.R. stage time and Post-op #1 stage time entered in CE set up. To change the time inputted by the anesthesia allowable algorithm, the user will need to change the time inputted in the CE set up for the O.R. stage and/or Post-op #1 stage.

Third, the “Interval” is entered/added which is determined by the prevailing payor contract. The algorithm divides minutes by the interval to calculate the sum and then provide user input choices of: round the sum up, round the sum down or use the exact calculation. Whether the sum is rounded up, rounded down or the exact calculation is used, depends on the payor being considered for the calculations. The final calculation is referred to as the Time Unit.

Fourth, the user enters or pulls from the fee schedule the base unit that is linked to the anesthesia CPT code using the anesthesia allowable algorithm.

Fifth, the user enters the physical status modifying unit (e.g., 0, 1, or 2). Hyperlink descriptions of how to determine the physical status are provided.

Sixth, the user enters a conversion factor which varies by payor and geographical location. Links for different conversion factors are provided.

Once all the variables are entered, the anesthesia allowable algorithm calculates the anesthesia allowable for the MDA.

The “line revenue” generated by the MDA for the surgical case is calculated using anesthesia revenue submodule algorithm as a percentage of the anesthesia allowable. The revenue table algorithm pulls the anesthesia “line revenue” for the MDA to the revenue table in the revenue module and to the dynamic revenue table in the profit module.

The anesthesia Allowable algorithm receives user inputs for anesthesia allowable formula for the CRNA acting alone (Certified Nurse anesthetist) and calculates the anesthesia allowable. The formula is filled out by the same method as it is for MDA. Once all variables are entered the anesthesia allowable algorithm will calculate the anesthesia allowable for the CRNA.

The revenue generated by the CRNA for the surgical case is calculated using anesthesia revenue submodule algorithm as a percentage of the anesthesia allowable. The revenue table algorithm pulls the anesthesia revenue for the CRNA and the modifier to the revenue table in the revenue module.

The anesthesia Allowable algorithm receives user inputs for MDA directing a CRNA. The anesthesia Allowable algorithm for MDA directing a CRNA with MDA directing one case calculates the anesthesia allowable. The formula is filled out by the same method as it is for the MDA acting alone or CRNA acting alone. Once all variables are entered, the anesthesia allowable algorithm will calculate the anesthesia allowable. Both anesthesia providers have the same anesthesia allowable.

The anesthesia revenue submodule algorithm then uses the anesthesia allowable to independently calculate the “line revenue” generated by each anesthesia provider for the surgical case. The “line revenue” generated by the CRNA directed by an MDA with MDA directing one case is calculated as a percentage of the anesthesia allowable entered/selected by the user. The “line revenue” generated by the MDA directing a CRNA with MDA directing one surgical case is also calculated as a percentage of the anesthesia Allowable chosen by the user. The anesthesia revenue submodule algorithm calculates the anesthesia provider “component revenue” for the surgical case as the sum of the “line revenues”.

The anesthesia allowable algorithm receives user inputs for MDA directing a CRNA with MDA directing 2-4 cases. The anesthesia allowable algorithm for MDA directing a CRNA with MDA directing 2-4 cases calculates the anesthesia allowable from the user input. The formula is filled out by the same method as it is for the MDA acting alone or CRNA acting alone. Once all variables are entered, the anesthesia allowable algorithm will calculate the anesthesia allowable.

The anesthesia revenue submodule algorithm then uses the anesthesia allowable to calculate the “line revenue” generated by each anesthesia provider for the surgical case. The “line revenue” generated by the CRNA directed by a MDA with MDA directing 2-4 cases is calculated as a percentage of the anesthesia allowable chosen by the user.

The “line revenue” generated by the MDA directing a CRNA with MDA directing 2-4 surgical cases is also calculated as a percentage of the anesthesia allowable as selected by the user. The anesthesia revenue submodule algorithm calculates the anesthesia provider “component revenue” for the surgical case as the sum of the “line revenues”.

The anesthesia allowable algorithm receives user inputs for MDA directing an AA (anesthesia assistant) with MDA directing 2-4 cases. The anesthesia allowable algorithm for MDA directing an AA (anesthesia assistant) with MDA directing 2-4 cases calculates the anesthesia allowable from the user input. The formula is filled out by the same method as it is for the MDA acting alone or CRNA acting alone. Once all variables are entered, the anesthesia allowable algorithm will calculate the anesthesia allowable. The anesthesia revenue submodule algorithm then uses the anesthesia allowable to calculate the “line revenue” generated by each anesthesia provider for the surgical case. The “line revenue” generated by the anesthesia assistant directed by an MDA with MDA directing 2-4 cases is calculated as a percentage of the anesthesia allowable chosen by the user. The “line revenue” generated by the MDA directing an anesthesia assistant with MDA directing 2-4 surgical cases is also calculated as a percentage of the anesthesia allowable as selected by the user.

The anesthesia revenue submodule algorithm calculates the anesthesia provider “component revenue” for the surgical case as the sum of the “line revenues”.

The anesthesia allowable algorithm receives user inputs for MDA Supervising a CRNA and calculates the anesthesia allowable separately for the MDA and for the CRNA using three different algorithms. The first algorithm is the same anesthesia allowable algorithm which calculates the anesthesia allowable for the CRNA in the surgical case using the same method as above to calculate the anesthesia allowable for the CRNA acting alone. To calculate the anesthesia allowable for the MDA there are two different algorithms based upon whether the MDA was present at induction or not. When the MDA is present at induction the anesthesia allowable induction/present sub algorithm calculates the anesthesia allowable as 3 base units plus 1 time unit multiplied by the “conversion factor” to calculate the anesthesia allowable for the MDA present at induction (i.e. 4*“conversion factor”=anesthesia allowable).

When the MDA is not present at induction the anesthesia allowable Induction/not Present sub algorithm calculates anesthesia allowable as 3 base units multiplied by the “conversion factor” to calculate the anesthesia allowable for the MDA not present at induction (i.e. 3*“conversion factor”=anesthesia Allowable).

The anesthesia revenue submodule algorithm calculates the “line revenue” for the CRNA supervised by an MDA as a percentage of the anesthesia allowable for the CRNA. The anesthesia revenue submodule algorithm calculates the “line revenue” for an MDA that supervises a CRNA as a percentage of the anesthesia allowable for the MDA. The anesthesia revenue submodule algorithm calculates the “component revenue” generated by both anesthesia providers together for the surgical case.

The anesthesia allowable algorithm receives user inputs MDA supervising an anesthesia assistant and calculates the anesthesia allowable separately for the MDA and the anesthesia assistant using the three different algorithms described above. The calculations for the anesthesia allowable for the MDA supervising an anesthesia assistant are the same as the calculations for an MDA supervising a CRNA. The revenue for the anesthesia assistant supervised by the MDA and the revenue for the MDA is also calculated the same way as above.

The anesthesia revenue submodule algorithm calculates the “line revenues” and “component revenue” for both anesthesia providers in a surgical case.

Revenue for conscious sedation is both in the anesthesia revenue submodule and the professional revenue submodule depending upon whether the conscious sedation is performed by an anesthesia provider or a professional provider. The revenue component of the conscious sedation option is either from the anesthesia provider or the professional provider.

The user selects the provider that generates conscious sedation revenue in the surgical case from the CE Set Up algorithm. With conscious sedation, the component that generates revenue can be either the professional provider in the professional revenue submodule or the anesthesia provider in the anesthesia revenue submodule. Either provider is selected from CE set up in their respective submodules when inputting conscious sedation revenue codes. However, the inputting of conscious sedation revenue codes into both submodules is prohibited by conscious sedation algorithm.

The conscious sedation algorithm's function in the professional revenue submodule is to allow for only CPT/HCPCS codes G0500, 99151, 99152, 99153 to be inputted for the professional provider. The conscious sedation algorithm does not prohibit other professional component or DME/orthotic component revenue codes to be inputted into the professional revenue submodule.

The conscious sedation algorithm's function in the anesthesia revenue submodule is to allow for only CPT/HCPCS 99155, 99156, 99157 codes to be inputted. The conscious sedation algorithm prohibits the application of any general anesthesia/MAC algorithms in the anesthesia revenue submodule if conscious sedation revenue codes are inputted.

The conscious sedation algorithm prohibits conscious sedation revenue codes to be inputted in both professional revenue submodule and anesthesia revenue submodule. If one set of allowed conscious sedation revenue codes is inputted in one submodule, the inputting of the other set of allowed conscious sedation codes in the other submodule is prohibited.

For the professional provider, the professional revenue submodule algorithm accepts user input of the allowed procedure code or codes (CPT/HCPSC codes) for the conscious sedation revenue component of the surgical case in the professional revenue submodule.

For the anesthesia provider, the anesthesia revenue submodule algorithm accepts user input of the allowed procedure code or codes (CPT/HCPSC codes) for the conscious sedation revenue component of the surgical case into the anesthesia revenue submodule.

The anesthesia revenue submodule algorithm and the professional revenue submodule algorithm accepts user input of one of three methods to enter the conscious sedation revenue procedure code or codes into their respective submodules. The first and second methods are the same as described above in the anesthesia revenue submodule section and the professional revenue submodule section.

The first method accepts user input to directly enter the CPT/HCPCS codes and associated modifiers as well as the billed amount and the allowable amount by payor for each line of code.

The second method accepts user input to pull the CPT/HCPCS codes and associated modifiers as well as the billed amount and the allowable amount by payor for each line of code.

The third method uses 3 Conscious sedation sub algorithms, described below, to assist the user in choosing the appropriate CPT/HCPCS codes.

Using any of the three methods, the user will input all lines of conscious sedation revenue codes for the surgical case into either the professional revenue submodule or the anesthesia revenue submodule. The codes will be entered sequentially as they would be formally billed and submitted to a payor on a HCFA form along with the associated billed and allowable amounts for each line of code.

From the revenue standpoint, each line of code generates revenue in one of two mutually exclusive ways. The first way is by the “billed amount”, or a percentage of the billed amount. The second way is by the “allowable amount” or the percentage of the allowable amount. Depending upon the submodule into which conscious sedation revenue codes were inputted, the anesthesia revenue submodule algorithm or professional revenue submodule algorithm is applied. The user selects one of the two mutually exclusive options of the “billed amount” or the “allowable amount” that will apply equally to all lines of conscious sedation revenue code. The user then enters a percentage for each line of code and the percentage applies only to that line of code. The percentage for each line of code is applied to the billed amount or the allowable amount for that line of code depending on the first choice that was made. The algorithm applies the percentage chosen and will then calculate the “line revenue” for that line of code. The revenue table algorithm pulls the “line revenue” or “line revenues” for conscious sedation for the anesthesia provider to the anesthesia revenue submodule section of the revenue table. The revenue table algorithm pulls the “line revenue” or “line revenues” for conscious sedation for the professional provider to the professional revenue submodule section of the revenue table.

The total revenue for all lines of code for the anesthesia provider or professional provider is the “component revenue” for the anesthesia provider or professional provider for conscious sedation. The revenue table algorithm pulls the “component revenue” for conscious sedation for the anesthesia provider or professional provider to the respective sections of the revenue table in the revenue module. The dynamic revenue table algorithm pulls the component revenue to dynamic revenue table in the profit module.

There are three separate sub algorithms to assist the user in choosing the appropriate HCPCS and/or CPT codes to be billed and the order in which they are billed for the conscious sedation. The user selects one of the 3 sub algorithms. Each of the sub algorithms has associated HCPCS and/or CPT codes that reflect the step wise ranges in time for conscious sedation. The user inputs the codes using one of the 3 sub algorithms. Each code or series of codes together represents a time range. Each of the 3 sub algorithms allocates the maximum conscious sedation time in the time range for the HCPCS and/or CPT code or codes chosen. This maximum time is allocated to the O.R. stage only. This background allocation is pushed to stage time variable sub algorithm in the profit module {PART II}, stage time variable sub algorithm, revenue, and stage time for conscious sedation.

The first sub algorithm is GI/CS same provider sub algorithm. The GI/CS same provider sub algorithm is for gastrointestinal endoscopy procedures where the professional provider is performing both the conscious sedation and the surgical procedure. The system prompts for a choice of two separate procedure code pathways. The first pathway is HCPCS/CPT pathway. The second pathway is the HCPCS Only pathway.

If the HCPCS/CPT code pathway is selected, the user then enters the time in minutes for conscious sedation. The GI/CS Same provider sub algorithm generates the HCPCS code G0500 for the first 0-15 min of conscious sedation time selected by the user. For any remaining time, greater than 15 min, the sub algorithm provides an option of adding additional CPT codes for the remaining time. If the user adds additional CPT codes for the remaining time, the sub algorithm will add CPT code 99153 for a user entered time from 16-31 min. The sub algorithm adds an additional 99153 CPT code for user entered time of 32-47 min. Additional 15 min time ranges are added by way of additional 99153 CPT codes.

If the HCPSC Only pathway is chosen, the sub algorithm will only generate HCPCS code G0500. The user will not be asked to enter a time and no other codes will be generated.

The second sub algorithm is called CS Same provider sub algorithm. In the second sub algorithm, the professional provider is performing both the conscious sedation and the surgical procedure. The professional provider generates conscious sedation revenue in the professional provider revenue submodule along with the other professional provider revenue.

The CS Same provider sub algorithm has two options. The first option is for clients less than 5 years old. Once the first option is chosen, the user will enter the time in minutes for the conscious sedation. For the first 0-9 min of conscious sedation time chosen by the user the algorithm will enter the phrase “NO CODE” as there is no code and no reimbursement for less than 10 min. For the 10-22 min of conscious sedation time chosen by the user the algorithm will enter CPT code 99151. The algorithm will add an additional 99153 CPT code for times of 23-37 min. The algorithm will add an additional 99153 CPT code for times of 38-52 min. The algorithm will add an additional 99153 CPT code for times of 53-67 min. The algorithm will add an additional 99153 CPT code for times of 68-82 min. The algorithm will add an additional 99153 CPT code for times of 83 min or anytime greater than 83 min.

The second option is for clients greater than 5 years old. Once the second option is selected, the user will enter the time in minutes for the conscious sedation. For the first 0-9 min of conscious sedation time entered by the user, the algorithm enters the phrase “NO CODE” as there is no code and no reimbursement for less than 10 min. For the 10-22 min of conscious sedation times, the algorithm will enter CPT code 99152. The algorithm will add an additional 99153 CPT code times of 23-37 min. The algorithm will add an additional 99153 CPT code for times of 38-52 min. The algorithm will add an additional 99153 CPT code for times of 53-67 min. The algorithm will add an additional 99153 CPT code for times of 68-82 min. The algorithm will add an additional 99153 CPT code for times of 83 min or anytime greater than 83 min.

The third sub algorithm is called CS Different provider sub algorithm. In the third sub algorithm the anesthesia provider is performing conscious sedation and the professional provider is performing the surgical procedure. The professional provider generates revenue for the professional component of the surgical case in the professional revenue submodule. The anesthesia provider generates conscious sedation revenue in the anesthesia revenue submodule. The CS Different provider sub algorithm is only for an anesthesia provider providing conscious sedation in the surgical case.

The CS Different provider sub algorithm has two pathways. The first pathway is for clients less than 5 years old. Once the first pathway is chosen, the user will enter the time in minutes for the conscious sedation. For the first 0-9 min of conscious sedation time chosen by the user the sub algorithm will enter the phrase “NO CODE” as there is no code and no reimbursement for less than 10 min. For the 10-22 min of conscious sedation time chosen by the user the algorithm will enter CPT code 99155. The algorithm will add an additional 99157 CPT code for user entered time of 23-37 min. The algorithm will add an additional 99157 CPT code for user entered time of 38-52 min. The algorithm will add an additional 99157 CPT code for user entered time of 53-67 min. The algorithm will add an additional 99157 CPT code for user entered time of 68-82 min. The algorithm will add an additional 99157 CPT code for user entered time of 83 min or anytime greater than 83 min.

The second pathway is for clients greater than 5 years old. Once the second pathway is chosen, the user will enter the time in minutes for the conscious sedation. For the first 0-9 min of conscious sedation time chosen by the user the sub algorithm will enter the phrase “NO CODE” as there is no code and no reimbursement for less than 10 min. For the 10-22 min of conscious sedation time chosen by the user the algorithm will enter CPT code 99156. The algorithm will add an additional 99157 CPT code for user entered time of 23-37 min. The algorithm will add an additional 99157 CPT code for user entered time of 38-52 min. The algorithm will add an additional 99157 CPT code for user entered time of 53-67 min. The algorithm will add an additional 99157 CPT code for user entered time of 68-82 min. The algorithm will add an additional 99157 CPT code for user entered time of 83 min or anytime greater than 83 min.

### Collection Discount:

The anesthesia revenue submodule, as in the professional revenue and surgery center submodules, also has a collection discount anesthesia sub algorithm that applies in the same manner to MAC/general anesthesia and conscious sedation revenue. The collection discount reflects the expected revenue the ASC is entitled to collect for the anesthesia component of a surgical case to the actual revenue collected. This difference can be for a variety of reasons and the algorithm allows the user to input a range of revenue discount percentages from 0-100%.

When the collection discount anesthesia sub algorithm is applied, the percentage discount is applied equally to all lines of codes in the anesthesia revenue submodule. The revenue generated by each line of code that was originally calculated using anesthesia submodule algorithm is displayed alongside the newly calculated discounted revenue for that line of code. The discounted revenue for each line of code is defined as the “corrected revenue” for that line of code.

As discussed in CE set up, the fifth possibility the payment rate algorithm considers is for an anesthesia provider paid as a percentage of the anesthesia revenue collected. The user chooses the lines of code for which the percentage applies. The revenue for the lines of code chosen will be the “corrected component revenue” if the collection discount anesthesia sub algorithm has been applied.

For the anesthesia provider in the anesthesia revenue submodule, the total revenue for all lines of corrected revenue is defined as the “corrected component revenue”. The “corrected component revenue” for all lines of codes for anesthesia provider is displayed alongside the “component revenue”.

The total revenue for all lines of “corrected component revenue” in the anesthesia revenue submodule is defined as the “corrected total anesthesia submodule revenue”. The “corrected total anesthesia submodule revenue” is displayed alongside the “total anesthesia submodule revenue”.

### Ancillary Revenue Submodule and Ancillary Component:

The ancillary revenue submodule allows for additional revenue sources not accounted for in the professional, anesthesia or surgery center revenue submodules to be included as a revenue source in the CE.

For purposes of this system, the ancillary revenue submodule has only one component that can potentially generate ancillary revenue. This ancillary revenue source component is the ancillary provider. All revenue, including both technical ancillary revenue and professional ancillary revenue are considered ancillary provider revenue.

The user will select from the CE Set Up algorithm the ancillary provider or providers that generate revenue within the ancillary component of the surgical case.

For each ancillary provider that generate revenue, the ancillary revenue submodule algorithm allows the user to input the associated procedure code or codes (CPT/HCPSC codes) for the ancillary revenue component of the surgical case for that ancillary provider. This algorithm will allow the user to enter either directly or pull from their fee schedule the CPT/HCPCS codes and associated modifiers, procedure code descriptions as well as the billed amount and the allowable amount by payor for each line of code.

There is an option to use existing CPT codes or HCPCS codes or to create unique codes for the ancillary items to be billed. The user will input all lines of code for the surgical case as well as the associated billed and allowable amounts for each line of code sequentially as they would be formally billed and submitted to a payor on an HCFA form.

From the revenue standpoint, each line of code generates revenue in one of two mutually exclusive ways. The first way is by the “billed amount” for that line of code, or a percentage of the billed amount for that line of code. The second way is by the “allowable amount” for that line of code or the percentage of the allowable amount for that line of code. Using the ancillary revenue submodule algorithm, the user will choose one of the two mutually exclusive options of the “billed amount” or the “allowable amount”. The first choice will apply to all lines of codes for the surgical case for that ancillary provider. Once the first choice is made the algorithm will require a second choice to be made. This second choice is made separately for each line of code and will apply only to that line of code. The percentage choice is from 0-100%. The user will choose a percentage for each line of code that will apply to the billed amount or the allowable amount for that line of code depending on the first choice that was made.

The application of the percentage to each line of code will be the “base revenue” for that line of code.

After the base revenue is calculated, the ancillary revenue submodule algorithm will allow the user to apply units to the base revenue with the default unit being 1. With the choice of units, the algorithm will multiply the base revenue for that line of code by the number of units chosen up to 10. The algorithm will then calculate the “line revenue” for that line of code by multiplying the number of units by the base revenue. The revenue table algorithm pulls the “line revenue” or “line revenues” for the ancillary provider to the revenue table in the revenue module.

Once the user chooses the base revenue and units for each of the lines of codes that make up the surgical case, the algorithm will calculate the total revenue for all lines of code for that ancillary provider. The total revenue for all lines of code for the ancillary provider is the “component revenue” for that ancillary provider. The revenue table algorithm pulls the “component revenue” for the ancillary provider to the revenue table in the revenue module. The dynamic revenue table algorithm pulls the “component revenue” to the dynamic revenue table in the profit module.

The user can select from the CE Set Up algorithm additional ancillary providers that generate revenue within the ancillary component of the surgical case. Each ancillary provider added will have their own ancillary provider “component revenue” calculated by the ancillary revenue submodule algorithm in the same manner as above. The revenue table algorithm pulls the “component revenue” for each additional ancillary provider to the revenue table in the revenue module. The dynamic revenue table algorithm pulls the “component revenue” for each additional ancillary provider to the dynamic revenue table in the profit module.

### Collection Discount:

The ancillary revenue submodule also has a collection discount ancillary sub algorithm that will calculate a collection discount on the ancillary revenue. The collection discount reflects the expected revenue the ASC is entitled to collect for the ancillary component of a surgical case to the actual revenue collected. This difference can be for a variety of reasons and the algorithm allows the user to input a range of revenue discount percentages from 0-100%.

When the collection discount ancillary sub algorithm is applied, the percentage discount is applied equally to all lines of codes in the ancillary revenue submodule. The revenue generated by each line of code that was originally calculated using ancillary revenue submodule algorithm is displayed alongside the newly calculated discounted revenue for that line of code. The discounted revenue for each line of code is defined as the “corrected revenue” for that line of code.

As discussed in CE set up, the fifth possibility the payment rate algorithm considers is for an ancillary provider paid as a percentage of ancillary revenue collected. The user chooses the lines of code for which the percentage applies. The revenue for the lines of code chosen will be the corrected revenue if the collection discount ancillary sub algorithm has been applied.

For each ancillary provider in the ancillary revenue submodule, the total revenue for all lines of corrected revenue is defined as the “corrected component revenue”. The “corrected component revenue” for all lines of codes for ancillary provider is displayed alongside the “component revenue”.

The total revenue for all lines of “corrected component revenue” in the ancillary revenue submodule is defined as the “corrected total ancillary submodule revenue”. The “corrected total ancillary submodule revenue” is displayed alongside the “total ancillary submodule revenue”.

### Revenue Table Submodule:

The revenue table algorithm accesses the data from the revenue submodules. The algorithm will disregard any of the revenue submodules if that submodule has no inputted data and therefore does not actively contribute to revenue. For only those submodules that have revenue data, the algorithm enters that data into the revenue table. The dynamic revenue table algorithm pulls selected data from the revenue table in the revenue module to the dynamic revenue table in the profit module.

### Columns in the Revenue Table:

Columns in the revenue table contain data pulled from the revenue submodules by the revenue table algorithm. The 1^{st} column/revenue generator: The first column are the revenue generators for the 4 revenue submodules divided by submodule and subdivided by the submodule's respective components. Only the revenue submodules that generate revenue and the components within a revenue submodule that generate revenue are included in the revenue table by the revenue table algorithm.

The professional revenue submodule contains the professional provider component and DME/Orthotics component. The surgery center revenue submodule contains the Surgery/Procedure component, DME/Orthotic component, Implant component and HCD component. The anesthesia revenue submodule contains the anesthesia provider components for the general anesthesia/MAC or the conscious sedation options. The ancillary revenue submodule contains the ancillary provider component.

2^{nd} column/CPT/HCPCS Code Description: The CPT/HCPCS code description is inputted in one of the revenue submodules as fully free text (FFT) or pulled from user Fee schedule.

3^{rd} column/CPT/HCPCS Code: The CPT/HCPCS code with or without associated modifier or modifiers is inputted in one of the revenue submodules as fully free text (FFT) or pulled from user Fee schedule or inputted by the system when anesthesia Allowable is calculated for the anesthesia CPT/HCPCS codes in the anesthesia revenue submodule

4^{th} column/modifiers: Additional modifiers are inputted in one of the revenue submodules as fully free text (FFT)

5^{th} column/Billed Amount: The billed amount for the CPT/HCPS codes is inputted in one of the revenue submodules as limited free text (LFT) or pulled from user Fee schedule.

6^{th} column/% Billed Amount: The percentage of the billed amount is calculated by the system in one of the revenue submodules if the percentage of “billed amount” option is chosen. This would be the “base revenue” for that line of code. If the percentage of “billed amount” option in not chosen, the column will be blank.

7^{th} column/Allowed Amount: The allowed amount for the CPT/HCPS codes is inputted in one of the revenue submodules as limited free text (LFT) or pulled from user Fee schedule.

8^{th} column/% of Allowable Amount: The percentage of the allowable amount is calculated by the system in one of the revenue submodules if the percentage of “allowed amount” option is chosen. This would be the “base revenue” for that line of code. If the percentage of “allowed amount” option is not chosen, the column will be blank.

9^{th} column/Base Units: Base units are inputted in the revenue submodules as a system option (SO) that acts as a multiplier on column 5 or column 7

10^{th} column/revenue: The revenue in this column is either line revenue, component revenue, total revenue for one of the revenue submodules or Total CE revenue depending upon the row. Line revenue is calculated for the CPT/HCPSC code in columns 6 & 9 or in columns 8 & 9 depending upon whether “billed amount” or “allowed amount” percentage is chosen. This would be the revenue for that line of code.

11^{th} column/Corrected revenue: The corrected revenue in this column is calculated by the system in one of the revenue submodules by the application of the collection discount algorithm in that submodule. The corrected revenue is either corrected line revenue, corrected component revenue, corrected total revenue for one of the revenue submodules or Corrected Total CE revenue depending upon the row.

### Rows in the Revenue Table:

Rows in the revenue table are filled with data accessed or calculated by revenue table algorithm. The rows are separated by revenue submodules and by the different components within each revenue submodule.

### Professional Revenue Submodule Section

### Professional Provider Component Row(s):

In the professional revenue submodule, the data for each line of code for each professional provider was sequentially inputted in the order that it would be formally billed and submitted to a payor on a HCFA form. In that same order, these professional providers and their job descriptions are pulled onto the revenue table and the data for each professional provider is entered into corresponding columns. The revenue data for each line of code pulled into the revenue column is called the “line revenue”. The corrected revenue data pulled into the corrected revenue column is called “corrected line revenue”. Each professional provider line or lines of code and its corresponding data are separated from other professional providers line or lines of code and their respective data.

The “component revenue” for each professional provider is pulled from the professional revenue submodule. The component revenue is the sum of the line revenue and is inputted in the revenue column, column 10, below the line revenue for that professional provider. The “corrected component revenue” data is the sum of the corrected line revenue and is inputted into the corrected revenue column, column 11, below the corrected line revenue for each professional provider.

### DME/Orthotic Component Row(s):

DME/Orthotic lines of code can, on occasion, generate revenue in the professional revenue submodule. When DME/Orthotic lines of code are inputted into the professional revenue submodule they are considered as a separate component and are inputted beneath the professional provider component. This order continues on the revenue table. In the professional revenue submodule, the DME/Orthotic data for each line of code was sequentially inputted in the order that it would be formally billed and submitted to a payor on a HCFA form. This data is pulled and entered into corresponding columns in that same order. The revenue data for each line of code pulled into the revenue column is called the “line revenue”. The corrected revenue data pulled to the corrected revenue column is called “corrected line revenue”.

Each DME/Orthotic line or lines of code and its corresponding data are considered together and are not separated from each other as different professional providers lines of code and their data are separated from each other. All DME/Orthotics are considered together.

The sum of the line revenues for each DME/Orthotic is considered the “component revenue” for DME/Orthotics. The “component revenue” for DME/Orthotic is pulled from the professional revenue submodule and entered into the revenue column, column 10, below the line revenue. The “corrected component revenue” data is pulled from the professional revenue submodule and is inputted into the corrected revenue column, column 11, below the corrected line revenue.

### Total of Professional Revenue Submodule Row:

The revenue table algorithm calculates the “total revenue” and the “corrected total revenue” for the professional revenue submodule and inputs it into the revenue column and Corrected revenue column. The “total revenue” for the professional revenue submodule is the sum of the professional provider “component revenue” for each professional provider plus the DME/Orthotic “component revenue”.

The “corrected total revenue” for the professional revenue submodule is the sum of the professional provider “corrected component revenue” for each professional provider plus the DME/Orthotic “corrected component revenue”.

### Surgery Center Revenue Submodule Section:

### Surgery/Procedure Component Row(s):

For the surgery/procedure component the CPT/HCPCS code or codes for that procedure is pulled from the surgery center revenue submodule into the second column. Following the manner in which the codes were inputted in the surgery center revenue submodule, each row of code is sequentially organized as it would be formally billed and submitted to a payor on a HCFA or UB-4 form. The data in the remaining columns that correspond to each row are inputted as outlined above with the “revenue” for each line of code and “corrected revenue” for each line of code inputted into columns 10 and 11 respectively.

### Implant Component Row(s):

For the Implant component the CPT/HCPCS code or codes if present is pulled from the surgery center revenue submodule into the second column. Following the manner in which the codes were inputted in the surgery center revenue submodule, each row of code is sequentially organized as it would be formally billed and submitted to a payor on a HCFA or UB-4 form. The data in the remaining columns that correspond to each row are inputted as outlined above with the “revenue” for each line of code and “corrected revenue” for each line of code inputted into columns 10 and 11 respectively.

### HCD Component Row(s):

For the HCD component the CPT/HCPCS code or codes if present are pulled from the surgery center revenue submodule into the second column. Following the manner in which the codes were inputted in the surgery center revenue submodule, each row of code is sequentially organized as it would be formally billed and submitted to a payor on a HCFA or UB-4 form. The data in the remaining columns that correspond to each row are inputted as outlined above with the “revenue” for each line of code and “corrected revenue” for each line of code inputted into columns 10 and 11 respectively.

### DME/Orthotic Component Row(s):

For the DME/Orthotic component the CPT/HCPCS code or codes if present is pulled from the surgery center revenue submodule into the second column. Following the manner in which the codes were inputted in the surgery center revenue submodule, each row of code is sequentially organized as it would be formally billed and submitted to a payor on a HCFA or UB-4 form. The data in the remaining columns that correspond to each row are inputted as outlined above with the “revenue” for each line of code and “corrected revenue” for each line of code inputted into columns 10 and 11 respectively.

On the table, the Surgery/Procedure component CPT/HCPCS lines of codes are separated from the Implant component lines of code, HCD component lines of code and DME/Orthotic component lines of code.

The “component revenue” for each of the components, namely the Surgery/Procedure component and Implant component, HCD component and DME/Orthotic component when present, is pulled from the surgery center revenue submodule. These component revenue or revenues is inputted below the “line revenue” for each of the respective components in the revenue column 10.

The “corrected component revenue” for each of the components, namely the Surgery/Procedure component and Implant component, HCD component and DME/Orthotic component when present, is pulled from the surgery center revenue submodule. These component revenue or revenues are inputted below the “corrected line revenue” for each of the respective components in the revenue column 11.

The “total revenue” for the surgery center revenue submodule is calculated by the revenue table algorithm as the sum of the “component revenues” and is inputted in revenue column 10.

The “corrected total revenue” for the surgery center revenue submodule is calculated by the revenue table algorithm as the sum of the “corrected component revenues” and is inputted into the Corrected revenue column 11.

### Anesthesia Revenue Submodule Section:

### Anesthesia Provider Component Row(s):

For each anesthesia provider with their job description, the CPT/HCPCS code or codes for that anesthesia provider is pulled from the anesthesia revenue submodule into the second column. Following the manner in which the codes were inputted in the anesthesia revenue submodule, each row of code is sequentially organized as it would be formally billed and submitted to a payor on a HCFA form. The data in the remaining columns that correspond to each row are inputted as outlined above with the “line revenue” for each line of code and “corrected line revenue” for each line of code inputted into columns 10 and 11 respectively.

Each anesthesia provider CPT/HCPSC lines of code are separated from each other.

The “component revenue” for each anesthesia provider is pulled from the anesthesia revenue submodule and is inputted below the revenue lines of code for each anesthesia provider in the revenue column 10.

The “corrected component revenue” for each anesthesia provider is pulled from the anesthesia revenue submodule and is inputted below the “corrected revenue” lines of code for each anesthesia provider in Corrected revenue column 11.

The “total revenue” for the anesthesia revenue submodule is calculated by the revenue table algorithm as the sum of the “component revenues” and is inputted in the revenue column 10.

The “corrected total revenue” for the anesthesia revenue submodule is calculated by the revenue table algorithm as the sum of the “corrected component revenues” and is inputted in the revenue column 11.

### Ancillary Revenue Submodule Section

### Ancillary Provider Component Row(s):

For each ancillary provider with their job description, the CPT/HCPCS code or codes for that ancillary provider is pulled from the ancillary revenue submodule into the second column. Following the manner in which the codes were inputted in the ancillary revenue submodule, each row of code is sequentially organized as it would be formally billed and submitted to a payor on a HCFA form. The data in the remaining columns that correspond to each row are inputted as outlined above with the “line revenue” for each line of code and “corrected line revenue” for each line of code inputted into columns 10 and 11 respectively.

Each ancillary provider CPT/HCPSC lines of code are separated from each other.

The “component revenue” for each ancillary provider is pulled from the ancillary revenue submodule and is inputted below the “line revenue” lines of code for each ancillary provider in the revenue column 10.

The “corrected component revenue” for each ancillary provider is pulled from the ancillary revenue submodule and is inputted below the “corrected line revenue” lines of code for each ancillary provider in corrected revenue column 11.

The “total revenue” for the ancillary revenue submodule is calculated by the revenue table algorithm as the sum of the “component revenues” and is inputted in the revenue column 10.

The “corrected total revenue” for the ancillary revenue submodule is calculated by the revenue table algorithm as the sum of the “corrected component revenues” and is inputted in the revenue column 11.

### CE Total Revenue Row:

The revenue table algorithm calculates “CE total revenue” as the sum of the “total revenue” for each submodule and inputs it into the revenue column 10. The revenue table algorithm calculates “corrected CE total revenue” as the sum of the “corrected total revenue” for each submodule and inputs it into the revenue column 11.

### Cost Module:

Cost as it is considered in the Cost Module has 3 Direct Cost components. The first Direct Cost component is Direct Labor Cost. Direct Labor Cost involves the cost of one or more Professional Providers, Anesthesia Providers, Team Members and Ancillary Providers that are directly engaged with client during the CE [PART1]. The second Direct Cost is Direct Supply Cost which includes inventory costs and supply rental costs [PART2]. The third component of Direct Cost is Direct Claim Costs. Direct Claim Costs involve the direct cost of submitting the revenue claim or claims for a particular CE [PART3].

Most of these costs occur during the defined time period known as the Temporal Period (TP) during which the CE occurs. The TP is made up of 4 stages. These 4 stages are the Pre-Op stage, O.R stage, Post-Op #1 stage and Post-Op #2 stage.

Separate from these 4 stages, Cost for direct labor can be outside the TP in one of two ways. The first way is by the Direct Labor costs of AO time. AO time is, by definition, time that is allocated for purely administrative work such as charting, and dictation done outside the TP by a Player that was engaged with the client during the TP. Unlike the stage times for the CE, AO time is different for the different Players in the CE and is therefore not part of the CE Set Up algorithm. AO time is part of Direct Labor algorithm. Not all players have AO time as will be discussed below. AO time also does not need to be consecutive. AO time for a Players that has AO time is the sum of all the administrative time that the Player performs outside the CE for the client.

The second way that Direct Labor cost can be outside the TP is for an Ancillary Provider that is not present during the TP but is directly engaged in an event that occurs during one or more of the stages of the TP and whose costs are allocated to that stage(s). Ancillary Providers are the only Player that can be engaged with the client outside the TP. As with other Players, Ancillary Providers can also be engaged with the client during the TP. This is discussed below.

Costs of Direct Labor [PART1] are calculated using the Direct Labor algorithm which allows for the input of the time in minutes that each of the Players in the “CE Set up” engages with the client during the different stages of CE or for AO time.

The Direct Labor algorithm also considers different kinds of time. These different kinds of time, discussed below, reflect the various ways that the Players can be engaged with the client. As will be detailed below, the ways the Players engage with the client affect the Direct Labor costs.

Cost for Direct inventory supplies [PART2] are calculated using the stage card algorithm discussed below in the Direct Supply Cost-Inventory Cost of the Surgery Center Cost Submodule. Cost for rental supplies is calculated using the Rental Supply algorithm discussed below in the Direct Supply Cost-Rental Supply section. Direct Supply costs are allocated to the entire CE regardless of when they occur and not to any particular stage.

Direct Claim Costs [PART3] are calculated by Third-party Claim Cost algorithm and In-House Claim Cost algorithm and allocated to the entire CE.

### Cost Module [PART1]—Direct Labor Cost:

The Players for Direct Labor algorithm are Professional Providers, Team Members, Anesthesia Providers and Ancillary Providers. Each type of Player has different job descriptions outlined in the Addendum.

The different kinds of time that the Players can be engaged with the client are: Active Locked Engaged Time (ALE), time performing administrative work outside the Client Experience (AO), Passive-Locked time (PLE), and Active Combined time (ACT). Different types of Players have different possible kinds of time in the different stages of the CE and outside the CE which is discussed below.

Active Locked Engaged Time (ALE) is defined as the time a Player is engaged with a client one on one and cannot engage with any other client.

Administration work performed Outside Client Experience (AO) is defined as administrative work performed outside Pre-op, O.R., Post-op #1, Post-op #2 stages. This administrative work can only be performed by a Player that was active in ALE and/or PLE during the Client Experience.

Passive-Locked Time (PLE) is defined as the time that a Player is passive and not actively involved with the client but is locked to that client experience. The Player is not performing administrative work for that client (AO) or performing administrative work for the ASC and is not actively engaged with another client. In other words, they are inactive but are tied to the client.

Active Combined time (ACT) is defined as the time a Player is not in ALE and can engage with more than one client at the same time.

The 4 time periods of stage time are inputted in CE set up using the CE Set Up algorithm. The time that any Player spends with the client in any of the 4 stages of the CE cannot exceed the time period for that stage. Except for the Ancillary Provider that is engaged with the client outside of the CE, if the user inputs time for any Player that exceeds the time period for that stage, the Direct Labor algorithm will give an ERROR.

The total combined time that all Players spend with the client in any particular stage must be equal to or greater than the time period for that stage. If the total time combined that all Players spend with the client in a particular stage is not equal to or greater than the time period for that stage, the Direct Labor algorithm will give an ERROR. Time for an Ancillary Provider that is engaged with Client outside CE is not considered part of the total combined time.

### Professional Provider Direct Labor Cost:

### Professional Provider, Pre-Op Stage (A.1):

A.1 In the Pre-op stage, the Direct Labor algorithm allows for the input of time in minutes that the Professional Provider spends with the client in ALE and PLE for payment types A.1.a, A.1.b and A.1.c described below. ALE and PLE cannot be inputted into payment types A.1.d and A.1.e described below. Professional Providers do not have ACT. For Professional Providers, the cost per hour for ALE and PLE is the same.

### Professional Provider, Pre-Op Stage, Payment Per Hour (A.1.a):

A.1.a After input of the time for “payment per hour” payment type possibility completed in “CE set up”, the Direct Labor algorithm will calculate and display to the user the cost for the Professional Provider in the Pre-op stage. The Cost Table algorithm will pull this cost to the Pre-op stage section of the Cost Table in the Cost Module.

The Direct Labor algorithm will also calculate and display to the user the running “line cost” for the Professional Provider. The running “line cost” will be pulled to the Cost Table in the Cost Module by Cost table algorithm for the Professional Provider.

The running “line cost”, as it relates to all Players, is the running total of the cost for a Player in the CE and changes on the Cost Table as additional direct labor costs are added or removed for that Player. When the user has finished inputting all the direct labor costs for a Player, the running “line cost” is referred to as the “line cost” for that Player.

### Professional Provider, Pre-Op Stage, Salary (A.1.b):

A.1.b After input of time for the “salary” payment type possibility completed in “CE set up” where the payment is converted to the hourly rate, the Direct Labor algorithm will calculate and display to the user the cost for the Professional Provider in the Pre-op stage. The Cost Table algorithm will pull this cost to the Pre-op stage section of the Cost Table in the Cost Module.

The Direct Labor algorithm will also calculate and display to the user the running “line cost” for the Professional Provider. The running “line cost” will be pulled to the Cost Table in the Cost Module by Cost table algorithm for the Professional Provider.

### Professional Provider, Pre-Op Stage, Payment Per Day (A.1.c):

A.1.c After input of time for the “payment per day” payment type possibility completed in “CE set up” where the payment is converted to the hourly rate, the Direct Labor algorithm will calculate and display to the user the cost for the Professional Provider in the Pre-op stage. The Cost Table algorithm will pull this cost to the Pre-op stage section of the Cost Table in the Cost Module.

The Direct Labor algorithm will also calculate and display to the user the running “line cost” for the Professional Provider. The running “line cost” will be pulled to the Cost Table in the Cost Module by Cost table algorithm for the Professional Provider.

### Professional Provider, Pre-Op Stage, Payment Per Case (A.1.d):

A.1.d After the “payment per case” payment type possibility is completed in “CE set up”, the cost for the Professional provider in the CE is complete. No further input is needed. The Direct Labor algorithm will display to the user this Professional Provider cost as the “line cost” for the Professional Provider in the CE. There is no running “line cost”. The Cost Table algorithm will pull this “line cost” to the Cost Table in the Cost Module.

### Professional Provider, Pre-Op Stage, Percentage of Professional Component (A.1.e):

A.1.e After the “percentage of professional component” payment type possibility is completed in “CE set up”, the cost for the Professional provider in the CE is complete. No further input is needed. Until the CE set up for “percentage of professional component” is complete the direct labor cost section for the Professional Provider will be INCOMPLETE. Once the CE setup has been completed, the Direct Labor algorithm will display to the user this Professional Provider cost as the “line cost” for the Professional Provider in the CE. There is no running “line cost”. The Cost Table algorithm will pull this “line cost” to the Cost Table in the Cost Module.

### Professional Provider, O.R. Stage (A.2):

A.2 In the O.R. stage, the Direct Labor algorithm allows for the input of time in minutes that the Professional Provider spends with the client in ALE only for payment types A.2.a, A.2.b and A.2.c described below. There is no PLE for the Professional Provider in the O.R. stage. Neither ALE nor PLE can be inputted into payment types A.2.d and A.2.e described below. Professional Providers do not have ACT.

### Professional Provider, O.R Stage, Payment Per Hour (A.2.a):

A.2.a After input of time for the “payment per hour” payment type possibility completed in “CE set up”, the Direct Labor algorithm will calculate and display to the user the cost for the Professional Provider in the O.R. stage. The Cost Table algorithm will pull this cost to the O.R. stage section of the Cost Table in the Cost Module.

The Direct Labor algorithm will also calculate and display to the user the “line cost” for the Professional Provider. The running “line cost” will be pulled to the Cost Table in the Cost Module by Cost Table algorithm for the Professional Provider.

### Professional Provider, O.R Stage, Salary (A.2.b):

A.2.b After input of time for the “salary” payment type possibility completed in “CE set up” where the payment is converted to the hourly rate, the Direct Labor algorithm will calculate and display to the user the cost for the Professional Provider in the O.R. stage. The Cost Table algorithm will pull this cost to the O.R. stage section of the Cost Table in the Cost Module.

The Direct Labor algorithm will also calculate and display to the user the “line cost” for the Professional Provider. The running “line cost” will be pulled to the Cost Table in the Cost Module by Cost Table algorithm for the Professional Provider.

### Professional Provider, O.R Stage, Payment Per Day (A.2.c):

A.2.c After input of time for the “payment per day” payment type possibility completed in “CE set up” where the payment is converted to the hourly rate, the Direct Labor algorithm will calculate and display to the user the cost for the Professional Provider in the O.R. stage. The Cost Table algorithm will pull this cost to the O.R. stage section of the Cost Table in the Cost Module.

The Direct Labor algorithm will also calculate and display to the user the “line cost” for the Professional Provider. The running “line cost” will be pulled to the Cost Table in the Cost Module by Cost Table algorithm for the Professional Provider.

### Professional Provider, O.R Stage, Payment Per Case (A.2.d):

A.2.d After the “payment per case” payment type possibility is completed in “CE set up”, the cost for the Professional provider in the CE is complete. No further input is needed. The Direct Labor algorithm will display to the user this Professional Provider cost as the “line cost” for the Professional Provider in the CE. There is no running “line cost”. The Cost Table algorithm will pull this “line cost” to the Cost Table in the Cost Module.

### Professional Provider, O.R Stage, Percentage of Professional Component (A.2.e):

A.2.e After the “percentage of professional component” payment type possibility is completed in “CE set up”, the cost for the Professional provider in the CE is complete. No further input is needed. Until the CE set up for “percentage of professional component” is complete the direct labor cost section for the Professional Provider will be INCOMPLETE. Once the CE set up has been completed the Direct Labor algorithm will display to the user this Professional Provider cost as the “line cost” for the Professional Provider in the CE. There is no running “line cost”. The Cost Table algorithm will pull this “line cost” to the Cost Table in the Cost Module.

### Professional Provider, Post-op #1 (A.3):

A.3 In the Post-op #1 stage, the Direct Labor algorithm allows for the input of time in minutes that the Professional Provider spends with the client in ALE only for payment types A.3.a, A.3.b and A.3.c described below. There is no PLE for the Professional Provider in the Post-op #1 stage. Neither ALE nor PLE can be inputted into payment types A.3.d and A.3.e described below. Professional Providers do not have ACT.

### Professional Provider, Post-Op #1, Payment Per Hour (A.3.a):

A.3.a After input of time for the “payment per hour” payment type possibility completed in “CE set up”, the Direct Labor algorithm will calculate and display to the user the cost for the Professional Provider in the Post-op #1 stage. The Cost Table algorithm will pull this cost to the Post-op #1 stage section of the Cost Table in the Cost Module. The Direct Labor algorithm will also calculate and display to the user the running “line cost” for the Professional Provider. The running “line cost” will be pulled to the Cost Table in the Cost Module by Cost table algorithm for the Professional Provider.

### Professional Provider, Post-op #1, Salary (A.3.b):

A.3.b After input of time for the “salary” payment type possibility completed in “CE set up” where the payment is converted to the hourly rate, the Direct Labor algorithm will calculate and display to the user the cost for the Professional Provider in the Post-op #1 stage. The Cost Table algorithm will pull this cost to the Post-op #1 stage section of the Cost Table in the Cost Module.

The Direct Labor algorithm will also calculate and display to the user the running “line cost” for the Professional Provider. The running “line cost” will be pulled to the Cost Table in the Cost Module by Cost table algorithm for the Professional Provider.

### Professional Provider, Post-Op #1, Payment Per Day (A.3.c):

A.3.c After input of time for the “payment per day” payment type possibility completed in “CE set up” where the payment is converted to the hourly rate, the Direct Labor algorithm will calculate and display to the user the cost for the Professional Provider in the Post-op #1 stage. The Cost Table algorithm will pull this cost to the Post-op #1 stage section of the Cost Table in the Cost Module.

The Direct Labor algorithm will also calculate and display to the user the running “line cost” for the Professional Provider. The running “line cost” will be pulled to the Cost Table in the Cost Module by Cost table algorithm for the Professional Provider.

### Professional Provider, Post-Op #1, Payment Per Case (A.3.d):

A.3.d After the “payment per case” payment type possibility is completed in “CE set up”, the cost for the Professional provider in the CE is complete. No further input is needed. The Direct Labor algorithm will display to the user this Professional Provider cost as the “line cost” for the Professional Provider in the CE. There is no running “line cost”. The Cost Table algorithm will pull this “line cost” to the Cost Table in the Cost Module.

### Professional Provider, Post-Op #1, Percentage of Professional Component (A.3.e):

A.3.e After the “percentage of professional component” payment type possibility is completed in “CE set up”, the cost for the Professional provider in the CE is complete. No further input is needed. Until the CE set up for “percentage of professional component” is complete the direct labor cost section for the Professional Provider will be INCOMPLETE. Once the CE setup has been completed, the Direct Labor algorithm will display to the user this Professional Provider cost as the “line cost” for the Professional Provider in the CE. There is no running “line cost”. The Cost Table algorithm will pull this “line cost” to the Cost Table in the Cost Module.

### Professional Provider, Post-op #2 (A.4):

A.4 In the Post-op #2 stage, the Direct Labor algorithm allows for the input of time in minutes that the Professional Provider spends with the client in ALE only for payment types A.3.a, A.3.b and A.3.c described below. There is no PLE for the Professional Provider in the Post-op #2 stage. Neither ALE nor PLE can be inputted into payment types A.3.d and A.3.e described below. Professional Providers do not have ACT.

### Professional Provider, Post-Op #2, Payment Per Hour (A.4.a):

A.4.a After input of time for the “payment per hour” payment type possibility completed in “CE set up”, the Direct Labor algorithm will calculate and display to the user the cost for the Professional Provider in the Post-op #2 stage. The Cost Table algorithm will pull this cost to the Post-op #2 stage section of the Cost Table in the Cost Module. The Direct Labor algorithm will also calculate and display to the user the running “line cost” for the Professional Provider. The running “line cost” will be pulled to the Cost Table in the Cost Module by Cost table algorithm for the Professional Provider.

### Professional Provider, Post-op #2, Salary (A.4.b):

A.4.b After input of time for the “salary” payment type possibility completed in “CE set up” where the payment is converted to the hourly rate, the Direct Labor algorithm will calculate and display to the user the cost for the Professional Provider in the Post-op #2 stage. The Cost Table algorithm will pull this cost to the Post-op #2 stage section of the Cost Table in the Cost Module. The Direct Labor algorithm will also calculate and display to the user the running “line cost” for the Professional Provider. The running “line cost” will be pulled to the Cost Table in the Cost Module by Cost table algorithm for the Professional Provider.

### Professional Provider, Post-Op #2, Payment Per Day (A.4.c):

A.4.c After input of time for the “payment per day” payment type possibility completed in “CE set up” where the payment is converted to the hourly rate, the Direct Labor algorithm will calculate and display to the user the cost for the Professional Provider in the Post-op #2 stage. The Cost Table algorithm will pull this cost to the Post-op #2 stage section of the Cost Table in the Cost Module. The Direct Labor algorithm will also calculate and display to the user the running “line cost” for the Professional Provider. The running “line cost” will be pulled to the Cost Table in the Cost Module by Cost table algorithm for the Professional Provider.

### Professional Provider, Post-Op #2, Payment Per Case (A.4.d):

A.4.d After the “payment per case” payment type possibility is completed in “CE set up”, the cost for the Professional provider in the CE is complete. No further input is needed. The Direct Labor algorithm will display to the user this Professional Provider cost as the “line cost” for the Professional Provider in the CE. There is no running “line cost”. The Cost Table algorithm will pull this “line cost” to the Cost Table in the Cost Module.

### Professional Provider, Post-Op #2, Percentage of Professional Component (A.4.e):

A.4.e After the “percentage of professional component” payment type possibility is completed in “CE set up”, the cost for the Professional provider in the CE is complete. No further input is needed. Until the CE set up for “percentage of professional component” is complete the direct labor cost section for the Professional Provider will be INCOMPLETE. Once the CE setup has been completed, the Direct Labor algorithm will display to the user this Professional Provider cost as the “line cost” for the Professional Provider in the CE. There is no running “line cost”. The Cost Table algorithm will pull this “line cost” to the Cost Table in the Cost Module.

### Professional Provider, AO Time (A.5):

A.5 In AO time, the Direct Labor algorithm allows for the input of time in minutes that the Professional Provider spends doing purely administrative work. The administrative work must occur outside the CE but the time to perform this work does not need to be consecutive. AO time input for payment types A.5.a, A.5.b and A.5.c are described below. Payment types A.5.d and A.5.e do not have input for AO time.

### Professional Provider, AO Time, Payment Per Hour (A.5.a):

A.5.a After input of the time for “payment per hour” payment type possibility completed in “CE set up”, the Direct Labor algorithm will calculate and display to the user the cost for the Professional Provider for the AO time. The Cost Table algorithm will pull this cost to the AO time section of the Cost Table in the Cost Module.

The Direct Labor algorithm will also calculate and display to the user the running “line cost” for the Professional Provider. The running “line cost” will be pulled to the Cost Table in the Cost Module by Cost table algorithm for the Professional Provider.

### Professional Provider, AO Time, Salary (A.5.b):

A.5.b After input of time for the “salary” payment type possibility completed in “CE set up” where the payment is converted to the hourly rate, the Direct Labor algorithm will calculate and display to the user the cost for the Professional Provider for the AO time. The Cost Table algorithm will pull this cost to the AO time section of the Cost Table in the Cost Module.

The Direct Labor algorithm will also calculate and display to the user the running “line cost” for the Professional Provider. The running “line cost” will be pulled to the Cost Table in the Cost Module by Cost table algorithm for the Professional Provider.

### Professional Provider, AO Time, Payment Per Day (A.5.c):

A.5.c After input of time for the “payment per day” payment type possibility completed in “CE set up” where the payment is converted to the hourly rate, the Direct Labor algorithm will calculate and display to the user the cost for the Professional Provider for the AO time. The Cost Table algorithm will pull this cost to the AO time section of the Cost Table in the Cost Module.

The Direct Labor algorithm will also calculate and display to the user the running “line cost” for the Professional Provider. The running “line cost” will be pulled to the Cost Table in the Cost Module by Cost table algorithm for the Professional Provider.

### Professional Provider, AO Time, Payment Per Case (A.5.d):

A.5.d After the “payment per case” payment type possibility is completed in “CE set up”, the cost for the Professional provider in the CE is complete. No further input is needed. The cost of AO time is included in the “payment per case”. The Direct Labor algorithm will display to the user this Professional Provider cost as the “line cost” for the Professional Provider in the CE. There is no running “line cost”. The Cost Table algorithm will pull this “line cost” to the Cost Table in the Cost Module.

### Professional Provider, AO Time, Percentage of Professional Component (A.5.e)

A.5.e After the “percentage of professional component” payment type possibility is completed in “CE set up”, the cost for the Professional provider in the CE is complete. No further input is needed. The cost of AO time is included in the “percentage of professional component” Until the CE set up for “percentage of professional component” is complete the direct labor cost section for the Professional Provider will be INCOMPLETE. Once the CE setup has been completed, the Direct Labor algorithm will display to the user this Professional Provider cost as the “line cost” for the Professional Provider in the CE. There is no running “line cost”. The Cost Table algorithm will pull this “line cost” to the Cost Table in the Cost Module.

### Team Member Direct Labor Cost:

### Team Member, Pre-Op Stage (B.1):

B.1 In the Pre-op stage, the Direct Labor algorithm allows for the input of time in minutes that a Team Member spends with the client in ALE and ACT. Team Members do not have PLE. The Direct Labor algorithm will calculate “ACT hourly rate” for a Team Member with input from the user. The user will input the number of other clients that are not part of the CE with which the Team Member is engaged at the same time they are engaged with the client in the CE. The hourly rate for the Team Member was pushed to the Cost Module from CE set up. The algorithm will divide the hourly rate for the Team Member from the “CE set up” by the number of clients in total the Team Member is engaged with during ACT in Pre-op stage to calculate the ACT hourly rate.

The hourly rate for ALE for Team Members is the hourly rate pushed from “CE set up” to the Cost Module.

### Team Member, Pre-Op Stage, Payment Per Hour (B.1.a):

B.1.a After input of time for the “payment per hour” payment type possibility completed in “CE set up”, the Direct Labor algorithm will add together the ALE and ACT costs for the Team Member and display to the user the cost for the Team Member in the Pre-op stage. The Cost Table algorithm will pull this cost to the Pre-op stage section of the Cost Table in the Cost Module.

The Direct Labor algorithm will also calculate and display to the user the running “line cost” for the Team Member. The running “line cost” will be pulled to the Cost Table in the Cost Module by the Cost table algorithm for the Team Member.

The running “line cost”, as it relates to all Players, is the running total of the cost for a Player in the CE and changes on the Cost Table as additional direct labor costs are added or removed for that Player. When the user has finished inputting all the direct labor costs for a Player, the running “line cost” is referred to as the “line cost” for that Player.

### Team Member, Pre-Op Stage, Salary (B.1.b):

B.1.b After input of time for the “salary” payment type possibility completed in “CE set up”, the Direct Labor algorithm will add together the ALE and ACT costs for the Team Member and display to the user the cost for the Team Member in the Pre-op stage. The Cost Table algorithm will pull this cost to the Pre-op stage section of the Cost Table in the Cost Module.

The Direct Labor algorithm will also calculate and display to the user the running “line cost” for the Team Member. The running “line cost” will be pulled to the Cost Table in the Cost Module by the Cost table algorithm for the Team Member.

### Team Member, Pre-Op Stage, Payment Per Day (B.1.c):

B.1.c After input of time for the “payment per day” payment type possibility completed in “CE set up”, the Direct Labor algorithm will add together the ALE and ACT costs for the Team Member and display to the user the cost for the Team Member in the Pre-op stage. The Cost Table algorithm will pull this cost to the Pre-op stage section of the Cost Table in the Cost Module. The Direct Labor algorithm will also calculate and display to the user the running “line cost” for the Team Member. The running “line cost” will be pulled to the Cost Table in the Cost Module by the Cost table algorithm for the Team Member.

### Team Member, O.R. (B.2):

B.2 In the O.R. stage, the Direct Labor algorithm allows for the input of time in minutes that a Team Member spends with the client in ALE. In the O.R. stage there is no ACT for Team Members and Team Members do not have PLE.

### Team Member, O.R., Payment Per Hour (B.2.a):

B.2.a After input of time for the “payment per hour” payment type possibility completed in “CE set up”, the Direct Labor algorithm will calculate and display to the user the cost for the Team Member in the O.R. stage. The Cost Table algorithm will pull this cost to the O.R. stage section of the Cost Table in the Cost Module. The Direct Labor algorithm will also calculate and display to the user the running “line cost” for the Team Member. The running “line cost” will be pulled to the Cost Table in the Cost Module by the Cost table algorithm for the Team Member.

### Team Member, O.R., Salary (B.2.b):

B.2.b After input of time for the “salary” payment type possibility completed in “CE set up” where the payment is converted to the hourly rate, the Direct Labor algorithm will calculate and display to the user the cost for the Team Member in the O.R. stage. The Cost Table algorithm will pull this cost to the O.R. stage section of the Cost Table in the Cost Module. The Direct Labor algorithm will also calculate and display to the user the running “line cost” for the Team Member. The running “line cost” will be pulled to the Cost Table in the Cost Module by the Cost table algorithm for the Team Member.

### Team Member, O.R., Payment Per Day (B.2.c):

B.2.c After input of time for the “payment per day” payment type possibility completed in “CE set up” where the payment is converted to the hourly rate, the Direct Labor algorithm will calculate and display to the user the cost for the Team Member in the O.R. stage. The Cost Table algorithm will pull this cost to the O.R. stage section of the Cost Table in the Cost Module. The Direct Labor algorithm will also calculate and display to the user the running “line cost” for the Team Member. The running “line cost” will be pulled to the Cost Table in the Cost Module by the Cost table algorithm for the Team Member.

### Team Member, Post-Op #1 (B.3):

B.3 In the Post-op #1 stage, the Direct Labor algorithm allows for the input of time in minutes that a Team Member spends with the client in ALE and ACT. Team Members do not have PLE. The Direct Labor algorithm will calculate “ACT hourly rate” for a Team Member with input from the user. The user will input the number of other clients that are not part of the CE with which the Team Member is engaged at the same time they are engaged with the client in the CE. The hourly rate for the Team Member was pushed to the Cost Module from CE set up. The algorithm will divide the hourly rate for the Team Member from the “CE set up” by the number of clients in total the Team Member is engaged with during ACT in Post-op #1 stage to calculate the ACT hourly rate.

The hourly rate for ALE for Team Members is the hourly rate pushed from “CE set up” to the Cost Module.

### Team Member, Post-Op #1, Payment Per Hour (B.3.a):

B.3.a After input of time for the “payment per hour” payment type possibility completed in “CE set up”, the Direct Labor algorithm will add together the ALE and ACT costs for the Team Member and display to the user the cost for the Team Member in the Post-op #1 stage. The Cost Table algorithm will pull this cost to the Post-op #1 stage section of the Cost Table in the Cost Module. The Direct Labor algorithm will also calculate and display to the user the running “line cost” for the Team Member. The running “line cost” will be pulled to the Cost Table in the Cost Module by the Cost table algorithm for the Team Member.

### Team Member, Post-Op #1, Salary (B.3.b):

B.3.b After input of time for the “salary” payment type possibility completed in “CE set up”, the Direct Labor algorithm will add together the ALE and ACT costs for the Team Member and display to the user the cost for the Team Member in the Post-op #1 stage. The Cost Table algorithm will pull this cost to the Post-op #1 stage section of the Cost Table in the Cost Module. The Direct Labor algorithm will also calculate and display to the user the running “line cost” for the Team Member. The running “line cost” will be pulled to the Cost Table in the Cost Module by the Cost table algorithm for the Team Member.

### Team Member, Post-Op #1, Payment Per Day (B.3.c):

B.3.c After input of time for the “payment per day” payment type possibility completed in “CE set up”, the Direct Labor algorithm will add together the ALE and ACT costs for the Team Member and display to the user the cost for the Team Member in the Post-op #1 stage. The Cost Table algorithm will pull this cost to the Post-op #1 stage section of the Cost Table in the Cost Module. The Direct Labor algorithm will also calculate and display to the user the running “line cost” for the Team Member. The running “line cost” will be pulled to the Cost Table in the Cost Module by the Cost table algorithm for the Team Member.

### Team Member, Post-Op #2 (B.4):

B.4 In the Post-op #2 stage, the Direct Labor algorithm allows for the input of time in minutes that a Team Member spends with the client in ALE and ACT. Team Members do not have PLE. The Direct Labor algorithm will calculate “ACT hourly rate” for a Team Member with input from the user. The user will input the number of other clients that are not part of the CE with which the Team Member is engaged at the same time they are engaged with the client in the CE. The hourly rate for the Team Member was pushed to the Cost Module from CE set up. The algorithm will divide the hourly rate for the Team Member from the “CE set up” by the number of clients in total the Team Member is engaged with during ACT Post-op #2 stage to calculate the ACT hourly rate.

The hourly rate for ALE for Team Members is the hourly rate pushed from “CE set up” to the Cost Module.

### Team Member, Post-Op #2, Payment Per Hour (B.4.a):

B.4.a After input of time for the “payment per hour” payment type possibility completed in “CE set up”, the Direct Labor algorithm will add together the ALE and ACT costs for the Team Member and display to the user the cost for the Team Member in the Post-op #2 stage. The Cost Table algorithm will pull this cost to the Post-op #2 stage section of the Cost Table in the Cost Module. The Direct Labor algorithm will also calculate and display to the user the running “line cost” for the Team Member. The running “line cost” will be pulled to the Cost Table in the Cost Module by the Cost table algorithm for the Team Member.

### Team Member, Post-Op #2, Salary (B.4.b):

B.4.b After input of time for the “salary” payment type possibility completed in “CE set up”, the Direct Labor algorithm will add together the ALE and ACT costs for the Team Member and display to the user the cost for the Team Member in the Post-op #2 stage. The Cost Table algorithm will pull this cost to the Post-op #2 stage section of the Cost Table in the Cost Module. The Direct Labor algorithm will also calculate and display to the user the running “line cost” for the Team Member. The running “line cost” will be pulled to the Cost Table in the Cost Module by the Cost table algorithm for the Team Member.

### Team Member, Post-Op #2, Payment Per Day (B.4.c):

B.4.c After input of time for the “payment per day” payment type possibility completed in “CE set up”, the Direct Labor algorithm will add together the ALE and ACT costs for the Team Member and display to the user the cost for the Team Member in the Post-op #2 stage. The Cost Table algorithm will pull this cost to the Post-op #2 stage section of the Cost Table in the Cost Module. The Direct Labor algorithm will also calculate and display to the user the running “line cost” for the Team Member. The running “line cost” will be pulled to the Cost Table in the Cost Module by the Cost table algorithm for the Team Member.

### Team Member, AO Time (B.5):

B.5 In AO time, the Direct Labor algorithm allows for the input of time in minutes that the Team Member spends doing purely administrative work. The administrative work must occur outside the CE but the time to perform this work does not need to be consecutive. AO time input for payment types B.5.a, B.5.b and B.5.c are described below.

### Team Member, AO Time, Payment Per Hour (B.5.a):

B.5.a After input of the time for “payment per hour” payment type possibility completed in “CE set up”, the Direct Labor algorithm will calculate and display to the user the cost for the Team Member for the AO time. The Cost Table algorithm will pull this cost to the AO time section of the Cost Table in the Cost Module. The Direct Labor algorithm will also calculate and display to the user the running “line cost” for the Team Member. The running “line cost” will be pulled to the Cost Table in the Cost Module by the Cost table algorithm for the Team Member.

### Team Members, AO Time, Salary (B.5.b):

B.5.b After input of time for the “salary” payment type possibility completed in “CE set up” where the payment is converted to the hourly rate, the Direct Labor algorithm will calculate and display to the user the cost for the Team Members for the AO time. The Cost Table algorithm will pull this cost to the AO time section of the Cost Table in the Cost Module. The Direct Labor algorithm will also calculate and display to the user the running “line cost” for the Team Member. The running “line cost” will be pulled to the Cost Table in the Cost Module by the Cost table algorithm for the Team Member.

### Team Members, AO Time, Payment Per Day (B.5.c):

B.5.c After input of time for the “payment per day” payment type possibility completed in “CE set up” where the payment is converted to the hourly rate, the Direct Labor algorithm will calculate and display to the user the cost for the Team Members for the AO time. The Cost Table algorithm will pull this cost to the AO time section of the Cost Table in the Cost Module. The Direct Labor algorithm will also calculate and display to the user the running “line cost” for the Team Member. The running “line cost” will be pulled to the Cost Table in the Cost Module by the Cost table algorithm for the Team Member.

### Anesthesia Provider Direct Labor Cost:

As discussed elsewhere, different types of Players have different possible kinds of time in the different stages of the CE. Unlike other Players, however, the Anesthesia Providers' job description when acting alone or in an allowed combination also has different possible kinds of time in the different stages of CE. The job descriptions for the Anesthesia Providers are MDA, MDA-Supervising, MDA-Directing CRNA, CRNA-Supervised, CRNA-Directed, AA-Supervised, AA-Directed. The allowed Anesthesia Providers job descriptions acting in combination or acting alone are outlined in CE set up section 1.A.3.

An Anesthesia Provider with any job description can only be engaged with the client in Pre-op stage, O.R. stage, Post-op #1 stage. No Anesthesia Provider is engaged with the client in Post-op #2 stage by definition. Allocation of Anesthesia Provider time to Post-op #2 is prohibited.

The time period inputted for Post-op #1 using the CE Set Up algorithm must be the same as the time period inputted for the Anesthesia Provider in ALE in post-op #1 using the Direct Labor algorithm. The time period inputted using CE Set Up algorithm in Post-op #1 is pushed to time in minutes that the Anesthesia Provider in ALE spends with the client in Post-op #1.

### Anesthesia Provider, Pre-Op Stage, MDA Acting Alone (C.1-m):

C.1-m In the Pre-op stage the Direct Labor algorithm allows for the input of time in minutes that the MDA acting alone spends with the client in ALE and PLE for payment types C.1-m.a, C.1-m.b and C.1-m.c described below. ALE and PLE cannot be inputted into payment types C.1-m.d and C.1-m.e described below. MDA acting alone does not have ACT. For MDA acting alone, the cost per hour for ALE and PLE is the same.

### Anesthesia Provider, Pre-Op Stage, CRNA Acting Alone (C.1-c):

C.1-c In the Pre-op stage the Direct Labor algorithm allows for the input of time in minutes that the CRNA acting alone spends with the client in ALE and PLE for payment types C.1-c.a, C.1-c.b and C.1-c.c described below. ALE and PLE cannot be inputted into payment types C.1-c.d and C.1-c.e described below. CRNA acting alone does not have ACT. For CRNA acting alone, the cost per hour for ALE and PLE is the same.

### Anesthesia Provider, Pre-Op Stage, MDA Acting Alone, Payment Per Hour (C.1-m.a):

Anesthesia Provider, Pre-Op stage, CRNA Acting Alone, Payment Per Hour (C.1-c.a):

C.1-m.a & C.1-c.a After input of the time for “payment per hour” payment type possibility completed in CE set up, the Direct Labor algorithm will calculate and display to the user the cost for the MDA acting alone or CRNA acting alone in the Pre-op stage. The Cost Table algorithm will pull this cost to the Pre-op stage section of the Cost Table in the Cost Module. The Direct Labor algorithm will also calculate and display to the user the “line cost” for the MDA acting alone or CRNA acting alone. The line cost will also be pulled by the Cost Table algorithm to the Cost Table in the Cost Module for the MDA acting alone or CRNA acting alone.

### Anesthesia Provider, Pre-Op Stage, MDA Acting Alone, Salary (C.1-m.b):

Anesthesia Provider, Pre-Op stage, CRNA Acting Alone, Salary (C.1-c.b):

C.1-m.b & C.1-c.b After input of time for the “salary” payment type possibility completed in “CE set up” where the payment is converted to the hourly rate, the Direct Labor algorithm will calculate and display to the user the cost for the MDA acting alone or CRNA acting alone in the Pre-op stage. The Cost Table algorithm will pull this cost to the Pre-op stage section of the Cost Table in the Cost Module. The Direct Labor algorithm will also calculate and display to the user the “line cost” for the MDA acting alone or CRNA acting alone. The line cost will also be pulled by Cost Table algorithm to the Cost Table in the Cost Module for the MDA acting alone or CRNA acting alone.

### Anesthesia Provider, Pre-Op Stage, MDA Acting Alone, Payment Per Day (C.1-m.c):

Anesthesia Provider, Pre-Op stage, CRNA Acting Alone, Payment Per Day (C.1-c.c):

C.1-m.c & C.1-c.c After input of time for the “payment per day” payment type possibility completed in “CE set up” where the payment is converted to the hourly rate, the Direct Labor algorithm will calculate and display to the user the cost for the MDA acting alone or CRNA acting alone in the Pre-op stage. The Cost Table algorithm will pull this cost to the Pre-op stage section of the Cost Table in the Cost Module. The Direct Labor algorithm will also calculate and display to the user the “line cost” for the MDA acting alone or CRNA acting alone. The line cost will also be pulled by Cost Table algorithm to Cost Table in the Cost Module for the MDA acting alone or CRNA acting alone.

### Anesthesia Provider, Pre-Op Stage, MDA Acting Alone, Payment Per Case (C.1-m.d):

Anesthesia Provider, Pre-Op stage, CRNA Acting Alone, Payment Per Case (C.1-c.d):

C.1-m.d & C.1-c.d After the “payment per case” payment type possibility is completed in “CE set up”, the cost for the MDA acting alone or CRNA acting alone in the CE is complete. No further input is needed. The Direct Labor algorithm will display this cost to the user as the line cost and Cost Table algorithm will pull this line cost to the Cost Table in the Cost Module.

### Anesthesia Provider, Pre-Op Stage, MDA Acting Alone, Percentage of Anesthesia Component (C.1-m.c):

Anesthesia Provider, Pre-Op stage, CRNA Acting Alone, Percentage of Anesthesia Component (C.1-c.c):

C.1-m.e & C.1-c.e After the “percentage of anesthesia component” payment type possibility is completed in “CE set up”, the cost for the MDA acting alone or CRNA acting alone in the CE is complete. No further input is needed. Until the CE set up for “percentage of anesthesia component” is complete, the direct labor cost section for the MDA acting alone or CRNA acting alone will be INCOMPLETE. Once the CE set up has been completed, the Direct Labor algorithm will display to the user this Anesthesia Provider cost as the “line cost” for the Anesthesia Provider in the CE. There is no running “line cost”. The Cost Table algorithm will pull this “line cost” to the Cost Table in the Cost Module.

### Anesthesia Provider, O.R Stage, MDA Acting Alone (C.2-m):

C.2-m In the O.R. stage, the Direct Labor algorithm allows for the input of time in minutes that the MDA acting alone spends with the client in ALE only for payment types C.2-m.a, C.2-m.b and C.2-m.c described below. There is no PLE for the MDA acting alone in the O.R. stage. Neither ALE nor PLE can be inputted into payment types C.2-m.d and C.2-m.e described below. MDA acting alone does not have ACT.

### Anesthesia Provider, O.R Stage, CRNA Acting Alone (C.2-c):

C.2-c In the O.R. stage, the Direct Labor algorithm allows for the input of time in minutes that the CRNA acting alone spends with the client in ALE only for payment types C.2-c.a, C.2-c.b and C.2-c.c described below. There is no PLE for the CRNA acting alone in the O.R. stage. Neither ALE nor PLE can be inputted into payment types C.2-c.d and C.2-c.e described below. CRNA acting alone does not have ACT.

### Anesthesia Provider, O.R. Stage, MDA Acting Alone, Payment Per Hour (C.2-m.a):

Anesthesia Provider, O.R. stage, CRNA Acting Alone, Payment Per Hour (C.2-c.a):

C.2-m.a & C.2-c.a After input of time for the “payment per hour” payment type possibility completed in CE set up, the Direct Labor algorithm will calculate and display to the user the cost for the MDA acting alone or CRNA acting alone in the O.R. stage. The Cost Table algorithm will pull this cost to the O.R stage section of the Cost Table in the Cost Module. The Direct Labor algorithm will also calculate and display to the user the running “line cost” for the MDA acting alone or CRNA acting alone. The running “line cost” will be pulled to the Cost Table in the Cost Module by Cost table algorithm for the MDA acting alone or CRNA acting alone.

### Anesthesia Provider, O.R. Stage, MDA Acting Alone, Salary (C.2-m.b):

Anesthesia Provider, O.R. stage, CRNA Acting Alone, Salary (C.2-c.b):

C.2-m.b & C.2-c.b After input of time for the “salary” payment type possibility completed in “CE set up” where the payment is converted to the hourly rate, the Direct Labor algorithm will calculate and display to the user the cost for the MDA acting alone or CRNA acting alone in the O.R. stage. The Cost Table algorithm will pull this cost to the O.R stage section of the Cost Table in the Cost Module. The Direct Labor algorithm will also calculate and display to the user the running “line cost” for the MDA acting alone or CRNA acting alone. The running “line cost” will be pulled to the Cost Table in the Cost Module by Cost table algorithm for the MDA acting alone or CRNA acting alone.

### Anesthesia Provider, O.R. Stage, MDA Acting Alone, Payment Per Day (C.2-m.c):

Anesthesia Provider, O.R. stage, CRNA Acting Alone, Payment Per Day (C.2-c.c):

C.2-m.c & C.2-c.c After input of time for the “payment per day” payment type possibility completed in “CE set up” where the payment is converted to the hourly rate, the Direct Labor algorithm will calculate and display to the user the cost for the MDA acting alone or CRNA acting alone in the O.R. stage. The Cost Table algorithm will pull this cost to the O.R stage section of the Cost Table in the Cost Module. The Direct Labor algorithm will also calculate and display to the user the running “line cost” for the MDA acting alone or CRNA acting alone. The running “line cost” will be pulled to the Cost Table in the Cost Module by Cost table algorithm for the MDA acting alone or CRNA acting alone.

### Anesthesia Provider, O.R. Stage, MDA Acting Alone, Payment Per Case (C.2-m.d):

Anesthesia Provider, O.R. stage, CRNA Acting Alone, Payment Per Case (C.2-c.d):

C.2-m.d & C.2-c.d After the “payment per case” payment type possibility is completed in “CE set up”, the cost for the MDA acting alone or CRNA acting alone in the CE is complete. No further input is needed. The Direct Labor algorithm will display this cost to the user as the “line cost”. There is no running “line cost”. The Cost Table algorithm will pull this “line cost” to the Cost Table in the Cost Module.

### Anesthesia Provider, O.R. Stage, MDA Acting Alone, Percentage of Anesthesia Component (C.2-m.e):

Anesthesia Provider, O.R. stage, CRNA Acting Alone, Percentage of Anesthesia Component (C.2-c.e):

C.2-m.e & C.2-c.e After the “percentage of anesthesia component” payment type possibility is completed in “CE set up”, the cost for the MDA acting alone or CRNA acting alone in the CE is complete. No further input is needed. Until the CE set up for “percentage of anesthesia component” is complete the direct labor cost section for the MDA acting alone or CRNA acting alone will be INCOMPLETE. Once the CE setup has been completed, the Direct Labor algorithm will display to the user this Anesthesia Provider cost as the “line cost” for the Anesthesia Provider in the CE. There is no running “line cost”. The Cost Table algorithm will pull this “line cost” to the Cost Table in the Cost Module. Once the CE set up has been completed, the Direct Labor algorithm will display to the user this cost as the “line cost”. The Cost Table algorithm will pull this “line cost” to the Cost Table in the Cost Module.

### Anesthesia Provider, Post-Op #1 Stage, MDA Acting Alone (C.3-m):

C.3-m In the Post-op #1 stage, the Direct Labor algorithm allows for the input of time in minutes that the MDA acting alone spends with the client in ALE only for payment types C.3-m.a, C.3-m.b and C.3-m.c described below. There is no PLE for the MDA acting alone in the Post-op #1 stage. Neither ALE nor PLE can be inputted into payment types C.3-m.d and C.3-m.e described below. MDA acting alone does not have ACT.

### Anesthesia Provider, Post-Op #1 Stage, CRNA Acting Alone (C.3-c):

C.3-c In the Post-op #1 stage, the Direct Labor algorithm allows for the input of time in minutes that the CRNA acting alone spends with the client in ALE only for payment types C.3-c.a, C.3-c.b and C.3-c.c described below. There is no PLE for the CRNA acting alone in the Post-op #1 stage. Neither ALE nor PLE can be inputted into payment types C.3-c.d and C.3-c.e described below. CRNA acting alone does not have ACT.

### Anesthesia Provider, Post-Op #1 Stage, MDA Acting Alone, Payment Per Hour (C.3-m.a):

Anesthesia Provider, Post-Op #1 stage, CRNA Acting Alone, Payment Per Hour (C.3-c.a):

C.3-m.a & C.3-c.a After input of time for the “payment per hour” payment type possibility completed in “CE set up”, the “Direct Labor” algorithm will calculate and display to the user the cost for the MDA acting alone or CRNA acting alone in the Post-op #1 stage. The Cost Table algorithm will pull this cost to the Post-op #1 stage section of the Cost Table in the Cost Module. The Direct Labor algorithm will also calculate and display to the user the running “line cost” for the MDA acting alone or CRNA acting alone. The running “line cost” will be pulled to the Cost Table in the Cost Module by Cost table algorithm for the MDA acting alone or CRNA acting alone.

### Anesthesia Provider, Post-Op #1 Stage, MDA Acting Alone, Salary (C.3-m.b):

Anesthesia Provider, Post-Op #1 stage, CRNA Acting Alone, Salary (C.3-c.b):

C.3-m.b & C.3-c.b After input of time for the “salary” payment type possibility completed in “CE set up” where the payment is converted to the hourly rate, the Direct Labor algorithm will calculate and display to the user the cost for the MDA acting alone or CRNA acting alone in the Post-op #1 stage.. The Cost Table algorithm will pull this cost to the Post-op #1 stage section of the Cost Table in the Cost Module. The Direct Labor algorithm will also calculate and display to the user the running “line cost” for the MDA acting alone or CRNA acting alone. The running “line cost” will be pulled to the Cost Table in the Cost Module by Cost table algorithm for the MDA acting alone or CRNA acting alone.

### Anesthesia Provider, Post-Op #1 Stage, MDA Acting Alone, Payment Per Day (C.3-m.c):

Anesthesia Provider, Post-Op #1 stage, CRNA Acting Alone, Payment Per Day (C.3-c.c):

C.3-m.c & C.3-c.c After input of time for the “payment per day” payment type possibility completed in “CE set up” where the payment is converted to the hourly rate, the Direct Labor algorithm will calculate and display to the user the cost for the MDA acting alone or CRNA acting alone in the Post-op #1 stage. The Cost Table algorithm will pull this cost to the Post-op #1 stage section of the Cost Table in the Cost Module. The Direct Labor algorithm will also calculate and display to the user the running “line cost” for the MDA acting alone or CRNA acting alone. The running “line cost” will be pulled to the Cost Table in the Cost Module by Cost table algorithm for the MDA acting alone or CRNA acting alone.

### Anesthesia Provider, Post-Op #1 Stage, MDA Acting Alone, Payment Per Case (C.3-m.d):

Anesthesia Provider, Post-Op #1 stage, CRNA Acting Alone, Payment Per Case (C.3-c.d):

C.3-m.d & C.3-c.d After the “payment per case” payment type possibility is completed in “CE set up”, the cost for the MDA acting alone or CRNA acting alone in the CE is complete. No further input is needed. The Direct Labor algorithm will display this cost to the user as the “line cost”. There is no running “line cost”. The Cost Table algorithm will pull this “line cost” to the Cost Table in the Cost Module.

### Anesthesia Provider, Post-Op #1 Stage, MDA Acting Alone, Percentage of Anesthesia Component (C.3-m.e):

Anesthesia Provider, Post-Op #1 stage, CRNA Acting Alone, Percentage of Anesthesia Component (C.3-c.e):

C.3-m.e & C.3-c.e After the “percentage of anesthesia component” payment type possibility is completed in “CE set up”, the cost for the MDA acting alone or CRNA acting alone in the CE is complete. No further input is needed. Until the CE set up for “percentage of anesthesia component” is complete the direct labor cost section for the MDA acting alone or CRNA acting alone will be INCOMPLETE. Once the CE setup has been completed, the Direct Labor algorithm will display to the user this MDA acting alone or CRNA acting alone cost as the “line cost” for the MDA acting alone or CRNA acting alone in the CE. There is no running “line cost”. The Cost Table algorithm will pull this “line cost” to the Cost Table in the Cost Module.

Anesthesia Provider, Pre-Op Stage MDA-Supervising (C.1-ms):

C.1-ms In the Pre-op stage, the Direct Labor algorithm allows for the input of time in minutes that the MDA-Supervising spends with the client in ALE and ACT for payment types C.1-ms.a, C.1-ms.b and C.1-ms.c described below. ALE and ACT cannot be inputted into payment types C.1-ms.d and C.1-ms.e described below. MDA-Supervising does not have PLE. The Direct Labor algorithm will calculate “ACT hourly rate” for a MDA-Supervising with input from the user. The user will input the number of other clients that are not part of the CE with which the MDA-Supervising is engaged at the same time they are engaged with the client in the CE. The hourly rate for the MDA-Supervising was pushed to the Cost Module from CE set up. The algorithm will divide the hourly rate for the MDA-Supervising from the “CE set up” by the number of clients in total the MDA-Supervising is engaged with during ACT in Pre-op stage to calculate the ACT hourly rate.

The hourly rate for ALE for MDA-Supervising is the hourly rate pushed from “CE set up” to the Cost Module.

Anesthesia Provider, Pre-Op Stage MDA-Directing (C.1-md):

C1-md In the Pre-op stage, the Direct Labor algorithm allows for the input of time in minutes that the MDA-Directing spends with the client in ALE and ACT for payment types C.1-md.a, C.1-md.b and C.1-md.c described below. ALE and ACT cannot be inputted into payment types C.1-md.d and C.1-md.e described below. MDA-Directing does not have PLE. The Direct Labor algorithm will calculate “ACT hourly rate” for an MDA-Directing with input from the user. The user will input the number of other clients that are not part of the CE with which the MDA-Directing is engaged at the same time they are engaged with the client in the CE. The hourly rate for the MDA-Directing was pushed to the Cost Module from CE set up. The algorithm will divide the hourly rate for the MDA-Directing from the “CE set up” by the number of clients in total the MDA-Directing is engaged with during ACT in Pre-op stage to calculate the ACT hourly rate.

The hourly rate for ALE for MDA-Directing is the hourly rate pushed from “CE set up” to the Cost Module.

### Anesthesia Provider, Pre-Op Stage MDA-Supervising, Payment Per Hour (C.1-ms.a):

Anesthesia Provider, Pre-Op stage MDA-Directing, Payment Per Hour (C.1-md.a):

C.1-ms.a & C.1-md.a After input of time for the “payment per hour” payment type possibility completed in CE set up, the Direct Labor algorithm will add together the ALE and ACT costs for the MDA-Supervising or MDA-Directing and display to the user the cost for the MDA-Supervising or MDA-Directing in the Pre-op stage. The Cost Table algorithm will pull this cost to the Pre-op stage section of the Cost Table in the Cost Module. The Direct Labor algorithm will also calculate and display to the user the running “line cost” for the MDA-Supervising and MDA-Directing. The running “line cost” will be pulled to the Cost Table in the Cost Module by Cost table algorithm for the for the MDA-Supervising and MDA-Directing.

### Anesthesia Provider, Pre-Op Stage MDA-Supervising, Salary (C.1-ms.b):

Anesthesia Provider, Pre-Op stage MDA-Directing, Salary (C.1-md.b):

C.1-ms.b & C.1-md.b After input of time for the “salary” payment type possibility completed in CE set up, the Direct Labor algorithm will add together the ALE and ACT costs for the MDA-Supervising or MDA-Directing and display to the user the cost for the MDA-Supervising or MDA-Directing in the Pre-op stage. The Cost Table algorithm will pull this cost to the Pre-op stage section of the Cost Table in the Cost Module. The Direct Labor algorithm will also calculate and display to the user the running “line cost” for the MDA-Supervising and MDA-Directing. The running “line cost” will be pulled to the Cost Table in the Cost Module by Cost table algorithm for the for the MDA-Supervising and MDA-Directing.

### Anesthesia Provider, Pre-Op Stage MDA-Supervising, Payment Per Day (C.1-ms.c):

Anesthesia Provider, Pre-Op stage MDA-Directing, Payment Per Day (C.1-md.c):

C.1-ms.c & C.1-md.c After input of time for the “payment per day” payment type possibility completed in CE set up, the Direct Labor algorithm will add together the ALE and ACT costs for the MDA-Supervising or MDA-Directing and display to the user the cost for the MDA-Supervising or MDA-Directing in the Pre-op stage. The Cost Table algorithm will pull this cost to the Pre-op stage section of the Cost Table in the Cost Module. The Direct Labor algorithm will also calculate and display to the user the running “line cost” for the MDA-Supervising and MDA-Directing. The running “line cost” will be pulled to the Cost Table in the Cost Module by Cost table algorithm for the for the MDA-Supervising and MDA-Directing.

### Anesthesia Provider, Pre-Op Stage MDA-Supervising, Payment Per Case (C.1-ms.d):

Anesthesia Provider, Pre-Op stage MDA-Directing, Payment Per Case (C.1-md.d):

C.1-ms.d & C.1-md.d After the “payment per case” payment type possibility is completed in “CE set up”, the cost for the MDA-Supervising or MDA-Directing in the CE is complete. No further input is needed. The Direct Labor algorithm will display this cost to the user as the “line cost”. There is no running “line cost”. The Cost Table algorithm will pull this “line cost” to the Cost Table in the Cost Module.

### Anesthesia Provider, Pre-Op Stage MDA-Supervising, Percentage of Anesthesia Component (C.1-ms.e):

Anesthesia Provider, Pre-Op stage MDA-Directing, Percentage of Anesthesia Component (C.1-md.e):

C.1-ms.e & C.1-md.e After the “percentage of anesthesia component” payment type possibility is completed in “CE set up”, the cost for the MDA-Supervising or MDA-Directing in the CE is complete. No further input is needed. Until the CE set up for “percentage of anesthesia component” is complete the direct labor cost section for the MDA-Supervising or MDA-Directing will be INCOMPLETE. Once the CE setup has been completed, the Direct Labor algorithm will display to the user this MDA-Supervising and MDA-Directing cost as the “line cost” for the MDA-Supervising and MDA-Directing in the CE. There is no running “line cost”. The Cost Table algorithm will pull this “line cost” to the Cost Table in the Cost Module.

Anesthesia Provider, O.R. Stage, MDA-Supervising (C.2-ms):

C.2-ms In the O.R. stage, the Direct Labor algorithm allows for the input of time in minutes that the MDA-Supervising spends with the client in ALE and ACT for payment types C.2-ms.a, C.2-ms.b and C.2-ms.c described below. ALE and ACT cannot be inputted into payment types C.2-ms.d and C.2-ms.e described below. MDA-Supervising does not have PLE. As above, the “Direct Labor” algorithm will calculate “ACT hourly rate” for an MDA-Supervising with input from the user.

The hourly rate for ALE for MDA-Supervising is the hourly rate pushed from “CE set up” to the Cost Module.

Anesthesia Provider, O.R. Stage, MDA-Directing (C.2-md):

C.2-md In the O.R. stage, the Direct Labor algorithm allows for the input of time in minutes that the MDA-Supervising spends with the client in ALE and ACT for payment types C.2-md.a, C.2-md.b and C.2-md.c described below. ALE and ACT cannot be inputted into payment types C.2-md.d and C.2-md.e described below. MDA-Supervising does not have PLE. As above, the Direct Labor algorithm will calculate “ACT hourly rate” for an MDA-Supervising with input from the user.

The hourly rate for ALE for MDA-Supervising is the hourly rate pushed from “CE set up” to the Cost Module.

### Anesthesia Provider, O.R. Stage MDA-Supervising, Payment Per Hour (C.2-ms.a):

Anesthesia Provider, O.R. stage MDA-Directing, Payment Per Hour (C.2-md.a):

C.2-ms.a & C.2-md.a After input of time for the “payment per hour” payment type possibility completed in “CE set up”, the Direct Labor algorithm will add together the ALE and ACT costs for the MDA-Supervising or MDA-Directing and display to the user the cost for the MDA-Supervising or MDA-Directing in the O.R. stage. The Cost Table algorithm will pull this cost to the O.R stage section of the Cost Table in the Cost Module. The Direct Labor algorithm will also calculate and display to the user the running “line cost” for the MDA-Supervising and MDA-Directing. The running “line cost” will be pulled to the Cost Table in the Cost Module by Cost table algorithm for the for the MDA-Supervising and MDA-Directing.

### Anesthesia Provider, O.R. Stage, MDA-Supervising, Salary (C.2-ms.b):

Anesthesia Provider, O.R. stage, MDA-Directing, Salary (C.2-md.b):

C.2-ms.b & C.2-md.b After input of time for the “salary” payment type possibility completed in “CE set up”, the Direct Labor algorithm will add together the ALE and ACT costs for the MDA-Supervising or MDA-Directing and display to the user the cost for the MDA-Supervising or MDA-Directing in the O.R. stage. The Cost Table algorithm will pull this cost to the O.R stage section of the Cost Table in the Cost Module. The Direct Labor algorithm will also calculate and display to the user the running “line cost” for the MDA-Supervising and MDA-Directing. The running “line cost” will be pulled to the Cost Table in the Cost Module by Cost table algorithm for the for the MDA-Supervising and MDA-Directing.

### Anesthesia Provider, O.R. Stage, MDA-Supervising, Payment Per Day (C.2-ms.c):

Anesthesia Provider, O.R. stage, MDA-Directing, Payment Per Day (C.2-md.c):

C.2-ms.c & C.2-md.c After input of time for the “payment per day” payment type possibility completed in “CE set up”, the Direct Labor algorithm will add together the ALE and ACT costs for the MDA-Supervising or MDA-Directing and display to the user the cost for the MDA-Supervising or MDA-Directing in the O.R. stage. The Cost Table algorithm will pull this cost to the O.R stage section of the Cost Table in the Cost Module. The Direct Labor algorithm will also calculate and display to the user the running “line cost” for the MDA-Supervising and MDA-Directing. The running “line cost” will be pulled to the Cost Table in the Cost Module by Cost table algorithm for the for the MDA-Supervising and MDA-Directing.

### Anesthesia Provider, O.R. Stage, MDA-Supervising, Payment Per Case (C.2-ms.d):

Anesthesia Provider, O.R. stage, MDA-Directing, Payment Per Case (C.2-md.d):

C.2-ms.d & C.2-md.d After the “payment per case” payment type possibility is completed in “CE set up”, the cost for the MDA-Supervising or MDA-Directing in the CE is complete. No further input is needed. The Direct Labor algorithm will display this cost to the user as the “line cost”. There is no running “line cost”. The Cost Table algorithm will pull this “line cost” to the Cost Table in the Cost Module.

### Anesthesia Provider, O.R. Stage, MDA-Supervising, Percentage of Anesthesia Component (C.2-ms.e):

Anesthesia Provider, O.R. stage, MDA-Directing, Percentage of anesthesia Component (C.2-md.e):

C.2-ms.e & C.2-md.e After the “percentage of anesthesia component” payment type possibility is completed in “CE set up”, the cost for the MDA-Supervising or MDA-Directing in the CE is complete. No further input is needed. Until the CE set up for “percentage of anesthesia component” is complete the direct labor cost section for the MDA-Supervising or MDA-Directing will be INCOMPLETE. Once the CE setup has been completed, the Direct Labor algorithm will display to the user this MDA-Supervising and MDA-Directing cost as the “line cost” for the MDA-Supervising and MDA-Directing in the CE. There is no running “line cost”. The Cost Table algorithm will pull this “line cost” to the Cost Table in the Cost Module.

Anesthesia Provider, Post-op #1, MDA-Supervising (C.3-ms):

C.3-ms In the Post-op #1 stage, the Direct Labor algorithm allows for the input of time in minutes that the MDA-Supervising spends with the client in ALE and ACT for payment types C.3-ms.a, C.3-ms.b and C.3-ms.c described below. ALE and ACT cannot be inputted into payment types C.3-ms.d and C.3-ms.e described below. MDA-Supervising does not have PLE. As above, the Direct Labor algorithm will calculate “ACT hourly rate” for an MDA-Supervising with input from the user.

The hourly rate for ALE for MDA-Supervising is the hourly rate pushed from “CE set up” to the Cost Module.

Anesthesia Provider, Post-op #1 Stage, MDA-Directing (C.3-md):

C.3-md In the Post-op #1 stage, the Direct Labor algorithm allows for the input of time in minutes that the MDA-Directing spends with the client in ALE and ACT for payment types C.3-md.a, C.3-md.b and C.3-md.c described below. ALE and ACT cannot be inputted into payment types C.3-md.d and C.3-md.e described below. MDA-Directing does not have PLE. As above, the Direct Labor algorithm will calculate “ACT hourly rate” for an MDA-Directing with input from the user.

The hourly rate for ALE for MDA-Directing is the hourly rate pushed from “CE set up” to the Cost Module.

### Anesthesia Provider, Post-Op #1 Stage, MDA-Supervising, Payment Per Hour (C.3-ms.a):

Anesthesia Provider, Post-Op #1 stage, MDA-Directing, Payment Per Hour (C.3-md.a):

C.3-ms.a & C.3-md.a After input of time for the “payment per hour” payment type possibility completed in “CE set up”, the Direct Labor algorithm will add together the ALE and ACT costs for the MDA-Supervising or MDA-Directing and display to the user the cost for the MDA-Supervising or MDA-Directing in the Post-op #1 stage. The Cost Table algorithm will pull this cost to the Post-op #1 stage section of the Cost Table in the Cost Module. The Direct Labor algorithm will also calculate and display to the user the running “line cost” for the MDA-Supervising and MDA-Directing. The running “line cost” will be pulled to the Cost Table in the Cost Module by Cost table algorithm for the MDA-Supervising and MDA-Directing.

### Anesthesia Provider, Post-Op #1 Stage, MDA-Supervising, Salary (C.3-ms.b):

Anesthesia Provider, Post-op #1 stage, MDA-Directing, Salary (C.3-md.b):

C.3-ms.b & C.3-md.b After input of time for the “salary” payment type possibility completed in “CE set up”, the Direct Labor algorithm will add together the ALE and ACT costs for the MDA-Supervising or MDA-Directing and display to the user the cost for the MDA-Supervising or MDA-Directing in the Post-op #1 stage. The Cost Table algorithm will pull this cost to the Post-op #1 stage section of the Cost Table in the Cost Module. The Direct Labor algorithm will also calculate and display to the user the running “line cost” for the MDA-Supervising and MDA-Directing. The running “line cost” will be pulled to the Cost Table in the Cost Module by Cost table algorithm for the for the MDA-Supervising and MDA-Directing.

### Anesthesia Provider, Post-Op #1 Stage, MDA-Supervising, Payment Per Day (C.3-ms.c):

Anesthesia Provider, Post-Op #1 stage, MDA-Directing, Payment Per Day (C.3-md.c):

C.3-ms.c & C.3-md.c After input of time for the “payment per day” payment type possibility completed in “CE set up”, the Direct Labor algorithm will add together the ALE and ACT costs for the MDA-Supervising or MDA-Directing and display to the user the cost for the MDA-Supervising or MDA-Directing in the Post-op #1 stage. The Cost Table algorithm will pull this cost to the Post-op #1 stage section of the Cost Table in the Cost Module. The Direct Labor algorithm will also calculate and display to the user the running “line cost” for the MDA-Supervising and MDA-Directing. The running “line cost” will be pulled to the Cost Table in the Cost Module by Cost table algorithm for the for the MDA-Supervising and MDA-Directing.

### Anesthesia Provider, Post-Op #1 Stage, MDA-Supervising, Payment Per Case (C.3-ms.d):

Anesthesia Provider, Post-Op #1 stage, MDA-Directing, Payment Per Case (C.3-md.d):

C.3-ms.d & C.3-md.d After the “payment per case” payment type possibility is completed in “CE set up”, the cost for the MDA-Supervising or MDA-Directing in the CE is complete. No further input is needed. The Direct Labor algorithm will display this cost to the user as the “line cost”. There is no running “line cost”. The Cost Table algorithm will pull this “line cost” to the Cost Table in the Cost Module.

### Anesthesia Provider, Post-Op #1 Stage, MDA-Supervising, Percentage of Anesthesia Component (C.3-ms.e):

Anesthesia Provider, Post-Op #1 stage, MDA-Directing, Percentage of Anesthesia Component (C.3-md.e):

C.3-ms.e & C.3-md.e After the “percentage of anesthesia component” payment type possibility is completed in “CE set up”, the cost for the MDA-Supervising or MDA-Directing in the CE is complete. No further input is needed. Until the CE set up for “percentage of anesthesia component” is complete the direct labor cost section for the MDA-Supervising or MDA-Directing will be INCOMPLETE. Once the CE setup has been completed, the Direct Labor algorithm will display to the user this MDA-Supervising and MDA-Directing cost as the “line cost” for the MDA-Supervising and MDA-Directing in the CE. There is no running “line cost”. The Cost Table algorithm will pull this “line cost” to the Cost Table in the Cost Module.

Anesthesia Provider, Pre-Op, CRNA-Supervised (C.1-sc):

C.1-sc In the Pre-op stage the Direct Labor algorithm allows for the input of time in minutes that the CRNA-Supervised spends with the client in ALE for payment types C.1-sc.a, C.1-sc.b and C.1-sc.c described below. ALE cannot be inputted into payment types C.1-sc.d and C.1-sc.e described below. CRNA-Supervised does not have PLE or ACT.

Anesthesia Provider, Pre-Op, CRNA-Directed (C.1-dc):

C.1-dc In the Pre-op stage the Direct Labor algorithm allows for the input of time in minutes that the CRNA-Directed spends with the client in ALE for payment types C.1-dc.a, C.1-dc.b and C.1-dc.c described below. ALE cannot be inputted into payment types C.1-dc.d and C.1-dc.e described below. CRNA-Directed does not have PLE or ACT.

Anesthesia Provider, Pre-op, AA-Supervised (C.1-sa):

C.1-sa In the Pre-op stage the Direct Labor algorithm allows for the input of time in minutes that the AA-Supervised spends with the client in ALE for payment types C.1-sa.a, C.1-sa.b and C.1-sa.c described below. ALE cannot be inputted into payment types C.1-sa.d and C.1-sa.e described below. AA-Supervised does not have PLE or ACT.

Anesthesia Provider, Pre-op, AA-Directed (C.1-da):

C.1-da In the Pre-op stage the Direct Labor algorithm allows for the input of time in minutes that the AA-Directed spends with the client in ALE for payment types C.1-da.a, C.1-da.b and C.1-da.c described below. ALE cannot be inputted into payment types C.1-da.d and C.1-da.e described below. AA-Directed does not have PLE or ACT.

### Anesthesia Provider, Pre-op, CRNA-Supervised, Payment Per Hour (C.1-sc.a):

### Anesthesia Provider, Pre-op, CRNA-Directed, Payment Per Hour (C.1-dc.a):

### Anesthesia Provider, Pre-op, AA-Supervised, Payment Per Hour (C.1-sa.a):

Anesthesia Provider, Pre-op, AA-Directed, Payment Per hour (C.1-da.a):

C.1-sc.a, C.1-dc.a, C.1-sa.a, C.1-da.a After input of the time for “payment per hour” payment type possibility completed in “CE set up”, the Direct Labor algorithm will calculate and display to the user the cost for the CRNA-Supervised, CRNA-Directed, AA-Supervised or AA-Directed in the Pre-op stage. The Cost Table algorithm will pull this cost to the Pre-op stage section of the Cost Table in the Cost Module. The Direct Labor algorithm will also calculate and display to the user the running “line cost” for the CRNA-Supervised, CRNA-Directed, AA-Supervised or AA-Directed. The running “line cost” will be pulled to the Cost Table in the Cost Module by Cost table algorithm for the for the CRNA-Supervised, CRNA-Directed, AA-Supervised or AA-Directed.

### Anesthesia Provider, Pre-op, CRNA-Supervised, Salary (C.1-sc.b):

### Anesthesia Provider, Pre-op, CRNA-Directed, Salary (C.1-dc.b):

### Anesthesia Provider, Pre-op, AA-Supervised, Salary (C.1-sa.b):

### Anesthesia Provider, Pre-op, AA-Directed, Salary (C.1-da.b):

C.1-sc.b, C.1-dc.b, C.1-sa.b, C.1-da.b After input of time for the “salary” payment type possibility completed in “CE set up” where the payment is converted to the hourly rate, the Direct Labor algorithm will calculate and display to the user the cost for the CRNA-Supervised, CRNA-Directed, AA-Supervised or AA-Directed in the Pre-op stage. The Cost Table algorithm will pull this cost to the Pre-op stage section of the Cost Table in the Cost Module. The Direct Labor algorithm will also calculate and display to the user the running “line cost” for the CRNA-Supervised, CRNA-Directed, AA-Supervised or AA-Directed. The running “line cost” will be pulled to the Cost Table in the Cost Module by Cost table algorithm for the for the CRNA-Supervised, CRNA-Directed, AA-Supervised or AA-Directed.

### Anesthesia Provider, Pre-op, CRNA-Supervised, Payment Per Day (C.1-sc.c):

### Anesthesia Provider, Pre-op, CRNA-Directed, Payment Per Day (C.1-dc.c):

### Anesthesia Provider, Pre-op, AA-Supervised, Payment Per Day (C.1-sa.c):

### Anesthesia Provider, Pre-op, AA-Directed, Payment Per Day (C.1-da.c):

C.1-sc.c, C.1-dc.c, C.1-sa.c, C.1-da.c After input of time for the “payment per day” payment type possibility completed in “CE set up” where the payment is converted to the hourly rate, the Direct Labor algorithm will calculate and display to the user the cost for the CRNA-Supervised, CRNA-Directed, AA-Supervised or AA-Directed in the Pre-op stage. The Cost Table algorithm will pull this cost to the Pre-op stage section of the Cost Table in the Cost Module. The Direct Labor algorithm will also calculate and display to the user the running “line cost” for the CRNA-Supervised, CRNA-Directed, AA-Supervised or AA-Directed. The running “line cost” will be pulled to the Cost Table in the Cost Module by Cost table algorithm for the for the CRNA-Supervised, CRNA-Directed, AA-Supervised or AA-Directed.

### Anesthesia Provider, Pre-op, CRNA-Supervised, Payment Per Case (C.1-sc.d):

### Anesthesia Provider, Pre-op, CRNA-Directed, Payment Per Case (C.1-dc.d):

### Anesthesia Provider, Pre-op, AA-Supervised, Payment Per Case (C.1-sa.d):

### Anesthesia Provider, Pre-op, AA-Directed, Payment Per Case (C.1-da.d):

C.1-sc.d, C.1-dc.d, C.1-sa.d, C.1-da.d After the “payment per case” payment type possibility is completed in “CE set up”, the cost for the CRNA-Supervised, CRNA-Directed, AA-Supervised or AA-Directed in the CE is complete. No further input is needed. The Direct Labor algorithm will display this cost to the user as the “line cost”. There is no running “line cost”. The Cost Table algorithm will pull this “line cost” to the Cost Table in the Cost Module.

### Anesthesia Provider, Pre-Op, CRNA-Supervised, Percentage of Anesthesia Component (C.1-sc.e):

### Anesthesia Provider, Pre-Op, CRNA-Directed, Percentage of Anesthesia Component (C.1-dc.e):

### Anesthesia Provider, Pre-Op, AA-Supervised, Percentage of Anesthesia Component (C.1-sa.e):

### Anesthesia Provider, Pre-Op, AA-Directed, Percentage of Anesthesia Component (C.1-da.e):

C.1-sc.e, C.1-dc.e, C.1-sa.e, C.1-da.e After the “percentage of anesthesia component” payment type possibility is completed in “CE set up”, the cost for the CRNA-Supervised, CRNA-Directed, AA-Supervised or AA-Directed in the CE is complete. No further input is needed. Until the CE set up for “percentage of anesthesia component” is complete, the direct labor cost section for the CRNA-Supervised, CRNA-Directed, AA-Supervised or AA-Directed will be INCOMPLETE. Once the CE setup has been completed, the Direct Labor algorithm will display to the user this CRNA-Supervised, CRNA-Directed, AA-Supervised or AA-Directed cost as the “line cost” for the CRNA-Supervised, CRNA-Directed, AA-Supervised or AA-Directed in the CE. There is no running “line cost”. The Cost Table algorithm will pull this “line cost” to the Cost Table in the Cost Module.

### Anesthesia Provider, O.R. Stage, CRNA-Supervised (C.2-Sc):

C.2-sc In the O.R. stage the Direct Labor algorithm allows for the input of time in minutes that the CRNA-Supervised spends with the client in ALE for payment types C.2-sc.a, C.2-sc.b and C.2-sc.c described below. ALE cannot be inputted into payment types C.2-sc.d and C.2-sc.e described below. CRNA-Supervised does not have PLE or ACT.

Anesthesia Provider, O.R. Stage, CRNA-Directed (C.2-dc):

C.2-dc In the O.R. stage the Direct Labor algorithm allows for the input of time in minutes that the CRNA-Directed spends with the client in ALE for payment types C.2-dc.a, C.2-dc.b and C.2-dc.c described below. ALE cannot be inputted into payment types C.2-dc.d and C.2-dc.e described below. CRNA-Directed does not have PLE or ACT.

Anesthesia Provider, O.R. Stage, AA-Supervised (C.2-sa):

C.2-sa In the O.R. stage the Direct Labor algorithm allows for the input of time in minutes that the AA-Supervised spends with the client in ALE for payment types C.2-sa.a, C.2-sa.b and C.2-sa.c described below. ALE cannot be inputted into payment types C.2-sa.d and C.2-sa.e described below. AA-Supervised does not have PLE or ACT.

Anesthesia Provider, O.R. Stage, AA-Directed (C.2-da):

C.2-da In the O.R. stage the Direct Labor algorithm allows for the input of time in minutes that the AA-Directed spends with the client in ALE for payment types C.2-da.a, C.2-da.b and C.2-da.c described below. ALE cannot be inputted into payment types C.2-da.d and C.2-da.e described below. AA-Directed does not have PLE or ACT.

### Anesthesia Provider, O.R. Stage, CRNA-Supervised, Payment Per Hour (C.2-sc.a):

Anesthesia Provider, O.R. stage, CRNA-Directed, Payment Per Hour (C.2-dc.a):

### Anesthesia Provider, O.R. Stage, AA-Supervised, Payment Per Hour (C.2-sa.a):

Anesthesia Provider, O.R. stage, AA-Directed, Payment Per Hour (C.2-da.a):

C.2-sc.a, C.2-dc.a, C.2-sa.a, C.2-da.a After input of the time for “payment per hour” payment type possibility completed in “CE set up”, the Direct Labor algorithm will calculate and display to the user the cost for the CRNA-Supervised, CRNA-Directed, AA-Supervised or AA-Directed in the O.R. stage. The Cost Table algorithm will pull this cost to the O.R stage section of the Cost Table in the Cost Module. The Direct Labor algorithm will also calculate and display to the user the running “line cost” for the CRNA-Supervised, CRNA-Directed, AA-Supervised or AA-Directed. The running “line cost” will be pulled to the Cost Table in the Cost Module by Cost table algorithm for the for the CRNA-Supervised, CRNA-Directed, AA-Supervised or AA-Directed.

### Anesthesia Provider, O.R. Stage, CRNA-Supervised, Salary (C.2-sc.b):

Anesthesia Provider, O.R. stage, CRNA-Directed, Salary (C.2-dc.b):

### Anesthesia Provider, O.R. Stage, AA-Supervised, Salary (C.2-sa.b):

Anesthesia Provider, O.R. stage, AA-Directed, Salary (C.2-da.b):

C.2-sc.b, C.2-dc.b, C.2-sa.b, C.2-da.b After input of time for the “salary” payment type possibility completed in “CE set up” where the payment is converted to the hourly rate, the Direct Labor algorithm will calculate and display to the user the cost for the CRNA-Supervised, CRNA-Directed, AA-Supervised or AA-Directed in the O.R. stage. The Cost Table algorithm will pull this cost to the O.R stage section of the Cost Table in the Cost Module. The Direct Labor algorithm will also calculate and display to the user the running “line cost” for the CRNA-Supervised, CRNA-Directed, AA-Supervised or AA-Directed. The running “line cost” will be pulled to the Cost Table in the Cost Module by Cost table algorithm for the for the CRNA-Supervised, CRNA-Directed, AA-Supervised or AA-Directed.

### Anesthesia Provider, O.R. Stage, CRNA-Supervised, Payment Per Day (C.2-sc.c):

Anesthesia Provider, O.R. stage, CRNA-Directed Payment Per Day (C.2-dc.c):

### Anesthesia Provider, O.R. Stage, AA-Supervised, Payment Per Day (C.2-sa.c):

Anesthesia Provider, O.R. stage, AA-Directed, Payment Per Day (C.2-da.c):

C.2-sc.c, C.2-dc.c, C.2-sa.c, C.2-da.c After input of time for the “payment per day” payment type possibility completed in “CE set up” where the payment is converted to the hourly rate, the Direct Labor algorithm will calculate and display to the user the cost for the CRNA-Supervised, CRNA-Directed, AA-Supervised or AA-Directed in the O.R. stage. The Cost Table algorithm will pull this cost to the O.R stage section of the Cost Table in the Cost Module. The Direct Labor algorithm will also calculate and display to the user the running “line cost” for the CRNA-Supervised, CRNA-Directed, AA-Supervised or AA-Directed. The running “line cost” will be pulled to the Cost Table in the Cost Module by Cost table algorithm for the for the CRNA-Supervised, CRNA-Directed, AA-Supervised or AA-Directed.

### Anesthesia Provider, O.R. Stage, CRNA-Supervised, Payment Per Case (C.2-sc.d):

Anesthesia Provider, O.R. stage, CRNA-Directed Payment Per Case (C.2-dc.d):

### Anesthesia Provider, O.R. Stage, AA-Supervised, Payment Per Case (C.2-sa.d):

Anesthesia Provider, O.R. stage, AA-Directed, Payment Per Case (C.2-da.d):

C.2-sc.d, C.2-dc.d, C.2-sa.d, C.2-da.d After the “payment per case” payment type possibility is completed in “CE set up”, the cost for the CRNA-Supervised, CRNA-Directed, AA-Supervised or AA-Directed in the CE is complete. No further input is needed. The Direct Labor algorithm will display this cost to the user as the “line cost”. There is no running “line cost”. The Cost Table algorithm will pull this “line cost” to the Cost Table in the Cost Module.

### Anesthesia Provider, O.R. Stage, CRNA-Supervised, Percentage of Anesthesia Component (C.2-sc.e):

ANESTHESIA Provider, O.R. stage, CRNA-Directed, Percentage of Anesthesia Component (C.2-dc.e):

### Anesthesia Provider, O.R. Stage, AA-Supervised, Percentage of Anesthesia Component (C.2-sa.e):

Anesthesia Provider, O.R. stage, AA-Directed, Percentage of Anesthesia Component (C.2-da.e):

C.2-sc.e, C.2-dc.e, C.2-sa.e, C.2-da.e After the “percentage of anesthesia component” payment type possibility is completed in “CE set up”, the cost for the CRNA-Supervised, CRNA-Directed, AA-Supervised or AA-Directed in the CE is complete. No further input is needed. Until the CE set up for “percentage of anesthesia component” is complete, the direct labor cost section for the CRNA-Supervised, CRNA-Directed, AA-Supervised or AA-Directed will be INCOMPLETE. Once the CE setup has been completed, the Direct Labor algorithm will display to the user this CRNA-Supervised, CRNA-Directed, AA-Supervised or AA-Directed cost as the “line cost” for the CRNA-Supervised, CRNA-Directed, AA-Supervised or AA-Directed in the CE. There is no running “line cost”. The Cost Table algorithm will pull this “line cost” to the Cost Table in the Cost Module.

Anesthesia Provider, Post-Op #1 Stage, CRNA-Supervised (C.3-sc):

C.3-sc In the Post-op #1 stage the Direct Labor algorithm allows for the input of time in minutes that the CRNA-Supervised spends with the client in ALE for payment types C.3-sc.a, C.3-sc.b and C.3-sc.c described below. ALE cannot be inputted into payment types C.3-sc.d and C.3-sc.e described below. CRNA-Supervised does not have PLE or ACT.

Anesthesia Provider, Post-Op #1 Stage, CRNA-Directed (C.3-dc):

C.3-dc In the Post-op #1 stage the Direct Labor algorithm allows for the input of time in minutes that the CRNA-Directed spends with the client in ALE for payment types C.3-dc.a, C.3-dc.b and C.3-dc.c described below. ALE cannot be inputted into payment types C.3-dc.d and C.3-dc.e described below. CRNA-Directed does not have PLE or ACT.

Anesthesia Provider, Post-Op #1 Stage, AA-Supervised (C.3-sa):

C.3-sa In the Post-op #1 stage the Direct Labor algorithm allows for the input of time in minutes that the AA-Supervised spends with the client in ALE for payment types C.3-sa.a, C.3-sa.b and C.3-sa.c described below. ALE cannot be inputted into payment types C.3-sa.d and C.3-sa.e described below. AA-Supervised does not have PLE or ACT.

Anesthesia Provider, Post-Op #1 Stage, AA-Directed (C.3-da):

C.3-da In the Post-op #1 stage the Direct Labor algorithm allows for the input of time in minutes that the AA-Directed spends with the client in ALE for payment types C.3-da.a, C.3-da.b and C.3-da.c described below. ALE cannot be inputted into payment types C.3-da.d and C.3-da.e described below. AA-Directed does not have PLE or ACT.

### Anesthesia Provider, Post-Op #1 Stage, CRNA-Supervised, Payment Per Hour (C.3-sc.a):

Anesthesia Provider, Post-Op #1 stage, CRNA-Directed Payment Per Hour (C.3-dc.a):

### Anesthesia Provider, Post-Op #1 Stage, AA-Supervised, Payment Per Hour (C.3-sa.a):

Anesthesia Provider, Post-op #1 stage, AA-Directed, Payment Per Hour (C.3-da.a):

C.3-sc.a, C.3-dc.a, C.3-sa.a, C.3-da.a After input of the time for “payment per hour” payment type possibility completed in “CE set up”, the Direct Labor algorithm will calculate and display to the user the cost for the CRNA-Supervised, CRNA-Directed, AA-Supervised or AA-Directed in the Post-op #1 stage. The Cost Table algorithm will pull this cost to the Post-op #1 stage section of the Cost Table in the Cost Module. The Direct Labor algorithm will also calculate and display to the user the running “line cost” for the CRNA-Supervised, CRNA-Directed, AA-Supervised or AA-Directed. The running “line cost” will be pulled to the Cost Table in the Cost Module by Cost table algorithm for the for the CRNA-Supervised, CRNA-Directed, AA-Supervised or AA-Directed.

### Anesthesia Provider, Post-Op #1 Stage, CRNA-Supervised, Salary (C.3-sc.b):

Anesthesia Provider, Post-op #1 stage, CRNA-Directed, Salary (C.3-dc.b):

### Anesthesia Provider, Post-Op #1 Stage, AA-Supervised, Salary (C.3-sa.b):

Anesthesia Provider, Post-op #1 stage, AA-Directed, Salary (C.3-da.b):

C.3-sc.b, C.3-dc.b, C.3-sa.b, C.3-da.b After input of time for the “salary” payment type possibility completed in “CE set up” where the payment is converted to the hourly rate, the Direct Labor algorithm will calculate and display to the user the cost for the CRNA-Supervised, CRNA-Directed, AA-Supervised or AA-Directed in the Post-op #1 stage. The Cost Table algorithm will pull this cost to the Post-op #1 stage section of the Cost Table in the Cost Module. The Direct Labor algorithm will also calculate and display to the user the running “line cost” for the CRNA-Supervised, CRNA-Directed, AA-Supervised or AA-Directed. The running “line cost” will be pulled to the Cost Table in the Cost Module by Cost table algorithm for the for the CRNA-Supervised, CRNA-Directed, AA-Supervised or AA-Directed.

### Anesthesia Provider, Post-Op #1 Stage, CRNA-Supervised, Payment Per Day (C.3-sc.c):

Anesthesia Provider, Post-Op #1 stage, CRNA-Directed Payment Per Day (C.3-dc.c):

### Anesthesia Provider, Post-Op #1 Stage, AA-Supervised, Payment Per Day (C.3-sa.c):

Anesthesia Provider, Post-op #1 stage, AA-Directed, Payment Per Day (C.3-da.c):

C.3-sc.c, C.3-dc.c, C.3-sa.c, C.3-da.c After input of time for the “payment per day” payment type possibility completed in “CE set up” where the payment is converted to the hourly rate, the Direct Labor algorithm will calculate and display to the user the cost for the CRNA-Supervised, CRNA-Directed, AA-Supervised or AA-Directed in the Post-op #1 stage. The Cost Table algorithm will pull this cost to the Post-op #1 stage section of the Cost Table in the Cost Module. The Direct Labor algorithm will also calculate and display to the user the running “line cost” for the CRNA-Supervised, CRNA-Directed, AA-Supervised or AA-Directed. The running “line cost” will be pulled to the Cost Table in the Cost Module by Cost table algorithm for the for the CRNA-Supervised, CRNA-Directed, AA-Supervised or AA-Directed.

### Anesthesia Provider, Post-Op #1 Stage, CRNA-Supervised, Payment Per Case (C.3-sc.d):

Anesthesia Provider, Post-Op #1 stage, CRNA-Directed Payment Per Case (C.3-dc.d):

### Anesthesia Provider, Post-Op #1 Stage, AA-Supervised, Payment Per Case (C.3-sa.d):

Anesthesia Provider, Post-op #1 stage, AA-Directed, Payment Per Case (C.3-da.d):

C.3-sc.d, C.3-dc.d, C.3-sa.d, C.3-da.d After the “payment per case” payment type possibility is completed in “CE set up”, the cost for the CRNA-Supervised, CRNA-Directed, AA-Supervised or AA-Directed in the CE is complete. No further input is needed. The Direct Labor algorithm will display this cost to the user as the “line cost”. There is no running “line cost”. The Cost Table algorithm will pull this “line cost” to the Cost Table in the Cost Module.

### Anesthesia Provider, Post-Op #1 Stage, CRNA-Supervised, Percentage of Anesthesia Component (C.3-sc.e):

Anesthesia Provider, Post-Op #1 stage, CRNA-Directed, Percentage of Anesthesia Component (C.3-dc.e):

### Anesthesia Provider, Post-Op #1 Stage, AA-Supervised, Percentage of Anesthesia Component (C.3-sa.e):

Anesthesia Provider, Post-Op #1 stage, AA-Directed, Percentage of Anesthesia Component (C.3-da.e):

C.3-sc.e, C.3-dc.e, C.3-sa.e, C.3-da.e After the “percentage of anesthesia component” payment type possibility is completed in “CE set up”, the cost for the CRNA-Supervised, CRNA-Directed, AA-Supervised or AA-Directed in the CE is complete. No further input is needed. Until the CE set up for “percentage of anesthesia component” is complete, the direct labor cost section for the CRNA-Supervised, CRNA-Directed, AA-Supervised or AA-Directed will be INCOMPLETE. Once the CE setup has been completed, the Direct Labor algorithm will display to the user this CRNA-Supervised, CRNA-Directed, AA-Supervised or AA-Directed cost as the “line cost” for the CRNA-Supervised, CRNA-Directed, AA-Supervised or AA-Directed in the CE. There is no running “line cost”. The Cost Table algorithm will pull this “line cost” to the Cost Table in the Cost Module.

### Anesthesia Provider, AO Time (C.5):

Anesthesia Providers of all job descriptions have AO time, and all are inputted in the same manner using the Direct Labor algorithm as described below.

C.5 In AO time, the Direct Labor algorithm allows for the input of time in minutes that the Anesthesia Provider(s) spends doing purely administrative work. The administrative work must occur outside the CE but the time to perform this work does not need to be consecutive. AO time input for payment types C.5.a, C.5.b and C.5.c are described below. Payment types C.5.d and C.5.e do not have input for AO time.

### Anesthesia Provider, AO Time, Payment Per Hour (C.5.a):

C.5.a After input of the time for “payment per hour” payment type possibility completed in “CE set up”, the Direct Labor algorithm will calculate and display to the user the cost for the Anesthesia Provider(s) for the AO time. The Cost Table algorithm will pull this cost to the AO time section of the Cost Table in the Cost Module. The Direct Labor algorithm will also calculate and display to the user the running “line cost” for the Anesthesia Provider. The running “line cost” will be pulled to the Cost Table in the Cost Module by Cost table algorithm for the Anesthesia Provider(s).

### Anesthesia Provider, AO Time, Salary (C.5.b):

C.5.b After input of time for the “salary” payment type possibility completed in “CE set up” where the payment is converted to the hourly rate, the Direct Labor algorithm will calculate and display to the user the cost for the Anesthesia Provider(s) for the AO time. The Cost Table algorithm will pull this cost to the AO time section of the Cost Table in the Cost Module. The Direct Labor algorithm will also calculate and display to the user the running “line cost” for the Anesthesia Provider. The running “line cost” will be pulled to the Cost Table in the Cost Module by Cost table algorithm for the Anesthesia Provider(s).

### Anesthesia Provider, AO Time, Payment Per Day (A.5.c):

C.5.c After input of time for the “payment per day” payment type possibility completed in “CE set up” where the payment is converted to the hourly rate, the Direct Labor algorithm will calculate and display to the user the cost for the Anesthesia Provider(s) for the AO time. The Cost Table algorithm will pull this cost to the AO time section of the Cost Table in the Cost Module. The Direct Labor algorithm will also calculate and display to the user the running “line cost” for the Anesthesia Provider. The running “line cost” will be pulled to the Cost Table in the Cost Module by Cost table algorithm for the Anesthesia Provider(s).

### Anesthesia Provider, AO Time, Payment Per Case (C.5.d):

C.5.d After the “payment per case” payment type possibility is completed in “CE set up”, the cost for the Anesthesia Provider(s) in the CE is complete. No further input is needed. The cost of AO time is included in the “payment per case”. The Direct Labor algorithm will display this cost to the user as the “line cost”. There is no running “line cost”. The Cost Table algorithm will pull this “line cost” to the Cost Table in the Cost Module.

### Anesthesia Provider, AO Time, Percentage of Professional Component (C.5.e):

C.5.e After the “percentage of anesthesia component” payment type possibility is completed in “CE set up”, the cost for the Anesthesia Provider(s) in the CE is complete. No further input is needed. The cost of AO time is included in the “percentage of anesthesia component”. Until the CE set up for “percentage of anesthesia component” is complete the direct labor cost section for the Anesthesia Provider(s) will be INCOMPLETE. Once the CE setup has been completed, the Direct Labor algorithm will display to the user this Anesthesia Provider cost as the “line cost”. There is no running “line cost”. The Cost Table algorithm will pull this “line cost” to the Cost Table in the Cost Module.

### D. Ancillary Provider Direct Labor Cost:

Ancillary Providers are either present during one or more stages of the CE or directly engaged with the event that occurs in one or more stages of the CE. If the Ancillary Provider is not present during the CE but directly engaged in an event that occurs during one of the stages of the CE, the Ancillary Provider's time is allocated to that stage.

During the CE set up, using the CE Set Up algorithm the Ancillary Provider job description is inputted. The inputted job description has a mandatory requirement to input one of two mutually exclusive options of “inside the CE” or “outside the CE”. The choice of inside the CE or outside the CE will determine the time allocation options available to the user as described below.

### Ancillary Provider, Pre-Op Stage, Inside CE (D.1-i):

D.1-i In the Pre-op stage for Ancillary Providers that are present during the CE, the Direct Labor algorithm allows for the input of time in minutes that the Ancillary Provider spends with the client in ALE and PLE for the payment types D.1-i.a, D.1-i.b and D.1-i.e described below. ALE and PLE cannot be inputted into the payment types D.1-i.d and D.1-i.e described below. Ancillary Providers that are present during the CE do not have ACT. For Ancillary Providers that are present during the CE, the cost per hour for ALE and PLE is the same.

### Ancillary Provider, Pre-Op Stage, Outside CE (D.1-o):

D.1-o In the Pre-op stage for Ancillary Providers that are not present during the CE but are directly engaged with an event that occurs during one or more stages of the CE, the Direct Labor algorithm allows for the input of time in minutes that the Ancillary Provider spends engaged with that event as ALE only. Ancillary Providers that are not present during the CE do not have ACT. ALE time can be inputted into payment types D.1-o.a, D.1-o.b and D.1-o.c described below. ALE time cannot be inputted into payment types D.1-o.d and D.1-o.e described below.

### Ancillary Provider, Pre-Op Stage, Inside CE, Payment Per Hour (D.1-i.a):

Ancillary Provider, Pre-Op stage, Outside CE, Payment Per Hour (D.1-o.a):

D.1-i.a & D.1-o.a For Ancillary Providers present or not present during the CE, after input of the time for “payment per hour” payment type possibility completed in “CE set up”, the Direct Labor algorithm will calculate and display to the user the cost for the Ancillary Provider in the Pre-op stage. The Cost Table algorithm will pull this cost to the Pre-op stage section of the Cost Table in the Cost Module. The Direct Labor algorithm will also calculate and display to the user the running “line cost” for the Ancillary Provider. The running “line cost” will be pulled to the Cost Table in the Cost Module by Cost table algorithm for the Ancillary Provider.

The running “line cost”, as it relates to all Players, is the running total of the cost for a Player in the CE and changes on the Cost Table as additional direct labor costs are added or removed for that Player. When the user has finished inputting all the direct labor costs for a Player, the running “line cost” is referred to as the “line cost” for that Player.

### Ancillary Provider, Pre-Op Stage, Inside CE, Salary (D.1-i.b):

Ancillary Provider, Pre-Op stage, Outside CE, Salary (D.1-o.b):

D.1-i.b & D.1-o.b After input of time for the “salary” payment type possibility completed in “CE set up” where the payment is converted to the hourly rate, the Direct Labor algorithm will calculate and display to the user the cost for the Ancillary Provider in the Pre-op stage. The Cost Table algorithm will pull this cost to the Pre-op stage section of the Cost Table in the Cost Module. The Direct Labor algorithm will also calculate and display to the user the running “line cost” for the Ancillary Provider. The running “line cost” will be pulled to the Cost Table in the Cost Module by Cost table algorithm for the Ancillary Provider.

### Ancillary Provider, Pre-Op Stage, Inside CE, Payment Per Day (D.1-i.e):

Ancillary Provider, Pre-Op stage, Outside CE, Payment Per Day (D.1-o.c):

D.1-i.e & D.1-o.c After input of time for the “payment per day” payment type possibility completed in “CE set up” where the payment is converted to the hourly rate, the Direct Labor algorithm will calculate and display to the user the cost for the Ancillary Provider in the Pre-op stage. The Cost Table algorithm will pull this cost to the Pre-op stage section of the Cost Table in the Cost Module. The Direct Labor algorithm will also calculate and display to the user the running “line cost” for the Ancillary Provider. The running “line cost” will be pulled to the Cost Table in the Cost Module by Cost table algorithm for the Ancillary Provider.

### Ancillary Provider, Pre-Op Stage, Inside CE, Payment Per Case (D.1-i.d):

Ancillary Provider, Pre-Op stage, Outside CE, Payment Per Case (D.1-o.d):

D.1-i.d & D.1-o.d After the “payment per case” payment type possibility is completed in “CE set up”, the cost for the Ancillary provider in the CE is complete. No further input is needed. The Direct Labor algorithm will display to the user this Ancillary Provider cost as the “line cost” for the Ancillary Provider in the CE. There is no running “line cost”. The Cost Table algorithm will pull this “line cost” to the Cost Table in the Cost Module.

### Ancillary Provider, Pre-Op Stage, Inside CE, Percentage of Ancillary Component (D.1-i.e):

Ancillary Provider, Pre-Op stage, Outside CE, Percentage of Ancillary Component (D.1-o.e):

D.1-i.e & D.1-o.e After the “percentage of ancillary component” payment type possibility is completed in “CE set up”, the cost for the Ancillary provider in the CE is complete. No further input is needed. Until the CE set up for “percentage of ancillary component” is complete the direct labor cost section for the Ancillary Provider will be INCOMPLETE. Once the CE setup has been completed, the Direct Labor algorithm will display to the user this Ancillary Provider cost as the “line cost” for the Ancillary Provider in the CE. There is no running “line cost”. The Cost Table algorithm will pull this “line cost” to the Cost Table in the Cost Module.

### Ancillary Provider, O.R. Stage, Inside CE, Payment Per Hour (D.2-i):

D.2-i In the O.R. stage for Ancillary Providers that are present during the CE, the Direct Labor algorithm allows for the input of time in minutes that the Ancillary Provider spends with the client in ALE only for payment types D.2-i.a, D.2-i.b and D.2-i.e described below. There is no PLE for the Professional Provider in the O.R. stage. Neither ALE nor PLE can be inputted into payment types D.2-i.d and D.2-i.e described below. Ancillary Providers that are present during the CE do not have ACT.

### Ancillary Provider, O.R. Stage, Outside CE, Payment Per Hour (D.2-o):

D.2-o In the O.R. stage for Ancillary Providers that are not present during the CE but are directly engaged with an event that occurs during one or more stages of the CE, the Direct Labor algorithm allows for the input of time in minutes that the Ancillary Provider spends engaged with that event as ALE only. Ancillary Providers that are not present during the CE do not have ACT. ALE time can be inputted into payment types D.2-o.a, D.2-o.b and D.2-o.c described below. ALE time cannot be inputted into payment types D.2-o.d and D.2-o.e described below

### Ancillary Provider, O.R. Stage, Inside CE, Payment Per Hour (D.2-i.a):

Ancillary Provider, O.R. stage, Outside CE, Payment Per Hour (D.2-o.a):

D.2-i.a & D.2-o.a After input of time for the “payment per hour” payment type possibility completed in “CE set up”, the Direct Labor algorithm will calculate and display to the user the cost for the Ancillary Provider present or not present in the O.R. stage. The Cost Table algorithm will pull this cost to the O.R stage section of the Cost Table in the Cost Module. The Direct Labor algorithm will also calculate and display to the user the running “line cost” for the Ancillary Provider. The running “line cost” will be pulled to the Cost Table in the Cost Module by Cost table algorithm for the Ancillary Provider.

### Ancillary Provider, O.R. Stage, Inside CE, Salary (D.2-i.b):

Ancillary Provider, O.R. stage, Outside CE, Salary (D.2-o.b):

D.2-i.b & D.2-o.b After input of time for the “salary” payment type possibility completed in “CE set up” where the payment is converted to the hourly rate, the Direct Labor algorithm will calculate and display to the user the cost for the Ancillary Provider present or not present in the O.R. stage. The Cost Table algorithm will pull this cost to the O.R stage section of the Cost Table in the Cost Module. The Direct Labor algorithm will also calculate and display to the user the running “line cost” for the Ancillary Provider. The running “line cost” will be pulled to the Cost Table in the Cost Module by Cost table algorithm for the Ancillary Provider.

### Ancillary Provider, O.R. Stage, Inside CE, Payment Per Day (D.2-i.e):

Ancillary Provider, O.R. stage, Outside CE, Payment Per Day (D.2-o.c):

D.2-i.e & D.2-o.c After input of time for the “payment per day” payment type possibility completed in “CE set up” where the payment is converted to the hourly rate, the Direct Labor algorithm will calculate and display to the user the cost for the Ancillary Provider present or not present in the O.R. stage. The Cost Table algorithm will pull this cost to the O.R stage section of the Cost Table in the Cost Module. The Direct Labor algorithm will also calculate and display to the user the running “line cost” for the Ancillary Provider. The running “line cost” will be pulled to the Cost Table in the Cost Module by Cost table algorithm for the Ancillary Provider.

### Ancillary Provider, O.R. Stage, Inside CE, Payment Per Case (D.2-i.d):

Ancillary Provider, O.R. stage, Outside CE, Payment Per Case (D.2-o.d):

D.2-i.d & D.2-o.d After the “payment per case” payment type possibility is completed in “CE set up”, the cost for the Ancillary provider present or not present in the CE is complete. No further input is needed. The Direct Labor algorithm will display to the user this Ancillary Provider cost as the “line cost” for the Ancillary Provider in the CE. There is no running “line cost”. The Cost Table algorithm will pull this “line cost” to the Cost Table in the Cost Module.

### Ancillary Provider, O.R. Stage, Inside CE, Percentage of Ancillary Component (D.2-i.e):

Ancillary Provider, O.R. stage, Outside CE, Percentage of Ancillary Component (D.2-o.e):

D.2-i.e & D.2-o.e After the “percentage of ancillary component” payment type possibility is completed in “CE set up”, the cost for the Ancillary provider present or not present in the CE is complete. No further input is needed. Until the CE set up for “percentage of ancillary component” is complete the direct labor cost section for the Ancillary Provider will be INCOMPLETE. Once the CE setup has been completed, the Direct Labor algorithm will display to the user this Ancillary Provider cost as the “line cost” for the Ancillary Provider in the CE. There is no running “line cost”. The Cost Table algorithm will pull this “line cost” to the Cost Table in the Cost Module.

### Ancillary Provider, Post-Op #1 Stage, Inside CE (D.3-i):

D.3-i In the Post-op #1 stage, the Direct Labor algorithm allows for the input of time in minutes that the Ancillary Providers that are present during the CE spend with the client in ALE only for payment types D.3.a, D.3.b and D.3.c described below. There is no PLE for the Ancillary Provider present during the CE in the Post-op #1 stage. Neither ALE nor PLE can be inputted into payment type D.3.d and D.3.e described below. Ancillary Providers present during the CE do not have ACT.

### Ancillary Provider, Post-Op #1 Stage, Outside CE (D.3-o):

D.3-o In the Post-op #1 stage for Ancillary Providers that are not present during the CE but are directly engaged with an event that occurs during one or more stages of the CE, the Direct Labor algorithm allows for the input of time in minutes that the Ancillary Provider spends engaged with that event as ALE only. Ancillary Providers that are not present during the CE do not have ACT. ALE time can be inputted into payment types D.3-o.a, D.3-o.b and D.3-o.c described below. ALE time cannot be inputted into payment types D.3-o.d and D.3-o.e described below.

### Ancillary Provider, Post-Op #1 Stage, Inside CE, Payment Per Hour (D.3-i.a):

Ancillary Provider, Post-Op #1 stage, Outside CE, Payment Per Hour (D.3-o.a):

D.3-i.a & D.3-o.a After input of time for the “payment per hour” payment type possibility completed in “CE set up”, the Direct Labor algorithm will calculate and display to the user the cost for the Ancillary Provider present or not present in the Post-op #1 stage. The Cost Table algorithm will pull this cost to the Post-op #1 stage section of the Cost Table in the Cost Module. The Direct Labor algorithm will also calculate and display to the user the running “line cost” for the Ancillary Provider. The running “line cost” will be pulled to the Cost Table in the Cost Module by Cost table algorithm for the Ancillary Provider.

### Ancillary Provider, Post-Op #1 Stage, Inside CE, Salary (D.3-i.b):

Ancillary Provider, Post-Op #1 stage, Outside CE, Salary (D.3-o.b):

D.3-i.b & D.3-o.b After input of time for the “salary” payment type possibility completed in “CE set up” where the payment is converted to the hourly rate, the Direct Labor algorithm will calculate and display to the user the cost for the Ancillary Provider present or not present in the Post-op #1 stage. The Cost Table algorithm will pull this cost to the Pre-op #1 stage section of the Cost Table in the Cost Module. The Direct Labor algorithm will also calculate and display to the user the running “line cost” for the Ancillary Provider. The running “line cost” will be pulled to the Cost Table in the Cost Module by Cost table algorithm for the Ancillary Provider.

### Ancillary Provider, Post-Op #1 Stage, Inside CE, Payment Per Day (D.3-i.e):

Ancillary Provider, Post-Op #1 stage, Outside CE, Payment Per Day (D.3-o.c):

D.3-i.e & D.3-o.c After input of time for the “payment per day” payment type possibility completed in “CE set up” where the payment is converted to the hourly rate, the Direct Labor algorithm will calculate and display to the user the cost for the Ancillary Provider present or not present in the Post-op #1 stage. The Cost Table algorithm will pull this cost to the Pre-op #1 stage section of the Cost Table in the Cost Module.

The Direct Labor algorithm will also calculate and display to the user the running “line cost” for the Ancillary Provider. The running “line cost” will be pulled to the Cost Table in the Cost Module by Cost table algorithm for the Ancillary Provider.

### Ancillary Provider, Post-Op #1 Stage, Inside CE, Payment Per Case (D.3-i.d):

Ancillary Provider, Post-Op #1 stage, Outside CE, Payment Per Case (D.3-o.d):

D.3-i.d & D.3-o.d After the “payment per case” payment type possibility is completed in “CE set up”, the cost for the Ancillary provider present or not present in the CE is complete. No further input is needed. The Direct Labor algorithm will display to the user this Ancillary Provider cost as the “line cost” for the Ancillary Provider in the CE. There is no running “line cost”. The Cost Table algorithm will pull this “line cost” to the Cost Table in the Cost Module

### Ancillary Provider, Post-Op #1 Stage, Inside CE, Percentage of Ancillary Component (D.3-i.e):

Ancillary Provider, Post-Op #1 stage, Outside CE, Percentage of Ancillary component (D.3-o.e):

D.3-i.e & D.3-o.e After the “percentage of ancillary component” payment type possibility is completed in “CE set up”, the cost for the Ancillary provider present or not present in the CE is complete. No further input is needed. Until the CE set up for “percentage of ancillary component” is complete the direct labor cost section for the Ancillary Provider will be INCOMPLETE. Once the CE setup has been completed, the Direct Labor algorithm will display to the user this Ancillary Provider cost as the “line cost” for the Ancillary Provider in the CE. There is no running “line cost”. The Cost Table algorithm will pull this “line cost” to the Cost Table in the Cost Module.

### Ancillary Provider, Post-Op #2 Stage, Outside CE (D.4-i):

D.4-i In the Post-op #2 stage, the Direct Labor algorithm allows for the input of time in minutes that the Ancillary Provider that are present during the CE spends with the client in ALE only for payment types D.4.a, D.4.b and D.4.c described below. There is no PLE for the Ancillary Provider present during the CE in the Post-op #2 stage. Neither ALE nor PLE can be inputted into payment type D.4.d and D.4.e described below. Ancillary Providers present during the CE do not have ACT.

### Ancillary Provider, Post-Op #2 Stage, Outside CE (D.4-o):

D.4-o In the Post-op #2 stage for Ancillary Providers that are not present during the CE but are directly engaged with an event that occurs during one or more stages of the CE, the Direct Labor algorithm allows for the input of time in minutes that the Ancillary Provider spends engaged with that event as ALE only. Ancillary Providers that are not present during the CE do not have ACT. ALE time can be inputted into payment types D.4-o.a, D.4-o.b and D.4-o.c described below. ALE time cannot be inputted into payment types D.4-o.d and D.4-o.e described below.

### Ancillary Provider, Post-Op #2 Stage, Inside CE, Payment Per Hour (D.4-i.a):

Ancillary Provider, Post-Op #2 stage, Outside CE, Payment Per Hour (D.4-o.a):

D.4-i.a & D.4-o.a After input of time for the “payment per hour” payment type possibility completed in “CE set up”, the Direct Labor algorithm will calculate and display to the user the cost for the Ancillary Provider present or not present in the Post-op #2 stage. The Cost Table algorithm will pull this cost to the Pre-op #2 stage section of the Cost Table in the Cost Module. The Direct Labor algorithm will also calculate and display to the user the running “line cost” for the Ancillary Provider. The running “line cost” will be pulled to the Cost Table in the Cost Module by Cost table algorithm for the Ancillary Provider.

### Ancillary Provider, Post-Op #2 Stage, Inside CE, Salary (D.4-i.b):

Ancillary Provider, Post-Op #2 stage, Outside CE, Salary (D.4-o.b):

D.4-i.b & D.4-o.b After input of time for the “salary” payment type possibility completed in “CE set up” where the payment is converted to the hourly rate, the Direct Labor algorithm will calculate and display to the user the cost for the Ancillary Provider present or not present in the Post-op #2 stage. The Cost Table algorithm will pull this cost to the Pre-op #2 stage section of the Cost Table in the Cost Module. The Direct Labor algorithm will also calculate and display to the user the running “line cost” for the Ancillary Provider. The running “line cost” will be pulled to the Cost Table in the Cost Module by Cost table algorithm for the Ancillary Provider.

### Ancillary Provider, Post-Op #2 Stage, Inside CE, Payment Per Day (D.4-i.e):

Ancillary Provider, Post-Op #2 stage, Outside CE, Payment Per Day (D.4-o.c):

D.4-i.e & D.4-o.c After input of time for the “payment per day” payment type possibility completed in “CE set up” where the payment is converted to the hourly rate, the Direct Labor algorithm will calculate and display to the user the cost for the Ancillary Provider present or not present in the Post-op #2 stage. The Cost Table algorithm will pull this cost to the Pre-op #2 stage section of the Cost Table in the Cost Module. The Direct Labor algorithm will also calculate and display to the user the running “line cost” for the Ancillary Provider. The running “line cost” will be pulled to the Cost Table in the Cost Module by Cost table algorithm for the Ancillary Provider.

### Ancillary Provider, Post-Op #2 Stage, Inside CE, Payment Per Case (D.4-i.d):

Ancillary Provider, Post-Op #2 stage, Outside CE, Payment Per Case (D.4-o.d):

D.4-i.d & D.4-o.d After the “payment per case” payment type possibility is completed in “CE set up”, the cost for the Ancillary provider present or not present in the CE is complete. No further input is needed. The Direct Labor algorithm will display to the user this Ancillary Provider cost as the “line cost” for the Ancillary Provider in the CE. There is no running “line cost”. The Cost Table algorithm will pull this “line cost” to the Cost Table in the Cost Module.

### Ancillary Provider, Post-Op #2 Stage, Inside CE, Percentage of Ancillary Component (D.4-i.e):

Ancillary Provider, Post-Op #2 stage, Outside CE, Percentage of Ancillary Component (D.4-o.e):

D.4-i.e & D.4-o.e After the “percentage of ancillary component” payment type possibility is completed in “CE set up”, the cost for the Ancillary provider present or not present in the CE is complete. No further input is needed. Until the CE set up for “percentage of ancillary component” is complete the direct labor cost section for the Ancillary Provider will be INCOMPLETE. Once the CE setup has been completed, the Direct Labor algorithm will display to the user this Ancillary Provider cost as the “line cost” for the Ancillary Provider in the CE. There is no running “line cost”. The Cost Table algorithm will pull this “line cost” to the Cost Table in the Cost Module.

### Ancillary Provider, AO Time, Inside CE (D.5-i):

Ancillary Providers that are present during one or more stages of the CE have AO time. There is no AO time for Ancillary Providers who are not present in the CE but are engaged with the event that occurs in one or more stages of the CE.

D.5-i For AO time, the Direct Labor algorithm allows for the input of time in minutes that the Ancillary Provider present during CE spends doing purely administrative work. The administrative work must occur outside the CE but the time to perform this work does not need to be consecutive. AO time input for payment types D.5-i.a, D.5-i.b and D.5-i.e are described below. Payment types D.5-i.d and D.5-i.e does not have input for AO time.

### Ancillary Provider, AO Time, Inside CE, Payment Per Hour (D.5.a):

D.5-i.a After input of the time for “payment per hour” payment type possibility completed in “CE set up”, the Direct Labor algorithm will calculate and display to the user the cost for the Ancillary Provider present during CE for the AO time. The Cost Table algorithm will pull this cost to the AO time section of the Cost Table in the Cost Module. The Direct Labor algorithm will also calculate and display to the user the running “line cost” for the Ancillary Provider. The running “line cost” will be pulled to the Cost Table in the Cost Module by Cost table algorithm for the Ancillary Provider.

### Ancillary Provider, AO Time, Inside CE, Salary (D.5.b):

D.5-i.b After input of time for the “salary” payment type possibility completed in “CE set up” where the payment is converted to the hourly rate, the Direct Labor algorithm will calculate and display to the user the cost for the Ancillary Provider present during CE for the AO time. The Cost Table algorithm will pull this cost to the AO time section of the Cost Table in the Cost Module. The Direct Labor algorithm will also calculate and display to the user the running “line cost” for the Ancillary Provider. The running “line cost” will be pulled to the Cost Table in the Cost Module by Cost table algorithm for the Ancillary Provider.

### Ancillary Provider, AO Time, Inside CE, Payment Per Day (D.5.c):

D.5-i.e After input of time for the “payment per day” payment type possibility completed in “CE set up” where the payment is converted to the hourly rate, the Direct Labor algorithm will calculate and display to the user the cost for the Ancillary Provider present during CE for the AO time. The Cost Table algorithm will pull this cost to the AO time section of the Cost Table in the Cost Module. The Direct Labor algorithm will also calculate and display to the user the running “line cost” for the Ancillary Provider. The running “line cost” will be pulled to the Cost Table in the Cost Module by Cost table algorithm for the Ancillary Provider.

### Ancillary Provider, AO Time, Inside CE, Payment Per Case (D.5.d):

D.5-i.d After the “payment per case” payment type possibility is completed in “CE set up”, the cost for the Ancillary Provider present during CE in the CE is complete. No further input is needed. The cost of AO time is included in the “payment per case”. The Direct Labor algorithm will display to the user this Ancillary Provider cost as the “line cost” for the Ancillary Provider in the CE. There is no running “line cost”. The Cost Table algorithm will pull this “line cost” to the Cost Table in the Cost Module.

### Ancillary Provider, AO Time, Inside CE, Percentage of Professional Component (D.5.e):

D.5-i.e After the “percentage of ancillary component” payment type possibility is completed in “CE set up”, the cost for the Ancillary Provider present during CE in the CE is complete. No further input is needed. The cost of AO time is included in the “percentage of ancillary component” Until the CE set up for “percentage of ancillary component” is complete the direct labor cost section for the Ancillary Provider present during CE will be INCOMPLETE. Once the CE setup has been completed, the Direct Labor algorithm will display to the user this Ancillary Provider cost as the “line cost” for the Ancillary Provider in the CE. There is no running “line cost”. The Cost Table algorithm will pull this “line cost” to the Cost Table in the Cost Module.

### Cost Module [PART2]—Direct Supply Cost—Inventory Cost:

The Goal of Inventory Section in Surgery Center Cost Module is to build the stage card. The stage card is a summary of all the inventory costs for a particular CE. The stage card is divided into 5 sections. Each section represents one of the 5 Classes (II-VI) of Inventory that generate a cost in a CE. Class I does not generate a cost and is not part of the stage card. All Classes are described and defined below. Each individual section of the stage card lists numerically one after the other all the supplies and supply costs in that Class for a particular CE.

On the stage card, the supply is identified by Brand name and/or Generic name and by the Total CE item cost for that supply in a particular CE. Brand name, Generic Name and Total CE item cost are defined and discussed below.

The stage card algorithm calculates the cost of all items in each Class of inventory individually and the total cost of all Classes of inventory combined in the CE. The Cost Table algorithm pulls the cost of each of those Classes and the total cost of all the Classes combined to the Cost Table in the Cost Module. As items are added to stage card in any of the 5 sections, the stage card algorithm calculates the running total inventory cost for all items in that section and the running total inventory cost for all items on the stage card. As items are removed from stage card, the stage card algorithm also pulls that item off the Dynamic Cost Table from the Cost Module and Profit Module. The stage card and the total cost of all inventory items on the stage card can be saved to the stage card table by the stage card algorithm. The stage card table is navigated using the stage card table algorithm described below. When the CE is saved to CE database the stage card that was built in that CE is saved along with the entire CE in the CE database. The CE database is navigated using the CE Database algorithm discussed below.

Each item on the stage card is added to the stage card using the IIS algorithm for all inventory Classes II-VI. Classes II and III have an additional Estimate algorithm for estimating the global inventory cost for the items in those Classes as a group rather than as separate individual items with separate individual single item costs. The Estimate algorithm and IIS algorithm can both be used for Class II and III on the same stage card in the same CE but cannot be used together on the same Class. The estimated cost is pulled by Cost Table algorithm to Cost Table as the “class cost” for category Class II and Class III supplies when the Estimate Algorithm is used.

### IIS Algorithm:

The IIS algorithm's function is to take the cost of purchasing a supply in whatever amount it is purchased, individually or in bulk and translate that cost into the cost to use that supply in any amount in a CE. The IIS algorithm uses a blank “IIS card” to enter the supply and the cost of the supply used in a CE directly into that particular CE. The IS card is a series of mandatory and optional data entry fields that differ depending upon the type of supply being considered. How a supply is entered into an IIS card as well as the different mandatory and optional fields to be completed are discussed in detail below. The purpose of the IIS card is to calculate the “single item cost” of a supply and the cost of using one or more of those “items” in a CE. “Items” and “single item cost” have formal definitions. Once the IIS card is completed, the cost of that supply in the amount it is being used in a particular CE is calculated by IIS algorithm and pushed to the “stage card”. Once on the stage card the IIS algorithm will allow any item on the stage card to be opened back to its original IIS card. Once reopened, the IIS card can be edited and saved again to the stage card with the edited information overwriting the previous “item's” information on the stage card.

Items on the stage card can be deleted and removed from the stage card using the Delete function of the stage card algorithm.

The stage card is a summary of all the inventory costs for a particular CE. The stage card, once completed, can be saved to the stage card table. The completed IIS card can also be saved to the “AIR table”. The data pushed to the stage card table and the data pushed to the AIR table are similar but with important differences. AIR table is navigated using the AIR table algorithm This will also be discussed further below.

Using the IIS algorithm, supplies are entered into the IIS card through series of formal steps. The first step is to use IIS algorithm to choose the Class of the supply. Supplies fall into one of 6 classes all of which have formal definitions. These Classes are: I. Reusable General Supplies (aka Reusables), II. Disposable General Supplies (aka Disposables), III. Medications, IV. High-Cost Disposables, V. Implants and VI. DME-Orthotics.

(I) The first Class is Reusable General Supplies (aka Reusable). Reusables are a General Inventory Class that include all physical objects used during a client experience that can be used again in other client experiences for an indefinite number of time.

The cost of Reusables is not considered in the Inventory section of the Surgery Center Cost Submodule and is not considered by the IIS algorithm. Purchase of Reusables are considered in the Overhead Module as a capital expenditure and their cost allocated by the Depreciation algorithm to a CE. Rental of Reusables is considered separately in the Surgery Center Cost Submodule and is not considered part of Inventory as Reusables that are being rented are owned by the third party.

The remaining 5 classes are considered by the IIS algorithm. Supplies in those classes are direct costs in a CE.

(II) The second Class is Disposable General Supplies (aka Disposables).

Disposables are a General Inventory Class that includes the physical object or objects used during a client experience that have a one-time use or a defined limited use (aka “Extended Use”). Disposable General supplies have 3 Subclasses. These Subclasses are Ordinary Disposables, Disposable Packs and Disposable Instruments.

(III) The third Class is Medications. A medication is a Pharmaceutical Drug administered during the client experience. A medication is a substance recognized by an official pharmacopeia or formulary.

(IV) The fourth Class is High Cost Disposables (HCD). High Cost Disposables are Ordinary Disposables, Disposable Instruments or Disposable Packs of higher cost that is potentially reimbursable. Any of Ordinary Disposable, Disposable Instrument or Disposable Pack can be considered a HCD if it can be reimbursed and generate revenue in a particular CE.

(V) The fifth Class is Implants. Implants are a foreign object that is put physically inside a client and remains wholly intact inside the client following discharge. Implants are potentially reimbursable. Implants are usually classified as FDA CLASS Ill. Sutures are foreign objects that are put physically inside a client and remains wholly intact inside the client following discharge but are generally not reimbursable. Sutures are usually classified as FDA CLASS II.

(VI) The sixth Class is DME-Orthotics. DME-Orthotics are medical equipment that was given to a client that is not disposable and not reusable but durable. The DME-Orthotics provides support for physical limitation. The client will generally leave the client experience with a DME-Orthotics.

Once the first step is completed and the Class is chosen using the IIS algorithm, the remaining steps to enter the supply onto the IIS card using the IIS algorithm are different for each Class.

(II) For the Disposable Class the IIS algorithm allows the choice of 3. Subclasses in which to enter the supply.

(IIA) The first Subclass of the Disposable Class is Ordinary Disposables. Ordinary Disposables are defined as one or more “articles” grouped together as one “item” that have one time or defined limited use. Articles and Items also have formal definitions. Ordinary Disposables are for general type use and not designed for delicate work. Ordinary disposables are usually classified as FDA CLASS II. Ordinary disposables are not Medications, Implants or DME-Orthotics all of which have different formal definitions and are discussed below.

Once the choice of the first Subclass of Ordinary Disposables is made there are 4 methods (i-iv) to enter the supply into the Ordinary Disposables Subclass and onto the IIS card.

The first method uses categories that are part of the IIS algorithm. Some categories of Ordinary Disposables in the IIS algorithm have subcategories, and some do not. The list of categories and subcategories for Ordinary Disposables is extensive and is outlined in the Addendum. In all 4 methods the choice of a category or the creation of a category is mandatory.

Once the category is chosen or the subcategory for those categories that have subcategories, the IIS card appears and can then be filled out with the information for the supply that is to be used in a particular CE. To enter the supply onto the IIS card using the IIS algorithm, the choice of a category is mandatory. The choice of subcategories is optional. For those supplies that have a category and subcategory as part of IIS algorithm, the user can enter the supply under the category or the category/subcategory. Additional mandatory and optional fields for an Ordinary Disposable are discussed below.

The second method uses the categories of the IIS algorithm and allows the user to create a unique subcategory within that category. The category chosen for the creation of the unique subcategory may or may not have other subcategories already in the IIS algorithm.

The third method allows the user to create a unique category in the Ordinary Disposable Subclass.

The fourth method allows the user to create a unique category and subcategory in the Ordinary Disposable Subclass.

As with the first method, when the second, third or fourth method is completed the IIS card will also appear and can then be filled out with the information for the supply that is to be used in a particular CE.

The IIS card for Ordinary Disposables has four sections.

(IIA-1) The first section of the IIS card for Ordinary Disposables lists Disposables, Ordinary Disposables and category and the subcategory (when present) in the form: Disposables/Ordinary Disposables/category/subcategory. This first section of the IIS card for Ordinary Disposables has been determined by the previous choices made for the category and subcategory (if present). For Ordinary Disposables there always must be a category chosen which either comes from IIS algorithm or is created as a unique category as was described above. Additional information will be added to this section of the IIS card for Ordinary Disposables after category/subcategory (when present) by fields completed in the second section.

For Ordinary Disposables, when the IIS card appears with the Disposables/Ordinary Disposables/category/subcategory (if present) in the first section of the IIS card, there will also be a “conversion link” in this first section. When chosen, the Conversion Link will change (IIA) Disposables/Ordinary Disposables/category/subcategory (if present) to (IVA) HCD/Ordinary Disposables/category/subcategory (if present).

(IIA-2) The second section of the IIS card for Ordinary Disposables has 4 mandatory fields and 5 optional fields to be filled out. The mandatory fields must be filled out in order for the IIS card to be complete and allow the total cost of the supply in the CE defined as “Total CE item cost” to be moved to the stage card. The mandatory fields also need to be filled out in order to save the “single item cost” to the AIR table. Optional fields do not have to be filled out to move the “single item cost” of the supply to the AIR table or “Total CE item cost” of the supply to the stage card. The optional fields are there to allow for additional information on the Ordinary Disposable to be included and serve as fields that can be searched in the AIR table and as background data on the stage card and in the stage card table.

) The mandatory fields are: Generic or Brand name, Bulk Item cost, number of items in a Bulk (#in Bulk) and number used (#used).

The Generic Name is the common and generally accepted designation for a supply that identifies that supply and distinguishes it from other supplies. Generic name is free form text field

The Brand Name is the proprietorial name given to a supply by the maker of that supply. Brand name is free form text field

A Bulk Item is Collection of single items purchased together. The cost of purchasing a bulk item is the Bulk Item Cost. Bulk item cost is a “free form text field” in dollars.

Number of items in a Bulk (#in Bulk) is the number of items contained in a Bulk when the items are purchased in bulk.

The “Number used” (#used) is the number of items used in the CE. The “number used” is a single-select list box option.

In order for IIS card to be complete, the Generic Name or Brand Name must be entered onto the IIS card. Both can be entered but at least one must be entered for the card to be complete and be able to be pushed to the stage card, stage card database or the AIR database. Once the Generic Name and/or Brand Name is entered onto the IIS card, the Generic Name and/or Brand Name will appear in Section One of the IIS card after the last category or subcategory that was previously chosen. If the Generic and Brand Name are both entered onto the IIS card, then the first section will read Disposables/Ordinary Disposables/Category/Subcategory (if chosen)/Generic Name/Brand Name.

ii) The optional fields are: Extended use, Body part, Specialty/Subspecialty, Manufacturer, Vendor.

“Extended use” is when a disposable can be used in one CE and reused again for a defined and limited number of times in other CEs. The field to enter extended use in the IIS card is a single select list box option with options #1-#20.

Body part are listed in the Addendum. Body part options are multi-select list box options where one or more option can be chosen from the list. There is no “free form text field”.

Specialty/Subspecialties are listed in the Addendum. Specialty/Subspecialties options are multi-select list box options where one or more option can be chosen from the list. There is no “free form text field”. Some Specialties on the list have nested Subspecialties and some do not.

Manufacturer is the company that manufactures the supply. The field to enter the manufacturer on the IIS Card is a “free form text field”. This optional free form text field can be saved to the AIR database. When the IIS card is pushed to the stage card and the stage card is saved to stage card database, the manufacturer in the free form text field will also be saved with the stage card. Both these instances will be covered in detail below when the AIR Database algorithm and stage card Database algorithm are discussed.

Vendor is the seller of the supply. As with the Manufacturer, the field to enter the vendor on the IIS Card is a “free form text field”. The optional free form text field can again be saved to the AIR database once the IIS card is complete and to the stage card Database once the stage card is saved to the stage card Database.

(IIA-3) The third section of the IIS card for Ordinary Disposables calculates the “single item cost” for the supply and the “Total CE item cost” to use that item in the amount that it is going to be used in the CE. The calculations are made using mandatory fields from the second part of the IIS card. The single item cost is the cost of the bulk item divided by the number of items in the bulk. The Total CE item cost are the number of items used in the CE times the single item cost divided by extended use number if the “extended use” option is chosen. The IIS algorithm will calculate Total CE item cost when “calculate cost” is chosen.

The third section of the IIS card for Ordinary disposables can be saved to the AIR database. The Bulk cost and the single item cost are saved to AIR database. The number used and Total CE item cost are not saved to the AIR database. The third section of the IIS card for Ordinary Disposables can be pushed to stage card and the stage card can be saved to the stage card database. Stage card database will include all fields in the third section of the IIS card including number used and Total CE item cost.

(IIA-4) The fourth section of the IIS card for Ordinary Disposables is a notes section. It is a free form text field. When the free form text written in the note section is saved to the AIR database or pushed to the stage card the free form text is dated. If the stage card is saved to the stage card database, the dated text written in the note section is also saved.

As will be discussed further in detail below, the AIR Database algorithm interfaces with the AIR database and the stage card Database algorithm interfaces with the stage card database. The free form text in the note section that was saved to those databases using the IIS algorithm will be present. If either the AIR database or stage card database is used to enter supplies into a new IIS card for a different CE the notes will follow. The new notes written using either the AIR Database algorithm or the stage card Database algorithm into a new IIS card are independent of each other. Once the new notes in the note section of a new IIS card built using either of the two different databases is saved to that database, it is saved only to that database and does not cross over to the other database.

(IIB) The second Subclass of the Disposables Class is Disposable Packs. Disposable Packs are defined as more than one Ordinary Disposable put into a Pack.

Disposable Packs have two Subdivisions, “Disposable Custom Packs” and “Disposable Vendor Packs”.

(IIB-d′) Disposable Custom Packs are more than one Ordinary Disposable put into a pack in which the pack is custom made for the buyer and purchased from a third party.

Once the choice of Disposable Custom Packs is made there is only one method to enter the supply onto the IIS card.

As with Ordinary Disposables, the IIS card for Disposable Custom Packs has 4 sections.

(IIB-d′-1) For Disposable Custom Packs, the first section of the IIS card lists Disposables, Disposable Pack, Disposable Custom Pack in the form: Disposable/Disposable Pack/Disposable Custom Pack. There are no categories or subcategories for Disposable Custom Packs.

For Disposables Custom Packs, when the IIS card appears with the Disposables/Disposables Custom Packs in the first section of the IIS card, there will also be a “conversion link” in this first section. When chosen, the Conversion Link will change (IIB-d′) Disposables/Disposables Custom Packs to (IVB-d′) HCD/Disposables Custom Packs.

(IIB-d′-2) As with Ordinary Disposables, the second section of the IIS card for Disposables Custom Packs has mandatory and optional fields to be filled out. Disposable Custom Packs has 3 mandatory fields and 3 optional fields. The mandatory fields must be filled out in order for the IIS card to be complete and allow the total cost of the Disposable Custom Pack defined as “Total CE item cost” to be moved to the stage card. The mandatory fields also need to be filled out to in order to save the “single item cost” of the Disposable Custom Pack to the AIR database. Optional fields do not have to be filled out to move the “single item cost” of the Disposable Custom Pack to the stage card or AIR database. The optional fields are there to allow for additional information on the Disposable Custom Packs to be included and serve as fields that can be searched in the AIR database and as background data on the stage card and in the stage card database.

Disposable Custom Packs are defined as an “Items”.

The mandatory fields are Generic name, Single Item Cost, and Number used (#used). There is no Brand name or Bulk Item cost.

The Generic Name is the name of the pack as it is designated by the Buyer or Vendor.

The Single Item Cost is a cost to purchase the pack from the vendor.

The Number used (#used) is the number of Disposable Custom Packs used in the CE.

In order for IIS card to be complete, the Generic Name must be entered onto the IIS card. Once complete, the IIS card can be pushed to the stage card, stage card database or the AIR database. Once the Generic Name is entered onto the IIS card, the Generic name will appear in Section One of the IIS card after the Subdivision of Disposable Custom Packs. Once the Generic name is entered onto the IIS card, the first section will read Disposables/Disposable Packs/Disposables Custom Packs/Generic Name.

The optional fields are: Body Part, Specialty/Subspecialty, Vendor. There is no Manufacturer.

(IIB-d′-3) As with Ordinary Disposables, the third section of the IIS card for Disposable Custom Packs calculates the “Total CE item cost” which is the cost to use a Disposable Pack in whatever number that it is going to be used in the CE. The calculations are made using the mandatory fields from the second part of the IIS card. The CE single item cost is the number of packs (“Items”) used in the CE times the single Item Cost. The IIS algorithm will calculate Total CE item cost when “calculate cost” is chosen.

“Total CE item cost” is not part of the AIR database and therefore “Total CE item cost” for Disposable Custom Packs is not saved to the AIR database. The third section of the IIS card for Disposable Custom Packs can be pushed to stage card and the stage card can be saved to the stage card database. Stage card database will include Total CE item cost.

(IIB-d′-4) The fourth section of the IIS card for Disposables Custom Packs is a notes section. It is a free form text field. The contents of the pack can be written here as free form text. When the note section is saved to the AIR database or pushed to the stage card the free form text is dated. If the stage card is saved to the stage card database, the dated text written in the note section is also saved. As discussed above, the AIR database notes' field, and the stage card notes' field are independent of each other.

(IIB-d″) Disposable Vendor Packs are more than one Ordinary Disposable put into a pack that is pre-made by the vendor with minimal or no customization from the buyer. A Disposable Vendor Pack is by definition an “Item”.

Once the choice of Disposable Vendor Packs is made there is only one method to enter the supply onto the IIS card.

As with Ordinary Disposables, the IIS card for Disposable Vendor Packs has 4 sections.

(IIB-d″-1) For Disposable Vendor Packs, the first section of the IIS card lists Disposables, Disposable Pack, Disposable Vendor Pack in the form: Disposable/Disposable Pack/Disposable Vendor Pack. There are no categories or subcategories for Disposable Vendor Packs.

For Disposables Vendor Packs, when the IIS card appears with the Disposables/Disposables Vendor Packs in the first section of the IIS card, there will also be a “conversion link” in this first section. When chosen, the Conversion Link will change (IIB-d″) Disposables/Disposables Vendor Packs to (IVB-d″) HCD/Disposables Custom Packs/category/subcategory (if present).

(IIB-d″-2) As with Ordinary Disposables, the second section of the IIS card for Disposables Vendor Packs has mandatory and optional fields to be filled out. Disposable Vendor Packs has 3 mandatory fields and 4 optional fields. The mandatory fields must be filled out in order for the IIS card to be complete and allow the total cost of the Disposable Vendor Pack defined as “Total CE item cost” to be moved to the stage card. The mandatory fields also need to be filled out to in order to save the “single item cost” of the Disposable Vendor Pack to the AIR database. Optional fields do not have to be filled out to move the “single item cost” of the Disposable Vendor Pack to the AIR database. Optional fields do not need to be filled out to move data from the IIS card to the stage card and to save stage card to stage card database. The optional fields are there to allow for additional information on the Disposable Vendor Packs to be included and serve as fields that can be searched in the AIR database and as background data on the stage card and in the stage card database. Disposable Vendor Packs are defined as “Items”.

i) The mandatory fields are Generic name and/or Brand Name, Single Item Cost, and Number used (#used). There is no Bulk Item cost.

The Generic Name is the name given by the Buyer.

The Brand Name is the name designated by the Vendor or Manufacturer.

The Single Item Cost is the cost to purchase the pack from the Vendor or Manufacturer.

The Number used (#used) is the number of Disposable Vendor Packs used in the CE.

In order for IIS card to be complete, the Generic Name or Brand Name must be entered onto the IIS card. Both can be entered but at least one needs to be entered for the card to be complete and be able to be pushed to stage card, stage card database or the AIR database. Once the Generic Name and/or Brand Name is entered onto the IIS card, the Generic name and/or Brand Name will appear in Section One of the IIS card after the Subdivision of Disposable Vendor Packs. If the Generic Name and Brand Name are both entered onto the IIS card then the first section will read Disposables/Disposable Packs/Disposables Vendor Packs/Generic Name/Brand Name.

ii) The optional fields are: Body Part, Specialty/Subspecialty, Manufacturer, Vendor.

(IIB-d′-3) As with Ordinary Disposables, the third section of the IIS card for Disposable Vendor Packs calculates the “Total CE item cost” which is the cost to use a Disposable Pack in whatever number that it is going to be used in the CE. The calculations are made using the mandatory fields from the second part of the IIS card. The Total CE item cost is the number of packs (“Items”) used in the CE times the Single Item Cost. The IIS algorithm will calculate Total CE item cost when “calculate cost” is chosen.

Total CE item cost is not part of the AIR database and therefore Total CE item cost for Disposable Vendor Packs is not saved to the AIR database. The third section of the IIS card for Disposable Vendor Packs can be pushed to stage card and the stage card can be saved to the stage card database. Stage card database will include Total CE item cost.

(IIB-d″-4) The fourth section of the IIS card for Disposables Vendor Packs is a notes section. It is a free form text field. The contents of the pack can be written here as free form text. When the note section is saved to the AIR database or pushed to the stage card, the free form text is dated. If the stage card is saved to the stage card database, the dated text written in the note section is also saved. As discussed above, the AIR database notes field, and the stage card notes field are independent of each other.

(IIC) The third Subclass of the Disposable Class is Disposable Instruments. Like the Ordinary Disposables Subclass, the Disposable Instruments Subclass has categories and subcategories.

Once the choice of the third Subclass of Disposables Instruments is made there are 4 methods (i-iv) to enter the supply into the Disposables Instruments Subclass and onto the IIS card as there is with the Ordinary Disposable Subclass.

i. The first method uses categories that are part of the IIS algorithm. Some categories of Disposables Instruments in the IIS algorithm have subcategories, and some do not. The list of categories and subcategories for Disposables Instruments is outlined in the Addendum.

Once the category is chosen or the subcategory for those categories that have subcategories, the IIS card appears and can then be filled out with the information for the supply that is to be used in a particular CE. The mandatory and optional fields for a Disposable Instruments are discussed below.

ii. The second method uses the categories of the IIS algorithm and allows for the creation of a unique subcategory within that category. The category chosen for the creation of the unique subcategory may or may not have other subcategories already in the IIS algorithm.

iii. The third method allows the unique creation of a category in the Disposable Instruments Subclass.

iv. The fourth method allows the unique creation of a category and subcategory in the Disposable Instruments Subclass.

As with the first method, when the second, third or fourth method is completed the IIS card will also appear and can then be filled out with the information for the Disposable Instrument that is to be used in a particular CE.

As with Ordinary Disposables and Disposable Packs, the IIS card for Disposables Instruments has four sections.

(IIC-1) The first section of the IIS card for Disposables Instruments lists Disposables, Disposables Instruments and category and the subcategory (when present) in the form: Disposables/Disposables Instruments/category/subcategory. This first section of the IIS card for Disposables Instruments has been determined by the previous choices made for the category and subcategory (if present). For Disposables Instruments there always must be a category chosen which either comes from IIS algorithm or is created as a unique category as was described above. Additional information will be added to this section of the IIS card for Disposables Instruments after category/subcategory (when present) by fields completed in the second section.

For Disposables Instruments, when the IIS card appears with the Disposables/Disposables Instruments/category/subcategory (if present) in the first section of the IIS card, there will also be a “conversion link” in this first section. When chosen, the Conversion Link will change (IIC) Disposables/Disposable Instruments/category/subcategory (if present) to (IVC) HCD/Disposable Instruments/category/subcategory (if present).

(IIC-2) The second section of the IIS card for Disposables Instruments has 4 mandatory fields and 5 optional fields to be filled out. The mandatory fields must be filled out in order for the IIS card to be complete and allow the total cost of the disposable instrument(s) in the CE defined as “Total CE item cost” to be moved to the stage card. The mandatory fields also need to be filled out to in order to save the “single item cost” to the AIR database. Optional fields do not have to be filled out to move the “single item cost” of the disposable instrument(s) to the AIR database. Optional fields do not have to be filled out to move the data from the IIS card to the stage card and save the stage card to the stage card database. The optional fields are there to allow for additional information on the disposable instrument to be included and serve as fields that can be searched in the AIR database and as background data on the stage card and in the stage card database.

i) The mandatory fields are: Generic or Brand name, Bulk Item cost, Number of items in a Bulk (#in Bulk) and number used (#used).

The Generic Name is the common and generally accepted designation for the disposable instrument that uniquely identifies that supply and distinguishes it from other disposable instruments. Generic name is free form text field

The Brand Name is the proprietorial name given to a disposable instrument by the maker of that disposable instrument. Brand name is free form text field

A Bulk Item is Collection of single disposable instruments “items” purchased together. The cost of purchasing a bulk item of disposable instruments is the Bulk Item Cost. Bulk item cost is a “free form text field” in dollars.

Number of items in a Bulk (#in Bulk) is the number of items contained in a Bulk when the items are purchased in bulk.

The “Number used” (#used) is the number of disposable instrument “items” used in the CE. The “number used” is a single-select list box option.

In order for IIS card to be complete, the Generic Name or Brand Name of the Disposable Instrument must be entered onto the IIS card. Both can be entered but at least one must be entered for the card to be complete and be able to be pushed to the stage card, stage card database or the AIR database. Once the Generic Name and/or Brand Name is entered onto the IIS card, the Generic Name and/or Brand Name will appear in Section One of the IIS card after the last category or subcategory that was previously chosen. If the Generic and Brand Name are both entered onto the IIS card, then the first section will read Disposables/Disposables Instruments/Category/Subcategory (if chosen)/Generic Name/Brand Name.

The optional fields are: “Extended use”, Body part, Specialty/Subspecialty, Manufacturer, Vendor.

“Extended use” is when a disposable instrument can be used in one CE and reused again for a defined and limited number of times in other CEs. The field to enter extended use in the IIS card is a single select list box option with options #1-#20.

Body part are listed in the Addendum. Body part options are multi-select list box options where one or more option can be chosen from the list. There is no “free form text field”.

Specialty/Subspecialties are listed in the Addendum. Specialty/Subspecialties options are multi-select list box options where one or more option can be chosen from the list. There is no “free form text field”. Some Specialties on the list have nested Subspecialties and some do not.

Manufacturer is the company that manufactures the supply. The field to enter the manufacturer on the IIS Card is a “free form text field”. This optional free form text field can be saved to the AIR database. When the IIS card is pushed to the stage card and the stage card is saved, the manufacturer in the free form text field will also be saved with the stage card. Both these instances will be covered in detail below when the AIR Database algorithm and stage card Database algorithm for Disposable Instruments are discussed.

Vendor is the seller of the disposable instrument. As with the Manufacturer, the field to enter the vendor on the IIS Card is a “free form text field”. The optional free form text field can again be saved to the AIR database once the IIS card is complete and to the stage card Database once the stage card is saved to the stage card Database.

(IIC-3) The third section of the IIS card for Disposables Instruments calculates the “single item cost” for the disposable instrument and the “Total CE item cost” to use that item in the amount that it is going to be used in the CE. The calculations are made using mandatory fields from the second part of the IIS card. The “single item cost” is the cost of the bulk item divided by the number of items in the bulk. The “Total CE item cost” are the number of disposable instruments used in the CE times the “single item cost” divided by extended use number if the “extended use” option is chosen. The IS algorithm will calculate “Total CE item cost” when “calculate cost” is chosen.

The third section of the IIS card for Disposables Instruments can be saved to the AIR database. The Bulk cost and the single item cost are saved to AIR database. The number used and Total CE item cost are not saved to the AIR database. The third section of the IIS card for Disposables Instruments can be pushed to stage card and the stage card can be saved to the stage card database. Stage card database will include all fields in the third section of the IIS card including number used and “Total CE item cost”.

(IIC-4) The fourth section of the IIS card for Disposable Instruments is a notes section. It is a free form text field. The contents of the pack can be written here as free form text. When the note section is saved to the AIR database or pushed to the stage card, the free form text is dated. If the stage card is saved to the stage card database, the dated text written in the note section is also saved. As discussed above, the AIR database notes field, and the stage card notes field are independent of each other.

(III) The third Class is Medications. Medications are defined as a Pharmaceutical Drug administered during the client experience. A medication is a substance recognized by an official pharmacopeia or formulary.

Once the choice of the third Class of Medications is made, there are two methods (i-ii) to enter the medication onto the IIS card for Medications. The Medication Class has no Subclasses. The Medication Class has categories but has no subcategories.

The first method uses categories that are part of the IIS algorithm. The list of categories for Medications is outlined in the Addendum.

Once the category is chosen, the IIS card appears and then can be filled out with the information for the medication that is to be used in a particular CE. The mandatory and optional fields for Medications are discussed below.

The second method allows for the unique creation of a new category in the Medication Class.

When a unique category is created, the IIS card appears and can be filled out with the information for the medication in that new category. The mandatory and optional fields for a new category of Medications are discussed below.

The IIS card for Medication has 4 sections.

(III-1) The first section of the IIS card lists Medications and category in the form: Medications/category. This first section of the IIS card for Medications has been determined by the previous choice made for the category. For Medications there always must be a category chosen which either comes from IIS algorithm or is created as a unique category as was described above. Additional information will be added to this section of IIS card for Medications after the category is chosen or created by fields completed in the second section.

(III-2) The second section of the IIS card for Medications has 4 mandatory fields and 4 optional fields to be filled out. The mandatory fields must be filled out in order for the IIS card to be complete and allow the total cost of the medication in the CE defined as “Total CE item cost” to be moved to the stage card. The mandatory fields also need to be filled out to in order to save the “single item cost” of the medication to the AIR database. Optional fields do not have to be filled out to move the “single item cost” of the medication to the AIR database. Optional fields do not have to be filled out to move the data from the IIS card to the stage card and saved to the stage card database. The optional fields are there to allow for additional information on the medication to be included and serve as fields that can be searched in the AIR database and as background data on the stage card and in the stage card database.

i) The mandatory fields are: Generic or Brand name, Bulk Item cost, Number of items in a Bulk (#in Bulk) and number used (#used) or the percentage of the Item that is used (% used).

The Generic Name of the medication is the chemical name of the medication as opposed to its advertised or brand name under the which medication is sold. The generic name of the medication identifies that medication and distinguishes it from other medications. Generic name is free form text field.

The Brand Name is the proprietorial or trademark name given to a medication by the manufacturer of that medication. Brand name is free form text field.

A Bulk Item is a collection of single items purchased together. The cost of purchasing a bulk item is the Bulk Item Cost. Bulk item cost is a “free form text field” in dollars.

Number of items in a Bulk (#in Bulk) is the number of items contained in a Bulk when the items are purchased in bulk.

The “Number used” (#used) is the number of items used in the CE. The “number used” is a single-select list box option.

The percentage of the Item that is used (% used) is the amount of the item that is used when the medication item constitutes 100% and less than 100% is used in the CE. The % used is a single-select list box option.

In order for IIS card to be complete, the Generic Name or Brand Name must be entered onto the IIS card. Both can be entered but at least one must be entered for the card to be complete and be able to be pushed to the stage card, stage card database or the AIR database. Once the Generic Name and/or Brand Name is entered onto the IIS card, the Generic Name and/or Brand Name will appear in Section One of the IIS card after the category that was previously chosen. If the Generic and Brand Name are both entered onto the IIS card, then the first section will read Medications/Category/Generic Name/Brand Name.

The optional fields are: Units of Measurements, CPT/HCPCS Code (J-code), Manufacturer, Vendor.

“Units of Measurements” are the measurements used when dispensing the medication to the client in the CE. Units of measurements are: grams, mg, mcg, mg/ml, mcg/ml, cc, vial, units, drops. The field to enter Units of measurements on the IIS card is a single-select list box option with an “other” option. The “other” option, when chosen, is a “free form text field”. Only one unit of measurement can be chosen either from single-select list box option or the free form text field “other” option.

CPT/HCPCS Codes (J-codes) are the standard supply codes for medications. The field to enter the J-codes on the IIS Card is a “free form text field”.

Manufacturer is the company that manufactures the medication. The field to enter the manufacturer on the IIS Card is a “free form text field”. This optional free form text field can be saved to the AIR database. When the IIS card is pushed to the stage card and the stage card is saved, the manufacturer in the free form text field will also be saved with the stage card. Both these instances will be covered in detail below when the AIR Database algorithm and stage card Database algorithm are discussed.

Vendor is the seller of the medication. As with the Manufacturer, the field to enter the vendor on the IIS Card is a “free form text field”. The optional free form text field can again be saved to the AIR database once the IIS card is complete and to the stage card Database once the stage card is saved to the stage card Database.

(III-3) The third section of the IIS card for Medication calculates the single “single item cost” for the medication and the “Total CE item cost” to use that item in the amount that it is going to be used in the CE. The calculations are made using mandatory fields from the second part of the IIS card. The “single item cost” is the cost of the bulk item divided by the number of items in the bulk.

The “Total CE item cost” is calculated by one of two mutually exclusive methods depending upon whether the “#used” or the “% used” mandatory field is chosen. If one field is chosen the other field cannot be chosen. When the “#used” is chosen the IIS algorithm will calculate the Total CE item cost by multiplying the “#used” by the single item cost. When the “% used” is chosen the IIS algorithm will calculate the “Total CE item cost” by multiplying the “single item cost” times the “% used”. The IIS algorithm will calculate “Total CE item cost” when “calculate cost” is chosen.

The third section of the IIS card for Medications can be saved to the AIR database. The Bulk cost and the single item cost are saved to AIR database. The “#used”, the “% used” and “Total CE item cost” are not saved to the AIR database. The third section of the IIS card for Medications can be pushed to stage card and the stage card can be saved to the stage card database. Stage card database will include all fields in the third section of the IIS card including the “#used”, the “% used” and “Total CE item cost”.

(III-4) The fourth section of the IIS card for Medications is a notes section. It is a free form text field. When the free form text written in the note section is saved to the AIR database or pushed to the stage card the free form text is dated. If the stage card is saved to the stage card database, the dated text written in the note section is also saved. As discussed, elsewhere, the AIR database notes field, and the stage card notes field are independent of each other.

### (IV) The Fourth Class is High-Cost Disposables (HCD)

High-Cost Disposables (HCD) are supplies that can be placed into the Disposable Class and into one of the Subclasses of Ordinary Disposables, Disposable Instruments or Disposable Packs but are of higher cost and are potentially reimbursable. Any supply that is in the Subclasses of Ordinary Disposables, Disposable Instruments or Disposable Packs can be considered a HCD if it can be reimbursed and generate revenue in a particular CE. The HCPCS/CPT code for HCD is 99070 and may include the “DI” modifier.

As with the Disposable Class there are 3 Subclasses of the HCD Class which mirror the Disposable Class. These are HCD/Ordinary Disposables, HCD/Disposable Packs, HCD/Disposable Instruments. HCD/Disposable Packs again mirror Disposable Packs and have two Subdivisions which are HCD/Disposable Custom Packs, HCD/Disposable Vendor Packs.

(IVA) As with the first subclass of Ordinary Disposables, once the choice of the first subclass of HCD/Ordinary Disposables are made there are 4 methods (i-iv) to enter the supply into the HCD/Ordinary Disposable into the IIS card. The methods are the same as for Ordinary Disposables. Refer to (IIA).

Unlike Ordinary Disposables, the IIS card for HCD/Ordinary Disposables has 5 sections.

(IVA-1) The first section of the IIS card for HCD lists HCD, Ordinary Disposables and category and the subcategory (when present) in the form: HCD/Ordinary Disposables/category/subcategory. This first section of the IIS card for HCD/Ordinary Disposables has been determined by the previous choices made for the category and subcategory (if present). For HCDs/Ordinary Disposables there always must be a category chosen which either comes from IIS algorithm or is created as a unique category as was described above. Additional information will be added to this section of the IIS card for HCDs/Ordinary Disposables after category/subcategory (when present) by fields completed in the second section.

(IVA-2) The second section of the IIS card for HCD/Ordinary Disposables has 4 mandatory fields and 4 optional fields to be filled out. The mandatory fields must be filled out in order for the IIS card to be complete and allow the total cost of the supply in the CE defined as “Total CE item cost” to be moved to the stage card. The mandatory fields also need to be filled out too in order to save the “single item cost” to the AIR database. Optional fields do not have to be filled out to move the “single item cost” of the supply to the AIR database. Optional fields do not have to be filled out to move the data from the IIS card to the stage card and saved to the stage card database. The optional fields are there to allow for additional information on the HCD/Ordinary Disposable to be included and serve as fields that can be searched in the AIR database and as background data on the stage card and in the stage card database.

i) The mandatory fields are: Generic or Brand name, Bulk Item cost, number of items in a Bulk (#in Bulk) and number used (#used).

The Generic Name is the common and generally accepted designation for a supply that identifies that supply and distinguishes it from other supplies. Generic name is free form text field.

The Brand Name is the proprietorial name given to a supply by the maker of that supply. Brand name is free form text field.

A Bulk Item is Collection of single items purchased together. The cost of purchasing a bulk item is the Bulk Item Cost. Bulk item cost is a “free form text field” in dollars.

Number of items in a Bulk (#in Bulk) is the number of items contained in a Bulk when the items are purchased in bulk.

The “Number used” (#used) is the number of items used in the CE. The “number used” is a single-select list box option.

In order for IIS card to be complete, the Generic Name or Brand Name must be entered onto the IIS card. Both can be entered but at least one must be entered for the card to be complete and be able to be pushed to the stage card, stage card database or the AIR database. Once the Generic Name and/or Brand Name is entered onto the IIS card, the Generic Name and/or Brand Name will appear in Section One of the IIS card after the last category or subcategory that was previously chosen. If the Generic and Brand Name are both entered onto the IIS card, then the first section will read HCD/Ordinary Disposables/Category/Subcategory (if chosen)/Generic Name/Brand Name.

ii) The optional fields are: Body part, Specialty/Subspecialty, Manufacturer, Vendor.

Body part are listed in the Addendum. Body part options are multi-select list box options where one or more options can be chosen from the list. There is no “free form text field”.

Specialty/Subspecialties are listed in the Addendum. Specialty/Subspecialties options are multi-select list box options where one or more options can be chosen from the list. There is no “free form text field”. Some Specialties on the list have nested Subspecialties and some do not.

Manufacturer is the company that manufactures the supply. The field to enter the manufacturer on the IIS Card is a “free form text field”. This optional free form text field can be saved to the AIR database. When the IIS card is pushed to the stage card and the stage card is saved, the manufacturer in the free form text field will also be saved with the stage card. Both these instances will be covered in detail below when the AIR Database algorithm and stage card Database algorithm are discussed.

Vendor is the seller of the supply. As with the Manufacturer, the field to enter the vendor on the IIS Card is a “free form text field”. The optional free form text field can again be saved to the AIR database once the IIS card is complete and to the stage Card Database once the stage Card is saved to the stage Card Database.

There is no “extended use” option for HCDs. HCDs are reimbursable per CE and are not used in more than one CE.

(IVA-3) The third section of the IIS card for HCD/Ordinary Disposables calculates the single “single item cost” for the supply and the “Total CE item cost” to use that item in the amount that it is going to be used in the CE. The calculations are made using mandatory fields from the second part of the IIS card. The “single item cost” is the cost of the bulk item divided by the number of items in the bulk. The “Total CE item cost” are the number of items used in the Total CE times the single item cost. The IIS algorithm will calculate “Total CE item cost” when “calculate cost” is chosen.

The third section of the IIS card for HCD/Ordinary disposables can be saved to the AIR database. The Bulk cost and the single item cost are saved to AIR database. The number used and Total CE item cost are not saved to the AIR database. The third section of the IIS card for Ordinary Disposables can be pushed to stage card and the stage card can be saved to the stage card database. Stage card database will include all fields in the third section of the IIS card including number used and Total CE item cost.

(IVA-4) The fourth section of the IIS card for HCD/Ordinary Disposables is a notes section. It is a free form text field. When the free form text written in the note section is saved to the AIR database or pushed to the stage card, the free form text is dated. If the stage card is saved to the stage card database, the dated text written in the note section is also saved.

As will be discussed further in detail below, the AIR Database algorithm interfaces with the AIR database and the stage card Database algorithm interfaces with the stage card database. The free form text in the note section that was saved to those databases using the IIS algorithm will be present. If either the AIR database or stage card database is used to enter supplies into a new IIS card for a different CE the notes will follow. The new notes written using either the second AIR Database algorithm or the stage card Database algorithm into a new IIS card are independent of each other. Once the new notes in the note section of a new IIS card built using either of the two different databases is saved to that database, it is saved only to that database and does not cross over to the other database.

(IVA-5) The fifth section for IIS card for HCD/Ordinary Disposables has one mandatory link, call Link to Revenue algorithm that must be completed in order for the IIS card to be complete and allow the total cost of the HCD in the CE defined as “Total CE item cost” to be moved to the stage card. This mandatory link also needs to be completed in order to save the “single item cost” of the HCD to the AIR database.

The link will connect to the Revenue Table of the Surgery Center Revenue Submodule where the procedure codes (CPT/HCPCS codes) that are billed in the surgical case have been organized sequentially as they would be formally billed by the ASC. The line of code that corresponds to the HCD is then chosen. That line of code is usually 99070 with or without “DI” or other modifier. Once that line of code is chosen, the description for that line of code as written on the Revenue Table is pulled to the fifth section of the IIS card. Once the IIS card is completed and pushed to the stage card, the “Total CE item cost” is linked to the “total revenue” or “corrected total revenue” if the Collection Discount algorithm was applied for the HCD. This link will allow changes in cost for that HCD to be linked to the changes in revenue or corrected revenue for that HCD in the Profit Module. This link will be discussed further in the profit module.

When the completed IIS card is pushed to stage card, a link is established between the Cost and Revenue for the HCD/Ordinary Disposables. This link is used in the Profit Module. When the IIS card is pushed to AIR database the link is broken and the fifth section of the IIS card is removed. This will be discussed below when using the AIR Database algorithm. When the stage card is saved to the stage card database the link and the fifth section of the IIS card is also removed. This will be discussed below when using the stage card Database algorithm. In both cases, this will allow new links to be established when the AIR database or stage card database is used for other CEs with different revenue tables. When the CE is saved to the CE database all links are initially maintained but can be changed. This will be discussed in the CE Database algorithm.

(IVB) The second Subclass of the HCD Class is Disposable Packs. HCD/Disposable Packs are defined as more than one Ordinary Disposable put into a Pack.

HCD/Disposable Packs have two Subdivisions, “Disposable Custom Packs” and “Disposable Vendor Packs”.

(IVB-d′) HCD/Disposable Custom Packs are more than one ordinary disposable put into a pack in which the pack is custom made for the buyer and purchased from a third party.

Once the choice of HCD/Disposable Custom Packs is made there is only one method to enter the supply onto the IIS card.

As with HCD/Ordinary Disposables, the IIS card for Disposable Custom Packs has 5 sections.

(IVB-d′-1) For Disposable Custom Packs, the first section of the IIS card lists HCD, Disposable Pack, Disposable Custom Pack in the form: HCD/Disposable Pack/Disposable Custom Pack. There are no categories or subcategories for Disposable Custom Packs.

(IVB-d′-2) As with HCD/Ordinary Disposables, the second section of the IIS card for HCD/Disposables Custom Packs has mandatory and optional fields to be filled out. HCD/Disposable Custom Packs has 3 mandatory fields and 3 optional fields. The mandatory fields must be filled out in order for the IIS card to be complete and allow the total cost of the HCD/Disposable Custom Pack defined as “Total CE item cost” to be moved to the stage card. The mandatory fields also need to be filled out too in order to save the “single item cost” of the HCD/Disposable Custom Pack to the AIR database. Optional fields do not have to be filled out to move the “single item cost” of the HCD/Disposable Custom Pack to the AIR database. Optional fields do not have to be filled out to move the data from the IIS card to the stage card and saved to the stage card database. The optional fields are there to allow for additional information on the HCD/Disposable Custom Pack to be included and serve as fields that can be searched in the AIR database and as background data on the stage card and in the stage card database.

The mandatory fields are Generic name, Single Item Cost, and Number used (#used). There is no Brand name or Bulk Item cost.

The Generic Name is the name of the pack as it is designated by the Buyer or Vendor.

The Single Item Cost is a cost to purchase the pack from the vendor.

The Number used (#used) is the number of HCD/Disposable Custom Packs used in the CE.

In order for IIS card to be complete, the Generic Name must be entered onto the IIS card. Once complete, the IIS card can be pushed to the stage card, stage card database or the AIR database. Once the Generic Name is entered onto the IIS card, the Generic name will appear in Section One of the IIS card after the Subdivision of HCD/Disposable Custom Packs. Once the Generic name is entered onto the IIS card, the first section will read HCD/Disposable Packs/Disposables Custom Packs/Generic Name.

The optional fields are: Body Part, Specialty/Subspecialty, Vendor. There is no Manufacturer.

(IVB-d′-3) As with HCD/Ordinary Disposables, the third section of the IIS card for HCD/Disposable Custom Packs calculates the “Total CE item cost” which is the cost to use an HCD/Disposable Pack in whatever number that it is going to be used in the CE. The calculations are made using the mandatory fields from the second part of the IIS card. The Total CE item cost is the number of packs (“Items”) used in the CE times the Single Item Cost. The IIS algorithm will calculate Total CE item cost when “calculate cost” is chosen.

Total CE item cost is not part of the AIR database and therefore Total CE item cost for HCD/Disposable Custom Packs is not saved to the AIR database. The third section of the IIS card for HCD/Disposable Custom Packs can be pushed to stage card and the stage card can be saved to the stage card database. Stage card database will include Total CE item cost.

(IVB-d′-4) The fourth section of the IIS card for HCD/Disposables Custom Packs is a notes section. It is a free form text field. The contents of the pack can be written here as free form text. When the note section is saved to the AIR database or pushed to the stage card the free form text is dated. If the stage card is saved to the stage card database, the dated text written in the note section is also saved. As discussed above, the AIR database notes field, and the stage card notes field are independent of each other.

(IVB-d′-5) The fifth section for IIS card for HCD/Disposable Custom Packs has one mandatory link that must be completed in order for the IIS card to be complete and allow the total cost of the HCD/Disposable Custom Packs in the CE defined as “Total CE item cost” to be moved to the stage card. This mandatory link also needs to be completed in order to save the “single item cost” of the HCD/Disposable Custom Packs to the AIR database.

The link will connect to the revenue table of the Surgery Center Revenue Submodule where the procedure codes (CPT/HCPCS codes) that are billed in the surgical case have been organized sequentially as they would be formally billed by the ASC. The line of code that corresponds to the HCD/Disposable Custom Packs is then chosen. Once that line of code is chosen, the description for that line of code as written on the revenue table is pulled to the fifth section of the IIS card. Once the IIS card is completed and pushed to the stage card, the “Total CE item cost” is linked to the “total revenue” or “corrected total revenue” if the Collection Discount algorithm was applied for the HCD. This link will allow changes in cost for that HCD to be linked to the changes in revenue or corrected revenue for that HCD in the Profit Module. This link will be discussed further in the Profit Module.

When the completed IIS card is pushed to stage card, a link is established between the Cost and Revenue for the HCD/Disposable Custom Pack. This link is used in the Profit Module. When the IIS card is pushed to AIR database the link is broken and the fifth section of the IIS card is removed. This will be discussed below when using the AIR Database algorithm. When the stage card is saved to the stage card database the link and the fifth section of the IIS card is also removed. This will be discussed below when using the stage card Database algorithm. In both cases, this will allow new links to be established when the AIR database or stage card database is used for other CEs with different revenue tables. When the CE is saved to the CE database all links are initially maintained but can be modified. This will be discussed in the fourth CE Database algorithm.

(IVB-d″) HCD/Disposable Vendor Packs are more than one ordinary disposable put into a pack that is pre-made by the vendor with minimal or no customization from the buyer. An HCD/Disposable Vendor Pack is by definition an “Item”.

Once the choice of HCD/Disposable Vendor Packs is made there is only one method to enter the supply onto the IIS card.

As with HCD/Ordinary Disposables, the IIS card for HCD/Disposable Vendor Packs has 5 sections.

(IVB-d″-1) For HCD/Disposable Vendor Packs, the first section of the IIS card lists HCD, Disposable Pack, Disposable Vendor Pack in the form: HCD/Disposable Pack/Disposable Vendor Pack. There are no categories or subcategories for HCD/Disposable Vendor Packs.

(IVB-d″-2) As with HCD/Disposable Vendor Packs, the second section of the IIS card for HCD/Disposables Vendor Packs has mandatory and optional fields to be filled out. HCD/Disposable Vendor Packs has 3 mandatory fields and 4 optional fields. The mandatory fields must be filled out in order for the IIS card to be complete and allow the total cost of the HCD/Disposable Vendor Pack defined as “Total CE item cost” to be moved to the stage card. The mandatory fields also need to be filled out too in order to save the “single item cost” of the HCD/Disposable Vendor Pack to the AIR database. Optional fields do not have to be filled out to move the “single item cost” of the HCD/Disposable Vendor Pack to the AIR database. Optional fields do not have to be filled out to move the data from the IIS card to the stage card and saved to the stage card database. The optional fields are there to allow for additional information on the HCD/Disposable Vendor Packs to be included and serve as fields that can be searched in the AIR database and as background data on the stage card and in the stage card database. HCD/Disposable Vendor Packs are defined as an “Items”.

i) The mandatory fields are Generic name and/or Brand Name, Single Item Cost, and Number used (#used). There is no Bulk Item cost.

The Generic Name is the name given by the Buyer.

The Brand Name is the name designated by the Vendor or Manufacturer.

The Single Item Cost is a cost to purchase the pack from the Vendor or Manufacturer.

The Number used (#used) is the number of HCD/Disposable Vendor Packs used in the CE.

In order for IIS card to be complete, the Generic Name or Brand Name must be entered onto the IIS card. Both can be entered but at least one needs to be entered for the card to be complete and be able to be pushed to stage card, stage card database or the AIR database. Once the Generic Name and/or Brand Name is entered onto the IIS card, the Generic name and/or Brand Name will appear in Section One of the IIS card after the Subdivision of HCD/Disposable Vendor Packs. If the Generic Name and Brand Name are both entered onto the IIS card, then the first section will read HCD/Disposable Packs/Disposables Vendor Packs/Generic Name/Brand Name.

ii) The optional fields are: Body Part, Specialty/Subspecialty, Manufacturer, Vendor.

(IVB-d″-3) As with HCD/Ordinary Disposables, the third section of the IIS card for HCD/Disposable Vendor Packs calculates the “Total CE item cost” which is the cost to use a Disposable Pack in whatever number that it is going to be used in the CE. The calculations are made using the mandatory fields from the second part of the IIS card. The “Total CE item cost” is the number of packs (“Items”) used in the CE times the “single item cost”. The IIS algorithm will calculate “Total CE item cost” when “calculate cost” is chosen.

“Total CE item cost” is not part of the AIR database and therefore “Total CE item cost” for HCD/Disposable Vendor Packs is not saved to the AIR database. The third section of the IIS card for HCD/Disposable Vendor Packs can be pushed to stage card and the stage card can be saved to the stage card database. The stage card database will include “Total CE item cost”.

(IVB-d″-4) The fourth section of the IIS card for HCD/Disposables Vendor Packs is a notes section. It is a free form text field. The contents of the pack can be written here as free form text. When the note section is saved to the AIR database or pushed to the stage card the free form text is dated. If the stage card is saved to the stage card database, the dated text written in the note section is also saved. As discussed above, the AIR database notes field, and the stage card notes field are independent of each other.

(IVB-d″-5) The fifth section for IIS card for HCD/Disposable Vendor Packs has one mandatory link that must be completed in order for the IIS card to be complete and allow the total cost of the HCD/Disposable Vendor Packs in the CE defined as “Total CE item cost” to be moved to the stage card. This mandatory link also needs to be completed in order to save the “single item cost” of the HCD/Disposable Vendor Packs to the AIR database.

The link will connect to the revenue table of the Surgery Center Revenue Submodule where the procedure codes (CPT/HCPCS codes) that are billed in the surgical case have been organized sequentially as they would be formally billed by the ASC. The line of code that corresponds to the HCD/Disposable Vendor Packs is then chosen. Once that line of code is chosen, the description for that line of code as written on the revenue table is pulled to the fifth section of the IIS card. Once the IIS card is completed and pushed to the stage card, the “Total CE item cost” is linked to the “total revenue” or “corrected total revenue” if the Collection Discount algorithm was applied for the HCD. This link will allow changes in cost for that HCD to be linked to the changes in revenue or corrected revenue for that HCD in the Profit Module. This link will be discussed further in the Profit Module.

When the completed IIS card is pushed to stage card, a link is established between the Cost and Revenue for the HCD/Disposable Vendor Pack. This link is used in the Profit Module. When the IIS card is pushed to AIR database the link is broken and the fifth section of the IIS card is removed. This will be discussed below when using the AIR Database algorithm. When the stage card is saved to the stage card database the link and the fifth section of the IIS card is also removed. This will be discussed below when using the stage card Database algorithm. In both cases, this will allow new links to be established when the AIR database or stage card database is used for other CEs with different revenue tables. When the CE is saved to the CE database all links are initially maintained but can be modified. This will be discussed in the CE Database algorithm.

(IVC) The third Subclass of the HCD Class is Disposable Instruments. Like the HCD/Ordinary Disposables Subclass, the HCD/Disposable Instruments Subclass has categories and subcategories.

Once the choice of the third Subclass of HCD/Disposables Instruments is made there are 4 methods (i-iv) to enter the supply into the HCD/Disposables Instruments Subclass and onto the IIS card as there is with the HCD/Ordinary Disposable Subclass.

i. The first method uses categories that are part of the IIS algorithm. Some categories of HCD/Disposables Instruments in the IIS algorithm have subcategories, and some do not. The list of categories and subcategories for HCD/Disposables Instruments is outlined in the Addendum.

Once the category is chosen or the subcategory for those categories that have subcategories, the IIS card appears and can then be filled out with the information for the disposable instrument that is to be used in a particular CE. The mandatory and optional fields for an HCD/Disposable Instruments are discussed below.

ii. The second method uses the categories of the IIS algorithm and allows for the creation of a unique subcategory within that category. The category chosen for the creation of the unique subcategory may or may not have other subcategories already in the IIS algorithm.

iii. The third method allows the unique creation of a category in the HCD/Disposable Instruments Subclass.

iv. The fourth method allows the unique creation of a category and subcategory in the HCD/Disposable Instruments Subclass.

As with the first method, when the second, third or fourth method is completed the IIS card will also appear and can then be filled out with the information for the HCD/Disposable Instrument that is to be used in a particular CE.

As with HCD/Ordinary Disposables and HCD/Disposable Packs, the IIS card for HCD/Disposables Instruments has four sections.

(IVC-1) The first section of the IIS card for HCD/Disposables Instruments lists HCD, Disposables Instruments and category and the subcategory (when present) in the form: HCD/Disposables Instruments/category/subcategory. This first section of the IIS card for HCD Disposables Instruments has been determined by the previous choices made for the category and subcategory (if present). For HCD/Disposables Instruments there always must be a category chosen which either comes from IIS algorithm or is created as a unique category as was described above. Additional information will be added to this section of the IIS card for HCD/Disposables Instruments after category/subcategory (when present) by fields completed in the second section.

(IVC-2) The second section of the IIS card for HCD/Disposables Instruments has 4 mandatory fields and 4 optional fields to be filled out. The mandatory fields must be filled out in order for the IIS card to be complete and allow the total cost of the disposable instrument(s) in the CE defined as “Total CE item cost” to be moved to the stage card. The mandatory fields also need to be filled out too in order to save the “single item cost” to the AIR database. Optional fields do not have to be filled out to move the “single item cost” of the disposable instrument(s) to the AIR database. Optional fields do not have to be filled out to move the data from the IIS card to the stage card and saved to the stage card database. The optional fields are there to allow for additional information on the HCD/Disposables Instruments to be included and serve as fields that can be searched in the AIR database and as background data on the stage card and in the stage card database.

i) The mandatory fields are: Generic or Brand name, Bulk Item cost, Number of items in a Bulk (#in Bulk) and number used (#used).

The Generic Name is the common and generally accepted designation for the disposable instrument that uniquely identifies that supply and distinguishes it from other disposable instruments. Generic name is free form text field.

The Brand Name is the proprietorial name given to a disposable instrument by the maker of that disposable instrument. Brand name is free form text field.

A Bulk Item is Collection of single disposable instruments “items” purchased together. The cost of purchasing a bulk item of disposable instruments is the Bulk Item Cost. Bulk item cost is a “free form text field” in dollars.

Number of items in a Bulk (#in Bulk) is the number of items contained in a Bulk when the items are purchased in bulk.

The “Number used” (#used) is the number of disposable instrument “items” used in the CE. The “number used” is a single-select list box option.

In order for IIS card to be complete, the Generic Name or Brand Name of the HCD/Disposable Instrument must be entered onto the IIS card. Both can be entered but at least one must be entered for the card to be complete and be able to be pushed to the stage card, stage card database or the AIR database. Once the Generic Name and/or Brand Name is entered onto the IIS card, the Generic Name and/or Brand Name will appear in Section One of the IIS card after the last category or subcategory that was previously chosen. If the Generic and Brand Name are both entered onto the IIS card, then the first section will read HCD/Disposables Instruments/Category/Subcategory (if chosen)/Generic Name/Brand Name.

ii) The optional fields are: Body part, Specialty/Subspecialty, Manufacturer, Vendor.

The definitions and fields for Body part, Specialty/Subspecialty, Manufacturer, Vendor on the IIS card for HCD/Disposable Instruments are the same as are on the IIS card for Disposable Instruments (IIC-2).

(IVC-3) The third section of the IIS card for HCD/Disposables Instruments calculates the “single item cost” for the disposable instrument and the “Total CE item cost” to use that item in the amount that it is going to be used in the CE. The calculations are made using mandatory fields from the second part of the IIS card. The single item cost is the cost of the bulk item divided by the number of items in the bulk. The “Total CE item cost” is the number of disposable instrument “items” used in the CE times the “single item cost”. The IIS algorithm will calculate “Total CE item cost” when “calculate cost” is chosen.

The third section of the IIS card for HCD/Disposables Instruments can be saved to the AIR database. The Bulk cost and the single item cost are saved to AIR database. The number used and Total CE item cost are not saved to the AIR database. The third section of the IIS card for Disposables Instruments can be pushed to stage card and the stage card can be saved to the stage card database. Stage card database will include all fields in the third section of the IIS card including number used and “Total CE item cost”. The IIS algorithm will calculate “Total CE item cost” when “calculate cost” is chosen.

There is no “extended use” option for HCD instruments. HCD instruments are reimbursable per CE and are not used in more than one CE.

(IVC-4) The fourth section of the IIS card for HCD/Disposables Instruments is a notes section. It is a free form text field. When the free form text written in the note section is saved to the AIR database or pushed to the stage card, the free form text is dated. If the stage card is saved to the stage card database, the dated text written in the note section is also saved. As discussed, elsewhere, the AIR database notes field, and the stage card notes field are independent of each other.

(IVC-5) The fifth section for IIS card for HCD/Disposable Instruments has one mandatory link that must be completed in order for the IIS card to be complete and allow the total cost of the HCD/Disposable Instruments in the CE defined as “Total CE item cost” to be moved to the stage card. This mandatory link also needs to be completed to save the “single item cost” of the HCD/Disposable Instruments to the AIR database.

The link will connect to the revenue table of the Surgery Center Revenue Submodule where the procedure codes (CPT/HCPCS codes) that are billed in the surgical case have been organized sequentially as they would be formally billed by the ASC. The line of code that corresponds to the HCD/Disposable Instruments is then chosen. Once that line of code is chosen, the description for that line of code as written on the revenue table is pulled to the fifth section of the IIS card. Once the IIS card is completed and pushed to the stage card, the “Total CE item cost” is linked to the “total revenue” or “corrected total revenue” if the Collection Discount algorithm was applied for the HCD. This link will allow changes in cost for that HCD to be linked to the changes in revenue or corrected revenue for that HCD in the Profit Module. This link will be discussed further in the Profit Module.

When the completed IIS card is pushed to stage card, a link is established between the Cost and Revenue for the HCD/Disposable Instruments. This link is used in the Profit Module. When the IIS card is pushed to AIR database the link is broken and the fifth section of the IIS card is removed. This will be discussed below when using the AIR Database algorithm. When the stage card is saved to the stage card database the link and the fifth section of the IIS card is also removed. This will be discussed below when using the stage card Database algorithm. In both cases, this will allow new links to be established when the AIR database or stage card database is used for other CEs with different revenue tables. When the CE is saved to the CE database all links are initially maintained but can be modified. This will be discussed in the CE Database algorithm.

(V) The fifth Class is Implants. Implants are defined as a foreign object that is put physically inside a client and remains wholly intact inside the client following discharge. Implants are potentially reimbursable. Implants are usually classified as FDA CLASS III. Sutures are foreign objects that are put physically inside a client and remain wholly intact inside the client following discharge but are generally not reimbursable. Sutures are usually classified as FDA CLASS II.

For the Implant Class the IIS algorithm allows the choice of 2. Subclasses.

(VA) The first Subclass of the Implants is Implant-Non-Biologic. Implant-Non-Biologic is defined as a foreign object that is put physically inside of a client, is biologically inactive and remains wholly intact and inert inside of client following discharge.

The first Subclass of Implant-Non-Biologic has 8 Branches. The branches are: Spine, Extremities, Gastrointestinal, Cosmetic, Cardiac-Vascular, Ocular and Auditory.

Once the choice of the first Subclass is made and the choice of the Branch is made there are 4 (i-iv) methods to enter the Implant-Non-Biologic/Branch onto the IIS card.

The first method uses categories that are part of the IIS algorithm. Some categories of Implant-Non-Biologic/Branch in the IIS algorithm have subcategories, and some do not. The list of categories and subcategories for Implant-Non-Biologic/Branch is extensive and is outlined in the Addendum.

Once the category is chosen or the subcategory for those categories that have subcategories, the IIS card appears and can then be filled out with the information for the supply that is to be used in a particular CE. The mandatory and optional fields for an Implant-Non-Biologic/Branch are discussed below.

The second method uses the categories of the IIS algorithm and allows for the creation of a unique subcategory within that category. The category chosen for the creation of the unique subcategory may or may not have other subcategories already in the IIS algorithm.

The third method allows the unique creation of a category in the Implant-Non-Biologic/Branch.

The fourth method allows the unique creation of a category and subcategory in the Implant-Non-Biologic/Branch.

As with the first method, when the second, third or fourth method is completed the IIS card will also appear and can then be filled out with the information for the supply that is to be used in a particular CE.

The IIS card for Implant-Non-Biologic/Branch has five sections.

(VA-1) The first section of the IIS card for Implants lists Implants, Implant-Non-Biologic, Branch and category and the subcategory (when present) in the form: Implants/Implant-Non-Biologic/Branch/category/subcategory. This first section of the IIS card for Implant-Non-Biologic has been determined by the previous choices made for the Branch, category and subcategory (if present). For Implant-Non-Biologic/Branch there always must be a category chosen which either comes from IIS algorithm or is created as a unique category as was described above. Additional information will be added to this section of the IIS card for Implant-Non-Biologic/Branch after category/subcategory (when present) by fields completed in the second section.

(VA-2) The second section of the IIS card for Implant-Non-Biologic/Branch has 3 mandatory fields and 4 optional fields to be filled out. The mandatory fields must be filled out in order for the IIS card to be complete and allow the total cost of the Implant-Non-Biologic in the CE defined as “Total CE item cost” to be moved to the stage card. The mandatory fields also need to be filled out too in order to save the “single item cost” to the AIR database. Optional fields do not have to be filled out to move the “single item cost” of the Implant-Non-Biologic to the AIR database. Optional fields do not have to be filled out to move the data from the IIS card to the stage card and saved to the stage card database. The optional fields are there to allow for additional information on the Implant-Non-Biologic/Branch to be included and serve as fields that can be searched in the AIR database and as background data on the stage card and in the stage card database.

i) The mandatory fields are: Generic or Brand name, Single Item cost, number used (#used).

The Generic Name is the common and generally accepted designation for an Implant-Non-Biologic that identifies it. Generic name is free form text field.

The Brand Name is the proprietorial name given to an Implant-Non-Biologic by the maker of that Implant-Non-Biologic. Brand name is free form text field.

The “Number used” (#used) is the number of items used in the CE. The “number used” is a single-select list box option.

Single Item cost is the cost to purchase the Implant-Non-Biologic as an “Item”.

In order for IIS card to be complete, the Generic Name or Brand Name must be entered onto the IIS card. Both can be entered but at least one must be entered for the card to be complete and be able to be pushed to the stage card, stage card database or the AIR database. Once the Generic Name and/or Brand Name is entered onto the IIS card, the Generic Name and/or Brand Name will appear in Section One of the IIS card after the last category or subcategory that was previously chosen. If the Generic and Brand Name are both entered onto the IIS card, then the first section will read Implants/Implants-Non-Biologic/Branch/Category/Subcategory (if chosen)/Generic Name/Brand Name.

ii) The optional fields are: Body part, Specialty/Subspecialty, Manufacturer, Vendor.

Body part are listed in the Addendum. Body part options are multi-select list box options where one or more options can be chosen from the list. There is no “free form text field”.

Specialty/Subspecialties are listed in the Addendum. Specialty/Subspecialties options are multi-select list box options where one or more options can be chosen from the list. There is no “free form text field”. Some Specialties on the list have nested Subspecialties and some do not.

Manufacturer is the company that manufactures the supply. The field to enter the manufacturer on the IIS Card is a “free form text field”. This optional free form text field can be saved to the AIR database. When the IIS card is pushed to the stage card and the stage card is saved, the manufacturer in the free form text field will also be saved with the stage card. Both these instances will be covered in detail below when the AIR Database algorithm and stage card Database algorithm are discussed.

Vendor is the seller of the supply. As with the Manufacturer, the field to enter the vendor on the IIS Card is a “free form text field”. The optional free form text field can again be saved to the AIR database once the IIS card is complete and to the stage Card Database once the stage Card is saved to the stage Card Database.

(VA-3) The third section of the IIS card for Implant-Non-Biologic calculates “Total CE item cost” to use that item in the amount that it is going to be used in the CE. Single Item Cost and #used has already been added as a mandatory field in section 2. The calculation is made by multiplying the “single item cost” by the number used (#used). The IIS algorithm will calculate “Total CE item cost” when “calculate cost” is chosen.

The third section of the IIS card for Implants-Non-Biologic can be saved to the AIR database. The single item cost is saved to AIR database. The number used and “Total CE item cost” are not saved to the AIR database. The third section of the IIS card for Implants-Non-Biologic can be pushed to stage card and the stage card can be saved to the stage card database. Stage card database will include all fields in the third section of the IIS card including number used and Total CE item cost.

(VA-4) The fourth section of the IIS card for Implants-Non-Biologic is a notes section. It is a free form text field. When the free form text written in the note section is saved to the AIR database or pushed to the stage card the free form text is dated. If the stage card is saved to the stage card database, the dated text written in the note section is also saved. As discussed, elsewhere, the AIR database notes field, and the stage card notes field are independent of each other.

(VA-5) The fifth section for IIS card for Implants-Non-Biologic has one mandatory field with two options. The first option is “Link to Revenue” and second option “Do Not Link to Revenue”. If “Do Not Link to Revenue” option is chosen, the IIS card will be complete and allow the total cost of the Implant-Non-Biologic in the CE defined as “Total CE item cost” to be moved to the stage card. It will also allow the “single item cost” of the Implants-Non-Biologic to be saved to the AIR database.

If “Link to Revenue” option is chosen, the link will connect to the revenue table of the Surgery Center Revenue Submodule where the procedure codes (CPT/HCPCS codes) that are billed in the surgical case have been organized sequentially as they would be formally billed by the ASC. The line of code that corresponds to the Implant-Non-Biologic is then chosen. Once that line of code is chosen, the description for that line of code as written on the Revenue Table is pulled to the fifth section of the IIS card. Once the IIS card is completed and pushed to the stage card, the “Total CE item cost” is linked to the “total revenue” or “corrected total revenue” if the Collection Discount algorithm was applied for the Implants. This link will allow changes in cost for that Implant to be linked to the changes in revenue or corrected revenue for that HCD in the Profit Module. This link will be discussed further in the Profit Module.

When the “Link to Revenue” option is chosen and IIS card is completed and is pushed to stage card, a link is established between the Cost and Revenue for the Implant-Non-Biologic. This link is used in the Profit Module. When the IIS card is pushed to AIR database the link is broken. This will be discussed below when using the AIR Database algorithm. When the stage card is saved to the stage card database the link is also broken. In both cases the fifth section of the IIS card is not removed. It returns to the unselected mandatory field with two options. This will be discussed below when using the stage card Database algorithm. In both cases, this will allow new links to be established when the AIR database or stage card database is used for other CEs with different revenue tables. When the CE is saved to the CE database all links are initially maintained but can be modified. This will be discussed in the CE Database algorithm.

The “Do Not Link to Revenue” and “Link to Revenue” options can be undone and changed to the other option.

(VB) The second Subclass of the Implants is Implant-Biologic. Implant-Biologic is defined

as a foreign object that is put physically inside of a client and remains wholly intact and is biologically active inside of client following discharge. Implant-Biologic is potentially reimbursable.

There are two Subsets of Implant-Biologics based upon what they are made of and/or what naturally occurring biological material they are trying to mimic.

The first Subset is “Implants-Biological-Natural” (I-B-N). I-B-N are defined as implants derived originally from a naturally occurring biological product. I-B-Ns are biologically active.

The second Subset is “Implants-Biological-Synthetic” (I-B-S). I-B-S are defined as implants which are not derived from an original biological product. I-B-Ss are biologically active.

(VB-s′) The first Subset of Implant-Biologic-Natural (I-B-N) has 8 Branches. The branches are: Spine, Extremities, Gastrointestinal, Cosmetic, Cardiac-Vascular, Ocular and Auditory.

Once the choice of the first Subset is made and the choice of the Branch is made there are 4 (i-iv) methods to enter the Implant-Biologic-Natural (I-B-N)/Branch onto the IIS card.

i. The first method uses categories that are part of the IIS algorithm. Some categories of Implant-Biologic-Natural (I-B-N)/Branch in the IIS algorithm have subcategories, and some do not. The list of categories and subcategories for Implant-Biologic-Natural (I-B-N)/Branch is extensive and is outlined in the Addendum.

Once the category is chosen or the subcategory for those categories that have subcategories, the IIS card appears and can then be filled out with the information for the supply that is to be used in a particular CE. The mandatory and optional fields for an Implant-Biologic-Natural (I-B-N)/Branch are discussed below.

ii. The second method uses the categories of the IIS algorithm and allows for the creation of a unique subcategory within that category. The category chosen for the creation of the unique subcategory may or may not have other subcategories already in the IIS algorithm.

iii. The third method allows the unique creation of a category in the Implant-Biologic-Natural/Branch.

iv. The fourth method allows the unique creation of a category and subcategory in the Implant-Biologic-Natural(I-B-N)/Branch.

As with the first method, when the second, third or fourth method is completed the IIS card will also appear and can then be filled out with the information for the Implant-Biologic-Natural (I-B-N) that is to be used in a particular CE.

The IIS card for Implant-Biologic-Natural/Branch (I-B-N) has five sections.

(VB-s′-1) The first section of the IIS card for Implants-Biologic-Natural lists Implants, Implant-Biologic, Subset, Branch and category and the subcategory (when present) in the form: Implants/Implant-Biologic/Implant-Biologic-Natural (I-B-N)/Branch/category/subcategory. This first section of the IIS card for Implant-Biologic-Natural (I-B-N) has been determined by the previous choices made for the Branch, category and subcategory (if present). For Implant-Biologic-Natural (I-B-N)/Branch there always must be a category chosen which either comes from IIS algorithm or is created as a unique category as was described above. Additional information will be added to this section of the IIS card for Implant-Biologic-Natural (I-B-N)/Branch after category/subcategory (when present) by fields completed in the second section.

(VB-s′-2) The second section of the IIS card for Implant-Biologic-Natural (I-B-N)/Branch has 3 mandatory fields and 4 optional fields to be filled out. The mandatory fields must be filled out in order for the IIS card to be complete and allow the total cost of the Implant-Biologic-Natural (I-B-N) in the CE defined as “Total CE item cost” to be moved to the stage card. The mandatory fields also need to be filled out to in order to save the “single item cost” to the AIR database. Optional fields do not have to be filled out to move the “single item cost” of the Implant-Biologic-Natural (I-B-N) to the AIR database.

Optional fields do not have to be filled out to move the data from the IIS card to the stage card and saved to the stage card database. The optional fields are there to allow for additional information on the Implant-Biologic-Natural to be included and serve as fields that can be searched in the AIR database and as background data on the stage card and in the stage card database.

The mandatory fields are: Generic or Brand name, Single Item cost, number used (#used).

The Generic Name is the common and generally accepted designation for an Implant-Biologic-Natural that identifies it. Generic name is free form text field.

The Brand Name is the proprietorial name given to an Implant-Biologic-Natural (I-B-N) by the maker of that Implant-Biologic-Natural (I-B-N). Brand name is free form text field.

The “Number used” (#used) is the number of items used in the CE. The “number used” is a single-select list box option.

Single Item cost is the cost to purchase the Implant-Biologic-Natural (I-B-N) as an “Item”.

In order for IIS card to be complete, the Generic Name or Brand Name must be entered onto the IIS card. Both can be entered but at least one must be entered for the card to be complete and be able to be pushed to the stage card, stage card database or the AIR database. Once the Generic Name and/or Brand Name is entered onto the IIS card, the Generic Name and/or Brand Name will appear in Section One of the IIS card after the last category or subcategory that was previously chosen. If the Generic and Brand Name are both entered onto the IIS card, then the first section will read Implants/Implants-Biologic/Implant-Biologic-Natural (I-B-N)/Branch/Category/Subcategory (if chosen)/Generic Name/Brand Name.

The optional fields are: Body part, Specialty/Subspecialty, Manufacturer, Vendor.

Body part are listed in the Addendum. Body part options are multi-select list box options where one or more option can be chosen from the list. There is no “free form text field”.

Specialty/Subspecialties are listed in the Addendum. Specialty/Subspecialties options are multi-select list box options where one or more options can be chosen from the list. There is no “free form text field”. Some Specialties on the list have nested Subspecialties and some do not.

Manufacturer is the company that manufactures the supply. The field to enter the manufacturer on the IIS Card is a “free form text field”. This optional free form text field can be saved to the AIR database. When the IIS card is pushed to the stage card and the stage card is saved, the manufacturer in the free form text field will also be saved with the stage card. Both these instances will be covered in detail below when the AIR Database algorithm and stage card Database algorithm are discussed.

Vendor is the seller of the supply. As with the Manufacturer, the field to enter the vendor on the IIS Card is a “free form text field”. The optional free form text field can again be saved to the AIR database once the IIS card is complete and to the stage Card Database once the stage Card is saved to the stage Card Database.

(VB-s′-3) The third section of the IIS card for Implant-Biologic-Natural (I-B-N) calculates “Total CE item cost” to use that item in the amount that it is going to be used in the CE. “Total Item Cost” and #used has already been added as mandatory fields in section 2. The calculation is made by multiplying the “single item cost” by the number used (#used). The IIS algorithm will calculate “Total CE item cost” when “calculate cost” is chosen.

The third section of the IIS card for Implant-Biologic-Natural (I-B-N) can be saved to the AIR database. The “single item cost” is saved to AIR database. The number used and Total CE item cost are not saved to the AIR database. The third section of the IIS card for Implant-Biologic-Natural (I-B-N) can be pushed to stage card and the stage card can be saved to the stage card database. Stage card database will include all fields in the third section of the IIS card including number used and Total CE item cost.

(VB-s′-4) The fourth section of the IIS card for Implant-Biologic-Natural (I-B-N) is a notes section. It is a free form text field. When the free form text written in the note section is saved to the AIR database or pushed to the stage card the free form text is dated. If the stage card is saved to the stage card database, the dated text written in the note section is also saved. As discussed, elsewhere, the AIR database notes field, and the stage card notes field are independent of each other.

(VB-s′-5) The fifth section for IIS card for Implant-Biologic-Natural (I-B-N) has one mandatory field with two options. The first option is “Link to Revenue” and second option “Do Not Link to Revenue”. If “Do Not Link to Revenue” option is chosen, the IIS card will be complete and allow the total cost of the Implant-Biologic-Natural (I-B-N) in the CE defined as “Total CE item cost” to be moved to the stage card. It will also allow the “single item cost” of the Implant-Biologic-Natural (I-B-N) to be saved to the AIR database.

If “Link to Revenue” option is chosen, the link will connect to the revenue table of the Surgery Center Revenue Submodule where the procedure codes (CPT/HCPCS codes) that are billed in the surgical case have been organized sequentially as they would be formally billed by the ASC. The line of code that corresponds to the Implant-Biologic-Natural (I-B-N) is then chosen. Once that line of code is chosen, the description for that line of code as written on the revenue table is pulled to the fifth section of the IIS card. Once the IIS card is completed and pushed to the stage card, the “Total CE item cost” is linked to the “total revenue” or “corrected total revenue” if the Collection Discount algorithm was applied for the Implants. This link will allow changes in cost for that Implant to be linked to the changes in revenue or corrected revenue for that HCD in the Profit Module. This link will be discussed further in the Profit Module.

When the “Link to Revenue” option is chosen and IIS card is completed and is pushed to stage card, a link is established between the Cost and Revenue for the Implant-Biologic-Natural (I-B-N). This link is used in the Profit Module. When the IIS card is pushed to AIR database the link is broken. This will be discussed below when using the AIR Database algorithm. When the stage card is saved to the stage card database the link is also broken. In both cases the fifth section of the IIS card is not removed. It returns to the unselected mandatory field with two options. This will be discussed below when using the stage card Database algorithm. In both cases, this will allow new links to be established when the AIR database or stage card database is used for other CEs with different revenue tables. When the CE is saved to the CE database all links are initially maintained but can be modified. This will be discussed in the CE Database algorithm.

The “Do Not Link to Revenue” and “Link to Revenue” options can be undone and changed to the other option.

(VB-s″) The second Subset of Implant-Biologic-Synthetic (I-B-S) has 8 Branches. The branches are: Spine, Extremities, Gastrointestinal, Cosmetic, Cardiac-Vascular, Ocular and Auditory.

Once the choice of the first Subset is made and the choice of the Branch is made there are 4 (i-iv) methods to enter the Implant-Biologic-Synthetic (I-B-S)/Branch onto the IIS card.

i. The first method uses categories that are part of the IIS algorithm. Some categories of Implant-Biologic-Synthetic (I-B-S)/Branch in the IIS algorithm have subcategories, and some do not. The list of categories and subcategories for Implant-Biologic-Synthetic (I-B-S)/Branch is extensive and is outlined in the Addendum.

Once the category is chosen or the subcategory for those categories that have subcategories, the IIS card appears and can then be filled out with the information for the supply that is to be used in a particular CE. The mandatory and optional fields for an Implant-Biologic-Synthetic (I-B-S)/Branch are discussed below.

ii. The second method uses the categories of the IIS algorithm and allows for the creation of a unique subcategory within that category. The category chosen for the creation of the unique subcategory may or may not have other subcategories already in the IIS algorithm.

The third method allows the unique creation of a category in the Implant-Biologic-Natural/Branch.

The fourth method allows the unique creation of a category and subcategory in the Implant-Biologic-Synthetic (I-B-S)/Branch.

As with the first method, when the second, third or fourth method is completed the IIS card will also appear and can then be filled out with the information for the Implant-Biologic-Natural (I-B-N) that is to be used in a particular CE.

The IIS card for Implant-Biologic-Synthetic (I-B-S) has five sections.

(VB-s″-1) The first section of the IIS card for Implants-Biologic-Synthetic lists Implants, Implant-Biologic, Subset, Branch and category and the subcategory (when present) in the form: Implants/Implant-Biologic/Implant-Biologic-Synthetic (I-B-S)/Branch/category/subcategory. This first section of the IIS card for Implant-Biologic-Synthetic (I-B-S) has been determined by the previous choices made for the Branch, category and subcategory (if present). For Implant-Biologic-Synthetic (I-B-S)/Branch there always must be a category chosen which either comes from IIS algorithm or is created as a unique category as was described above. Additional information will be added to this section of the IIS card for Implant-Biologic-Synthetic (I-B-S)/Branch after category/subcategory (when present) by fields completed in the second section.

(VB-s″-2) The second section of the IIS card for Implant-Biologic-Synthetic (I-B-S)/Branch has 3 mandatory fields and 4 optional fields to be filled out. The mandatory fields must be filled out in order for the IIS card to be complete and allow the total cost of the Implant-Biologic-Synthetic (I-B-S) in the CE defined as “Total CE item cost” to be moved to the stage card. The mandatory fields also need to be filled out to in order to save the “single item cost” to the AIR database. Optional fields do not have to be filled out to move the “single item cost” of the Implant-Biologic-Synthetic (I-B-S) to the AIR database. Optional fields do not have to be filled out to move the data from the IIS card to the stage card and saved to the stage card database. The optional fields are there to allow for additional information on the Implants-Biologic-Synthetic to be included and serve as fields that can be searched in the AIR database and as background data on the stage card and in the stage card database.

The mandatory fields are: Generic or Brand name, Single Item cost, number used (#used).

The Generic Name is the common and generally accepted designation for an Implant-Biologic-Synthetic that identifies it. Generic name is free form text field.

The Brand Name is the proprietorial name given to a Implant-Biologic-Synthetic (I-B-S) by the maker of that Implant-Biologic-Synthetic (I-B-S). Brand name is free form text field.

The “Number used” (#used) is the number of items used in the CE. The “number used” is a single-select list box option.

Single Item cost is the cost to purchase the Implant-Biologic-Synthetic (I-B-S) as an “Item”.

In order for IIS card to be complete, the Generic Name or Brand Name must be entered onto the IIS card. Both can be entered but at least one must be entered for the card to be complete and be able to be pushed to the stage card, stage card database or the AIR database. Once the Generic Name and/or Brand Name is entered onto the IIS card, the Generic Name and/or Brand Name will appear in Section One of the IIS card after the last category or subcategory that was previously chosen. If the Generic and Brand Name are both entered onto the IIS card, then the first section will read Implants/Implants-Biologic/Implant-Biologic-Synthetic (I-B-S)/Branch/Category/Subcategory (if chosen)/Generic Name/Brand Name.

The optional fields are: Body part, Specialty/Subspecialty, Manufacturer, Vendor.

Body part are listed in the Addendum. Body part options are multi-select list box options where one or more options can be chosen from the list. There is no “free form text field”.

Specialty/Subspecialties are listed in the Addendum. Specialty/Subspecialties options are multi-select list box options where one or more options can be chosen from the list. There is no “free form text field”. Some Specialties on the list have nested Subspecialties and some do not.

Manufacturer is the company that manufactures the supply. The field to enter the manufacturer on the IIS Card is a “free form text field”. This optional free form text field can be saved to the AIR database. When the IIS card is pushed to the stage card and the stage card is saved, the manufacturer in the free form text field will also be saved with the stage card. Both these instances will be covered in detail below when the AIR Database algorithm and stage card Database algorithm are discussed.

Vendor is the seller of the supply. As with the Manufacturer, the field to enter the vendor on the IIS Card is a “free form text field”. The optional free form text field can again be saved to the AIR database once the IIS card is complete and to the stage Card Database once the stage Card is saved to the stage Card Database.

(VB-s″-3) The third section of the IIS card for Implant-Biologic-Synthetic (I-B-S) calculates “Total CE item cost” to use that item in the amount that it is going to be used in the CE. “Single Item Cost” and #used has already been added as mandatory fields in section 2. The calculation is made by multiplying the “single item cost” by the number used (#used). The IIS algorithm will calculate “Total CE item cost” when “calculate cost” is chosen.

The third section of the IIS card for Implant-Biologic-Synthetic (I-B-S) can be saved to the AIR database. The single item cost is saved to AIR database. The number used and “Total CE item cost” are not saved to the AIR database. The third section of the IIS card for Implant-Biologic-Synthetic (I-B-S) can be pushed to stage card and the stage card can be saved to the stage card database. Stage card database will include all fields in the third section of the IIS card including number used and Total CE item cost.

(VB-s″-4) The fourth section of the IIS card for Implant-Biologic-Synthetic (I-B-S) is a notes section. It is a free form text field. When the free form text written in the note section is saved to the AIR database or pushed to the stage card the free form text is dated. If the stage card is saved to the stage card database, the dated text written in the note section is also saved. As discussed, elsewhere, the AIR database notes field, and the stage card notes field are independent of each other.

(VB-s″-5) The fifth section for IIS card for Implant-Biologic-Synthetic (I-B-S) has one mandatory field with two options. The first option is “Link to Revenue” and second option “Do Not Link to Revenue”. If “Do Not Link to Revenue” option is chosen, the IIS card will be complete and allow the total cost of the Implant-Biologic-Synthetic (I-B-S) in the CE defined as “Total CE item cost” to be moved to the stage card. It will also allow the “single item cost” of the Implant-Biologic-Synthetic (I-B-S) to be saved to the AIR database.

If “Link to Revenue” option is chosen, the link will connect to the Revenue Table of the Surgery Center Revenue Submodule where the procedure codes (CPT/HCPCS codes) that are billed in the surgical case have been organized sequentially as they would be formally billed by the ASC. The line of code that corresponds to the Implant-Biologic-Synthetic (I-B-S) is then chosen. Once that line of code is chosen, the description for that line of code as written on the revenue table is pulled to the fifth section of the IIS card. Once the IIS card is completed and pushed to the stage card, the “Total CE item cost” is linked to the “total revenue” or “corrected total revenue” if the Collection Discount algorithm was applied for the Implants. This link will allow changes in cost for that Implant to be linked to the changes in revenue or corrected revenue for that Implant in the Profit Module. This link will be discussed further in the Profit Module.

When the “Link to Revenue” option is chosen and IIS card is completed and is pushed to stage card, a link is established between the Cost and Revenue for the Implant-Biologic-Synthetic (I-B-S). This link is used in the Profit Module. When the IIS card is pushed to AIR database the link is broken. This will be discussed below when using the AIR Database algorithm. When the stage card is saved to the stage card database the link is also broken. In both cases the fifth section of the IIS card is not removed. It returns to the unselected mandatory field with two options. This will be discussed below when using the stage card Database algorithm. In both cases, this will allow new links to be established when the AIR database or stage card database is used for other CEs with different revenue tables. When the CE is saved to the CE database all links are initially maintained but can be modified. This will be discussed in the CE Database algorithm.

The “Do Not Link to Revenue” and “Link to Revenue” options can be undone and changed to the other option.

(VI) The six Class is DME-Orthotics. DME-Orthotics is defined as medical equipment that was given to a client that is not disposable and not reusable but durable. The DME-Orthotics provides support for a physical limitation. The client will generally leave the client experience with a DME-Orthotics.

A DME-Orthotics may be reimbursable and generate revenue but will always have a cost. DMEs and orthotics are by definition “Items”.

Once the choice of sixth Class of DME-Orthotics is made, there are four methods (i-iv) to enter the DME or orthotic onto the IIS card for DME-Orthotics. The DME-Orthotics Class has no Subclasses. The DME-Orthotics Class has categories and may have subcategories.

Once the choice of the Class of DME-Orthotics is made there are 4 methods (i-iv) to enter the supply into the DME-Orthotics Class and onto the IIS card.

The first method uses categories that are part of the IIS algorithm. Some categories of DME-Orthotics in the IIS algorithm have subcategories, and some do not. The list of categories and subcategories for DME-Orthotics is extensive and is outlined in the Addendum.

Once the category is chosen or the subcategory for those categories that have subcategories, the IIS card appears and can then be filled out with the information for the supply that is to be used in a particular CE. The mandatory and optional fields for an DME-Orthotics are discussed below.

The second method uses the categories of the IIS algorithm and allows for the creation of a unique subcategory within that category. The category chosen for the creation of the unique subcategory may or may not have other subcategories already in the IIS algorithm.

The third method allows the unique creation of a category in the DME-Orthotics Class.

The fourth method allows the unique creation of a category and subcategory in the DME-Orthotics Class.

As with the first method, when the second, third or fourth method is completed the IIS card will also appear and can then be filled out with the information for the DME or orthotic that is to be used in a particular CE.

The IIS card for DME-Orthotics has 5 sections.

(VI-1) The first section of the IIS card for DME-Orthotics lists DME-Orthotics, category and the subcategory (when present) in the form: DME-Orthotics/category/subcategory. This first section of the IIS card for DME-Orthotics has been determined by the previous choices made for the category and subcategory (if present). For DME-Orthotics there always must be a category chosen which either comes from IIS algorithm or is created as a unique category as was described above. Additional information will be added to this section of the IIS card for DME-Orthotics after category/subcategory (when present) by fields completed in the second section.

(VI-2) The second section of the IIS card for DME-Orthotics has 4 mandatory fields and 6 optional fields to be filled out. The mandatory fields must be filled out in order for the IIS card to be complete and allow the total cost of the supply in the CE defined as “Total CE item cost” to be moved to the stage card. The mandatory fields also need to be filled out too in order to save the “single item cost” to the AIR database. Optional fields do not have to be filled out to move the “single item cost” of the DME or orthotic to the AIR database. Optional fields do not have to be filled out to move the data from the IIS card to the stage card and saved to the stage card database. The optional fields are there to allow for additional information on the DME/Orthotics to be included and serve as fields that can be searched in the AIR database and as background data on the stage card and in the stage card database.

i) The mandatory fields are: Generic or Brand name, Bulk Item cost, number of items in a Bulk (#in Bulk) and number used (#used).

The Generic Name is the common and generally accepted designation for a DME or orthotic that identifies it. Generic name is free form text field.

The Brand Name is the proprietorial name given to a DME or orthotic by the maker of that DME or orthotic. Brand name is free form text field.

A Bulk Item is collection of single items purchased together. The cost of purchasing a bulk item is the Bulk Item Cost. Bulk item cost is a “free form text field” in dollars.

Number of items in a Bulk (#in Bulk) is the number of items contained in a Bulk when the items are purchased in bulk.

The “Number used” (#used) is the number of items used in the CE. The “number used” is a single-select list box option.

In order for IIS card to be complete, the Generic Name or Brand Name must be entered onto the IIS card. Both can be entered but at least one must be entered for the card to be complete and be able to be pushed to the stage card, stage card database or the AIR database. Once the Generic Name and/or Brand Name is entered onto the IIS card, the Generic Name and/or Brand Name will appear in Section One of the IIS card after the last category or subcategory that was previously chosen. If the Generic and Brand Name are both entered onto the IIS card, then the first section will read DME-Orthotics/Category/Subcategory (if chosen)/Generic Name/Brand Name.

ii) The optional fields are: Body part, Specialty/Subspecialty, CPT/HCPCS Code, Manufacturer, Vendor, “Link to Revenue”.

Body part are listed in the Addendum. Body part options are multi-select list box options where one or more options can be chosen from the list. There is no “free form text field”.

Specialty/Subspecialties are listed in the Addendum. Specialty/Subspecialties options are multi-select list box options where one or more options can be chosen from the list. There is no “free form text field”. Some Specialties on the list have nested Subspecialties and some do not.

CPT/HCPCS Codes are the standard supply codes for DME or orthotics. The field to enter the CPT/HCPCS code on the IIS Card is a “free form text field”.

Manufacturer is the company that manufactures the DME or orthotic. The field to enter the manufacturer on the IIS Card is a “free form text field”. This optional free form text field can be saved to the AIR database. When the IIS card is pushed to the stage card and the stage card is saved, the manufacturer in the free form text field will also be saved with the stage card. Both these instances will be covered in detail below when the AIR Database algorithm and stage card Database algorithm are discussed.

Vendor is the seller of the DME or orthotic. As with the Manufacturer, the field to enter the vendor on the IIS Card is a “free form text field”. The optional free form text field can again be saved to the AIR database once the IIS card is complete and to the stage Card Database once the stage Card is saved to the stage Card Database.

“Link to Revenue” is an optional field that when chosen will create a fifth section on the IIS card that will be discussed below. It is used in the uncommon situation when the DME or orthotic has a revenue component in the Professional Revenue Submodule or the Surgery Center Revenue Submodule. The “Link to Revenue” optional field in the second section of the IIS card of DME-Orthotics has a different function than the “Link to Revenue” in the fifth section of the IIS card for Implants.

(VI-3) The third section of the IIS card for DME-Orthotics calculates the single “single item cost” for the DME or orthotic and the “Total CE item cost” to use that item in the amount that it is going to be used in the CE. The calculations are made using mandatory fields from the second part of the IIS card. The single item cost is the cost of the bulk item divided by the number of items in the bulk. The “Total CE item cost” is the number of items used in the CE times the “single item cost”. The IIS algorithm will calculate “Total CE item cost” when “calculate cost” is chosen.

The third section of the IIS card for DME-Orthotics can be saved to the AIR database. The Bulk cost and the single item cost are saved to AIR database. The number used and “Total CE item cost” are not saved to the AIR database. The third section of the IIS card for DME-Orthotics can be pushed to stage card and the stage card can be saved to the stage card database. Stage card database will include all fields in the third section of the IIS card including number used and Total CE item cost.

(VI-4) The fourth section of the IIS card for DME-Orthotics is a notes section. It is a free form text field. When the free form text written in the note section is saved to the AIR database or pushed to the stage card, the free form text is dated. If the stage card is saved to the stage card database, the dated text written in the note section is also saved.

As will be discussed further in detail below, the AIR Database algorithm interfaces with the AIR database and the stage card Database algorithm interfaces with the stage card database. The free form text in the note section that was saved to those databases using the IIS algorithm will be present. If either the AIR database or stage card database is used to enter supplies into a new IIS card for a different CE the notes will follow. The new notes written using either the AIR Database algorithm or the stage card Database algorithm into a new IIS card are independent of each other. Once the new notes in the note section of a new IIS card built using either of the two different databases is saved to that database, it is saved only to that database and does not cross over to the other database.

(VI-5) When the “Link to Revenue” option is chosen in the second section of the IIS card the fifth section of the IIS card will appear. The fifth section for IIS card for DME-Orthotic has one mandatory fields with two mutually exclusive options. The first option is “Link to Professional Revenue Submodule” and the second option is to “Link to Surgery Center Revenue Submodule”. One of the two mutually exclusive options, described below, must be completed in order for the IIS card to be complete.

When the first option “Link to Professional Revenue Submodule” is chosen, a link will connect to the Revenue Table of the Professional Revenue Submodule using the

Link to Professional Revenue Submodule sub algorithm. The Revenue Table contains the procedure codes (CPT/HCPCS codes) that are billed in the surgical case and that have been organized sequentially as they would be formally billed by the ASC. The Revenue Table also contains the DME-Orthotic HCPCS codes. The line of code that corresponds to the DME-Orthotic is then chosen. Once that line of code is chosen, the description for that line of code as written on the Revenue Table is pulled to the fifth section of the IIS card. Once the IIS card is completed and pushed to the stage card, the “Total CE item cost” is linked to the “total revenue” or “corrected total revenue” if the Collection Discount algorithm was applied for the DME/Orthotics. This link will allow changes in cost for that DME/Orthotic to be linked to the changes in revenue or corrected revenue for that DME/Orthotic in the Profit Module. This link will be discussed further in the profit module.

When the second option “Link to Surgery Center Revenue Submodule” is chosen, a link will connect to the Revenue Table of the Surgery Center Revenue Submodule using the Link to Surgery Center Revenue Submodule sub algorithm. The Revenue Table contains the procedure codes (CPT/HCPCS codes) that are billed in the surgical case and that have been organized sequentially as they would be formally billed by the ASC. The Revenue Table also contains the DME-Orthotic HCPCS codes. The line of code that corresponds to the DME-Orthotic is then chosen. Once that line of code is chosen, the description for that line of code as written on the Revenue Table is pulled to the fifth section of the IIS card. Once the IIS card is completed and pushed to the stage card, the “Total CE item cost” is linked to the “total revenue” or “corrected total revenue” if the Collection Discount algorithm was applied for the DME/Orthotics. This link will allow changes in cost for that DME/Orthotic to be linked to the changes in revenue or corrected revenue for that DME/Orthotic in the Profit Module. This link will be discussed further in the Profit Module.

When the completed IIS card is pushed to stage card, a link is established between the Cost and Revenue for the DME/Orthotic. The link is either to the Professional Revenue Submodule or Surgery Center Revenue Submodule. This link is used in the Profit Module. When the IIS card is pushed to AIR database the link is broken and the fifth section of the IIS card is removed. This will be discussed below when using the AIR Database algorithm. When the stage card is saved to the stage card database the link and the fifth section of the IIS card is also removed. This will be discussed below when using the stage card Database algorithm. In both cases, this will allow new links to be established when the AIR database or stage card database is used for other CEs with different revenue tables. When the CE is saved to the CE database all links are initially maintained but can be modified. This will be discussed in the CE Database algorithm.

### AIR Database Algorithm:

The AIR Database algorithm searches the AIR database and pulls the information from that database on a particular supply onto the IIS card data entry fields for the supply being considered for a particular CE. There are two ways to search AIR database. The first way is to search by the Class of the supply and second way is to search the database by using key terms.

Searching the AIR database by Class begins by choosing the Class. Depending upon the Class chosen, there may be Subclasses, Branches, subdivisions, subsets, categories and subcategories in that Class. Searching for a supply in a particular Class involves navigating through those Subclasses, Branches, subdivisions, subsets, categories and subcategories until the Generic and/or the Brand name is reached.

Once the Generic and/or Brand name is reached, the supply entry in the AIR database for the supply being searched for can be chosen. Once the supply entry is chosen, the IIS card appears and is filled out with the saved data from the AIR database for that supply. The data entered onto the IIS card using the AIR database will include the mandatory fields that were required to originally save the supply to the AIR database and any additional optional fields that were completed for that supply when it was originally saved to the AIR database.

To save an IIS card generated using the AIR database to a stage card or as a new entry into the AIR database requires that all mandatory fields on the IIS card be completed. The mandatory fields are different for different Classes, Subclasses, Branches, subdivisions and subsets. These mandatory fields are outlined above in the section detailing the use of the IIS algorithm to enter a supply onto an IIS card.

The IIS card generated from the AIR database has an “Edit” function. All fields in the second section of the IIS card can be edited including the Generic and/or Brand name. If the Generic and/or Brand name is edited this will change the first section of the IIS card from the original Generic and/or Brand name to new Generic and/or Brand name. The remainder of the first section of the IIS card will remain unchanged. All other fields in the second section of the IIS card can be edited.

The third section of the IIS card is the section that calculates the “Total CE item cost”. This section is not filled out when using the AIR Database algorithm and the AIR database. It is a mandatory field and must be completed for the IIS card to be complete.

The fourth section of the IIS card is the notes section and includes the notes that were saved in the AIR database. The notes section can be edited or erased. Any changes to the notes section is dated when the IIS card is saved to the AIR database.

The fifth section of the IIS card is not filled out by using the AIR Database algorithm and the AIR database. When the fifth section of the IIS card is present it is a mandatory section that must be completed for the IIS card to be complete.

The AIR Database algorithm has a “Delete” and an “Overwrite” feature. The Delete feature will delete a supply from the AIR database. The Overwrite feature will allow a supply that has been edited using the Edit feature above to overwrite and replace the original supply in the AIR database with the new edited supply.

The second way is to search the AIR database is by using key terms.

Data fields that are present in the AIR database that cannot be searched for by using key terms are: “Single Item cost”, “Bulk Item cost”, “number in Bulk”, and any free text dated in the note section. Date of entry of the supply into the AIR database also cannot be searched.

Data fields that are not present in the AIR database and therefore cannot be searched are: “Total CE item cost”, “number used”, “percentage used” and any fields in the fifth section of the IIS card.

Data fields that can be searched that are present in the AIR database using key terms are: “Generic name”, “Brand name”, “Specialty/Subspecialty”, “Manufacturer”, “Vendor”, “Body part”, “CPT/HCPSC codes”.

For the data fields that can be searched using key terms, the information displayed will be for the supplies in the AIR database that contain the key term. The supply containing the key term will be displayed in the manner in which the supply is recorded in the AIR database. This will include the Class of the supply and depending upon the Class may include Subclasses, Branches, subdivisions, subsets, categories and subcategories down to Generic and/or Brand name. In all cases it will include the Generic and/or Brand name as the Generic and/or Brand name is always part of the AIR database. If the key term is not a Brand and/or Generic name, it will not appear in search return but will appear highlighted when IIS card is opened using the AIR Database algorithm. The search return will be ordered in reverse chronological order to the date of entry of the supply into the AIR database.

stage Card Database Algorithm

The stage card Database algorithm searches the stage card database. The stage card database is a list of the saved stage cards for different CEs. The stage card database is organized alphabetically by CE from which the stage cards were originally pushed. CEs for stage cards saved to stage card database also includes the date that stage card was saved. The database can be viewed in its entirety and can be searched in one of two ways.

The first way is to search by the date the stage card was saved to the stage card database. The second way is to search by the CE.

Using either method, once the stage card is identified and opened it lists numerically one after the other all the supplies and supply costs in a particular CE. On the stage card, the supply is identified by Brand name and/or Generic name and by the “Total CE item cost” for that supply in a particular CE.

The stage card Database algorithm will allow any item on the stage card from the stage card database to be opened to the original IIS card that was used to save the item to stage card database. All sections of the IIS card are included for every supply listed on the stage card with the exception of the fifth section which is excluded from stage card database.

A supply on the stage card that was opened using the stage card Database algorithm can be pushed to a new stage card in a new CE once all mandatory fields are completed. The new IIS card can also be pushed to the AIR database once the mandatory fields are completed.

An entire stage card for a CE from the stage card database can be pushed to a new CE. To save the stage card in the new CE, all mandatory fields for every item on the stage card must be completed.

The IIS card opened from the stage card database has an “Edit” feature. All fields in the second section of the IIS card can be edited including the Generic and/or Brand name. If the Generic and/or Brand name is edited this will change the first section of the IIS card from the original Generic and/or Brand name to new Generic and/or Brand name. The remainder of the first section of the IIS card will remain unchanged. All other fields in the second section of the IIS card can be edited.

Supplies items on the stage card from the stage card Data Base can be edited individually and the entire stage card with the edited supply items can be pushed to a new CE once all mandatory fields for every item on the stage card, both edited and unedited, are completed.

The stage card database has a Delete function. If a stage card is deleted from stage card database, there is an option to delete the entire CE from which the stage card was originally pushed. If the entire CE from which the stage card was originally pushed is not deleted, the stage card will remain part of that original CE and the original CE will remain intact in the CE database.

Since the stage card database is organized by CE, the stage card database can be used to link to the CE database.

There is no Overwrite function in the stage card database. If an IIS card for a supply item from the stage card database is opened and edited using the “Edit” feature and the new edited stage card is saved to the stage card database, it is saved using a different CE. The original CE remains linked to the original stage card. Even if the name of both CEs are the same, there will be a different date associated with the new CE once it is saved to the stage card database and this different date along with the name of the CE would constitute a new CE.

The first way to search the stage card database for a supply is to search by the date the stage card was saved to the stage card database. All entries that have been made into the stage card database can be displayed in their entirety in reverse chronological order. Entries can also be displayed in reverse chronological order by week, by month and by year or for a selected time frame.

The second way to search the stage card database is to search by the name of CE. The search will return the CEs in reverse chronological order after the first 3 letters are entered into the search field and continue to narrow the search as more letters are entered. The letters searched can be anywhere in the CE name.

### CE Database Algorithm

The CE Database algorithm searches the CE database. The CE database contains all CEs that were saved to CE database. CEs saved to CE database include the name of the CE and the date that the CE was saved to CE database. The CE database can be searched by one of two main methods. The first method is to search the CE database is by the name of the CE and the second method is to search the database by the date the CE was saved to the CE database.

Searching the CE database by the name of the CE will return the CEs saved in the CE database in reverse chronological order beginning when the first 3 letters that are entered into the search field. The search field will continue to narrow the search as more letters are entered. The letters searched can be anywhere in the CE name.

Searching the CE database by the date the CE was entered into the CE database will display CEs in reverse chronological order by week, by month and by year or for a selected time frame.

Once a particular CE from the CE database is opened, the entire CE with all the completed Modules and Submodules are available for review including the stage card for that CE.

Once the stage card is opened for that CE, it lists numerically one after the other all the supplies and supply costs for that particular CE. On the stage card, the supply is identified by Brand name and/or Generic name and by the Total CE item cost for that supply for that particular CE.

The CE Database algorithm will allow any item on the stage card for that particular CE to be opened to the original IIS card that was used when the CE was saved to the CE database. As that particular CE contains all the Modules and Submodules it also includes all sections of the IIS card for every supply listed on the stage card including the fifth section when present.

A supply on the stage card that was opened using the CE Database algorithm can be pushed to a new stage card in a new CE once all mandatory fields are completed. The new IIS card can also be pushed to the AIR database once the mandatory fields are completed.

The stage card for a particular CE may include HCD, Implant and/or DME/Orthotics. As above, all HCDs, most Implants and some DMEs/Orthotics will have a fifth section present on the IIS card. This fifth section is not pushed to the new CE. When the IIS card for the HCD or Implant is saved in the new CE, a new mandatory fifth section must be completed. For DME/Orthotics if the optional Link to Revenue in the second section of IIS card is chosen for the new CE, the fifth section to IIS card must be completed.

An entire stage card for a particular CE from the CE database can be pushed to a new CE. To save the stage card in the new CE, all mandatory fields for every item on the stage card must be completed.

As with the stage card database, the IIS card opened using the CE database has an “Edit” feature. All fields in the second section of the IIS card can be edited including the Generic and/or Brand name. If the Generic and/or Brand name is edited this will change the first section of the IIS card from the original Generic and/or Brand name to new Generic and/or Brand name. The remainder of the first section of the IIS card will remain unchanged. All other fields in the second section of the IIS card can be edited.

Supply items on the stage card for particular CE from the CE database can be edited. The supplies can be edited individually and the entire stage card with the edited supply items pushed to a new CE once all mandatory fields for every item on the stage card, both edited and unedited, are completed.

The CE database algorithm allows for a CE to be saved to the CE database. The CE is saved alphabetically by the CE name and the date it was saved and can be searched by either of those variables. The CE database algorithm has a Delete function. An entire CE can be deleted from the CE database. Deleting a CE will not delete a stage card originally saved to stage card database from that CE or any item originally saved to AIR database from that CE. The CE database, stage card database and AIR database are independent.

The CE database algorithm has no Overwrite function on the CE database. Changing a data entry in any of the Modules or Submodules and saving those changes to CE database will create a different CE with the different date and time even if the same CE name is used.

### Cost Module [PART2]—Direct Supply Cost—Rental Supply Section

The Surgery Center Cost submodule has a section for rental of supplies. A Supply is defined as any inanimate physical object used during the CE. If the supply is owned by the ASC, it is Inventory. Inventory is defined as any supply used during the CE which is owned by the ASC. If the supply is rented by the ASC, it is a Rental Supply. Rental Supply is defined as any supply used during the CE which is rented from a third party and not owned by the ASC.

The Rental Supply algorithm has two options which consider the rental of a supply per case and the rental of the supply per day.

For rental of the supply per case, the cost of the rental and the description of rental supply is entered into Rental Supply algorithm. The Cost Table algorithm pulls the cost and description to the Cost table in the Cost Module. The algorithm enters the cost of the supply as the “line cost” for that supply into the Cost column of the Cost table. There is no option to enter the cost of the supply into any other columns of the Cost table.

For rental of supply per day, the cost and description of the rental is entered into the Rental Supply algorithm and the number of “surgical cases” in which the supply is used per day is also entered into the algorithm. The algorithm divides the cost of the supply by the number of surgical cases which the supply is used per day. The Cost Table algorithm pulls the result into the Cost column on the Cost table as the “line cost” of the supply. There is no option to enter the cost into any other columns of the Cost table.

### Cost Module [PART3]—Direct Claim Costs

Claim Costs are an additional cost apart from Direct Labor cost and Direct Supply costs. The user can choose to include the costs for submitting the claims necessary to generate Professional revenue, Anesthesia revenue, Surgery Center revenue and Ancillary revenue. Each of these 4 revenue sources must submit its own claims and each will have its own Claim cost. As described in the Revenue Module, each of the revenue sources has its own revenue component or components which may require the submission of additional claims and add to Claim cost for that revenue source.

Submitting a claim can be done in-house or by a third party. The cost to submit a claim in-house and by third party are different. The Third-Party Claim Cost algorithm considers the cost to submit the claim by third party. The In-house Claim Cost algorithm considers the cost to submit the claim in house. Both algorithms consider each of the 4 revenue sources separately.

Third-Party Claim Cost algorithm involves third party claim cost submission and considers the cost to submit the initial claim or claims for the revenue source being considered as well as the additional costs of resubmitting the same claim or claims more than one time.

For each revenue source, the user will enter the direct cost to submit one claim for that revenue source when using a third-party. As more than one initial claim may be submitted in any one revenue source for a particular CE, the user will enter the number of claims submitted in each revenue source when the third-party is used. The sub algorithm will multiply the number of claims submitted in the revenue source by the cost of submitting one claim. The result will be the initial third-party claim cost for that revenue source.

As claims are often resubmitted more than one time until revenue is received, the user will enter the number of times the claims are resubmitted. This is inputted for each of the 4 revenue sources separately when the third-party is used. The sub algorithm will multiply the initial claim cost for each revenue source by the number of times the claims are resubmitted. The result is the “Third-Party Claim Cost” calculated using the Third-Party Claim Cost algorithm. Each revenue source will have their own separate “third-party claim cost”. These “third-party claim cost” are “Professional third-party claim cost”, “Anesthesia third-party claim cost”, “Surgery Center third-party claim cost”, “Ancillary third-party claim cost”. The Cost Table algorithm will pull these “third-party Claim Costs” to the Cost Table in the Cost Module.

In-House Claim Cost algorithm considers the initial direct cost to submit a claim in-house and as well as the additional costs of resubmitting the same claim more than one time.

As in house claims are often combined together by revenue source, the user will enter a single cost to submit the initial claim or claims for that revenue source.

As claims are often resubmitted more than one time until revenue is received, the user will enter the number of times the in-house claims are resubmitted. This is inputted for each of the 4-revenue source separately. The algorithm will multiply the initial in-house claim cost for each revenue source by the number of times the claims are resubmitted. The result is the “In House Claim Cost” calculated by In-House Claim Cost algorithm. The Cost table algorithm pulls the “in house claim cost” for each revenue source that has a claim cost to the Cost Table in the Cost Module.

The user can apply either the Third-Party Claim Cost algorithm or In-House Claim Cost algorithm to any of the 4 revenue sources independently. The user is prohibited from applying both sub algorithms to the same revenue source.

The Cost Table algorithm will sum the “In-house claim cost” and/or “third-party claim cost” for each of the revenue sources that have a claim cost. The results are displayed as the Claim Cost “component cost” on the Cost Table in the Cost Module.

### Cost Table Submodule

The Cost Table algorithm accesses the data from [PART1], [PART2] and [PART3] which are the 3 Direct Cost components in the Cost Module.

The Dynamic Cost Table algorithm pulls the data from the Cost Table in the Cost Module to the Dynamic Cost Table in the Profit Module.

### Columns in the Cost Table:

First column/Cost Drivers: The first column are the cost drivers divided into the 3 components of Direct cost. These 3 components are the Players, the Supplies and the Claim Costs.

The Players component is divided into Professional Providers, Anesthesia Providers, Team Members and Ancillary Providers. Professional Providers are sub divided into the Professional Provider job descriptions. The Anesthesia Providers are sub divided into the Anesthesia Provider job descriptions. Team Members are sub divided into the Team Members job descriptions. Ancillary Providers are sub divided into the Ancillary Providers job descriptions.

The Supply component is divided into Inventory and Rental Supplies. Inventory is further divided into Classes. These Classes are Disposables, HCD, Medications, DME/Orthotics and Implants.

Disposables are divided into Ordinary Disposables, Disposable Instruments and Disposable Packs. All Ordinary Disposables are considered together on the Cost Table and the costs are not separated out as they are on the stage card. All Disposable Instruments are considered together and are not separated out as they would be on the stage card. All Disposable Packs are considered together and are not separated out as they would be on the stage card into “Disposable Custom Packs” and “Disposable Vendor Packs”.

All HCDs are separated out by HCD description on the cost table. The description is the brand name and/or the generic name of the HCD that was entered in the second section of the IIS card for the HCD, pushed to stage card by the IIS algorithm. Cost table algorithm pulls the description to the Cost table. HCDs are not subdivided by HCD/Ordinary Disposable, HCD/Disposable Packs, HCD/Disposable instruments on the Cost Table as the number of HCD are few and the description of HCD is all that is needed. The HCD/Disposable description is entered onto the Cost Table in the same order as appears on the stage card.

All Medications are considered together on the Cost Table and the costs are not separated out as they are on the stage card.

All DME/Orthotics are separated out by DME/Orthotics description on the Cost Table. The description is the brand name and/or the generic name of the DME/Orthotic that was entered in the second section of the IIS card for the DME/Orthotic, pushed to stage card by the IIS algorithm. Cost table algorithm pulls the description to the Cost table. The DME/Orthotic description is entered into Cost Table in the same order as appears on the stage card.

All Implants are separated out by Implants description on the Cost Table. The description is the brand name and/or the generic name of the Implant that was entered in the second section of the IIS card for the Implants, pushed to stage card by the IIS algorithm. Cost table algorithm pulls the description to the Cost table. Implants are not sub divided by Implant-Biologic and Implant-Non Biologic on the Cost Table as they are on the stage card. The Implant description is entered into the Cost Table in the same order as appears on the stage card.

The Rental supply description for each rental supply is pulled by Cost Table algorithm to the first column of the cost table under Rental Supplies in the order the rental supplies were entered into the Rental Supply algorithm.

“The Claim costs” component is divided by Revenue source. These divisions are Professional Claim Costs, Surgery Center Claim Costs, Anesthesia Claim Costs and Ancillary Claim Costs.

Only those components, divisions and subdivisions that contain cost drivers are included in the Cost Table.

Second column/Pre-op stage: This column includes only Direct Labor Costs for Players that engage with the Client in the Pre-op stage that was pulled into this column by Cost Table algorithm.

Third column/O.R stage: This column includes only Direct Labor Costs for Players that engage with the Client in the O.R stage that was pulled into this column by Cost Table algorithm.

Forth column/Post-op #1: This column includes only Direct Labor Costs for Players that engage with the Client in the Post-op #1 stage that was pulled into this column by Cost Table algorithm.

Fifth column/Post-op #2: This column includes only Direct Labor Costs for Players that engage with the Client in the Post-op #2 stage that was pulled into this column by Cost Table algorithm.

Sixth column/AO time: This column includes only Direct Labor Costs for Players that have AO time. The AO time is pulled into this column by Cost Table algorithm.

Seventh column/cost: Depending upon respective rows, this column includes “line cost”, “class cost”, “division cost”, “component cost” and “CE total Cost”.

### Rows in the Cost Table:

Rows in the cost table are filled with Data accessed or calculated by the Cost Table algorithm.

### Player Cost Component Rows:

Professional Provider Division includes a row for each Professional provider job description pulled by Cost Table algorithm to the Cost Table. For each Professional Provider, the Pre-op stage, O.R. stage, Post-op #1 stage, Post-op #2 stage and AO time columns are filed with cost data pulled by Cost Table algorithm to the Cost table. If there is no cost data in the particular column, the column for that row is left blank. The data in the Cost column is calculated by the Direct Labor algorithm and pulled by Cost Table algorithm to the Cost column on the Cost table. This is the “line cost” for that Professional Provider job description. If there is more than one Professional Provider each will have their own “line cost”. The sum of all Professional Providers “line costs” are calculated by Cost Table algorithm and its call Professional Provider “division cost”.

Anesthesia Provider Division includes a row for each Anesthesia provider job description pulled by Cost Table algorithm to the Cost Table. For each Anesthesia Provider, the Pre-op stage, O.R. stage, Post-op #1 stage and AO time columns are filed with cost data pulled by Cost Table algorithm to the Cost table. If there is no cost data in the particular column, the column for that row is left blank. The data in the Cost column is calculated by the Direct Labor algorithm and pulled by Cost Table algorithm to the Cost column on the Cost table. This is the “line cost” for that Anesthesia Provider job description. If there is more than one Anesthesia Provider each will have their own “line cost”. The sum of all Anesthesia Providers “line costs” are calculated by Cost Table algorithm and its call Anesthesia Provider “division cost”.

Team Member Division includes a row for each Team Member job description pulled by Cost Table algorithm to the Cost Table. For each Team Member, the Pre-op stage, O.R. stage, Post-op #1 stage and Post-op #2 stage columns are filed with cost data pulled by Cost Table algorithm to the Cost table. If there is no cost data in the particular column, the column for that row is left blank. The data in the Cost column is calculated by the Direct Labor algorithm and pulled by Cost Table algorithm to the Cost column on the Cost table. This is the “line cost” for that Team Member job description. Each Team Member will have their own “line cost”. The sum of all Team Members “line costs” are calculated by Cost Table algorithm and its call Team Member “division cost”.

Ancillary Provider Division includes a row for each Ancillary provider job description pulled by Cost Table algorithm to the Cost Table. For each Ancillary Provider, the Pre-op stage, O.R. stage, Post-op #1 stage, Post-op #2 stage and AO time columns are filed with cost data pulled by Cost Table algorithm to the Cost table. If there is no cost data in the particular column, the column for that row is left blank. The data in the Cost column is calculated by the Direct Labor algorithm and pulled by Cost Table algorithm to the Cost column on the Cost table. This is the “line cost” for that Ancillary Provider job description. If there is more than one Ancillary Provider each will have their own “line cost”. The sum of all Ancillary Providers “line costs” are calculated by Cost Table algorithm and its call Ancillary Provider “division cost”.

The sum of all the Player “division costs” rows is calculated by Cost Table algorithm, and it is called Player “component cost” and it is displayed beneath the Player divisions.

### Supply Cost Component Rows:

Inventory Division rows on the Cost Table are separated into 5 Classes.

Disposable Class row includes a row for each subclass of Ordinary Disposables, Disposable Instruments and Disposable Packs. The cost of each subclass in pulled by the Cost Table algorithm from stage card to its respective row on the Cost column in the Cost Table. The cost for each subclass is the “line cost” for that subclass.

The sum total of all “line cost” for the disposable subclasses is calculated by the Cost Table algorithm and entered into the Cost column below the“line costs”. This cost is called the Disposable “class cost”. The Estimate algorithm allows the user to estimate the global cost for the Disposable Class in the Cost Module. The estimated cost is pulled to the Cost Table by the Cost Table algorithm as the “class cost” for the Disposable Class, there are no “line cost”.

Medications Class row is a single row with medications cost pulled by the Cost Table algorithm from the stage card into Cost column on the Cost Table. This cost is both the “line cost” and the “class cost” for medications. The Estimate algorithm allows the user to estimate the global cost for the Medications Class in the Cost Module. The estimated cost is pulled to the Cost Table as the “class cost” for the Medications Class.

HCD Class row includes a row for each HCD description entered onto stage card and pulled by Cost Table algorithm from stage card to Cost Table. The Cost Table algorithm enters the respective cost of each HCD into the Cost column in Cost Table. Cost for each HCD is a “line cost” for that HCD.

The sum total of all “line cost” for the HCDs is calculated by the Cost Table algorithm and entered into the Cost column below the“line costs”. This cost is called the HCD “class cost”.

Implant Class row includes a row for each Implant description entered onto stage card and pulled by Cost Table algorithm to Cost Table. The Cost Table algorithm enters the respective cost of each Implant into the Cost column in Cost Table. Cost for each Implant is a “line cost” for that Implant.

The sum total of all “line cost” for the Implants is calculated by the Cost Table algorithm and entered into the Cost column below the“line costs”. This cost is called the Implant “class cost”.

DME/Orthotic Class row includes a row for each DME/Orthotic description entered onto stage card and pulled to Cost Table by Cost Table algorithm. The Cost Table algorithm enters the respective cost of each DME/Orthotic into the Cost column in Cost Table. Cost for each DME/Orthotic is a “line cost” for that DME/Orthotic.

The sum total of all “line cost” for the DME/Orthotics is calculated by the Cost Table algorithm and entered into the Cost column below the“line costs”. This cost is called the DME/Orthotic “class cost”.

The sum total of all “class cost” for the Inventory Division is calculated by the Cost Table algorithm and entered into the Cost column as the last Inventory row. This cost is called the Inventory “division cost”.

### Rental Supply Division Rows on the Cost Table:

Rental supply row includes a row for each Rental supply description entered onto stage card and pulled to Cost Table by Cost Table algorithm. The Cost Table algorithm enters the respective cost of each rental supply into the Cost column in Cost Table. Cost for each rental supply is a “line cost” for that rental supply.

The sum total of all “line cost” for the rental supplies is calculated by the Cost Table algorithm and entered into the Cost column below the“line costs”. This cost is called the Rental supply “division cost”.

The sum total of all “division cost” for the Supply component rows is calculated by the Cost Table algorithm and is displayed beneath the last division row. This cost is called the Supply cost “component cost”.

### Claim Cost Component Rows:

Professional Claim cost row is a single row with the Professional claim cost pulled to the cost column in the Cost Table by the Cost Table algorithm. This cost is the “line cost” for this row.

Surgery Center Claim cost row is a single row with the Surgery Center claim cost pulled to the cost column in the Cost Table by the Cost Table algorithm. This cost is the “line cost” for this row.

Anesthesia Claim cost row is a single row with the Anesthesia claim cost pulled to the cost column in the Cost Table by the Cost Table algorithm. This cost is the “line cost” for this row.

Ancillary Claim cost row is a single row with the Ancillary claim cost pulled to the cost column in the Cost Table by the Cost Table algorithm. This cost is the “line cost” for this row.

The sum total of all “line cost” for the Claim cost component rows is calculated by the Cost Table algorithm and entered into the Cost column below the“line costs”. This cost is called the Claim cost “component cost”.

### CE Total Cost Row:

The Cost Table algorithm calculates “CE total cost” as the sum of the “component costs” and inputs it into the cost column.

### Profit Module:

### The Profit Module has a Dynamic Revenue Table and a Dynamic Cost Table:

The Revenue Table was completed in the Revenue Module using the Revenue Table algorithm. The Dynamic Revenue Table algorithm pulls data from the Revenue Table into the Dynamic Revenue Table and allows the data in the Dynamic Revenue Table to be accessed by the algorithms in the Profit Module.

### Columns in the Dynamic Revenue Table:

Columns in the Dynamic Revenue Table contain data pulled from the Revenue Table by the Dynamic Revenue Table algorithm. If there is no revenue generated by one of revenue submodules or one of the components within a revenue submodule, that section will not appear on the Revenue Table and will not appear on the Dynamic Revenue Table.

1^{st} column/Revenue Generator: The first column are the Revenue generators for the 4 Revenue submodules divided by submodule and subdivided by the submodule's respective components.

As on the Revenue Table, the Professional Revenue submodule contains the Professional Provider component and DME/Orthotics component. The Surgery Center Revenue submodule contains the Surgery/Procedure component, DME/Orthotic component, Implant component and HCD component. The Anesthesia Revenue submodule contains the Anesthesia Provider component for the General Anesthesia/MAC or the Conscious sedation option. The Ancillary Revenue submodule contains the Ancillary Provider component.

2^{nd} column/Revenue: The revenue in this column is either the “component revenue”, the “total revenue” for one of the revenue submodules or “Total CE revenue” depending upon the row. There is no “line revenue” on the Dynamic Revenue Table.

3^{rd} column/Dynamic Revenue: The revenue in this column is initially a mirror image of the revenue in the second column and will dynamically change to reflect changes in revenue that occur by application of the graphic toggle function in the Profit Module.

4^{th} column/Corrected Revenue: The corrected revenue in this column is either the “corrected component revenue”, “corrected total revenue” for one of the revenue submodules or “Corrected Total CE revenue” depending upon the row. There is no “corrected line revenue” on Dynamic Revenue Table.

5^{th} column/Dynamic Corrected Revenue: The “corrected revenue” in this column is initially a mirror image of the “corrected revenue” in the fourth column and will dynamically change to reflect changes in “corrected revenue” that occur by application of the graphic toggle function in the Profit Module.

### Rows on the Dynamic Revenue Table:

Rows on the Dynamic Revenue Table contain data pulled from the Revenue Table by the Dynamic Revenue Table algorithm.

Rows are separated by revenue submodules and by the different components within each revenue submodule.

### Professional Revenue Submodule Section:

### Professional Provider Component Row(s):

The Professional Provider or Providers and their job descriptions are pulled from the Revenue Table in the same order as they appear on the Revenue Table.

The “component revenue” for each Professional Provider is pulled from the Revenue Table and entered to the Revenue column on the Dynamic Revenue Table. If present on the Revenue Table, the “corrected component revenue” for each Professional Provider is entered into the Corrected Revenue column on the Dynamic Revenue Table.

### DME/Orthotic Component Row:

DME/Orthotic(s) are considered one component and labeled as DME/Orthotic on the Dynamic Revenue Table. The “component revenue” is pulled from the Revenue Table and entered into the Revenue column on the Dynamic Revenue Table. If present on the Revenue Table, the “corrected component revenue” for the DME/Orthotic is entered into the Corrected Revenue column on the Dynamic Revenue Table.

### Total of Professional Revenue Submodule Row:

The “total revenue” for the professional revenue submodule is the sum of the Professional Provider “component revenue” for each Professional Provider plus the DME/Orthotic “component revenue”. This is pulled from the Revenue Table by Dynamic Revenue Table algorithm to the Dynamic Revenue Table into column 2.

The “corrected total revenue” for the professional revenue submodule is the sum of the Professional Provider “corrected component revenue” for each Professional Provider plus the DME/Orthotic “corrected component revenue”. If present, this is pulled from the Revenue Table by Dynamic Revenue Table algorithm to the Dynamic Revenue Table into column 3.

### Surgery Center Revenue Submodule Section:

### Surgery/Procedure Component Row(s):

Surgery/Procedure “component revenue” is pulled by Dynamic Revenue Table algorithm from the Revenue Table and entered into the Revenue column on the Dynamic Revenue Table. If present on the Revenue Table, the “corrected component revenue” for the Surgery/Procedure is entered into the “Corrected Revenue” column on the Dynamic Revenue Table.

### Implant Component Row(s):

Implant “component revenue” is pulled by Dynamic Revenue Table algorithm from the Revenue Table and entered into the Revenue column on the Dynamic Revenue Table. If present on the Revenue Table, the “corrected component revenue” for the Surgery/Procedure is entered into the “Corrected Revenue” column on the Dynamic Revenue Table.

### HCD Component Row(s):

HCD “component revenue” is pulled by Dynamic Revenue Table algorithm from the Revenue Table and entered into the Revenue column on the Dynamic Revenue Table. If present on the Revenue Table, the “corrected component revenue” for the Surgery/Procedure is entered into the “Corrected Revenue” column on the Dynamic Revenue Table.

### DME/Orthotic Component Row(s):

DME/Orthotic “component revenue” is pulled by Dynamic Revenue Table algorithm from the Revenue Table and entered into the Revenue column on the Dynamic Revenue Table. If present on the Revenue Table, the “corrected component revenue” for the Surgery/Procedure is entered into the “Corrected Revenue” column on the Dynamic Revenue Table.

The “total revenue” for the Surgery Center revenue submodule is the sum of the Surgery/Procedure “component revenue” and if present the Implant “component revenue”, HCD “component revenue” and DME/Orthotic “component revenue”. This is pulled from the Revenue Table by Dynamic Revenue Table algorithm to the Dynamic Revenue Table into column 2.

The “corrected total revenue” for the Surgery Center revenue submodule is the sum of the Surgery/Procedure “corrected component revenue” and if present the Implant “corrected component revenue”, HCD “corrected component revenue” and DME/Orthotic “corrected component revenue”. This is pulled from the Revenue Table by Dynamic Revenue Table algorithm to the Dynamic Revenue Table into column 2.

### Anesthesia Revenue Submodule Section:

### Anesthesia Provider Component Row(s):

The Anesthesia Provider or Providers and their job descriptions are pulled from the Revenue Table in the same order as they appear on the Revenue Table.

The “component revenue” for each Anesthesia Provider is pulled from the Revenue Table and entered to the Revenue column on the Dynamic Revenue Table. If present on the Revenue Table, the “corrected component revenue” for each Anesthesia Provider is entered into the “Corrected Revenue” column on the Dynamic Revenue Table.

The “total revenue” for the Anesthesia revenue submodule is the sum of the Anesthesia Provider “component revenue” for each Anesthesia Provider. This is pulled from the Revenue Table by Dynamic Revenue Table algorithm to the Dynamic Revenue Table into column 2.

The “corrected total revenue” for the Anesthesia revenue submodule is the sum of the Anesthesia Provider “corrected component revenue” for each Anesthesia Provider. If present, this is pulled from the Revenue Table by Dynamic Revenue Table algorithm to the Dynamic Revenue Table into column 3.

### Ancillary Revenue Submodule Section:

### Ancillary Provider Component Row(s):

The Ancillary Provider or Providers and their job descriptions are pulled from the Revenue Table in the same order as they appear on the Revenue Table.

The “component revenue” for each Ancillary Provider is pulled from the Revenue Table and entered to the Revenue column on the Dynamic Revenue Table. If present on the Revenue Table, the “corrected component revenue” for each Ancillary Provider is entered into the Corrected Revenue column on the Dynamic Revenue Table.

The “total revenue” for the Ancillary revenue submodule is the sum of the Ancillary Provider “component revenue” for each Ancillary Provider. This is pulled from the Revenue Table by Dynamic Revenue Table algorithm to the Dynamic Revenue Table into column 2.

The “corrected total revenue” for the Ancillary revenue submodule is the sum of the Ancillary Provider “corrected component revenue” for each Ancillary Provider. If present, this is pulled from the Revenue Table by Dynamic Revenue Table algorithm to the Dynamic Revenue Table into column 3.

### D) CE Total Revenue Row:

The Dynamic Revenue Table algorithm pulls the “CE total revenue” from the Revenue Table and inputs it into the revenue column 2.

The Dynamic Revenue Table algorithm pulls the “corrected CE total revenue” from the Revenue Table and inputs it into the revenue column 3.

The Cost table was completed in the Cost Module using the Cost Table algorithm. The Cost Table algorithm pushes the Cost Table to the Profit Module where one additional 8^{th} column is added.

8^{th} column/Dynamic Cost: The cost in this column is initially a mirror image of the seventh column on the Cost Table. The costs in this column will dynamically change to reflect changes in costs that occur by application of the graphic toggle function in the Profit Module. This Table in the Profit Module is called the Dynamic Cost Table.

[PART ONE] Gross Profit algorithm calculates the Gross Profit for a CE by subtracting the “CE total cost” pulled from the Cost Table from the “CE total revenue” or the “corrected CE total revenue” pulled from the Revenue Table depending upon the algorithm choices made in the Revenue Module. Adjacent to the calculated Gross Profit is the Dynamic Gross Profit which is initially the same as the Gross Profit. The Dynamic Gross Profit will dynamically change to reflect changes that occur by application of the graphic toggle function in the Profit Module.

[PART TWO] Dynamic Variable algorithm calculates the change in the Gross profit for a CE when certain defined variables are dynamically changed. For each variable, there is a graphic scale which can be toggled to the right or the left. Toggling the scale to the right will increase value of that variable and toggling to the left will decrease value of that variable. Movement of the toggle on the graphic scale to the right or left has an effect on revenue and/or cost. This effect of changing one variable will be visualized dynamically on the affected section of the Dynamic revenue table and/or the Dynamic Cost Table and on the Dynamic Gross profit. The effect of changing more than one variable at the same time will also be separately visualized dynamically on the affected section of the Dynamic Revenue Table and/or the Dynamic Cost Table and on the Dynamic Gross profit as a “combined running variable change”.

The Dynamic Variable algorithm has 3 sub algorithms (I-III) for the 3 variables which can be dynamically changed. The first sub algorithm is the Stage Time Variable sub algorithm. The second sub algorithm is the Inventory Variable sub algorithm. The third sub algorithm is the Player cost variable sub algorithm.

[PART TWO]1. Stage Time Variable sub algorithm.

### Cost:

Changing the stage time in any of the 4 stages will have an effect on those direct costs for which time is a factor in the calculation of the cost. In those cases, the direct cost would be considered to have a “time element”.

For purposes of the system, Direct Supply costs including Inventory and Supply Rental DO NOT have a time element.

Claim costs DO NOT have a time element.

Certain Direct Labor costs for Players DO have a time element based upon the Player's payment type and are affected by changes in stage time.

As described in the Cost Module, the Direct Labor algorithm calculates the cost of a Player in any of the stages of the CE where the Player is engaged with the client. Depending upon the Player and on the stage, the cost of a Player in any given stage of the CE is a combination of one or more of the costs of ALE, PLE or ACT for that Player in that stage. The separate costs of ALE, ACT or PLE used by Direct Labor algorithm to calculate the cost of a Player in any stage of the CE are saved in the background.

The Stage Time variable sub algorithm uses this background data from the Direct Labor algorithm to calculate the effect that changes in the stage time have on Player costs in that stage. For those Players whose cost is based on time there will be a change in cost when stage time is changed. For those Players whose cost is not based on time, changing stage time will have no effect on that Player's cost.

In addition to calculating any changes in individual Player cost when changing stage time, the Stage Time Variable sub algorithm also calculates the sum total cost of all the Players in that stage when the stage time is changed.

The changes in individual Player cost as well as sum total change of all Player costs that results from changing the stage in any of the 4 stages are displayed to the user.

The user can dynamically change the stage time using the graphic toggle function. The effect of dynamically changing stage time on Direct costs is displayed visually on the Dynamic Cost Table.

The Gross profit algorithm calculates and displays the new resulting Dynamic Gross profit.

When changing stage time, the Stage Time Variable sub algorithm prohibits the user from decreasing the stage time in Pre-op, Post-op #1, Post-op #2 below the maximum ALE of any Player in that stage. The O.R. stage is all ALE time and can be decreased to zero.

Changing stage time does not affect AO time. AO time is not part of stage time and cannot be dynamically changed. Changes in AO time are done in CE set up.

### Revenue:

Changing stage time in the O.R. stage or Post-op #1 stage will have an effect on revenue when time is a factor in the calculation of the revenue in those stages. In these cases, the revenue would be considered to have a “time element”.

Changing stage time in Pre-op stage or Post-op #2 stage has no effect on revenue. By definition the Pre-op stage and the Post-op #2 stage have no revenue component.

Surgery Center component revenue including implant, HCD and DME/Orthotics revenue when present DOES NOT have a time element.

Ancillary provider component revenue DOES NOT have a time element.

Professional Provider component revenue for Conscious sedation when the Professional Provider is performing both the conscious sedation and performing the “surgical procedure” as defined in Definitions DOES have a time element in the O.R. stage only. Professional Provider component revenue DOES NOT have a time element in the Post-op #1 stage for Conscious sedation. This system considers conscious sedation to occur only in O.R. stage.

All other Professional Provider component revenue including DME/Orthotic revenue when present DOES NOT have a time element.

Anesthesia Provider component revenue DOES NOT have a time element for General Anesthesia/MAC when using the Anesthesia Revenue Submodule algorithm to calculate anesthesia revenue as a percentage of the billed amount for an anesthesia CPT/HCPCS procedure code.

Anesthesia Provider component revenue DOES have a time element for General anesthesia/MAC in the O.R. stage and Post-op #1 stage when using the Anesthesia Allowable algorithm to calculate an Anesthesia Allowable that has a time variable. This is the case with the Anesthesia Provider job descriptions: MDA-acting alone, CRNA-acting alone, CRNA-directed, CRNA-supervised, AA-directed, AA-supervised.

When the Anesthesia Allowable does not contain a time variable as with revenue generated by the Anesthesia Provider with the job description of MDA supervising or MDA directing, the Anesthesia Provider component revenue also DOES NOT have time element.

Anesthesia Provider component revenue DOES have a time element in the O.R. stage for Conscious sedation when using the Anesthesia Revenue Submodule algorithm that calculates anesthesia revenue as a percentage of the billed amount for an anesthesia CPT/HCPCS procedure code for conscious sedation when a different provider is performing the “surgical procedure”. Anesthesia Provider component revenue DOES NOT have a time element in the Post-op #1 stage for Conscious sedation. This system considers conscious sedation to occur only in O.R. stage.

Revenue and stage time for General anesthesia/MAC when Anesthesia revenue has a time element:

The Stage Time Variable sub algorithm changes stage times by stage and will only change one stage time at a time.

The time variable in the anesthesia allowable formula is the sum of the O.R. stage time and Post-op #1 stage time.

With changes in stage time in the O.R. stage or Post-op #1 stage, the Stage Time Variable sub algorithm enters the new time for the stage that was changed added together with the stage time for the stage that was not changed into the anesthesia allowable formula. The Anesthesia Allowable algorithm will calculate the new anesthesia allowable using the new time.

The Anesthesia Revenue Submodule algorithm is then applied to calculate the new total anesthesia revenue for all lines of code using the new time.

If the Collection Discount Anesthesia algorithm had been previously applied by the user to the total anesthesia revenue, it is also applied to the new total anesthesia revenue using the new time to calculate the new total corrected anesthesia revenue.

The user can dynamically change the stage time in O.R. stage or Post-op #1 stage using the graphic toggle function and any resulting changes in anesthesia revenue are displayed to the user.

The effect of these changes is displayed visually on the Dynamic Revenue table and the Dynamic Cost table. The Gross Profit algorithm calculates and displays the new resulting Dynamic Gross profit.

Revenue and stage time for Conscious Sedation when Anesthesia revenue has a time element:

Anesthesia Provider—MDA acting alone and CRNA acting alone are the only two Anesthesia Provider job descriptions allowed for Conscious Sedation when the conscious sedation is administered by an Anesthesia Provider separate from the Professional Provider performing the procedure.

The user has committed in the Anesthesia Revenue submodule to the CS Different Provider sub algorithm with one of two pathways for conscious sedation. In the Anesthesia Revenue submodule, the CS Different Provider sub algorithm generates in a step wise manner HCPSC and/or CPT codes based on time. Increasing or decreasing stage time above or below the step wise threshold in the sub algorithm will add or remove corresponding HCPCS and/or CPT codes. If decreasing stage time removes a HCPCS and/or CPT code the revenue associated with that code will be removed from the Dynamic Revenue Table. If increasing stage time adds a HCPCS and/or CPT code, the user will be required to input new data. When the HCPCS and/or CPT codes added are a duplicate of a code previously entered in the anesthesia revenue submodule, the billed and allowable amount for that line of code will be entered and displayed onto the Dynamic Revenue Table. If the code has not been previously entered in the anesthesia submodule, the user will need to add the billed amount and the allowable amount for that code. In both cases the user must choose percentage billed or percentage allowed for that line of code using the Conscious Sedation algorithm.

Professional Provider—when the Professional provider is both administering the conscious sedation and performing the surgical procedure:

The user has committed in the Anesthesia Revenue submodule to the GI/CS Same Provider sub algorithm or CS Same Provider sub algorithm both of which have one of two pathways for conscious sedation. In the Anesthesia Revenue submodule, the GI/CS Same Provider sub algorithm and CS Same Provider sub algorithm generate in a step wise manner HCPSC and/or CPT codes based on time and the sub algorithm chosen by the user. Increasing or decreasing stage time above or below the step wise threshold in each of the 2 sub algorithms will add or remove corresponding HCPCS and/or CPT codes. If decreasing stage time removes a HCPCS and/or CPT code the revenue associated with that code will be removed from the Dynamic Revenue Table. If increasing stage time adds a HCPCS and/or CPT code, the user will be required to input new data. When the HCPCS and/or CPT codes added are a duplicate of a code previously entered in the anesthesia revenue submodule, the billed and allowable amount for that line of code will be entered and displayed onto the Dynamic Revenue Table. If the code has not been previously entered in the anesthesia submodule, the user will need to add the billed amount and the allowable amount for that code. In both cases the user must choose percentage billed or percentage allowed for that line of code using the Conscio

### Changing Stage Time's Effect on Cost by Player Type and by Stage & Changing Stage Time's Effect on Revenue by Player Type and by Stage:

### Stage Time Variable Sub Algorithm, Pre-Op Stage, Professional Provider (I.1.A):

### Cost:

I.1.A Dynamically changing the time of the Pre-op stage for Professional Provider has no effect on ALE. Changes will only affect PLE. Dynamically changing stage time in the Pre-op stage will affect the Professional Provider for payment types: payment per hour, salary and payment per day. The Direct Labor algorithm in the Cost Module calculated the cost per hour for the Professional Provider for these 3 payment types. The Cost Table algorithm pulled the cost to the Cost Table in the Cost Module and the Dynamic Cost Table algorithm pulls this cost to the Dynamic Cost Table in the Profit Module.

Dynamically changing stage time in the Pre-op stage will not affect the cost for the payment types: payment per case or percentage of professional component.

The effect of changing stage time on the PLE and the resulting change in the cost of the Professional Provider in the Pre-op stage is calculated by the Stage Time Variable sub algorithm. The cost of PLE in Pre-op stage for the Professional Provider is background data saved by the Direct Labor algorithm. The Stage Time Variable sub algorithm multiplies this background PLE data by the percentage change in the Pre-op stage time from the time that was inputted in CE set up.

By this multiplication, the Stage Time Variable sub algorithm will calculate the new PLE cost with changes in Pre-op stage time for the Professional Provider. The ALE cost in the Pre-op stage for the Professional Provider is also background data saved by the Direct Labor algorithm. The Stage Time Variable sub algorithm will add this new PLE cost to the saved ALE cost. The resulting sum is the new cost of the Professional Provider in the Pre-op stage. The Stage Time Variable sub algorithm can perform these calculations dynamically with dynamically inputted changes in stage time.

Using the graphic toggle function, the user can input changes in pre-op stage time for the Professional Provider and the Stage Time Variable sub algorithm will dynamically display the results.

### Revenue:

I.1.A Dynamically changing the time of the Pre-op stage for Professional Provider has no effect on revenue.

### Stage Time Variable Sub Algorithm, O.R. Stage, Professional Provider (I.2.A):

### Cost:

I.2.A Dynamically changing the time of the O.R. stage for Professional Provider only effects ALE. There is no PLE in the O.R. stage.

Dynamically changing stage time in the O.R. stage will affect the Professional Provider for payment types: payment per hour, salary and payment per day. The Direct Labor algorithm in the Cost Module calculated the cost per hour for the Professional Provider for these 3 payment types. The Cost Table algorithm pulled the cost to the Cost Table in the Cost Module and the Dynamic Cost Table algorithm pulls this cost to the Dynamic Cost Table in the Profit Module.

Dynamically changing stage time in the O.R. stage will not affect the cost for the payment types: payment per case or percentage of professional component.

The effect of changing stage time on the ALE and the resulting change in the cost of the Professional Provider in the O.R. stage is calculated by the Stage Time Variable sub algorithm. The cost of ALE in O.R. stage for the Professional Provider is background data saved by the Direct Labor algorithm. The Stage Time Variable sub algorithm multiplies this background ALE data by the percentage change in the O.R. stage time from the time that was inputted in CE set up.

By this multiplication, the Stage Time Variable sub algorithm will calculate the new ALE cost with changes in O.R. stage time for the Professional Provider. The stage Time Variable sub algorithm can perform these calculations dynamically with dynamically inputted changes in stage time.

Using the graphic toggle function, the user can input changes in O.R. stage time for the Professional Provider and the Stage Time Variable sub algorithm will dynamically display the results.

### Revenue:

I.2.A Dynamically changing the time of the O.R. stage for Professional Provider has an effect on conscious sedation revenue when the Professional Provider is performing conscious sedation and the surgical procedure.

Conscious sedation time occurs during the O.R. stage only. Changing stage time in the O.R. stage will proportionately change the conscious sedation time for the Professional Provider. The HCPCS/CPT codes entered by the user in Professional Provider submodule using either the GI/CS Same Provider sub algorithm or CS Same Provider sub algorithm for conscious sedation are both associated with conscious sedation time ranges. The time ranges are kept as background data. These time ranges have a minimum time and the maximum time. If the user has chosen the third method to enter CPT/HCPCS codes into the Professional Provider Submodule for the conscious sedation component, the user has chosen the time for conscious sedation and this time is used as the conscious sedation time. If the user has chosen the first or second method to enter CPT/HCPCS codes into the Professional Provider Submodule for the conscious sedation component for Professional Provider, the user has not chosen the time for conscious sedation but a range of times. For purposes of the Stage Time Variable sub algorithm, the conscious sedation time when the user has chosen the first or second method is the average time in the HCPCS/CPT code time range between the minimum and maximum time range values.

The O.R. stage time is inputted independently by the user in CE set up from ALE time for the Professional Provider in the O.R. stage. For conscious sedation, the ALE time for the Professional Provider in the O.R. stage must be equal to or greater than the minimum time in the conscious sedation time range. This is a system requirement.

The Stage Time Variable sub algorithm creates a 1:1 ratio between conscious sedation time and O.R. stage time. The percentage increase or decrease in stage time will result in the same percentage increase or decrease in conscious sedation time. Changes in conscious sedation time with changes in stage time may add or remove HCPCS/CPT codes and their associated revenue as described above if a time range threshold is crossed. Which sub algorithm applies is determined by the sub algorithm chosen by the user.

The user can dynamically change the stage time in the O.R. stage using the graphic toggle function and any resulting changes in anesthesia revenue are displayed to the user.

Other than for conscious sedation revenue, dynamically changing the time of the O.R. stage for the Professional Provider has no effect on revenue.

### Stage Time Variable Sub Algorithm, Post-Op #1 Stage, Professional Provider (I.3.A):

### Cost:

I.3.A Dynamically changing the time of the Post-op #1 stage for Professional Provider has no effects on ALE. There is no PLE in the Post-op #1 stage.

Using the graphic toggle function, the user can input changes in Post-op #1 stage time for the Professional Provider and the Stage Time Variable sub algorithm will not change cost or Gross Profit.

### Revenue:

I.3.A Dynamically changing the time of the Post-op #1 stage for Professional Provider has no effect on revenue.

### Stage Time Variable Sub Algorithm, Post-Op #2 Stage, Professional Provider (I.4.A):

### Cost:

I.4.A Dynamically changing the time of the Post-op #2 stage for Professional Provider has no effects on ALE. There is no PLE in the Post-op #2 stage.

Using the graphic toggle function, the user can input changes in Post-op #2 stage time for the Professional Provider and the Stage Time Variable sub algorithm will not change cost or Gross Profit.

### Revenue:

I.4.A Dynamically changing the time of the Post-op #2 stage for Professional Provider has no effect on revenue.

### Stage Time Variable Sub Algorithm, Pre-Op Stage, Team Member (I.1.B):

### Cost:

I1.B Dynamically changing the time of the Pre-op stage for Team Member has no effect on ALE. Changes will only affect ACT. There is no PLE for Team Members.

Dynamically changing stage time in the Pre-op stage will affect the Team Members for payment types: payment per hour, salary and payment per day. The Direct Labor algorithm in the Cost Module calculated the cost per hour for the Team Member for these 3 payment types. The Cost Table algorithm pulled the cost to the Cost Table in the Cost Module and the Dynamic Cost Table algorithm pulls this cost to the Dynamic Cost Table in the Profit Module.

The effect of changing stage time on the ACT and the resulting change in the cost of the Team Members in the Pre-op stage is calculated by the Stage Time Variable sub algorithm. The cost of ACT in Pre-op stage for the Team Members is background data saved by the Direct Labor algorithm. The Stage Time Variable sub algorithm multiplies this background ACT data by the percentage change in the Pre-op stage time from the time that was inputted in CE set up.

By this multiplication, the Stage Time Variable sub algorithm will calculate the new ACT cost with changes in Pre-op stage time for the Team Member. The ALE cost in the Pre-op stage for the Team Member is also background data saved by the Direct Labor algorithm. The Stage Time Variable sub algorithm will add this new ACT cost to the saved ALE cost. The resulting sum is the new cost of the Team Members in the Pre-op stage. The Stage Time Variable sub algorithm can perform these calculations dynamically with dynamically inputted changes in stage time.

Using the graphic toggle function, the user can input changes in pre-op stage time for the Team Members and the Stage Time Variable sub algorithm will dynamically display the results.

### Revenue:

I.1.B Dynamically changing the time of the Pre-op stage for Team Member has no effect on revenue.

### Stage Time Variable Sub Algorithm, O.R. Stage, Team Member (I.2.B):

### Cost:

I.2.B Dynamically changing the time of the O.R. stage for Team Members only affects ALE. There is no ACT or PLE in the O.R. stage.

Dynamically changing stage time in the O.R. stage will affect the Team Members for payment types: payment per hour, salary and payment per day. The Direct Labor algorithm in the Cost Module calculated the cost per hour for the Team Member for these 3 payment types. The Cost Table algorithm pulled the cost to the Cost Table in the Cost Module and the Dynamic Cost Table algorithm pulls this cost to the Dynamic Cost Table in the Profit Module.

The effect of changing stage time on the ALE and the resulting change in the cost of the Team Members in the O.R. stage is calculated by the Stage Time Variable sub algorithm. The cost of ALE in O.R. stage for the Team Members is background data saved by the Direct Labor algorithm. The Stage Time Variable sub algorithm multiplies this background ALE data by the percentage change in the O.R. stage time from the time that was inputted in CE set up.

By this multiplication, the Stage Time Variable sub algorithm will calculate the new ALE cost with changes in O.R. stage time for the Team Members. The Stage Time Variable sub algorithm can perform these calculations dynamically with dynamically inputted changes in stage time.

Using the graphic toggle function, the user can input changes in O.R. stage time for the Team Members and the Stage Time Variable sub algorithm will dynamically display the results.

### Revenue:

I.2.B Dynamically changing the time of the O.R. stage for Team Member has no effect on revenue.

### Stage Time Variable Sub Algorithm, Post-Op #1 Stage, Team Member (I.3.B):

### Cost:

1.3.B Dynamically changing the time of the Post-op #1 stage for Team Member has no effect on ALE. Changes will only affect ACT. There is no PLE for Team Members.

Dynamically changing stage time in the Post-op #1 stage will affect the Team Members for payment types: payment per hour, salary and payment per day. The Direct Labor algorithm in the Cost Module calculated the cost per hour for the Professional Provider for these 3 payment types. The Cost Table algorithm pulled the cost to the Cost Table in the Cost Module and the Dynamic Cost Table algorithm pulls this cost to the Dynamic Cost Table in the Profit Module.

The effect of changing stage time on the ACT and the resulting change in the cost of the Team Members in the Post-op #1 stage is calculated by the Stage Time Variable sub algorithm. The cost of ACT in Post-op #1 stage for the Team Members is background data saved by the Direct Labor algorithm. The Stage Time Variable sub algorithm multiplies this background ACT data by the percentage change in the Post-op #1 stage time from the time that was inputted in CE set up.

By this multiplication, the Stage Time Variable sub algorithm will calculate the new ACT cost with changes in Post-op #1 stage time for the Team Member. The ALE cost in the Post-op #1 stage for the Team Member is also background data saved by the Direct Labor algorithm. The Stage Time Variable sub algorithm will add this new ACT cost to the saved ALE cost. The resulting sum is the new cost of the Team Members in the Post-op #1 stage. The Stage Time Variable sub algorithm can perform these calculations dynamically with dynamically inputted changes in stage time.

Using the graphic toggle function, the user can input changes in Post-op #1 stage time for the Team Members and the Stage Time Variable sub algorithm will dynamically display the results.

### Revenue:

I.3.B Dynamically changing the time of the Post-op #1 stage for Team Member has no effect on revenue.

### Stage Time Variable Sub Algorithm, Post-Op #2 Stage, Team Member (I.4.B):

### Cost:

I.4.B Dynamically changing the time of the Post-op #2 stage for Team Member has no effect on ALE. Changes will only affect ACT. There is no PLE for Team Members.

Dynamically changing stage time in the Post-op #2 stage will affect the Team Members for payment types: payment per hour, salary and payment per day. The Direct Labor algorithm in the Cost Module calculated the cost per hour for the Team Member for these 3 payment types. The Cost Table algorithm pulled the cost to the Cost Table in the Cost Module and the Dynamic Cost Table algorithm pulls this cost to the Dynamic Cost Table in the Profit Module.

The effect of changing stage time on the ACT and the resulting change in the cost of the Team Members in the Post-op #1 stage is calculated by the Stage Time Variable sub algorithm. The cost of ACT in Post-op #2 stage for the Team Members is background data saved by the Direct Labor algorithm. The Stage Time Variable sub algorithm multiplies this background ACT data by the percentage change in the Post-op #2 stage time from the time that was inputted in CE set up.

By this multiplication, the Stage Time Variable sub algorithm will calculate the new ACT cost with changes in Post-op #2 stage time for the Team Member. The ALE cost in the Post-op #2 stage for the Team Member is also background data saved by the Direct Labor algorithm. The Stage Time Variable sub algorithm will add this new ACT cost to the saved ALE cost. The resulting sum is the new cost of the Team Members in the Post-op #2 stage. The Stage Time Variable sub algorithm can perform these calculations dynamically with dynamically inputted changes in stage time.

Using the graphic toggle function, the user can input changes in Post-op #2 stage time for the Team Members and the Stage Time Variable sub algorithm will dynamically display the results.

### Revenue:

I.4.B Dynamically changing the time of the Post-op #2 stage for Team Member has no effect on revenue.

### Stage Time Variable Sub Algorithm, Pre-Op Stage, Anesthesia Provider, MDA-Acting Alone (I.1.C-m):

### Stage Time Variable Sub Algorithm, Pre-Op Stage, Anesthesia Provider, CRNA-Acting Alone (I.1.C-c):

### Cost:

I.1.C-m & I.1.C-c Dynamically changing the time of the Pre-op stage for MDA acting alone or CRNA acting alone has no effect on ALE. Changes will only affect PLE.

Dynamically changing stage time in the Pre-op stage will affect the MDA acting alone or CRNA acting alone for payment types: payment per hour, salary and payment per day. The Direct Labor algorithm in the Cost Module calculated the cost per hour for the MDA acting alone or CRNA acting alone for these 3 payment types. The Cost Table algorithm pulled the cost to the Cost Table in the Cost Module and the Dynamic Cost Table algorithm pulls this cost to the Dynamic Cost Table in the Profit Module.

Dynamically changing stage time in the Pre-op stage will not affect the cost for the payment types: payment per case or percentage of anesthesia component.

The effect of changing stage time on the PLE and the resulting change in the cost of the MDA acting alone or CRNA acting alone in the Pre-op stage is calculated by the Stage Time Variable sub algorithm. The cost of PLE in Pre-op stage for the MDA acting alone or CRNA acting alone is background data saved by the Direct Labor algorithm. The Stage Time Variable sub algorithm multiplies this background PLE data by the percentage change in the Pre-op stage time from the time that was inputted in CE set up.

By this multiplication, the Stage Time Variable sub algorithm will calculate the new PLE cost with changes in Pre-op stage time for the MDA acting alone or CRNA acting alone. The ALE cost in the Pre-op stage for the MDA acting alone or CRNA acting alone is also background data saved by the Direct Labor algorithm. The Stage Time Variable sub algorithm will add this new PLE cost to the saved ALE cost. The resulting sum is the new cost of the MDA acting alone or CRNA acting alone in the Pre-op stage. The Stage Time Variable sub algorithm can perform these calculations dynamically with dynamically inputted changes in stage time.

Using the graphic toggle function, the user can input changes in pre-op stage time for the MDA acting alone or CRNA acting alone, and the Stage Time Variable sub algorithm will dynamically display the results.

### Revenue:

I.1.C-m & I.1.C-c Dynamically changing the time of the Pre-op stage for MDA acting alone or CRNA acting alone has no effect on revenue.

### Stage Time Variable Sub Algorithm, O.R. Stage, Anesthesia Provider, MDA-Acting Alone (I.2.C-m):

### Stage Time Variable Sub Algorithm, O.R. Stage, Anesthesia Provider, CRNA-Acting Alone (I.2.C-c):

### Cost for General Anesthesia/Mac & Conscious Sedation:

I.2.C-m & I.2.C-c Dynamically changing the time of the O.R. stage for MDA acting alone or CRNA acting alone affects ALE. There is no PLE in O.R. stage.

Dynamically changing stage time in the O.R. stage will affect the cost of an MDA acting alone or CRNA acting alone for payment types: payment per hour, salary and payment per day. The Direct Labor algorithm in the Cost Module calculated the cost per hour for the MDA acting alone or CRNA acting alone for these 3 payment types. The Cost Table algorithm pulled the cost to the Cost Table in the Cost Module and the Dynamic Cost Table algorithm pulls this cost to the Dynamic Cost Table in the Profit Module.

Dynamically changing stage time in the O.R. stage will not affect the cost for the payment types: payment per case or percentage of anesthesia component.

The cost of ALE in the O.R. stage for the MDA acting alone or CRNA acting alone is background data saved by the Direct Labor algorithm. The Stage Time Variable sub algorithm multiplies this background ALE data by the percentage change in the O.R. stage time from the time that was inputted in CE set up.

By this multiplication, the Stage Time Variable sub algorithm will calculate the new ALE cost with changes in O.R. stage time for the MDA acting alone or CRNA acting alone. The Stage Time Variable sub algorithm can perform these calculations dynamically with dynamically inputted changes in stage time.

Using the graphic toggle function, the user can input changes in O.R. stage time for the MDA acting alone or CRNA acting alone and the Stage Time Variable sub algorithm will dynamically display the results.

### Revenue General Anesthesia/Mac:

I.2.C-m & I.2.C-c The Stage Time Variable sub algorithm uses the changes in stage time for the O.R. stage and enters the new time into the anesthesia allowable formula for MDA acting alone or CRNA acting alone using the Anesthesia Allowable algorithm.

The Anesthesia Revenue Submodule algorithm is then applied to calculate the new total anesthesia revenue for all lines of code using the new time.

If the Collection Discount Anesthesia algorithm had been previously applied by the user to the total anesthesia revenue, it is also applied to the new total anesthesia revenue using the new time to calculate the new total corrected anesthesia revenue.

The user can dynamically change the stage time in the O.R. stage using the graphic toggle function and any resulting changes in anesthesia revenue are displayed to the user.

### Revenue Conscious Sedation:

I.2.C-m & I.2.C-c MDA acting alone and CRNA acting alone are only two Anesthesia Provider job descriptions allowed for conscious sedation.

Conscious sedation time occurs during the O.R. stage only. Changing stage time in the O.R. stage will proportionately change the conscious sedation time for the MDA acting alone or the CRNA acting alone. The HCPCS/CPT codes entered by the user in Anesthesia Revenue submodule using the CS Different Provider sub algorithm for conscious sedation is associated with conscious sedation time ranges. The time ranges are kept as background data. These time ranges have a minimum time and the maximum time. If the user has chosen the third method to enter CPT/HCPCS codes into the Anesthesia Revenue Submodule for the conscious sedation component, the user has chosen the time for conscious sedation and this time is used as the conscious sedation time. If the user has chosen the first or second method to enter CPT/HCPCS codes into the Anesthesia Revenue Submodule for the conscious sedation component, the user has not chosen the time for conscious sedation but a range of times. For purposes of the Stage Time Variable sub algorithm, the conscious sedation time when the user has chosen the first or second method is the average time in the HCPCS/CPT code time range between the minimum and maximum time range values.

The O.R. stage time is inputted independently by the user in CE set up from ALE time for the Anesthesia Provider in the O.R. stage. For General anesthesia/MAC, the ALE time in the O.R. stage for MDA acting alone, CRNA acting alone as well as other Anesthesia Provider job descriptions discussed in CE set up must be equal to the O.R. stage time. This is a system requirement. For conscious sedation the ALE time for the MDA acting alone, CRNA acting alone do not have to be equal to O.R. stage time because billing for conscious sedation is tied to the time that conscious sedation occurs and not entering the O.R. stage or the O.R. area.

The Stage Time Variable sub algorithm creates a 1:1 ratio between conscious sedation time and O.R. stage time. The percentage increase or decrease in stage time will result in the same percentage increase or decrease in conscious sedation time. Changes in conscious sedation time with changes in stage time may add or remove HCPCS/CPT codes and their associated revenue as described above if a time range threshold is crossed. Which sub algorithm applies is determined by the sub algorithm chosen by the user in the Anesthesia Revenue submodule conscious sedation section.

The user can dynamically change the stage time in the O.R. stage using the graphic toggle function and any resulting changes in anesthesia revenue are displayed to the user.

### Stage Time Variable Sub Algorithm, Post-Op #1 Stage, Anesthesia Provider, MDA-Acting Alone (I.3.C-m):

### Stage Time Variable Sub Algorithm, Post-Op #1 Stage, Anesthesia Provider, CRNA-Acting Alone (I.3.C-c):

### Cost for General Anesthesia/Mac & Conscious Sedation

I.3.C-m & I.3.C-c Dynamically changing the time of the Post-op #1 stage for MDA acting alone or CRNA acting alone affects ALE. There is no PLE in Post-op #1 stage for Anesthesia Providers.

Dynamically changing stage time in the Post-op #1 stage will affect the cost of an MDA acting alone or CRNA acting alone for payment types: payment per hour, salary and payment per day. The Direct Labor algorithm in the Cost Module calculated the cost per hour for the MDA acting alone or CRNA acting alone for these 3 payment types and pushed it to Profit Module.

Dynamically changing stage time in the Post-op #1 stage will not affect the cost for the payment types: payment per case or percentage of anesthesia component.

The cost of ALE in the Post-op #1 stage for the MDA acting alone or CRNA acting alone is background data saved by the Direct Labor algorithm. The Stage Time Variable sub algorithm multiplies this background ALE data by the percentage change in the Post-op #1 stage time from the time that was inputted in CE set up.

By this multiplication, the Stage Time Variable sub algorithm will calculate the new ALE cost with changes in Post-op #1 stage time for the MDA acting alone or CRNA acting alone. The Stage Time Variable sub algorithm can perform these calculations dynamically with dynamically inputted changes in stage time.

Using the graphic toggle function, the user can input changes in Post-op #1 stage time for the MDA acting alone or CRNA acting alone, and the Stage Time Variable sub algorithm will dynamically display the results.

### Revenue General Anesthesia/Mac

I.3.C-m & I.3.C-c The Stage Time Variable sub algorithm uses the changes in stage time for the Post-o #1 stage and enters the new time into the anesthesia allowable formula for MDA acting alone or CRNA acting alone using the Anesthesia Allowable algorithm.

The Anesthesia Revenue Submodule algorithm is then applied to calculate the new total anesthesia revenue for all lines of code using the new time.

If the Collection Discount Anesthesia algorithm had been previously applied by the user to the total anesthesia revenue, it is also applied to the new total anesthesia revenue using the new time to calculate the new total corrected anesthesia revenue.

The user can dynamically change the stage time in the Post-op #1 stage using the graphic toggle function and any resulting changes in anesthesia revenue are displayed to the user.

### Revenue Conscious Sedation

I.3.C-m & I.3.C-c There is no revenue in Post-op #1 conscious sedation, and changes in Stage Time for Post-Op #1 Will have No have Effect on Revenue or Gross Profit.

### Stage Time Variable Sub Algorithm, Post-Op #2 Stage, Anesthesia Provider, MDA-Acting Alone (I.4.C-m)

### Stage Time Variable Sub Algorithm, Post-Op #2 Stage, Anesthesia Provider, CRNA-Acting Alone (I.4.C-c)

### Cost & Revenue

I.4.C-m & I.4.C-c Changes in the stage time for Post-op #2 have no effect on Anesthesia Provider cost, revenue or profit.

### Stage Time Variable Sub Algorithm, Pre-Op Stage, Anesthesia Provider, MDA-Supervising (I.1.C-ms)

### Stage Time Variable Sub Algorithm, Pre-Op Stage, Anesthesia Provider, MDA-Directing (I.1.C-Cd)

### Cost

I.1.C-ms & I.1.C-md Dynamically changing the time of the Pre-op stage for MDA-Supervising or MDA-Directing has no effect on ALE. Changes will only affect ACT. There is no PLE for MDA-Supervising or MDA-Directing.

Dynamically changing stage time in the Pre-op stage will affect the MDA-Supervising or MDA-Directing for payment types: payment per hour, salary and payment per day. The Direct Labor algorithm in the Cost Module calculated the cost per hour for the MDA-Supervising or MDA-Directing for these 3 payment types. The Cost Table algorithm pulled the cost to the Cost Table in the Cost Module and the Dynamic Cost Table algorithm pulls this cost to the Dynamic Cost Table in the Profit Module.

The effect of changing stage time on the ACT and the resulting change in the cost of the MDA-Supervising or MDA-Directing in the Pre-op stage is calculated by the stage Time Variable sub algorithm. The cost of ACT in Pre-op stage for the MDA-Supervising or MDA-Directing is background data saved by the Direct Labor algorithm. The Stage Time Variable sub algorithm multiplies this background ACT data by the percentage change in the Pre-op stage time from the time that was inputted in CE set up.

By this multiplication, the Stage Time Variable sub algorithm will calculate the new ACT cost with changes in Pre-op stage time for the MDA-Supervising or MDA-Directing. The ALE cost in the Pre-op stage for the MDA-Supervising or MDA-Directing is also background data saved by the Direct Labor algorithm. The stage time variable sub algorithm will add this new ACT cost to the saved ALE cost. The resulting sum is the new cost of the MDA-Supervising or MDA-Directing in the Pre-op stage. The Stage Time Variable sub algorithm can perform these calculations dynamically with dynamically inputted changes in stage time.

Using the graphic toggle function, the user can input changes in pre-op stage time for the MDA-Supervising or MDA-Directing and the Stage Time Variable sub algorithm will dynamically display the results.

### Revenue

I.1.C-ms & I.1.C-md Dynamically changing the time of the Pre-op stage for MDA-supervising or MDA-directing has no effect on revenue.

### Stage Time Variable Sub Algorithm, O.R. Stage, Anesthesia Provider, MDA-Supervising (I.2.C-ms):

### Stage Time Variable Sub Algorithm, O.R. Stage, Anesthesia Provider, MDA-Directing (I.2.C-cd):

### Cost for General Anesthesia/Mac

I.2.C-ms & I.2.C-cd Dynamically changing the time of the O.R. stage for MDA-Supervising or MDA-Directing affects ALE and ACT. There is no PLE in O.R. stage.

Dynamically changing stage time in the O.R. stage will affect the cost of an MDA-Supervising or MDA-Directing for payment types: payment per hour, salary and payment per day. The Direct Labor algorithm in the Cost Module calculated the cost per hour for the MDA-Supervising or MDA-Directing for these 3 payment types. The Cost Table algorithm pulled the cost to the Cost Table in the Cost Module and the Dynamic Cost Table algorithm pulls this cost to the Dynamic Cost Table in the Profit Module.

Dynamically changing stage time in the O.R. stage will not affect the cost for the payment types: payment per case or percentage of anesthesia component.

The cost of ALE and ACT in the O.R. stage for the MDA-Supervising or MDA-Directing is background data saved by the Direct Labor algorithm. The Stage Time Variable sub algorithm multiplies this background ALE and ACT data by the percentage change in the O.R. stage time from the time that was inputted in CE set up.

By this multiplication, the Stage Time Variable sub algorithm will calculate the new ALE and ACT cost with changes in O.R. stage time for the MDA-Supervising or MDA-Directing. The Stage Time Variable sub algorithm can perform these calculations dynamically with dynamically inputted changes in stage time.

Using the graphic toggle function, the user can input changes in O.R. stage time for the MDA-Supervising or MDA-Directing and the Stage Time Variable sub algorithm will dynamically display the results.

### Revenue General Anesthesia/Mac

I.2.C-ms & I.2.C-cd The effect of changing stage time on the revenue for General anesthesia/MAC for MDA-Directing (I.2.C-cd) and MDA-Supervising (I.2.C-ms) are different.

I.2.C-ms Changes in stage time have no effect on anesthesia revenue for MDA-Supervising.

I.2.C-md For MDA-Directing, the Stage Time Variable sub algorithm uses the changes in stage time for the O.R. stage and enters the new time into the anesthesia allowable formula using the Anesthesia Allowable algorithm.

The Anesthesia Revenue Submodule algorithm is then applied to calculate the new total anesthesia revenue for all lines of code using the new time.

If the Collection Discount Anesthesia algorithm had been previously applied by the user to the total anesthesia revenue, it is also applied to the new total anesthesia revenue using the new time to calculate the new total corrected anesthesia revenue.

The user can dynamically change the stage time in the O.R. stage using the graphic toggle function and any resulting changes in anesthesia revenue are displayed to the user.

Stage Time Variable sub algorithm, Post-Op #1 Stage, Anesthesia Provider, MDA-Supervising (I.3.C-ms):

### Stage Time Variable Sub Algorithm, Post-Op #1 Stage, Anesthesia Provider, MDA-Directing (I.3.C-cd):

### Cost for General Anesthesia/Mac

I.3.C-ms & I.3.C-cd Dynamically changing the time of the Post-op #1 stage for MDA-Supervising or MDA-Directing affects ALE and ACT. There is no PLE in Post-op #1 stage.

Dynamically changing stage time in the Post-op #1 stage will affect the cost of an MDA-Supervising or MDA-Directing for payment types: payment per hour, salary and payment per day. The Direct Labor algorithm in the Cost Module calculated the cost per hour for the MDA-Supervising or MDA-Directing for these 3 payment types. The Cost Table algorithm pulled the cost to the Cost Table in the Cost Module and the Dynamic Cost Table algorithm pulls this cost to the Dynamic Cost Table in the Profit Module.

Dynamically changing stage time in the Post-op #1 stage will not affect the cost for the payment types: payment per case or percentage of anesthesia component.

The cost of ALE and ACT in the Post-op #1 stage for the MDA-Supervising or MDA-Directing is background data saved by the Direct Labor algorithm. The Stage Time Variable sub algorithm multiplies this background ALE and ACT data by the percentage change in the Post-op #1 stage time from the time that was inputted in CE set up.

By this multiplication, the Stage Time Variable sub algorithm will calculate the new ALE and ACT cost with changes in Post-op #1 stage time for the MDA-Supervising or MDA-Directing. The Stage Time Variable sub algorithm can perform these calculations dynamically with dynamically inputted changes in stage time.

Using the graphic toggle function, the user can input changes in Post-op #1 stage time for the MDA-Supervising or MDA-Directing and the Stage Time Variable sub algorithm will dynamically display the results.

### Revenue General Anesthesia/Mac

I.3.C-ms & I.3.C-cd The effect of changing stage time on the revenue for General anesthesia/MAC for MDA-Directing (I.3.C-cd) and MDA-Supervising (I.3.C-ms) are different.

I.3.C-ms Changes in stage time have no effect on anesthesia revenue for MDA-Supervising.

I.3.C-md For MDA-Directing, the Stage Time Variable sub algorithm uses the changes in stage time for the Post-op #1 stage and enters the new time into the anesthesia allowable formula using the Anesthesia Allowable algorithm.

The Anesthesia Revenue Submodule algorithm is then applied to calculate the new total anesthesia revenue for all lines of code using the new time.

If the Collection Discount Anesthesia algorithm had been previously applied by the user to the total anesthesia revenue, it is also applied to the new total anesthesia revenue using the new time to calculate the new total corrected anesthesia revenue.

The user can dynamically change the stage time in the Post-op #1 stage using the graphic toggle function and any resulting changes in anesthesia revenue are displayed to the user.

### Stage Time Variable Sub Algorithm, Post-Op #2 Stage, Anesthesia Provider, MDA-Supervising (I.4.C-ms):

### Stage Time Variable Sub Algorithm, Post-Op #2 Stage, Anesthesia Provider, MDA-Directing (I.4.C-cd):

### Cost & Revenue

I.4.C-ms & I.4.C-md Changes in the stage time for Post-op #2 have no effect on Anesthesia Provider cost, revenue or profit.

### Stage Time Variable Sub Algorithm, Pre-Op Stage, Anesthesia Provider, CRNA-Supervised (I.1.C-sc):

### Stage Time Variable Sub Algorithm, Pre-Op Stage, Anesthesia Provider, AA-Supervised (I.1.C-sa):

### Stage Time Variable Sub Algorithm, Pre-Op Stage, Anesthesia Provider, CRNA-Directed (I.1.C-dc):

### Stage Time Variable Sub Algorithm, Pre-Op Stage, Anesthesia Provider, AA-Directed (I.1.C-da):

### Cost

I.1.C-sc, I.1.C-sa, I.1C-dc & I.1.C-da Dynamically changing the time of the Pre-op stage for CRNA-Supervised, AA-Supervised, CRNA-Directed or AA-Directed only effects ALE. There is no ACT or PLE in the Pre-op stage.

Dynamically changing stage time in the Pre-op stage will affect the CRNA-Supervised, AA-Supervised, CRNA-Directed or AA-Directed for payment types: payment per hour, salary and payment per day. The Direct Labor algorithm in the Cost Module calculated the cost per hour for the CRNA-Supervised, AA-Supervised, CRNA-Directed or AA-Directed for these 3 payment types. The Cost Table algorithm pulled the cost to the Cost Table in the Cost Module and the Dynamic Cost Table algorithm pulls this cost to the Dynamic Cost Table in the Profit Module.

Dynamically changing stage time in the Pre-op stage will not affect the cost for the payment types: payment per case or percentage of professional component.

The effect of changing stage time on the ALE and the resulting change in the cost of the CRNA-Supervised, AA-Supervised, CRNA-Directed or AA-Directed in the Pre-op stage is calculated by the Stage Time Variable sub algorithm. The cost of ALE in Pre-op stage for the CRNA-Supervised, AA-Supervised, CRNA-Directed or AA-Directed is background data saved by the Direct Labor algorithm. The Stage Time Variable sub algorithm multiplies this background ALE data by the percentage change in the Pre-op stage time from the time that was inputted in CE set up.

By this multiplication, the Stage Time Variable sub algorithm will calculate the new ALE cost with changes in Pre-op stage time for the CRNA-Supervised, AA-Supervised, CRNA-Directed or AA-Directed. The Stage Time Variable sub algorithm can perform these calculations dynamically with dynamically inputted changes in stage time.

Using the graphic toggle function, the user can input changes in Pre-op stage time for the CRNA-Supervised, AA-Supervised, CRNA-Directed or AA-Directed and the Stage Time Variable sub algorithm will dynamically display the results.

### Revenue

I.1.C-sc, I.1.C-sa, I.1C-dc & I.1.C-da Dynamically changing the time of the Pre-op stage for CRNA-Supervised, AA-Supervised, CRNA-Directed or AA-Directed present during the CE has no effect on revenue.

### Stage Time Variable Sub Algorithm, O.R. Stage, Anesthesia Provider, CRNA-Supervised (I.2.C-sc):

### Stage Time Variable Sub Algorithm, O.R. Stage, Anesthesia Provider, AA-Supervised (I.2.C-sa):

### Stage Time Variable Sub Algorithm, O.R. Stage, Anesthesia Provider, CRNA-Directed (I.2.C-dc):

### Stage Time Variable Sub Algorithm, O.R. Stage, Anesthesia Provider, AA-Directed (I.2.C-da):

### Cost for General Anesthesia/Mac

I.2.C-sc, I.2.C-sa, I.2.C-dc & I.2.C-da Dynamically changing the time of the O.R. stage for CRNA-Supervised, AA-Supervised, CRNA-Directed or AA-Directed affects ALE. There is no PLE in O.R. stage.

Dynamically changing stage time in the O.R. stage will affect the cost of an CRNA-Supervised, AA-Supervised, CRNA-Directed or AA-Directed for payment types: payment per hour, salary and payment per day. The Direct Labor algorithm in the Cost Module calculated the cost per hour for the CRNA-Supervised, AA-Supervised, CRNA-Directed or AA-Directed for these 3 payment types. The Cost Table algorithm pulled the cost to the Cost Table in the Cost Module and the Dynamic Cost Table algorithm pulls this cost to the Dynamic Cost Table in the Profit Module.

Dynamically changing stage time in the O.R. stage will not affect the cost for the payment types: payment per case or percentage of anesthesia component.

The cost of ALE in the O.R. stage for the CRNA-Supervised, AA-Supervised, CRNA-Directed or AA-Directed is background data saved by the Direct Labor algorithm. The Stage Time Variable sub algorithm multiplies this background ALE data by the percentage change in the O.R. stage time from the time that was inputted in CE set up.

By this multiplication, the Stage Time Variable sub algorithm will calculate the new ALE cost with changes in O.R. stage time for the CRNA-Supervised, AA-Supervised, CRNA-Directed or AA-Directed. The Stage Time Variable sub algorithm can perform these calculations dynamically with dynamically inputted changes in stage time.

Using the graphic toggle function, the user can input changes in O.R. stage time for the CRNA-Supervised, AA-Supervised, CRNA-Directed or AA-Directed and the stage Time Variable sub algorithm will dynamically display the results.

### Revenue General Anesthesia/Mac

I.2.C-sc, I.2.C-sa, I.2.C-dc & I.2.C-da The Stage Time Variable sub algorithm uses the changes in stage time for the O.R. stage and enters the new time into the anesthesia allowable formula for CRNA-Supervised, AA-Supervised, CRNA-Directed or AA-Directed using the Anesthesia Allowable algorithm.

The Anesthesia Revenue Submodule algorithm is then applied to calculate the new total anesthesia revenue for all lines of code using the new time.

If the Collection Discount Anesthesia algorithm had been previously applied by the user to the total anesthesia revenue, it is also applied to the new total anesthesia revenue using the new time to calculate the new total corrected anesthesia revenue.

The user can dynamically change the stage time in the O.R. stage using the graphic toggle function and any resulting changes in anesthesia revenue are displayed to the user.

### Stage Time Variable Sub Algorithm, Post-Op #1 Stage, Anesthesia Provider, CRNA-Supervised (I.3.C-sc):

### Stage Time Variable Sub Algorithm, Post-Op #1 Stage, Anesthesia Provider, AA-Supervised (I.3.C-sa):

### Stage Time Variable Sub Algorithm, Post-Op #1 Stage, Anesthesia Provider, CRNA-directed (I.3.C-dc):

### Stage Time Variable Sub Algorithm, Post-Op #1 Stage, Anesthesia Provider, AA-Directed (I.3.C-da):

### Cost General Anesthesia/Mac

I.3.C-sc, I.3.C-sa, I.3.C-dc & I.3.C-da Dynamically changing the time of the Post-op #1 stage for CRNA-Supervised, AA-Supervised, CRNA-Directed or AA-Directed affects ALE. There is no PLE in Post-op #1 stage.

Dynamically changing stage time in the Post-op #1 stage will affect the cost of an CRNA-Supervised, AA-Supervised, CRNA-Directed or AA-Directed for payment types: payment per hour, salary and payment per day. The Direct Labor algorithm in the Cost Module calculated the cost per hour for the CRNA-Supervised, AA-Supervised, CRNA-Directed or AA-Directed for these 3 payment types. The Cost Table algorithm pulled the cost to the Cost Table in the Cost Module and the Dynamic Cost Table algorithm pulls this cost to the Dynamic Cost Table in the Profit Module.

Dynamically changing stage time in the Post-op #1 stage will not affect the cost for the payment types: payment per case or percentage of anesthesia component.

The cost of ALE in the Post-op #1 stage for the CRNA-Supervised, AA-Supervised, CRNA-Directed or AA-Directed is background data saved by the Direct Labor algorithm. The Stage Time Variable sub algorithm multiplies this background ALE data by the percentage change in the Post-op #1 stage time from the time that was inputted in CE set up.

By this multiplication, the Stage Time Variable sub algorithm will calculate the new ALE cost with changes in Post-op #1 stage time for the CRNA-Supervised, AA-Supervised, CRNA-Directed or AA-Directed. The Stage Time Variable sub algorithm can perform these calculations dynamically with dynamically inputted changes in stage time.

Using the graphic toggle function, the user can input changes in Post-op #1 stage time for the CRNA-Supervised, AA-Supervised, CRNA-Directed or AA-Directed and the Stage Time Variable sub algorithm will dynamically display the results.

### Revenue General Anesthesia/Mac

I.3.C-sc, I.3.C-sa, I.3.C-dc & I.3.C-da The Stage Time Variable sub algorithm uses the changes in stage time for the Post-op #1 stage and enters the new time into the anesthesia allowable formula for CRNA-Supervised, AA-Supervised, CRNA-Directed or AA-Directed using the Anesthesia Allowable algorithm.

The Anesthesia Revenue Submodule algorithm is then applied to calculate the new total anesthesia revenue for all lines of code using the new time.

If the Collection Discount Anesthesia algorithm had been previously applied by the user to the total anesthesia revenue, it is also applied to the new total anesthesia revenue using the new time to calculate the new total corrected anesthesia revenue.

The user can dynamically change the stage time in the Post-op #1 stage using the graphic toggle function and any resulting changes in anesthesia revenue are displayed to the user.

### Stage Time Variable Sub Algorithm, Post-Op #2 Stage, Anesthesia Provider, CRNA-Supervised (I.4.C-Sc):

### Stage Time Variable Sub Algorithm, Post-Op #2 Stage, Anesthesia Provider, AA-Supervised (I.4.C-sa):

### Stage Time Variable Sub Algorithm, Post-Op #2 Stage, Anesthesia Provider, CRNA-Directed (I.4.C-dc):

### Stage Time Variable Sub Algorithm, Post-Op #2 Stage, Anesthesia Provider, AA-Directed (I.4.C-da):

### Cost & Revenue

I.4.C-sc, I.4.C-sa, I.4.C-dc & I.4.C-da Changes in the stage time for Post-op #2 have no effect on Anesthesia Provider cost, revenue or profit.

### Stage Time Variable Sub Algorithm, Pre-Op Stage, Ancillary Provider Inside CE (I.1.D-i):

### Cost

I.1.D-i Dynamically changing the time of the Pre-op stage for Ancillary Provider present during the CE has no effect on ALE. Changes will only affect PLE.

Dynamically changing stage time in the Pre-op stage will affect the Ancillary Provider present during the CE for payment types: payment per hour, salary and payment per day. The Direct Labor algorithm in the Cost Module calculated the cost per hour for the Ancillary Provider for these 3 payment types. The Cost Table algorithm pulled the cost to the Cost Table in the Cost Module and the Dynamic Cost Table algorithm pulls this cost to the Dynamic Cost Table in the Profit Module.

Dynamically changing stage time in the Pre-op stage will not affect the cost for the payment types: payment per case or percentage of ancillary component.

The effect of changing stage time on the PLE and the resulting change in the cost of the Ancillary Provider present during the CE in the Pre-op stage is calculated by the Stage Time Variable sub algorithm. The cost of PLE in Pre-op stage for the Ancillary Provider present during the CE is background data saved by the Direct Labor algorithm. The Stage Time Variable sub algorithm multiplies this background PLE data by the percentage change in the Pre-op stage time from the time that was inputted in CE set up.

By this multiplication, the Stage Time Variable sub algorithm will calculate the new PLE cost with changes in Pre-op stage time for the Ancillary Provider present during the CE. The ALE cost in the Pre-op stage for the Ancillary Provider present during the CE is also background data saved by the Direct Labor algorithm. The stage time variable sub algorithm will add this new PLE cost to the saved ALE cost. The resulting sum is the new cost of the Ancillary Provider present during the CE in the Pre-op stage. The Stage Time Variable sub algorithm can perform these calculations dynamically with dynamically inputted changes in stage time.

Using the graphic toggle function, the user can input changes in pre-op stage time for the Professional Provider and the Stage Time Variable sub algorithm will dynamically display the results.

### Revenue

I.1.D-i Dynamically changing the time of the Pre-op stage for Ancillary Provider present during CE has no effect on revenue.

### Stage Time Variable Sub Algorithm, O.R. Stage, Ancillary Provider Inside CE (I.2.D-i):

### Cost

I.2.D-i Dynamically changing the time of the O.R. stage for Ancillary Provider present during the CE only effects ALE. There is no PLE in the O.R. stage.

Dynamically changing stage time in the O.R. stage will affect the Ancillary Provider present during the CE for payment types: payment per hour, salary and payment per day. The Direct Labor algorithm in the Cost Module calculated the cost per hour for the Ancillary Provider for these 3 payment types. The Cost Table algorithm pulled the cost to the Cost Table in the Cost Module and the Dynamic Cost Table algorithm pulls this cost to the Dynamic Cost Table in the Profit Module.

Dynamically changing stage time in the O.R. stage will not affect the cost for the payment types: payment per case or percentage of ancillary component.

The effect of changing stage time on the ALE and the resulting change in the cost of the Ancillary Provider present during the CE in the O.R. stage is calculated by the Stage Time Variable sub algorithm. The cost of ALE in O.R. stage for the Ancillary Provider present during the CE is background data saved by the Direct Labor algorithm. The Stage Time Variable sub algorithm multiplies this background ALE data by the percentage change in the O.R. stage time from the time that was inputted in CE set up.

By this multiplication, the Stage Time Variable sub algorithm will calculate the new ALE cost with changes in O.R. stage time for the Ancillary Provider present during the CE. The Stage Time Variable sub algorithm can perform these calculations dynamically with dynamically inputted changes in stage time.

Using the graphic toggle function, the user can input changes in O.R. stage time for the Ancillary Provider present during the CE and the Stage Time Variable sub algorithm will dynamically display the results.

### Revenue

I.2.D-i Dynamically changing the time of the O.R. stage for Ancillary Provider present during the CE has no effect on revenue.

### Stage Time Variable Sub Algorithm, Post-Op #1 Stage, Ancillary Provider Inside CE (I.3.D-i):

### Cost

I.3.d-i Dynamically changing the time of the Post-op #1 stage for Ancillary Provider present during the CE has no effects on ALE. There is no PLE in the Post-op #1 stage.

Using the graphic toggle function, the user can input changes in Post-op #1 stage time for the Ancillary Provider present during the CE and the Stage Time Variable sub algorithm will not change cost or Gross Profit.

### Revenue

I.3.D-i Dynamically changing the time of the Post-op #1 stage for Ancillary Provider present during the CE has no effect on revenue.

### Stage Time Variable Sub Algorithm, Post-Op #2 Stage, Ancillary Provider Inside CE (I.4.D-i)

### Cost

I.4.D-i Dynamically changing the time of the Post-op #2 stage for Ancillary Provider present during the CE has no effects on ALE. There is no PLE in the Post-op #2 stage.

Using the graphic toggle function, the user can input changes in Post-op #2 stage time for the Ancillary Provider present during the CE and the Stage Time Variable sub algorithm will not change cost or Gross Profit.

### Revenue

I.3.D-i Dynamically changing the time of the Post-op #1 stage for Ancillary Provider present during the CE has no effect on revenue.

### Stage Time Variable Sub Algorithm, Pre-Op Stage, Ancillary Provider Outside CE (I.1.D-o):

### Cost

I.1.D-o Dynamically changing the time of the Pre-op stage for Ancillary Provider not present during the CE has no effect on ALE. There is no PLE.

Using the graphic toggle function, the user can input changes in Pre-op stage time for the Ancillary Provider not present during the CE and the Stage Time Variable sub algorithm will not change cost or Gross Profit.

### Revenue

I.1.D-o Dynamically changing the time of the Pre-op stage for Ancillary Provider not present during the CE has no effect on revenue.

### Stage Time Variable Sub Algorithm, O.R. Stage, Ancillary Provider Outside CE (I.2.D-o):

### Cost

I.2.D-o Dynamically changing the time of the O.R. stage for Ancillary Provider not present during the CE has no effect on ALE. There is no PLE.

Using the graphic toggle function, the user can input changes in O.R. stage time for the Ancillary Provider not present during the CE and the Stage Time Variable sub algorithm will not change cost or Gross Profit.

### Revenue

I.2.D-o Dynamically changing the time of the O.R. stage for Ancillary Provider not present during the CE has no effect on revenue.

### Stage Time Variable Sub Algorithm, Post-Op #1 Stage, Ancillary Provider Outside CE (I.3.D-o):

### Cost

I.3.D-o Dynamically changing the time of the Post-op #1 stage for Ancillary Provider not present during the CE has no effect on ALE. There is no PLE.

Using the graphic toggle function, the user can input changes in Pre-op stage time for the Ancillary Provider not present during the CE and the Stage Time Variable sub algorithm will not change cost or Gross Profit.

### Revenue

I.3.D-o Dynamically changing the time of the Post-op #1 stage for Ancillary Provider not present during the CE has no effect on revenue.

### Stage Time Variable Sub Algorithm, Post-Op #2 Stage, Ancillary Provider Outside CE (I.4.D-o):

### Cost

I.4.D-o Dynamically changing the time of the Post-op #2 stage for Ancillary Provider not present during the CE has no effect on ALE. There is no PLE.

Using the graphic toggle function, the user can input changes in Post-op #2 stage time for the Ancillary Provider not present during the CE and the Stage Time Variable sub algorithm will not change cost or Gross Profit.

### Revenue

I.4.D-o Dynamically changing the time of the Post-op #2 stage for Ancillary Provider not present during the CE has no effect on revenue.

### [PART TWO] II. Inventory Variable Sub Algorithm.

Changes in inventory cost for items in each of the 5 Inventory Classes (Disposable, Medications, HCD, Implants, DME/Orthotics) affect the line cost for that item and the total line cost on the Dynamic Cost Table and on Dynamic Gross profit.

Changes in inventory cost for an item or items in the Implant Class may also have an effect on the implant revenue for that item or items on the Dynamic Revenue Table.

Changes in inventory cost for an item or items in the HCD Class will have an effect on the HCD revenue for that item or items on the Dynamic Revenue Table.

### Class II Disposables

The “line cost” of class II disposable items in a particular CE is the sum cost of all class II disposable items including ordinary disposables, disposable instruments, disposable vendor packs and/or disposable custom packs used in that CE and Dynamic Cost Table algorithm pulls this “line cost” to the Dynamic Cost Table.

The Inventory Variable sub algorithm allows the line cost to be dynamically changed using the graphic toggle function. The Inventory Variable sub algorithm allows for changes in the line cost for the disposables and the resulting changes in total line cost on Dynamic Cost Table to be calculated and dynamically displayed.

The Gross Profit algorithm dynamically calculates and displays the new Dynamic Gross Profit.

### Class III, Medications

The line cost of class III medication items in a particular CE is the sum cost of all medication items used in that CE and pulls to the Dynamic Cost Table by Dynamic Cost Table algorithm.

The Inventory Variable sub algorithm allows the line cost to be dynamically changed using the graphic toggle function. The Inventory Variable sub algorithm allows for changes in the line cost for the medications and the resulting changes in total line cost on Dynamic Cost Table to be calculated and dynamically displayed.

The Gross Profit algorithm dynamically calculates and displays the new Dynamic Gross Profit.

### Class IV, HCD

The line cost of class IV HCD items used in a particular CE is the cost of HCD item or items pulled by the Dynamic Cost Table algorithm to the Dynamic Cost Table. The cost of HCD items is listed separately in the Dynamic Cost Table.

### Linked Revenue and Cost for HCD

HCD by definition have a cost and revenue component.

When the mandatory Link to Revenue algorithm is completed in the fifth section of the IIS Card algorithm, the cost of the HCD item is linked to the revenue for that item in the Surgery Center Revenue submodule. The link establishes a 1:1 relationship between changes in cost and revenue for the HCD item. Each HCD “single item cost” for different HCD items are linked to revenue for that item separately using the Link to Revenue algorithm.

When the HCD cost is linked to revenue, changes in HCD cost will change the HCD revenue. The Inventory Variable sub algorithm allows the linked cost and revenue for the HCD item to be dynamically and simultaneously changed and displayed using the graphic toggle function. The resulting changes in total line cost on the Dynamic Cost Table and on total revenue or “corrected total revenue” on the Dynamic Revenue table are calculated and dynamically displayed.

The Gross Profit algorithm dynamically calculates and displays the new Dynamic Gross Profit with changes in the linked HCD cost and revenue.

### Class V, Implants

Cost only for Implants.

The line cost of class V Implant items used in a particular CE is the cost of Implant item or items pulled by the Dynamic Cost Table algorithm to the Dynamic Cost Table. The cost of Implant items are listed separately in the Dynamic Cost Table.

The Inventory Variable sub algorithm allows the line cost of the Implant item to be dynamically changed using the graphic toggle function. The Inventory Variable sub algorithm allows for the changes in the line cost for the Implant item and the resulting changes in total line cost on Dynamic Cost Table to be calculated and dynamically displayed.

The Gross Profit algorithm dynamically calculates and displays the new Dynamic Gross Profit with changes in implant cost.

### Linked Revenue and Cost for Implants:

When the Link to Revenue algorithm option is chosen in the fifth section of the IIS Card sub algorithm, the cost of the implant item is linked to the revenue for that item in the Surgery Center Revenue submodule. The link establishes a 1:1 relationship between changes in cost and revenue for the implant item. Each implant “single item cost” for different implant items are linked to revenue for that item separately using the Link to Revenue algorithm.

When the implant cost is linked to revenue, changes in implant cost will change the implant revenue. The Inventory Variable sub algorithm allows the linked cost and revenue for the implant item to be dynamically and simultaneously changed and displayed using the graphic toggle function. The resulting changes in total line cost on the Dynamic Cost Table and on total revenue or “corrected total revenue” on the Dynamic Revenue table are calculated and dynamically displayed.

The Gross Profit algorithm dynamically calculates and displays the new Dynamic Gross Profit with changes in linked implant cost and revenue.

### Class VI, DME/Orthotics:

### Cost Only for DME/Orthotics:

The line cost of Class VI DME/Orthotic items used in a particular CE is the cost of DME/Orthotic item or items pulls by the Dynamic Cost Table algorithm to the Dynamic Cost Table. The cost of DME/Orthotic items are listed separately in the Dynamic Cost Table. The Inventory Variable sub algorithm allows the line cost of the DME/Orthotic item to be dynamically changed using the graphic toggle function. The Inventory Variable sub algorithm allows for the changes in the line cost for the DME/Orthotic item and the resulting changes in total line cost on Dynamic Cost Table to be calculated and dynamically displayed.

The Gross Profit algorithm dynamically calculates and displays the new Dynamic Gross Profit with changes in DME/Orthotic cost.

### Linked Revenue and Cost for DME/Orthotics:

When the Link to Revenue algorithm option is chosen in the second section of the IIS Card sub algorithm, the cost of the DME/Orthotic item is linked to the revenue for that item. The Link to Professional Revenue sub algorithm links the cost of the DME/Orthotic item to the revenue generated for that item in the Professional Revenue submodule. The Link to Surgery Center Revenue sub algorithm links the cost of the DME/Orthotic item to the revenue generated for that item in the Surgery Center Revenue submodule. In both cases, the link establishes a 1:1 relationship between changes in cost and revenue for the DME/Orthotic item. Each DME/Orthotic single item cost for different DME/Orthotic items are linked to revenue for that item separately using the Link to Revenue algorithm.

When the DME/Orthotic cost is linked to revenue, changes in DME/Orthotic cost will change the DME/Orthotic revenue. The Inventory Variable sub algorithm allows the linked cost and revenue for the DME/Orthotic item to be dynamically and simultaneously changed and displayed using the graphic toggle function. The resulting changes in total line cost on the Dynamic Cost Table and on total revenue or “corrected total revenue” on the Dynamic Revenue table are calculated and dynamically displayed.

The Gross Profit algorithm dynamically calculates and displays the new Dynamic Gross Profit with changes in linked DME/Orthotic cost and revenue.

### [PART TWO] III. Player Cost Variable Sub Algorithm.

Changing a Player's cost for all payment types will affect the cost of that Player in all stages of the CE in which that Player is engaged with the client. Changing a Player's cost also affects the Player's AO time when present.

### Professional Provider:

### Professional Provider, Payment Per Hour (A.a):

A.a The Professional Provider hourly rate was entered in CE set up using the Payment Type sub algorithm with or without FICA. The hourly rate cost is pushed to Direct Labor algorithm by the CE Set Up algorithm which calculates the line cost for the Professional Provider. The line cost includes the cost of the Professional Provider in all stages of the CE where the Professional Provider is engaged with the client and the Professional Provider's AO time if present. The Cost Table algorithm pulls the line cost calculated by the Direct Labor algorithm to the Cost Table. The Dynamic Cost Table algorithm pulls the line cost to the Dynamic Cost Table in the Profit Module. The Player Cost Variable sub algorithm accesses the line cost on the Dynamic Cost Table.

The Player Cost Variable sub algorithm allows the line cost to be dynamically changed using the graphic toggle function. The Player Cost Variable sub algorithm allows for changes in the line cost for a Player and the resulting changes in total line cost on Dynamic Cost Table to be calculated and dynamically displayed.

The Gross Profit algorithm dynamically calculates and displays the new Dynamic Gross Profit.

### Professional Provider, Salary (A.b):

A.b The Professional Provider salary was entered in CE set up using the Payment Type sub algorithm and converted to an hourly rate with or without FICA. The hourly rate cost is pushed to Direct Labor algorithm by the CE Set Up algorithm which calculates the line cost for the Professional Provider. The line cost includes the cost of the Professional Provider in all stages of the CE where the Professional Provider is engaged with the client and the Professional Provider's AO time if present. The Cost Table algorithm pulls the line cost calculated by the Direct Labor algorithm to the Cost Table. The Dynamic Cost Table algorithm pulls the line cost to the Dynamic Cost Table in the Profit Module. The Player Cost Variable sub algorithm accesses the line cost on the Dynamic Cost Table.

The Player Cost Variable sub algorithm allows the line cost to be dynamically changed using the graphic toggle function. The Player Cost Variable sub algorithm allows for changes in the line cost for a Player and the resulting changes in the total line cost on the Dynamic Cost Table to be calculated and dynamically displayed.

The Gross Profit algorithm dynamically calculates and displays the new Dynamic Gross Profit.

### Professional Provider, Payment per day (A.c):

A.c. The Professional Provider, payment per day was entered in CE set up using the Payment Rate algorithm and converted to an hourly rate. There is no FICA. The hourly rate cost is pushed to Direct Labor algorithm by the CE Set Up algorithm which calculates the line cost for the Professional Provider. The line cost includes the cost of the Professional Provider in all stages of the CE where the Professional Provider is engaged with the client and the Professional Provider's AO time if present. The Cost Table algorithm pulls the line cost calculated by the Direct Labor algorithm to the Cost Table. The Dynamic Cost Table algorithm pulls the line cost to the Dynamic Cost Table in the Profit Module. The Player Cost Variable sub algorithm accesses the line cost on the Dynamic Cost Table.

The Player Cost Variable sub algorithm allows the line cost to be dynamically changed using the graphic toggle function. The Player Cost Variable sub algorithm allows for changes in the line cost for a Player and the resulting changes in the total line cost on the Dynamic Cost Table to be calculated and dynamically displayed.

The Gross profit algorithm dynamically calculates and displays the new Dynamic Gross Profit.

### Professional Provider, Payment Per Case (A.d):

A.d. The Professional Provider, payment per case was entered in CE set up using the Payment Type sub algorithm. There is no FICA or AO time. The payment per case cost is pushed to Direct Labor algorithm by the CE Set Up algorithm as the line cost for the Professional Provider. The Cost Table algorithm pulls the line cost from Direct Labor algorithm to the Cost Table. The Dynamic Cost Table algorithm pulls the line cost to the Dynamic Cost Table in the Profit Module. The Player Cost Variable sub algorithm accesses the line cost on the Dynamic Cost Table.

The Player Cost Variable sub algorithm allows the line cost to be dynamically changed using the graphic toggle function. The Player Cost Variable sub algorithm allows for changes in the line cost for a Player and the resulting changes in the total line cost on the Dynamic Cost Table to be calculated and dynamically displayed.

The Gross Profit algorithm dynamically calculates and displays the new Dynamic Gross Profit.

### Professional Provider, Percentage of Professional Component (A.e):

A.e. The Professional Provider, percentage of professional component was entered in CE set up using the Payment Type sub algorithm. There is no FICA or AO time. The percentage of professional component cost is pushed to Direct Labor algorithm by the CE Set Up algorithm as the line cost for the Professional Provider. The Cost Table algorithm pulls the line cost from the Direct Labor algorithm to the Cost Table. The Dynamic Cost Table algorithm pulls the line cost to the Dynamic Cost Table in the Profit Module. The Player Cost Variable sub algorithm accesses the line cost on the Dynamic Cost Table.

The Player Cost Variable sub algorithm allows the line cost to be dynamically changed using the graphic toggle function. The Player Cost Variable sub algorithm allows for changes in the line cost for a Player and the resulting changes in the total line cost on the Dynamic Cost Table to be calculated and dynamically displayed.

The Gross Profit algorithm dynamically calculates and displays the new Dynamic Gross Profit.

### Team Members:

### Team Members, Payment Per Hour (B.a):

B.a The Team Member, hourly rate was entered in CE set up using the Payment Type sub algorithm with or without FICA. The hourly rate cost is pushed to Direct Labor algorithm by the CE Set Up algorithm which calculates the line cost for the Team Member. The line cost includes the cost of the Team Member in all stages of the CE where the Team Member is engaged with the client and the Team Member's AO time if present. The Cost Table algorithm pulls the line cost calculated by the Direct Labor algorithm to the Cost Table. The Dynamic Cost Table algorithm pulls the line cost to the Dynamic Cost Table in the Profit Module. The Player Cost Variable sub algorithm accesses the line cost on the Dynamic Cost Table.

The Player Cost Variable sub algorithm allows the line cost to be dynamically changed using the graphic toggle function. The Player Cost Variable sub algorithm allows for changes in the line cost for a Player and the resulting changes in the total line cost on the Dynamic Cost Table to be calculated and dynamically displayed.

The Gross Profit algorithm dynamically calculates and displays the new Dynamic Gross Profit.

### Team Member, Salary (B.b):

B.b The Team Member, salary was entered in CE set up using the Payment Type Sub algorithm and converted to an hourly rate with or without FICA. The hourly rate cost is pushed to Direct Labor algorithm by the CE Set Up algorithm which calculates the line cost for the Team Member. The line cost includes the cost of the Team Member in all stages of the CE where the Team Member is engaged with the client and the Team Member's AO time if present. The Cost Table algorithm pulls the line cost calculated by the Direct Labor algorithm to the Cost Table. The Dynamic Cost Table algorithm pulls the line cost to the Dynamic Cost Table in the Profit Module. The Player Cost Variable sub algorithm accesses the line cost on the Dynamic Cost Table.

The Player Cost Variable sub algorithm allows the line cost to be dynamically changed using the graphic toggle function. The Player Cost Variable sub algorithm allows for changes in the line cost for a Player and the resulting changes in the total line cost on the Dynamic Cost Table to be calculated and dynamically displayed.

The Gross Profit algorithm dynamically calculates and displays the new Dynamic Gross Profit.

### Team Member, Payment Per Day (B.c):

B.c. The Team Member, payment per day was entered in CE set up using the Payment Rate Algorithm and converted to an hourly rate. There is no FICA. The hourly rate cost is pushed to Direct Labor algorithm by the CE set up algorithm which calculates the line cost for the Team Member. The line cost includes the cost of the Team Member in all stages of the CE where the Team Member is engaged with the client and the Team Member's AO time if present. The Cost Table algorithm pulls the line cost calculated by the Direct Labor algorithm to the Cost Table. The Dynamic Cost Table algorithm pulls the line cost to the Dynamic Cost Table in the Profit Module. The Player Cost Variable sub algorithm accesses the line cost on the Dynamic Cost Table.

The Player Cost Variable sub algorithm allows the line cost to be dynamically changed using the graphic toggle function. The Player Cost Variable sub algorithm allows for changes in the line cost for a Player and the resulting changes in the total line cost on the Dynamic Cost Table to be calculated and dynamically displayed.

The Gross Profit algorithm dynamically calculates and displays the new Dynamic Gross Profit.

### Anesthesia Provider:

The cost of the Anesthesia Provider in the CE for all job descriptions with the same payment type are calculated the same way.

### Anesthesia Provider, Payment Per Hour (C.a):

C.a The Anesthesia Provider hourly rate was entered in CE set up using the Payment Type sub algorithm with or without FICA. The hourly rate cost is pushed to Direct Labor algorithm by the CE Set Up algorithm which calculates the line cost for the Anesthesia Provider. The line cost includes the cost of the Anesthesia Provider in all stages of the CE where the Anesthesia Provider is engaged with the client and the Anesthesia Provider's AO time if present. The Cost Table algorithm pulls the line cost calculated by the Direct Labor algorithm to the Cost Table. The Dynamic Cost Table algorithm pulls the line cost to the Dynamic Cost Table in the Profit Module. The Player Cost Variable sub algorithm accesses the line cost on the Dynamic Cost Table.

The Player Cost Variable sub algorithm allows the line cost to be dynamically changed using the graphic toggle function. The Player Cost Variable sub algorithm allows for changes in the line cost for a Player and the resulting changes in the total line cost on the Dynamic Cost Table to be calculated and dynamically displayed.

The Gross Profit algorithm dynamically calculates and displays the new Dynamic Gross Profit.

### Anesthesia Provider, Salary (C.b):

C.b The Anesthesia Provider salary was entered in CE set up using the Payment Type sub algorithm and converted to an hourly rate with or without FICA. The hourly rate cost is pushed to Direct Labor algorithm by the CE Set Up algorithm which calculates the line cost for the Anesthesia Provider. The line cost includes the cost of the Anesthesia Provider in all stages of the CE where the Anesthesia Provider is engaged with the client and the Anesthesia Provider's AO time if present. The Cost Table algorithm pulls the line cost calculated by the Direct Labor algorithm to the Cost Table. The Dynamic Cost Table algorithm pulls the line cost to the Dynamic Cost Table in the Profit Module. The Player Cost Variable sub algorithm accesses the line cost on the Dynamic Cost Table.

The Player Cost Variable sub algorithm allows the line cost to be dynamically changed using the graphic toggle function. The Player Cost Variable sub algorithm allows for changes in the line cost for a Player and the resulting changes in the total line cost on the Dynamic Cost Table to be calculated and dynamically displayed.

The Gross Profit algorithm dynamically calculates and displays the new Dynamic Gross Profit.

### Anesthesia Provider, Payment Per Day (C.c):

C.c. The Anesthesia Provider, payment per day was entered in CE set up using the Payment Rate algorithm and converted to an hourly rate. There is no FICA. The hourly rate cost is pushed to Direct Labor algorithm by the CE Set Up algorithm which calculates the line cost for the Anesthesia Provider. The line cost includes the cost of the Anesthesia Provider in all stages of the CE where the Anesthesia Provider is engaged with the client and the Professional Provider's AO time if present. The Cost Table algorithm pulls the line cost calculated by the Direct Labor algorithm to the Cost Table. The Dynamic Cost Table algorithm pulls the line cost to the Dynamic Cost Table in the Profit Module. The Player Cost Variable sub algorithm accesses the line cost on the Dynamic Cost Table.

The Player Cost Variable sub algorithm allows the line cost to be dynamically changed using the graphic toggle function. The Player Cost Variable sub algorithm allows for changes in the line cost for a Player and the resulting changes in the total line cost on the Dynamic Cost Table to be calculated and dynamically displayed.

The Gross Profit algorithm dynamically calculates and displays the new Dynamic Gross Profit.

### Anesthesia Provider, Payment Per Case (C.d):

C.d. The Anesthesia Provider, payment per case was entered in CE set up using the Payment Type sub algorithm. There is no FICA or AO time. The payment per case cost is pushed to Direct Labor algorithm by the CE Set Up algorithm as the line cost for the Anesthesia Provider. The Cost Table algorithm pulls the line cost from the Direct Labor algorithm to the Cost Table. The Dynamic Cost Table algorithm pulls the line cost to the Dynamic Cost Table in the Profit Module. The Player Cost Variable sub algorithm accesses the line cost on the Dynamic Cost Table.

The Player Cost Variable sub algorithm allows the line cost to be dynamically changed using the graphic toggle function. The Player Cost Variable sub algorithm allows for changes in the line cost for a Player and the resulting changes in the total line cost on the Dynamic Cost Table to be calculated and dynamically displayed.

The Gross Profit algorithm dynamically calculates and displays the new Dynamic Gross Profit.

### Anesthesia Provider, Percentage of Anesthesia Component (C.e):

C.e. The Anesthesia Provider, percentage of anesthesia component was entered in CE set up using the Payment Type sub algorithm. There is no FICA or AO time. The percentage of anesthesia component cost is pushed to Direct Labor algorithm by the CE Set Up algorithm as the line cost for the Anesthesia Provider. The Cost Table algorithm pulls the line cost from the Direct Labor algorithm to the Cost Table. The Dynamic Cost Table algorithm pulls the line cost to the Dynamic Cost Table in the Profit Module. The Player Cost Variable sub algorithm accesses the line cost on the Dynamic Cost Table.

The Player Cost Variable sub algorithm allows the line cost to be dynamically changed using the graphic toggle function. The Player Cost Variable sub algorithm allows for changes in the line cost for a Player and the resulting changes in the total line cost on the Dynamic Cost Table to be calculated and dynamically displayed.

The Gross Profit algorithm dynamically calculates and displays the new Dynamic Gross Profit.

### Ancillary Provider:

The cost of the Ancillary Provider present or not present during the CE with the same payment type are calculated the same way.

### Ancillary Provider, Payment Per Hour (D.a):

D.a The Ancillary Provider hourly rate was entered in CE set up using the Payment Type sub algorithm with or without FICA. There is no AO time for the Ancillary Provider not present during the CE. The hourly rate cost is pushed to Direct Labor algorithm by the CE Set Up algorithm which calculates the line cost for the Ancillary Provider. The line cost includes the cost of the Ancillary Provider in all stages of the CE where the Ancillary Provider is engaged with the client and the Ancillary Provider's AO time if present. The Cost Table algorithm pulls the line cost calculated by the Direct Labor algorithm to the Cost Table. The Dynamic Cost Table algorithm pulls the line cost to the Dynamic Cost Table in the Profit Module. The Player Cost Variable sub algorithm accesses the line cost on the Dynamic Cost Table.

The Player Cost Variable sub algorithm allows the line cost to be dynamically changed using the graphic toggle function. The Player Cost Variable sub algorithm allows for changes in the line cost for a Player and the resulting changes in the total line cost on the Dynamic Cost Table to be calculated and dynamically displayed.

The Gross Profit algorithm dynamically calculates and displays the new Dynamic Gross Profit.

### Ancillary Provider, Salary (D.b):

D.b The Ancillary Provider salary was entered in CE set up using the Payment Type sub algorithm and converted to an hourly rate with or without FICA. There is no AO time for the Ancillary Provider not present during the CE. The hourly rate cost is pushed to Direct Labor algorithm by the CE Set Up algorithm which calculates the line cost for the Ancillary Provider. The line cost includes the cost of the Ancillary Provider in all stages of the CE where the Ancillary Provider is engaged with the client and the Ancillary Provider's AO time if present. The Cost Table algorithm pulls the line cost calculated by the Direct Labor algorithm to the Cost Table. The Dynamic Cost Table algorithm pulls the line cost to the Dynamic Cost Table in the Profit Module. The Player Cost Variable sub algorithm accesses the line cost on the Dynamic Cost Table.

The Player Cost Variable sub algorithm allows the line cost to be dynamically changed using the graphic toggle function. The Player Cost Variable sub algorithm allows for changes in the line cost for a Player and the resulting changes in the total line cost on the Dynamic Cost Table to be calculated and dynamically displayed.

The Gross Profit algorithm dynamically calculates and displays the new Dynamic Gross Profit.

### Ancillary Provider, Payment Per Day (D.c):

D.c. The Ancillary Provider, payment per day was entered in CE set up using the Payment Rate algorithm and converted to an hourly rate. There is no FICA. There is no AO time for the Ancillary Provider not present during the CE. The hourly rate cost is pushed to Direct Labor algorithm by the CE Set Up algorithm which calculates the line cost for the Ancillary Provider. The line cost includes the cost of the Ancillary Provider in all stages of the CE where the Ancillary Provider is engaged with the client and the Ancillary Provider's AO time if present. The Cost Table algorithm pulls the line cost calculated by the Direct Labor algorithm to the Cost Table. The Dynamic Cost Table algorithm pulls the line cost to the Dynamic Cost Table in the Profit Module. The Player Cost Variable sub algorithm accesses the line cost on the Dynamic Cost Table.

The Player Cost Variable sub algorithm allows the line cost to be dynamically changed using the graphic toggle function. The Player Cost Variable sub algorithm allows for changes in the line cost for a Player and the resulting changes in the total line cost on the Dynamic Cost Table to be calculated and dynamically displayed.

The Gross Profit algorithm dynamically calculates and displays the new Dynamic Gross Profit.

### Ancillary Provider, Payment Per Case (D.d):

D.d. The Ancillary Provider, payment per case was entered in CE set up using the Payment Type sub algorithm. There is no FICA or AO time. The payment per case cost is pushed to Direct Labor algorithm by the CE Set Up algorithm as the line cost for the Ancillary Provider. The Cost Table algorithm pulls the line cost from the Direct Labor algorithm to the Cost Table. The Dynamic Cost Table algorithm pulls the line cost to the Dynamic Cost Table in the Profit Module. The Player Cost Variable sub algorithm accesses the line cost on the Dynamic Cost Table.

The Player Cost Variable sub algorithm allows the line cost to be dynamically changed using the graphic toggle function. The Player Cost Variable sub algorithm allows for changes in the line cost for a Player and the resulting changes in the total line cost on the Dynamic Cost Table to be calculated and dynamically displayed.

The Gross Profit algorithm dynamically calculates and displays the new Dynamic Gross Profit.

### Ancillary Provider, Percentage of Ancillary Component (D.e):

D.e. The Ancillary Provider, percentage of ancillary component was entered in CE set up using the Payment Type sub algorithm. There is no FICA or AO time. The percentage of ancillary component cost is pushed to Direct Labor algorithm by the CE Set Up algorithm as the line cost for the Ancillary Provider. The Cost Table algorithm pulls the line cost calculated by the Direct Labor algorithm to the Cost Table. The Dynamic Cost Table algorithm pulls the line cost to the Dynamic Cost Table in the Profit Module. The Player Cost Variable sub algorithm accesses the line cost on the Dynamic Cost Table.

The Player Cost Variable sub algorithm allows the line cost to be dynamically changed using the graphic toggle function. The Player Cost Variable sub algorithm allows for changes in the line cost for a Player and the resulting changes in the total line cost on the Dynamic Cost Table to be calculated and dynamically displayed.

The Gross Profit algorithm dynamically calculates and displays the new Dynamic Gross Profit.

[PART THREE] The third profit algorithm, TVM algorithm considers the Time Value of Money (TVM) for implants and HCDs. The algorithm's function is to determine the revenue generated by an implant or HCD when the cost of the implant or HCD is incurred at one point in time and reimbursement for the implant or HCD occurs at another distant point in time. This time difference in the “time value of money” acts as a revenue discount on the revenue ultimately generated by the implant and/or HCD.

For implants, the TVM algorithm can only be applied to implants that have had the “Link to Revenue” option created between the Cost and Revenue Modules in the fifth section of the IIS card. This link established a connection between the cost to purchase the implant and the revenue generated by reimbursement of the implant. The cost to purchase the implant is P. The revenue generated by the implant is M. M is either the revenue or corrected revenue for the implant depending on whether the Collection Discount Surgery Center sub algorithm was applied.

For HCDs, the TVM algorithm can be applied in all cases as by definition all HCDs have a cost and a revenue component. A mandatory link was established in the fifth section of the IIS card between the Cost and Revenue Modules. The cost to purchase the HCD is P. The revenue generated by the HCD is M. M is either the revenue or corrected revenue for the HCD depending on whether the Collection Discount Surgery Center sub algorithm was applied. M and P are displayed to the user.

The TVM algorithm does not consider revenue generated by DME/Orthotics.

The TVM algorithm considers each implant or HCD chosen separately.

To calculate the time value of money, the TVM algorithm uses the following formula:

$$A = P {( 1 + \frac{R}{N} )}^{( N ) ( T )}$$

For each implant and/or HCD the user will input the average time in days between paying for the implant and/or HCD to the time reimbursement is received. The TVM algorithm will divide this time by 365 as the time in years over which the TVM is calculated. The calculations allow for two decimal places. This value is T.

The user will input an annual percentage interest rate from 0-20%. This is a limited free text field that allows all entries including one decimal place to be entered. This value is R.

The user will input the number of times interest on the money paid for the implant or HCD is compounded on a yearly basis if the money had instead remained in a bank or other investment opportunity. This value is N. The user choice for N is a single-select list box option with the following options:

| | |
|---|---|
| Daily 365 | N = 365 |
| Daily 360 | N = 360 |
| Weekly | N = 52 |
| Bi-weekly | N = 13 |
| Semi-Monthly | N = 26 |
| Monthly | N = 12 |
| Bi-Monthly | N = 6 |
| Quarterly | N = 4 |
| Semi-annually | N = 2 |
| Annually | N = 1 |

Once the other values were inputted or selected, the TVM algorithm will calculate A. The value of A is the value of the money paid for the implant on the time it was paid to what the value of that money would be at the time the implant was reimbursed. A is displayed to the user.

The TVM algorithm will subtract M (revenue generated by the implant or HCD at a defined future time) from A (cost of the implant or HCD at the defined future time using the future value of the money that was used to make the purchase).

M−A=the implant or HCD profit when the “time value of money” is considered. This is referred to as “time value profit” (TVProfit) of the implant and/or HCD. TVProfit is displayed to the user.

Using the graphic toggle function, the user can change the value of T and the TVM algorithm will dynamically display changes in cost for the implant/HCD (A) on the Dynamic Cost Table and the changes in Dynamic Gross Profit.

The algorithm will also display implant/HCD gross profit (M−P) next to implant/HCD TVProfit. The TVM algorithm will allow implant/HCD TVProfit to be dynamically changed with changes in the value of T.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction, and arrangement of the components thereof without departing from the scope and spirit of the invention or without sacrificing all of its material advantages. The form herein before described being merely exemplary and explanatory embodiment thereof. It is the intention of the following claims to encompass and include such changes.

1. A system for calculating a profit/loss of a surgery center comprising:
a computer, the computer having a non-transient memory and storage;
software stored in the non-transient memory;
during an input stage, the software causes the computer to accept inputs regarding costs and revenue for a plurality of items and storing the inputs as records in the storage of the computer, each of the plurality of items regard an aspect of the surgery center;
after the input stage, the software causes the computer to execute a plurality of algorithms, each algorithm calculating a subset of the profit/loss associated with a single client experience determined from at least one record corresponding to at least one item of the plurality of items, and the software combining each of the subset of the profit/loss into a profit/loss for a client experience; and
the software causes the computer to output the profit/loss.

2. The system of claim 1, wherein the profit/loss is for the single client experience.

3. The system of claim 2, wherein an overall profit/loss for the surgery center having a mix of client experiences is calculated as a sum of the profit/loss for each of the single client experience in the mix of the client experiences.

4. The system of claim 2, wherein the plurality of algorithms comprises:
a first algorithm that calculates a professional component associated with the single client experience;
a second algorithm that calculates a surgery center component that includes implants and high cost disposables associated with the single client experience;
a third algorithm that calculates an anesthesia component associated with the single client experience;
a fourth algorithm that calculates other ancillary revenue associated with the single client experience;
a fifth algorithm that calculates direct costs associated with the single client experience, the direct costs include labor costs, supply costs and administrative claim costs; and
a sixth algorithm that calculates the profit/loss for the single client experience based upon the professional component, the surgery center component, the anesthesia component, the other ancillary revenue, and the direct costs.

5. The system of claim 2, wherein in an update mode, the software causes the computer to accept changes to the inputs regarding costs and revenue for any of the plurality of items and stores the inputs in the records in the storage of the computer, each of the plurality of items regarding the aspect of the surgery center; and the software causes the computer to execute the plurality of algorithms, generating the profit/loss for each of the single client experience from the at least one record and the software combining the profit/loss for each of the single client experience into an overall profit/loss for the surgery center.

6. The system of claim 5, wherein in the update mode, the software causes the computer to loop and in each loop, accept the changes to the inputs regarding the costs and the revenue for any of the plurality of items and stores the inputs in the records in the storage of the computer, each of the plurality of items regarding the aspect of the surgery center; and after receiving one or more of the changes to the inputs the software causes the computer to execute the plurality of algorithms, generating the profit/loss for each of the single client experience from the at least one record and the software combining the profit/loss for a mix of the single client experience into the overall profit/loss and the software causes the computer to display the overall profit/loss for the surgery center.

7. The system of claim 6, further comprising the software causes the computer to display the profit/loss for each of the single client experience.

8. A method of calculating a profit/loss of a surgery center comprising:
accepting inputs regarding costs and revenue for a plurality of items, each of the plurality of items regarding an aspect of the surgery center;
storing the inputs as records in a storage of a computer;
after accepting all of the inputs, executing a plurality of algorithms, each algorithm calculating a subset of the profit/loss associated with a single client experience determined from at least one record corresponding to at least one item of the plurality of items;
combining each of the subset of the profit/loss into the profit/loss for a client experience; and
outputting the profit/loss.

9. The method of claim 8, wherein the profit/loss is for the single client experience.

10. The method of claim 9, further comprising calculating an overall profit/loss for the surgery center, the surgery center having a mix of client experiences, as a sum of the profit/loss for each of the single client experience in the mix of the client experiences.

11. The method of claim 9, wherein the plurality of algorithms comprises:
a first algorithm calculating a professional component associated with the single client experience;
a second algorithm calculating a surgery center component that includes implants and high cost disposables associated with the single client experience;
a third algorithm calculating an anesthesia component associated with the single client experience;
a fourth algorithm calculating other ancillary revenue associated with the single client experience;
a fifth algorithm calculating direct costs associated with the single client experience, the direct costs include labor costs, supply costs and administrative claim costs; and
a sixth algorithm calculating the profit/loss for the single client experience based upon the professional component, the surgery center component, the anesthesia component, the other ancillary revenue, and the direct costs.

12. The method of claim 9, further comprising accepting changes to the inputs regarding the costs and/or the revenue for any of the plurality of items and storing the inputs in the records in the storage of the computer, each of the plurality of items regarding the aspect of the surgery center; and again executing the plurality of algorithms, generating the profit/loss for each of the single client experience from the at least one record and combining the profit/loss for each of the single client experience into an overall profit/loss for the surgery center.

13. The method of claim 12, further comprising looping and in each loop, accepting the changes to the inputs regarding the costs and the revenue for any of the plurality of items and storing the inputs in the records in the storage of the computer, each of the plurality of items regarding the aspect of the surgery center; and after receiving one or more of the changes to the inputs, again executing the plurality of algorithms, generating the profit/loss for each of the single client experience from the at least one record and combining the profit/loss for a mix of the single client experience into the overall profit/loss and storing the overall profit/loss for the surgery center.

14. The method of claim 13, further comprising displaying the profit/loss for each of the single client experience.

15. The method of claim 13, further comprising storing the profit/loss for each of the single client experience in a history of prior profit/loss for each of the single client experience and displaying the profit/loss for each of the single client experience along with one or more of the prior profit/loss for each of the single client experience.

16. The method of claim 13, further comprising storing the overall profit/loss in a history of prior overall profit/loss and displaying the overall profit/loss along with one or more of the prior overall profit/loss.

17. A method of calculating a profit/loss of a surgery center comprising:
accepting inputs regarding costs and revenue for a plurality of items, each of the plurality of items regarding an aspect of the surgery center;
storing the inputs as records in a storage of a computer;
after accepting all of the inputs, executing a first algorithm calculating a professional component associated with a single client experience, executing a second algorithm calculating a surgery center component that includes implants and high cost disposables associated with the single client experience, executing a third algorithm calculating an anesthesia component associated with the single client experience,, executing a fourth algorithm calculating other ancillary revenue associated with the single client experience, and executing a fifth algorithm calculating direct costs associated with the single client experience, the direct costs include labor costs, supply costs and administrative claim costs;
calculating the profit/loss for the single client experience based upon a sum of the professional component, the surgery center component, the anesthesia component, the other ancillary revenue, and the direct costs; and
outputting the profit/loss for the single client experience.

18. The method of claim 17, further comprising calculating an overall profit/loss for the surgery center as a sum of the profit/loss for each of the single client experience in a mix of the client experience.

19. The method of claim 18, wherein the mix of the client experiences comprises a count of each possible client experience that is expected to be performed over a period of time.
